SEQUENCE LISTING (71 sequences)

SEQ ID NO: 1 - protein, 12 aa, unidentified
GFSITTGGYWWT

SEQ ID NO: 2 - protein, 17 aa, unidentified
GYIFSSGNTNYNPSIKS

SEQ ID NO: 3 - protein, 12 aa, unidentified
CARAYGKLGFDY

SEQ ID NO: 4 - protein, 12 aa, unidentified
TLSSQHSTYTIG

SEQ ID NO: 5 - protein, 11 aa, unidentified
VNSDGSHSKGD

SEQ ID NO: 6 - protein, 9 aa, unidentified
GSSDSSGYV

SEQ ID NO: 7 - protein, 5 aa, unidentified
SDGIS

SEQ ID NO: 8 - protein, 16 aa, unidentified
IISSGGNTYYASWAKG

SEQ ID NO: 9 - protein, 8 aa, unidentified
VVGGTYSI

SEQ ID NO: 10 - protein, 13 aa, unidentified
QSSQSVYNNDFLS

SEQ ID NO: 11 - protein, 7 aa, unidentified
YASTLAS

SEQ ID NO: 12 - protein, 13 aa, unidentified
TGTYGNSAWYEDA

SEQ ID NO: 13 - protein, 5 aa, unidentified
SNAMI

SEQ ID NO: 14 - protein, 16 aa, unidentified
AMDSNSRTYYATWAKG

SEQ ID NO: 15 - protein, 11 aa, unidentified
GDGGSSDYTEM

SEQ ID NO: 16 - protein, 13 aa, unidentified
QSSQSVYGNNELS

SEQ ID NO: 17 - protein, 7 aa, unidentified
QASSLAS

SEQ ID NO: 18 - protein, 11 aa, unidentified
LGEYSISADNH

SEQ ID NO: 19 - protein, 120 aa, unidentified
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSS

SEQ ID NO: 20 - protein, 111 aa, unidentified
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 21 - protein, 454 aa, unidentified
METGLRWLLLVAVLKGVQCQSVEESGGRLVTPGTPLTLTCTVSGFSLSNNAINWVRQAPGKGLEWIGYIWSGGLTYYANWAEGRFTISKTSTTVDLKMTSPTIEDTATYFCARGINNSALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 22 - protein, 239 aa, unidentified
MDMRAPTQLLGLLLLWLPGARCADVVMTQTPASVSAAVGGTVTINCQASESISNYLSWYQQKPGQPPKLLIYRTSTLASGVSSRFKGSGSGTEYTLTISGVQCDDVATYYCQCTSGGKFISDGAAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 23 - protein, 452 aa, unidentified
METGLRWLLLVAVLKGVQCQSVEESGGRLVTPGTPLTLTCTVSGFTISDYDLSWVRQAPGEGLKYIGFIAIDGNPYYATWAKGRFTISKTSTTVDLKITAPTTEDTATYFCARGAGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 24 - protein, 238 aa, unidentified
MDTREPTQLLGLLLLWLPGARCADVVMTQTPASVSAAVGGTVTINCQSSKNVYNNNWLSWFQQKPGQPPKLLIYYASTLASGVPSRFRGSGSGTQFTLTISDVQCDDAATYYCAGDYSSSSDNGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 25 - protein, 454 aa, unidentified
METGLRWLLLVAVLKGVHCQSVEESGGRLVTPGTPLTLTCTASGFSRSSYDMSWVRQAPGKGLEWVGVISTAYNSHYASWAKGRFTISRTSTTVDLKMTSLTTEDTATYFCARGGSWLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 26 - protein, 239 aa, unidentified
  SITE 3: note = misc_feature - Xaa can be any naturally occurring amino acid
MDXRAPTQLLGLLLLWLPGARCALVMTQTPASVSAAVGGTVTINCQSSQSVYDNDELSWYQQKPGQPPKLLIYALASKLASGVPSRFKGSGSGTQFALTISGVQCDDAATYYCQATHYSSDWYLTFGGGTEVVVKGFPVAPTVLLFPPSSDEVATGTVTIVCVANKYFPDVTVTWEVDGTTQTTGTENSKTPQNSADCTYNLSSTLTLTSTQYNSHKEYTCKVTQGTTSVVQSFSRKNC

SEQ ID NO: 27 - protein, 456 aa, unidentified
METGLRWLLLVAVLKGVQCQSVEESGGRLVTPGTPLTLTCTVSGFSLSSYDMTWVRQAPGKGLEWIGIIYASGTTYYANWAKGRFTISKTSTTVDLKVTSPTIGDTATYFCAREGAGVSMTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 28 - protein, 236 aa, unidentified
MDMRAPTQLLGLLLLWLPGARCAYDMTQTPASVEVAVGGTVTIKCQASQSISTYLDWYQQKPGQPPKLLIYDASDLASGVPSRFKGSGSGTQFTLTISDLECADAATYYCQQGYTHSNVDNVFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 29 - protein, 459 aa, unidentified
METGLRWLLLVAVLKGVQCQSVEESGGRLVSPGTPLTLTCTASGFSLSSYDMSWVRQAPGKGLEYIGIISSSGSTYYASWAKGRFTISKTSTTVDLEVTSLTTEDTATYFCSREHAGYSGDTGHLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVGIGPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 30 - protein, 236 aa, unidentified
MDMRAPTQLLGLLLLWLPGARCAYDMTQTPASVEVAVGGTVAIKCQASQSVSSYLAWYQQKPGQPPKPLIYEASMLAAGVSSRFKGSGSGTDFTLTISDLECDDAATYYCQQGYSISDIDNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 31 - protein, 455 aa, unidentified
METGLRWLLLVAVLKGVQCQSVEESGGRLVTPGTPLTLTCTVSGIDLSSDGISWVRQAPGKGLEWIGIISSGGNTYYASWAKGRFTISRTSTTVDLKMTSLTTEDTATYFCARVVGGTYSIWGQGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 240 aa, unidentified
MDTRAPTQLLGLLLLWLPGATFAQVLTQTASPVSAPVGGTVTINCQSSQSVYNNDFLSWYQQKPGQPPKLLIYYASTLASGVPSRFKGSGSGTQFTLTISDLECDDAATYYCTGTYGNSAWYEDAFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 33 - protein, 460 aa, unidentified
METGLRWLLLVAVLKGVQCQSVEESGGRLVTPGTPLTLTCTVSGIDLSSNAMIWVRQAPREGLEWIGAMDSNSRTYYATWAKGRFTISRTSSITVDLKITSPTTEDTATYFCARGDGGSSDYTEMWGPGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 239 aa, unidentified
MDTRAPTQLLGLLLLWLPGATFAQAVVTQTTSPVSAPVGGTVTINCQSSQSVYGNNELSWYQQKPGQPPKLLIYQASSLASGVPSRFKGSGSGTQFTLTISDLECDDAATYYCLGEYSISADNHFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 35 - protein, 221 aa, unidentified
QVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 36 - protein, 110 aa, unidentified
LTQPPPASGTPGQQRVTISCSGSSSNIGSNTVNWYQQLPGTAPKLLIYYGNDQRPSGVPDRFSASKSGTSASLAISGLQSEDEAHYYCAAWDGSLNGGVIFGGGTKVTLG

SEQ ID NO: 37 - protein, 109 aa, unidentified
VTQPPSASGTPGQRVTISCSGSSSNIGTNPVNWYQQLPGTAPKLLIYTTDQRPSGVPDRFSGSKSGTSASLAISGLQSEDEADYYCAAWDDSLSGLYVFGTGTKVTVLG

SEQ ID NO: 38 - protein, 109 aa, unidentified
MTHTPLSLSVTPGQPASISCKSSQSLLHSDGKTYLYWYLQRPGQSPQPLIYEVSNRFSGVPDRFSGSGSGTDFTLKISRVQAEDVGVYYCMQSLQLWTFGQGTKVEIKR

SEQ ID NO: 39 - protein, 110 aa, unidentified
MTQSPLSLPVTLGQPASISCRSSQSLIHSDGNTYLDWFQQRPGQSPRRLIYKVSNRDSGVPDRFSGSGSGTDFTLRISRVEAEDIGVYYCMQATHWPRTFGQGTKVEIKR

SEQ ID NO: 40 - protein, 110 aa, unidentified
MTQSPLSLPVTLGQPASISCRSSQSLVDSAGNTFLHWFHQRPGQSPRRLIYKVSNRDSGVPDRFSGSGSGTDFTLKISRVEAEDVGVYYCMQGTHWPRTFGQGTKVEIKR

SEQ ID NO: 41 - protein, 110 aa, unidentified
LTQSPLSLPVTLGQPASISCKSSQSLVDSDGNTYLNWFQQRPGQSPRRLIYKVSNRDSGVPDRFSGSGSGTDFTLKISRVEAEDVGVYYCMQGTHWPRTFGQGTKVEIKR

SEQ ID NO: 42 - protein, 110 aa, unidentified
MTQSPLSLPVTLGQPASISCRSSQSLVHSDGNMYLNWFQQRPGQSPRRLIYKVSNRDSGVPDRFSGSGSGTDFTLKISRVEAEDVGVYYCMQATQPTWTFGQGTKLEIKR

SEQ ID NO: 43 - protein, 92 aa, unidentified
MTQSPSSLSASVGDRVTITCQASQDISNYLNWYQQKPGKAPKLLIYDASNLETGVPSRFSGSGSGTDFTFTISSATYYCQQTYQGTKLEIKR

SEQ ID NO: 44 - protein, 105 aa, unidentified
MTQSPSSLSASVGHPVTITCRASQSLISYLNWYHQKPGKAPKLLIYAASILQSGVPSRFSGSGSGTDFTLTISSLQPENFASYYCQHTDSFPRTFGHGTKVEIKR

SEQ ID NO: 45 - protein, 108 aa, unidentified
LTQPPSASGTPGQGVTISCRGSTSNIGNNVVNWYQHVPGSAPKLLIWSNIQRPSGIPDRFSGSKSGTSASLAISGLQSEDQAVYYCAVWDDGLAGWVFGGGTTVTVLS

SEQ ID NO: 46 - protein, 105 aa, unidentified
MTQAPVVSVALEQTVRITCQGDSLAIYYDFWYQHKPGQAPVLVIYGKNNRPSGIPHRFSGSSSNTDSLTITGAQAEDEADYYCNSRDSSGNHWVFGGGTNLTVLG

SEQ ID NO: 47 - protein, 111 aa, unidentified
LTQSPLSLPVTLGQPASISCKSNQSLVHSDGNTYLNWFQQRPGQSPRRLIYKVSNRDSGVPDRFSGSGSGTDFTLKINRVEAEDVGVYYCMQGTQWPRTFGGQGTKLDIKR

SEQ ID NO: 48 - protein, 119 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 49 - protein, 119 aa, unidentified
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSS

SEQ ID NO: 50 - protein, 119 aa, unidentified
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 51 - protein, 119 aa, unidentified
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSS

SEQ ID NO: 52 - protein, 119 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSS

SEQ ID NO: 53 - protein, 120 aa, unidentified
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSS

SEQ ID NO: 54 - protein, 111 aa, unidentified
QLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 55 - protein, 111 aa, unidentified
LPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 56 - protein, 15 aa, unidentified
GGGGSGGGGSGGGGS

SEQ ID NO: 57 - protein, 247 aa, unidentified
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAASSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAA

SEQ ID NO: 58 - protein, 253 aa, unidentified
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTV

SEQ ID NO: 59 - protein, 245 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 60 - protein, 245 aa, unidentified
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 61 - protein, 245 aa, unidentified
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 62 - protein, 245 aa, unidentified
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 63 - protein, 245 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 64 - protein, 246 aa, unidentified
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 65 - protein, 245 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 66 - protein, 245 aa, unidentified
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 67 - protein, 245 aa, unidentified
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 68 - protein, 245 aa, unidentified
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 69 - protein, 245 aa, unidentified
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 70 - protein, 246 aa, unidentified
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 71 - protein, 246 aa, unidentified
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

(19) United States
(12) Patent Application Publication
(10) Pub. No.: US 2024/0197779 A1
(43) Pub. Date: Jun. 20, 2024

(54) ANTI-CD83 CHIMERIC ANTIGEN RECEPTOR EXPRESSING T REGULATORY CELLS

(71) Applicant: H. Lee Moffitt Cancer Center and Research Institute, Inc., Tampa, FL (US)

(72) Inventors: Marco Davila, Tampa, FL (US); Brian Betts, Minneapolis, MN (US)

(73) Assignee: H. Lee Moffitt Cancer Center and Research Institute, Inc., Tampa, FL (US)

(21) Appl. No.: 18/478,247

(22) Filed: Sep. 29, 2023

Related U.S. Application Data
(63) Continuation of application No. 16/717,537, filed on Dec. 17, 2019, now abandoned.
Application No. 16/717,537 is a continuation-in-part of application No. PCT/US2019/019065, filed on Feb. 22, 2019.
(60) Provisional application No. 62/888,055, filed on Aug. 16, 2019.
(60) Provisional application No. 62/677,783, filed on May 30, 2018.
(60) Provisional application No. 62/634,435, filed on Feb. 23, 2018.

(51) Int. Cl. A61K 35/17; C07K 14/705; C07K 16/28

(52) U.S. Cl. CPC A61K 35/17; C07K 14/70596; C07K 16/2803; C07K 2317/622

(57) ABSTRACT

Disclosed are compositions and methods for preventing graft versus host disease (GVHD) in subjects receiving donor cells. In particular, chimeric antigen receptor (CAR) polypeptides are disclosed that can be used with adoptive cell transfer to suppress alloreactive donor cells. Therefore, also disclosed are methods of suppressing alloreactive donor cells in a subject receiving transplant donor cells that involves adoptive transfer of the disclosed regulatory T cells engineered to express the disclosed CARs. Also disclosed is a method of preventing rejection of off-the-shelf therapeutic immune effector cells, such as CAR-T cells, in a subject that involves administering to the subject an effective amount of a regulatory T cell genetically modified with a disclosed CD83-specific CAR.

### CROSS-REFERENCE TO RELATED APPLICATIONS

This application is a continuation of copending application Ser. No. 16/717,537, filed Dec. 17, 2018, which is a continuation-in-part of copending Application Serial No. PCT/US2019/019065, filed Feb. 22, 2019, which claims benefit of U.S. Provisional Application No. 62/634,435, filed Feb. 23, 2018, and Application Ser. No. 62/677,783, filed May 30, 2018, which are hereby incorporated herein by reference in their entirety. This application also claims benefit of U.S. Provisional Application No. 62/888,055, filed Aug. 16, 2019, which is hereby incorporated herein by reference in its entirety.

### SEQUENCE LISTING

This application contains a sequence listing filed in electronic form as an ST.26 file entitled “320805-1031 Sequence Listing” created on Feb. 21, 2019 Mar. 8, 2024, having 73,363 bytes. The content of the sequence listing is incorporated herein in its entirety.

### BACKGROUND

Allogeneic hematopoietic cell transplantation (HCT) is an effective therapy for hematological malignancies but it is limited by acute graft-versus-host disease (GVHD). GVHD arises when donor T cells respond to genetically defined proteins on host cells, and is a key contributor to the high mortality associated with HCT. Current immunosuppressive measures to control GVHD broadly suppress alloreactive T cells that mediate GVHD, but also impair beneficial regulatory T cells (Treg) that facilitate immune tolerance. Moreover, the use of calcineurin-inhibitors, such as tacrolimus, even limits graft-versus-leukemia (GVL) mediated by donor immunity, which is a key tenet of allo-HCT to prevent disease relapse.

### SUMMARY

Disclosed herein is a method of suppressing alloreactive donor cells in a subject receiving transplanted donor hematopoietic cells or solid organ allografts that involves administering to the subject an effective amount of a immune effector cell, such as a T cell, genetically modified to express a chimeric antigen receptor (CAR) targeting CD83. This is referred to herein as a CAR-T cell. CD83 is differentially expressed on alloreactive T cells. Thus, the disclosed anti-CD83 CAR-T cells will target T cells that cause GVHD and spare GVL. Even when donors are fully HLA matched, the minor HLA disparity or the presence of H-Y antigens are sufficient to cause GVHD. Additionally, not all donors are HLA-matched to recipients, such as HLA-DP mismatch, which can also result in severe GVHD. A unique benefit of the anti-CD83 CAR-T cells is that it can suppress rather than kill alloreactive T cells, to avoid lymphopenia.

In some embodiments, the subject will receive an immunosuppressant, such as a calcineurin-inhibitor or glucocorticoid, to prevent or treat GVHD. A common side effect of immunosuppressants is an increased susceptibility to infection and malignancy. Commonly used immunosuppressants include calcineurin-inhibitors (e.g. cyclosporine A, tacrolimus), glucocorticoids, cyclophosphamide, ruxolitinib, and methotrexate.

In some embodiments, solid organ recipients require life-long immunosuppression to prevent rejection of the allograft. Similar to alloHCT recipients, patients that receive a solid organ may require a calcineurin-inhibitor or glucocorticoids to prevent or treat allograft rejection. This exposes the patient to extended risk for infections, secondary malignancies, and impaired tissue tolerance.

Also disclosed is a method of preventing GVHD in a subject that involves administering to the subject an effective amount of anti-CD83 CAR-T cells. In some embodiments, the subject is receiving a tissue transplantation. In some embodiments, the tissue transplantation comprises a bone marrow transplantations. In some embodiments, the tissue transplantation comprises a solid organ transplant, including but not limited to, face transplant, abdominal wall transplant, limb transplant, upper extremity transplant, vascularized composite allograft, or whole tissue graft. In some embodiments, the subject has an autoimmune diseases, sepsis, rheumatological diseases, diabetes, and/or asthma. Also disclosed is a method of treating autoimmunity in a subject that involves administering to the subject an effective amount of a regulatory T cell genetically modified with a disclosed anti-CD83 CAR-T cells.

Therefore, disclosed herein is a method of suppressing alloreactive donor cells in a subject receiving transplant donor cells, the method comprising administering to the subject an effective amount of a first immune effector cell genetically modified to express a first chimeric antigen receptor (CAR) polypeptide, comprising a CD83 antigen binding domain, a transmembrane domain, an intracellular signaling domain, and a co-stimulatory signaling region, thereby suppressing alloreactive donor cells in the subject.

Also disclosed is a method of preventing rejection of “off-the-shelf” therapeutic immune effector cells, such as CAR-T cells, in a subject that involves administering to the subject an effective amount of a regulatory T cell genetically modified with a disclosed anti-CD83 CAR-T cells. Therefore, in some embodiments, the transplant donor cells comprise a second immune effector cell genetically modified to express a second CAR polypeptide. In some embodiments, the second immune effector cell is selected from the group consisting of a T cell, a Natural Killer (NK) cell, a B cell, an innate lymphoid cell (ILC), a cytokine induced killer (CIK) cell, a cytotoxic T lymphocyte (CTL), a lymphokine activated killer (LAK) cell, and a regulatory T (Treg) cell.

In some embodiments, the transplant donor cells, such as off-the-shelf CAR-T cells, are not HLA matched to the subject. For example, in some embodiments, the transplant donor cells have less than 3, 4, 5, or 6 HLA-matched markers as the subject. Likewise, in some embodiments, the subject receiving the transplant donor cells has not received, and does not need, an immunosuppressant.

Also disclosed is a method for treating a subject that involves co-administering to the subject a therapeutic amount of a first non-autologous, non-HLA-matched immune effector cell genetically modified to express a first CAR polypeptide and a second non-autologous, non-HLA-matched immune effector cell genetically modified to express a second CAR polypeptide comprising a CD83 antigen binding domain.

The disclosed methods can in some embodiments be used with any type of therapeutic immune effector cells, such as CAR-T cells, tumor infiltration lymphocytes (TILs), natural killer (NK) cells, and engineered T cell receptors (TCR). In some embodiments, the therapeutic immune effector cell is a CAR-T cell comprising an ectodomain that targets an antigen, such as a tumor antigen.

Examples of antigens that can be targeted using a CAR include CD19, CD22, CD123, CD38, CLL-1, TEM8/ANTXR1, CD56, NKG2D, B7H6, CD4, Gp120, Erb-B, SLAMF7, CD7, EGFR, TAG-72, MMG49, Integrin β7, FLT3, MESO, CD70, FOLR1, CD33, CD171, CD20, GD2, HER2, EGFRvIII, CSPG4, DNAX, BCMA, LeY, CD30, GPC3, CS1, CD138, Nectin4/FAP, CEA, EpCAM, PSCA, MUC1, CD80, CD86, CTLA-4, PD-1, CD10, EGFR806, IL 12, TLR-9, CD83, CLEC12A, CD99, IL13Ra2, SSTR2, and GPC3.

Additional non-limiting examples of tumor antigens include the following: differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET. Tumor antigens include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, mesothelin, RPSA, HPV, HPV E7, BING-4, CLCA2, 9D7, SAP-1, BAGE family (BAGE1), CAGE family (CAGE1), GAGE family (GAGE1-8), LAGE family (LAGE1-2), MAGE family (MAGEA1-A12; MAGEB1-B2; MAGEC2; MAGED4), SAGE family (SAGE1), XAGE family (XAGE1 included), PRAME, Pme117, OCA2, BRCA1/2, CML66, FN1, MART-2, TGF-@Rll, TCR, Cyclin-A1, D393-CD20n, GnTV, HERV-K-MEL, KK-LC-1, KM-HN-1, MUC1, NA88-A, SSX-4, TAG-1, TAG-2, TRAG-3, TRP2-INT2, MCC, CLPP, IGF2B3, KLK4, KIF20A, LGSN, MELOE-1 and -2, MUC5AC, Gp 100, EBV, ERBB2IP, CD62E, CD63, CD99, CD104, CD107b, CD164, CD175s, CD178, CD243, CD248, CD261, CD262, CD263, CD264, CD318, and any combination thereof.

The disclosed anti-CD83 chimeric antigen receptor (CAR) polypeptides contain in an ectodomain an anti-CD83 binding agent that can bind CD83-expressing cells. Also disclosed is an immune effector cell genetically modified to express the disclosed CAR polypeptide.

The anti-CD83 binding agent is in some embodiments an antibody fragment that specifically binds CD83. For example, the antigen binding domain can be a Fab or a single-chain variable fragment (scFv) of an antibody that specifically binds CD83. The anti-CD83 binding agent is in some embodiments an aptamer that specifically binds CD83. For example, the anti-CD83 binding agent can be a peptide aptamer selected from a random sequence pool based on its ability to bind CD83. The anti-CD83 binding agent can also be a natural ligand of CD83, or a variant and/or fragment thereof capable of binding CD83.

In some embodiments, the anti-CD83 scFv can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GFSITTGGYWWT (SEQ ID NO:1), SDGIS (SEQ ID NO:7), or SNAMI (SEQ ID NO:13); CDR2 sequence of the V_{H} domain comprises the amino acid sequence GYIFSSGNTNYNPSIKS (SEQ ID NO:2), IISSGGNTYYASWAKG (SEQ ID NO:8), orAMDSNSRTYYATWAKG (SEQ ID NO:14); CDR3 sequence of the V_{H} domain comprises the amino acid sequence CARAYGKLGFDY (SEQ ID NO:3), VVGGTYSI (SEQ ID NO:9), or GDGGSSDYTEM (SEQ ID NO:15); CDR1 sequence of the V_{L} comprises the amino acid sequence TLSSQHSTYTIG (SEQ ID NO:4), QSSQSVYNNDFLS (SEQ ID NO:10), or QSSQSVYGNNELS (SEQ ID NO:16); CDR2 sequence of the V_{L} domain comprises the amino acid sequence VNSDGSHSKGD (SEQ ID NO:5), YASTLAS (SEQ ID NO: 11), or QASSLAS (SEQ ID NO:17); and CDR3 sequence of the V_{L} domain comprises the amino acid sequence GSSDSSGYV (SEQ ID NO:6), TGTYGNSAWYEDA (SEQ ID NO:12), or LGEYSISADNH (SEQ ID NO:18).

For example, in some embodiments, the CDR1 sequence of the V_{H} domain comprises the amino acid sequence GFSITTGGYWWT (SEQ ID NO:1), CDR2 sequence of the V_{H} domain comprises the amino acid sequence GYIFSSGNTNYNPSIKS (SEQ ID NO:2), CDR3 sequence of the V_{H} domain comprises the amino acid sequence CARAYGKLGFDY (SEQ ID NO:3), CDR1 sequence of the V_{L} comprises the amino acid sequence TLSSQHSTYTIG (SEQ ID NO:4), CDR2 sequence of the V_{L} domain comprises the amino acid sequence VNSDGSHSKGD (SEQ ID NO:5), and CDR3 sequence of the V_{L} domain comprises the amino acid sequence GSSDSSGYV (SEQ ID NO:6).

For example, in some embodiments, the CDR1 sequence of the V_{H} domain comprises the amino acid sequence SDGIS (SEQ ID NO:7), CDR2 sequence of the V_{H} domain comprises the amino acid sequence IISSGGNTYYASWAKG (SEQ ID NO:8), CDR3 sequence of the V_{H} domain comprises the amino acid sequence VVGGTYSI (SEQ ID NO:9), CDR1 sequence of the V_{L} comprises the amino acid sequence QSSQS VYNNDFLS (SEQ ID NO:10), CDR2 sequence of the V_{L} domain comprises the amino acid sequence YASTLAS (SEQ ID NO:11), and CDR3 sequence of the V_{L} domain comprises the amino acid sequence TGTYGNSAWYEDA (SEQ ID NO:12).

For example, in some embodiments, the CDR1 sequence of the V_{H} domain comprises the amino acid sequence SNAMI (SEQ ID NO:13), CDR2 sequence of the V_{H} domain comprises the amino acid sequence AMDSNSRTYYATWAKG (SEQ ID NO:14), CDR3 sequence of the V_{H} domain comprises the amino acid sequence GDGGSSDYTEM (SEQ ID NO:15), CDR1 sequence of the V_{L} comprises the amino acid sequence QSSQSVYGNNELS (SEQ ID NO:16), CDR2 sequence of the V_{L} domain comprises the amino acid sequence QASSLAS (SEQ ID NO:17), and CDR3 sequence of the V_{L} domain comprises the amino acid sequence LGEYSISADNH (SEQ ID NO:18).

In some embodiments, the anti-CD83 scFv V_{H} domain comprises the amino acid sequence:

(SEQ ID NO: 19, VH-GBM00)
QVQLKESGPGLVKPSQSLSLTCSVTGFSITTGGYWWTWIRQFPGQKLEWM
GYIFSSGNTNYNPSIKSRISITRDTSKNQFFLQLNSVTTEGDTARYYCAR
AYGKLGFDYWGQGTLVTVSS.

In some embodiments, the anti-CD83 scFv V_{L} domain comprises the amino acid sequence:

(SEQ ID NO: 20, VL-GBM00)
QPVLTQSPSASASLGNSVKITCTLSSQHSTYTIGWYQQHPDKAPKYVMYV
NSDGSHSKGDGIPDRFSGSSSGAHRYLSISNIQPEDEADYFCGSSDSSGY
VFGSGTQLTVL.

In some embodiments, the anti-CD83 scFv V_{H} domain comprises the amino acid sequence:

(SEQ ID NO: 21, 20D04)
METGLRWLLLVAVLKGVQCQSVEESGGRLVTPGTPLTLTCTVSGFSLSNN
AINWVRQAPGKGLEWIGYIWSGGLTYYANWAEGRFTISKTSTTVDLKMTS
PTIEDTATYFCARGINNSALWGPGTLVTVSSGQPKAPSVFPLAPCCGDTP
SSTVTLGCLVKGYLPEPVTVTWNSGTLTNGVRTFPSVRQSSGLYSLSSVV
SVTSSSQPVTCNVAHPATNTKVDKTVAPSTCSKPTCPPPELLGGPSVFIF
PPKPKDTLMISRTPEVTCVVVDVSQDDPEVQFTWYINNEQVRTARPPLRE
QQFNSTIRVVSTLPIAHQDWLRGKEFKCKVHNKALPAPIEKTISKARGQP
LEPKVYTMGPPREELSSRSVSLTCMINGFYPSDISVEWEKNGKAEDNYKT
TPAVLDSDGSYFLYNKLSVPTSEWQRGDVFTCSVMHEALHNHYTQKSISR
SPGK.

In some embodiments, the anti-CD83 scFv V_{L} domain comprises the amino acid sequence:

(SEQ ID NO: 22, 20D04)
MDMRAPTQLLGLLLLWLPGARCADVVMTQTPASVSAAVGGTVTINCQASE
SISNYLSWYQQKPGQPPKLLIYRTSTLASGVSSRFKGSGSGTEYTLTISG
VQCDDVATYYCQCTSGGKFISDGAAFGGGTEVVVKGDPVAPTVLLFPPSS
DEVATGTVTIVCVANKYFPDVTVTWEVDGTTQTTGIENSKTPQNSADCTY
NLSSTLTLTSTQYNSHKEYTCKVTQGTTSVVQSFSRKNC.

In some embodiments, the anti-CD83 scFv V_{H} domain comprises the amino acid sequence:

(SEQ ID NO: 23, 11G05)
METGLRWLLLVAVLKGVQCQSVEESGGRLVTPGTPLTLTCTVSGFTISDY
DLSWVRQAPGEGLKYIGFIAIDGNPYYATWAKGRFTISKTSTTVDLKITA
PTTEDTATYFCARGAGDLWGPGTLVTVSSGQPKAPSVFPLAPCCGDTPSS
TVTLGCLVKGYLPEPVTVTWNSGTLTNGVRTFPSVRQSSGLYSLSSVVSV
TSSSQPVTCNVAHPATNTKVDKTVAPSTCSKPTCPPPELLGGPSVFIFPP
KPKDTLMISRTPEVTCVVVDVSQDDPEVQFTWYINNEQVRTARPPLREQQ
FNSTIRVVSTLPIAHQDWLRGKEFKCKVHNKALPAPIEKTISKARGQPLE
PKVYTMGPPREELSSRSVSLTCMINGFYPSDISVEWEKNGKAEDNYKTTP
AVLDSDGSYFLYNKLSVPTSEWQRGDVFTCSVMHEALHNHYTQKSISRSP
GK.

In some embodiments, the anti-CD83 scFv V_{L} domain comprises the amino acid sequence:

(SEQ ID NO: 24, 11G05)
MDTREPTQLLGLLLLWLPGARCADVVMTQTPASVSAAVGGTVTINCQSSK
NVYNNNWLSWFQQKPGQPPKLLIYYASTLASGVPSRFRGSGSGTQFTLTI
SDVQCDDAATYYCAGDYSSSSDNGFGGGTEVVVKGDPVAPTVLLFPPSSD
EVATGTVTIVCVANKYFPDVTVTWEVDGTTQTTGIENSKTPQNSADCTYN
LSSTLTLTSTQYNSHKEYTCKVTQGTTSVVQSFSRKNC.

In some embodiments, the anti-CD83 scFv V_{H} domain comprises the amino acid sequence:

(SEQ ID NO: 25, 14C12)
METGLRWLLLVAVLKGVHCQSVEESGGRLVTPGTPLTLTCTASGFSRSSY
DMSWVRQAPGKGLEWVGVISTAYNSHYASWAKGRFTISRTSTTVDLKMTS
LTTEDTATYFCARGGSWLDLWGQGTLVTVSSGQPKAPSVFPLAPCCGDTP
SSTVTLGCLVKGYLPEPVTVTWNSGTLTNGVRTFPSVRQSSGLYSLSSVV
SVTSSSQPVTCNVAHPATNTKVDKTVAPSTCSKPTCPPPELLGGPSVFIF
PPKPKDTLMISRTPEVTCVVVDVSQDDPEVQFTWYINNEQVRTARPPLRE
QQFNSTIRVVSTLPIAHQDWLRGKEFKCKVHNKALPAPIEKTISKARGQP
LEPKVYTMGPPREELSSRSVSLTCMINGFYPSDISVEWEKNGKAEDNYKT
TPAVLDSDGSYFLYNKLSVPTSEWQRGDVFTCSVMHEALHNHYTQKSISR
SPGK.

In some embodiments, the anti-CD83 scFv V_{L} domain comprises the amino acid sequence:

(SEQ ID NO: 26, 14C12)
MDXRAPTQLLGLLLLWLPGARCALVMTQTPASVSAAVGGTVTINCQSSQS
VYDNDELSWYQQKPGQPPKLLIYALASKLASGVPSRFKGSGSGTQFALTI
SGVQCDDAATYYCQATHYSSDWYLTFGGGTEVVVKGFPVAPTVLLFPPSS
DEVATGTVTIVCVANKYFPDVTVTWEVDGTTQTTGTENSKTPQNSADCTY
NLSSTLTLTSTQYNSHKEYTCKVTQGTTSVVQSFSRKNC.

In some embodiments, the anti-CD83 scFv V_{H} domain comprises the amino acid sequence:

(SEQ ID NO: 27, 020B08)
METGLRWLLLVAVLKGVQCQSVEESGGRLVTPGTPLTLTCTVSGFSLSSY
DMTWVRQAPGKGLEWIGIIYASGTTYYANWAKGRFTISKTSTTVDLKVTS
PTIGDTATYFCAREGAGVSMTLWGPGTLVTVSSGQPKAPSVFPLAPCCGD
TPSSTVTLGCLVKGYLPEPVTVTWNSGTLTNGVRTFPSVRQSSGLYSLSS
VVSVTSSSQPVTCNVAHPATNTKVDKTVAPSTCSKPTCPPPELLGGPSVF
IFPPKPKDTLMISRTPEVTCVVVDVSQDDPEVQFTWYINNEQVRTARPPL
REQQFNSTIRVVSTLPIAHQDWLRGKEFKCKVHNKALPAPIEKTISKARG
QPLEPKVYTMGPPREELSSRSVSLTCMINGFYPSDISVEWEKNGKAEDNY
KTTPAVLDSDGSYFLYNKLSVPTSEWQRGDVFTCSVMHEALHNHYTQKSI
SRSPGK.

In some embodiments, the anti-CD83 scFv V_{L} domain comprises the amino acid sequence:

(SEQ ID NO: 28, 020B08)
MDMRAPTQLLGLLLLWLPGARCAYDMTQTPASVEVAVGGTVTIKCQASQS
ISTYLDWYQQKPGQPPKLLIYDASDLASGVPSRFKGSGSGTQFTLTISDL
ECADAATYYCQQGYTHSNVDNVFGGGTEVVVKGDPVAPTVLLFPPSSDEV
ATGTVTIVCVANKYFPDVTVTWEVDGTTQTTGIENSKTPQNSADCTYNLS
STLTLTSTQYNSHKEYTCKVTQGTTSVVQSFSRKNC

In some embodiments, the anti-CD83 scFv V_{H} domain comprises the amino acid sequence:

(SEQ ID NO: 29, 006G05)
METGLRWLLLVAVLKGVQCQSVEESGGRLVSPGTPLTLTCTASGFSLSSY
DMSWVRQAPGKGLEYIGIISSSGSTYYASWAKGRFTISKTSTTVDLEVTS
LTTEDTATYFCSREHAGYSGDTGHLWGPGTLVTVSSGQPKAPSVFPLAPC
CGDTPSSTVTLGCLVKGYLPEPVTVTWNSGTLTNGVRTFPSVRQSSGLYS
LSSVVSVTSSSQPVTCNVAHPATNTKVDKTVAPSTCSKPTCPPPELLGGP
SVGIGPPKPKDTLMISRTPEVTCVVVDVSQDDPEVQFTWYINNEQVRTAR
PPLREQQFNSTIRVVSTLPIAHQDWLRGKEFKCKVHNKALPAPIEKTISK
ARGQPLEPKVYTMGPPREELSSRSVSLTCMINGFYPSDISVEWEKNGKAE
DNYKTTPAVLDSDGSYFLYNKLSVPTSEWQRGDVFTCSVMHEALHNHYTQ
KSISRSPGK.

In some embodiments, the anti-CD83 scFv V_{L} domain comprises the amino acid sequence:

(SEQ ID NO: 30, 006G05)
MDMRAPTQLLGLLLLWLPGARCAYDMTQTPASVEVAVGGTVAIKCQASQS
VSSYLAWYQQKPGQPPKPLIYEASMLAAGVSSRFKGSGSGTDFTLTISDL
ECDDAATYYCQQGYSISDIDNAFGGGTEVVVKGDPVAPTVLLFPPSSDEV
ATGTVTIVCVANKYFPDVTVTWEVDGTTQTTGIENSKTPQNSADCTYNLS
STLTLTSTQYNSHKEYTCKVTQGTTSVVQSFSRKNC

In some embodiments, the anti-CD83 scFv V_{H} domain comprises the amino acid sequence:

(SEQ ID NO: 31, 96G08)
METGLRWLLLVAVLKGVQCQSVEESGGRLVTPGTPLTLTCTVSGIDLSSD
GISWVRQAPGKGLEWIGIISSGGNTYYASWAKGRFTISRTSTTVDLKMTS
LTTEDTATYFCARVVGGTYSIWGQGTLVTVSSASTKGPSVYPLAPGSAAQ
TNSMVTLGCLVKGYFPEPVTVTWNSGSLSSGVHTFPAVLQSDLYTLSSSV
TVPSSTWPSETVTCNVAHPASSTKVDKKIVPRDCGCKPCICTVPEVSSVF
IFPPKPDVLTITLTPKVTCVVVDISKDDPEVQFSWFVDDVEVHTAQTQPR
EEQFNSTFRSVSELPIMHQDWLNGKEFKCRVNSAAFPAPIEKTISKTKGR
PKAPQVYTIPPPKEQMAKDKVSLTCMITDFFPEDITVEWQWNGQPAENYK
NTQPIMDTDGSYFVYSKLNVQKSNWEAGNTFTCSVLHEGLHNHHTEKSLS
HSPGK.

In some embodiments, the anti-CD83 scFv V_{L} domain comprises the amino acid sequence:

(SEQ ID NO: 32, 96G08)
MDTRAPTQLLGLLLLWLPGATFAQVLTQTASPVSAPVGGTVTINCQSSQS
VYNNDFLSWYQQKPGQPPKLLIYYASTLASGVPSRFKGSGSGTQFTLTIS
DLECDDAATYYCTGTYGNSAWYEDAFGGGTEVVVKRTPVAPTVLLFPPSS
AELATGTATIVCVANKYFPDGTVTWKVDGITQSSGINNSRTPQNSADCTY
NLSSTLTLSSDEYNSHDEYTCQVAQDSGSPVVQSFSRKSC

In some embodiments, the anti-CD83 scFv V_{H} domain comprises the amino acid sequence:

(SEQ ID NO: 33, 95F04)
METGLRWLLLVAVLKGVQCQSVEESGGRLVTPGTPLTLTCTVSGIDLSSN
AMIWVRQAPREGLEWIGAMDSNSRTYYATWAKGRFTISRTSSITVDLKIT
SPTTEDTATYFCARGDGGSSDYTEMWGPGTLVTVSSASTKGPSVYPLAPG
SAAQTNSMVTLGCLVKGYFPEPVTVTWNSGSLSSGVHTFPAVLQSDLYTL
SSSVTVPSSTWPSETVTCNVAHPASSTKVDKKIVPRDCGCKPCICTVPEV
SSVFIFPPKPKDVLTITLTPKVTCVVVDISKDDPEVQFSWFVDDVEVHTA
QTQPREEQFNSTFRSVSELPIMHQDWLNGKEFKCRVNSAAFPAPIEKTIS
KTKGRPKAPQVYTIPPPKEQMAKDKVSLTCMITDFFPEDITVEWQWNGQP
AENYKNTQPIMDTDGSYFVYSKLNVQKSNWEAGNTFTCSVLHEGLHNHHT
EKSLSHSPGK.

In some embodiments, the anti-CD83 scFv V_{L} domain comprises the amino acid sequence:

(SEQ ID NO: 34, 95F04)
MDTRAPTQLLGLLLLWLPGATFAQAVVTQTTSPVSAPVGGTVTINCQSSQ
SVYGNNELSWYQQKPGQPPKLLIYQASSLASGVPSRFKGSGSGTQFTLTI
SDLECDDAATYYCLGEYSISADNHFGGGTEVVVKRTPVAPTVLLFPPSSA
ELATGTATIVCVANKYFPDGTVTWKVDGITQSSGINNSRTPQNSADCTYN
LSSTLTLSSDEYNSHDEYTCQVAQDSGSPVVQSFSRKSC

In some embodiments, the anti-CD83 scFv V_{H} domain comprises the amino acid sequence:

(SEQ ID NO: 35)
QVQLVQSGGAVVQPGRSLRLSCAASGFTFSTYGMHWVRQAPGKGLEWVAA
VSYDGSNKYYADFVKGRFTISRDNPKNTLYLQMNSLRADDTAVYYCARRG
GLDIWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCAAA.

In some embodiments, the anti-CD83 scFv V_{L} domain comprises the amino acid sequence:

(SEQ ID NO: 36)
LTQPPPASGTPGQQRVTISCSGSSSNIGSNTVNWYQQLPGTAPKLLIYYG
NDQRPSGVPDRFSASKSGTSASLAISGLQSEDEAHYYCAAWDGSLNGGVI
FGGGTKVTLG.

In some embodiments, the anti-CD83 scFv V_{L} domain comprises the amino acid sequence:

(SEQ ID NO: 37)
VTQPPSASGTPGQRVTISCSGSSSNIGTNPVNWYQQLPGTAPKLLIYTTD
QRPSGVPDRFSGSKSGTSASLAISGLQSEDEADYYCAAWDDSLSGLYVFG
TGTKVTVLG.

In some embodiments, the anti-CD83 scFv VL domain comprises the amino acid sequence:

(SEQ ID NO: 38)
MTHTPLSLSVTPGQPASISCKSSQSLLHSDGKTYLYWYLQRPGQSPQPLI
YEVSNRFSGVPDRFSGSGSGTDFTLKISRVQAEDVGVYYCMQSLQLWTFG
QGTKVEIKR.

In some embodiments, the anti-CD83 scFv V_{L} domain comprises the amino acid sequence:

(SEQ ID NO: 39)
MTQSPLSLPVTLGQPASISCRSSQSLIHSDGNTYLDWFQQRPGQSPRRLI
YKVSNRDSGVPDRFSGSGSGTDFTLRISRVEAEDIGVYYCMQATHWPRTF
GQGTKVEIKR.

In some embodiments, the anti-CD83 scFv V_{L} domain comprises the amino acid sequence:

(SEQ ID NO: 40)
MTQSPLSLPVTLGQPASISCRSSQSLVDSAGNTFLHWFHQRPGQSPRRLI
YKVSNRDSGVPDRFSGSGSGTDFTLKISRVEAEDVGVYYCMQGTHWPRTF
GQGTKVEIKR.

In some embodiments, the anti-CD83 scFv V_{L} domain comprises the amino acid sequence:

(SEQ ID NO: 41)
LTQSPLSLPVTLGQPASISCKSSQSLVDSDGNTYLNWFQQRPGQSPRRLI
YKVSNRDSGVPDRFSGSGSGTDFTLKISRVEAEDVGVYYCMQGTHWPRTF
GQGTKVEIKR.

In some embodiments, the anti-CD83 scFv V_{L} domain comprises the amino acid sequence:

(SEQ ID NO: 42)
MTQSPLSLPVTLGQPASISCRSSQSLVHSDGNMYLNWFQQRPGQSPRRLI
YKVSNRDSGVPDRFSGSGSGTDFTLKISRVEAEDVGVYYCMQATQPTWTF
GQGTKLEIKR.

In some embodiments, the anti-CD83 scFv V_{L} domain comprises the amino acid sequence:

(SEQ ID NO: 43)
MTQSPSSLSASVGDRVTITCQASQDISNYLNWYQQKPGKAPKLLIYDASN
LETGVPSRFSGSGSGTDFTFTISSATYYCQQTYQGTKLEIKR.

In some embodiments, the anti-CD83 scFv V_{L} domain comprises the amino acid sequence:

(SEQ ID NO: 44)
MTQSPSSLSASVGHPVTITCRASQSLISYLNWYHQKPGKAPKLLIYAASI
LQSGVPSRFSGSGSGTDFTLTISSLQPENFASYYCQHTDSFPRTFGHGTK
VEIKR.

In some embodiments, the anti-CD83 scFv V_{L} domain comprises the amino acid sequence:

(SEQ ID NO: 45)
LTQPPSASGTPGQGVTISCRGSTSNIGNNVVNWYQHVPGSAPKLLIWSNI
QRPSGIPDRFSGSKSGTSASLAISGLQSEDQAVYYCAVWDDGLAGWVFGG
GTTVTVLS.

In some embodiments, the anti-CD83 scFv V_{L} domain comprises the amino acid sequence:

(SEQ ID NO: 46)
MTQAPVVSVALEQTVRITCQGDSLAIYYDFWYQHKPGQAPVLVIYGKNNR
PSGIPHRFSGSSSNTDSLTITGAQAEDEADYYCNSRDSSGNHWVFGGGTN
LTVLG.

In some embodiments, the anti-CD83 scFv V_{L} domain comprises the amino acid sequence:

(SEQ ID NO: 47)
LTQSPLSLPVTLGQPASISCKSNQSLVHSDGNTYLNWFQQRPGQSPRRLI
YKVSNRDSGVPDRFSGSGSGTDFTLKINRVEAEDVGVYYCMQGTQWPRTF
GGQGTKLDIKR.

In some embodiments, the anti-CD83 scFv V_{H} domain has been humanized and comprises the amino acid sequence:

(SEQ ID NO: 48, VH-GBM01)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSS.

In some embodiments, the anti-CD83 scFv V_{H} domain has been humanized and comprises the amino acid sequence:

(SEQ ID NO: 49, VH-GBM02)
QVQLQESGPGLVKPSQTLSLTCTVSGFSITTGGYWWTWIRQHPGKGLEWI
GYIFSSGNTNYNPSIKSLVTISVDTSKNQFSLKLSSVTAADTAVYYCARA
YGKLGFDYWGQGTLVTVSS.

In some embodiments, the anti-CD83 scFv V_{H} domain has been humanized and comprises the amino acid sequence:

(SEQ ID NO: 50, VH-GBM03)
QVQLQESGPGLVKPSQTLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSS.

In some embodiments, the anti-CD83 scFv V_{H} domain has been humanized and comprises the amino acid sequence:

(SEQ ID NO: 51, VH-GBM04)
QVQLQESGPGLVKPSETLSLTCTVSGFSITTGGYWWTWIRQPPGKGLEWI
GYIFSSGNTNYNPSIKSRVTISRDTSKNQFSLKLSSVTAADTAVYYCARA
YGKLGFDYWGQGTLVTVSS.

In some embodiments, the anti-CD83 scFv V_{H} domain has been humanized and comprises the amino acid sequence:

(SEQ ID NO: 52, VH-GBM05)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTARYYCARA
YGKLGFDYWGQGTLVTVSS.

In some embodiments, the anti-CD83 scFv V_{H} domain has been humanized and comprises the amino acid sequence:

(SEQ ID NO: 53, VH-GBM06)
QVQLQESGPGLVKPSETLSLTCTVSGFSITTGGYWWTWIRQPPGKGLEWI
GYIFSSGNTNYNPSIKSRISITRDTSKNQFFLQLNSVTTEGDTARYYCAR
AYGKLGFDYWGQGTLVTVSS.

In some embodiments, the anti-CD83 scFv V_{L} domain has been humanized and comprises the amino acid sequence:

(SEQ ID NO: 54, VL-GBM01)
QLVLTQSPSASASLGASVKLTCTLSSQHSTYTIGWHQQQPEKGPRYLMKV
NSDGSHSKGDGIPDRFSGSSSGAERYLTISSLQSEDEADYYCGSSDSSGY
VFGSGTKVTVL.

In some embodiments, the anti-CD83 scFv V_{L} domain has been humanized and comprises the amino acid sequence:

(SEQ ID NO: 55, VL-GBM02)
LPVLTQPPSASALLGASIKLTCTLSSQHSTYTIGWYQQRPGRSPQYIMKV
NSDGSHSKGDGIPDRFMGSSSGADRYLTFSNLQSDDEAEYHCGSSDSSGY
VFGSGTKVTVL.

The heavy and light chains are preferably separated by a linker. Suitable linkers for scFv antibodies are known in the art. In some embodiments, the linker comprises the amino acid sequence GGGGSGGGGSGGGGS (SEQ ID NO:56).

In some embodiments, the anti-CD83 scFv comprises an amino acid sequence:

(SEQ ID NO: 57)
QPVLTQSPSASASLGNSVKITCTLSSQHSTYTIGWYQQHPDKAPKYVMYV
NSDGSHSKGDGIPDRFSGSSSGAHRYLSISNIQPEDEADYFCGSSDSSGY
VFGSGTQLTVLRAAASSGGGGSGGGGSGGGGSQPVLTQSPSASASLGNSV
KITCTLSSQHSTYTIGWYQQHPDKAPKYVMYVNSDGSHSKGDGIPDRFSG
SSSGAHRYLSISNIQPEDEADYFCGSSDSSGYVFGSGTQLTVLRAAA.

In some embodiments, the anti-CD83 scFv comprises an amino acid sequence:

(SEQ ID NO: 58)
QVQLKESGPGLVKPSQSLSLTCSVTGFSITTGGYWWTWIRQFPGQKLEWM
GYIFSSGNTNYNPSIKSRISITRDTSKNQFFLQLNSVTTEGDTARYYCAR
AYGKLGFDYWGQGTLVTVSSGGGGSGGGGSGGGGSQVQLKESGPGLVKPS
QSLSLTCSVTGFSITTGGYWWTWIRQFPGQKLEWMGYIFSSGNTNYNPSI
KSRISITRDTSKNQFFLQLNSVTTEGDTARYYCARAYGKLGFDYWGQGTL
VTV.

In some embodiments, the anti-CD83 scFv comprises an amino acid sequence:

(SEQ ID NO: 59)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 60
QVQLQESGPGLVKPSQTLSLTCTVSGFSITTGGYWWTWIRQHPGKGLEWI
GYIFSSGNTNYNPSIKSLVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 61)
QVQLQESGPGLVKPSQTLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 62)
QVQLQESGPGLVKPSETLSLTCTVSGFSITTGGYWWTWIRQPPGKGLEWI
GYIFSSGNTNYNPSIKSRVTISRDTSKNQFSLKLSSVTAADTAVYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 63)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTARYYCARA
YGKLGFDYWGQGTLVTVSSGGGGSGGGGSGGGGSQLVLTQSPSASASLGA
SVKLTCTLSSQHSTYTIGWHQQQPEKGPRYLMKVNSDGSHSKGDGIPDRF
SGSSSGAERYLTISSLQSEDEADYYCGSSDSSGYVFGSGTKVTVL.

In some embodiments, the anti-CD83 scFv comprises an amino acid sequence:

(SEQ ID NO: 64)
QVQLQESGPGLVKPSETLSLTCTVSGFSITTGGYWWTWIRQPPGKGLEWI
GYIFSSGNTNYNPSIKSRISITRDTSKNQFFLQLNSVTTEGDTARYYCAR
AYGKLGFDYWGQGTLVTVSSGGGGSGGGGSGGGGSQLVLTQSPSASASLG
ASVKLTCTLSSQHSTYTIGWHQQQPEKGPRYLMKVNSDGSHSKGDGIPDR
FSGSSSGAERYLTISSLQSEDEADYYCGSSDSSGYVFGSGTKVTVL.

In some embodiments, the anti-CD83 scFv comprises an amino acid sequence:

(SEQ ID NO: 65)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL.

In some embodiments, the anti-CD83 scFv comprises an amino acid sequence:

(SEQ ID NO: 66)
QVQLQESGPGLVKPSQTLSLTCTVSGFSITTGGYWWTWIRQHPGKGLEWI
GYIFSSGNTNYNPSIKSLVTISVDTSKNQFSLKLSSVTAADTAVYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL.

In some embodiments, the anti-CD83 scFv comprises an amino acid sequence:

(SEQ ID NO: 67)
QVQLQESGPGLVKPSQTLSLTCTVSGFSITTGGYWWTWIRQPPGKGLEWI
GYIFSSGNTNYNPSIKSRVTISVDTSKNQFSLKLSSVTAADTAVYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL.

In some embodiments, the anti-CD83 scFv comprises an amino acid sequence:

(SEQ ID NO: 68)
QVQLQESGPGLVKPSETLSLTCTVSGFSITTGGYWWTWIRQPPGKGLEWI
GYIFSSGNTNYNPSIKSRVTISRDTSKNQFSLKLSSVTAADTAVYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL.

In some embodiments, the anti-CD83 scFv comprises an amino acid sequence:

(SEQ ID NO: 69)
QVQLQESGPGLVKPSETLSLTCTVSGFSITTGGYWWTWIRQPPGKGLEWI
GYIFSSGNTNYNPSIKSRVTISVDTSKNQFSLKLSSVTAADTARYYCARA
YGKLGFDYWGQGTLVTVSSGGGGSGGGGSGGGGSLPVLTQPPSASALLGA
SIKLTCTLSSQHSTYTIGWYQQRPGRSPQYIMKVNSDGSHSKGDGIPDRF
MGSSSGADRYLTFSNLQSDDEAEYHCGSSDSSGYVFGSGTKVTVL.

In some embodiments, the anti-CD83 scFv comprises an amino acid sequence:

(SEQ ID NO: 70)
QVQLQESGPGLVKPSETLSLTCTVSGFSITTGGYWWTWIRQPPGKGLEWI
GYIFSSGNTNYNPSIKSRISITRDTSKNQFFLQLNSVTTEGDTARYYCAR
AYGKLGFDYWGQGTLVTVSSGGGGSGGGGSGGGGSLPVLTQPPSASALLG
ASIKLTCTLSSQHSTYTIGWYQQRPGRSPQYIMKVNSDGSHSKGDGIPDR
FMGSSSGADRYLTFSNLQSDDEAEYHCGSSDSSGYVFGSGTKVTVL.

In some embodiments, the anti-CD83 scFv comprises an amino acid sequence:

(SEQ ID NO: 71)
QVQLKESGPGLVKPSQSLSLTCSVTGFSITTGGYWWTWIRQFPGQKLEWM
GYIFSSGNTNYNPSIKSRISITRDTSKNQFFLQLNSVTTEGDTARYYCAR
AYGKLGFDYWGQGTLVTVSSGGGGSGGGGSGGGGSQPVLTQSPSASASLG
NSVKITCTLSSQHSTYTIGWYQQHPDKAPKYVMYVNSDGSHSKGDGIPDR
FSGSSSGAHRYLSISNIQPEDEADYFCGSSDSSGYVFGSGTQLTVL.

As with other CARs, the disclosed polypeptides can also contain a transmembrane domain and an endodomain capable of activating an immune effector cell. For example, the endodomain can contain a signaling domain and one or more co-stimulatory signaling regions.

In some embodiments, the intracellular signaling domain is a CD3 zeta (CD3ζ) signaling domain. In some embodiments, the costimulatory signaling region comprises the cytoplasmic domain of CD28, 4-1BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1 BB that enhance signaling.

In some embodiments, the CAR polypeptide contains an incomplete endodomain. For example, the CAR polypeptide can contain only an intracellular signaling domain or a co-stimulatory domain, but not both. In these embodiments, the immune effector cell is not activated unless it and a second CAR polypeptide (or endogenous T-cell receptor) that contains the missing domain both bind their respective antigens. Therefore, in some embodiments, the CAR polypeptide contains a CD3 zeta (CD3ζ) signaling domain but does not contain a costimulatory signaling region (CSR). In other embodiments, the CAR polypeptide contains the cytoplasmic domain of CD28, 4-1 BB, or a combination thereof, but does not contain a CD3 zeta (CD3ζ) signaling domain (SD).

Also disclosed are isolated nucleic acid sequences encoding the disclosed CAR polypeptides, vectors comprising these isolated nucleic acids, and regulatory T cells containing these vectors. In some embodiments, the cell suppresses alloreactive donor cells, such as T cells, when the antigen binding domain of the CAR binds to CD83.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1G show human CD83-targeted CART construct and functional characteristics. FIG. 1A shows an anti-CD83 single chain variable fragment is followed by a CD8 hinge and transmembrane domain, as well as a 41BB costimulatory domain and CD3s activation domain. The CAR is tagged with a fluorescent reporter at the 3′ end. The CAR Reporter gene is cloned into a SFG retroviral vector. FIG. 1B is a bar graph showing the amount (mean±SEM) of T cells expressing the eGFP reporter post production among mock transduced (eGFP negative) or the CD83 CAR (eGFP positive) T cells. FIG. 1C is a bar graph demonstrating the relative amount (mean±SEM) of CD4 or CD8 expression among the mock transduced or the CD83 CART cells, Sidak's test. FIGS. 1D and 1E shows the amount of IFNy and IL-2 released by mock transduced or CD83 CART cells after stimulation with CD83+ DCs. FIG. 1F shows CD83 CART cells or mock transduced T cells co-cultured with CD83+ DCs and cytotoxicity measured on a realtime cell analysis system. The data are presented as the average normalized cell index over time for duplicate wells. Normalized cell index is calculated as cell index at a given time point divided by cell index at the normalized time point which is day 1 after addition of T cells. 1 representative experiment of 2 is shown, Dunnett's test. FIG. 1G shows CD83 CART cells or mock transduced T cells were stimulated by CD83+ DCs and the absolute number of T cells was calculated weekly over a 14 day period. 1 representative experiment of 2 shown, Sidak's test. **P=0.001-.01, ***P=0.0001-.001, and ****P<0.0001.

FIG. 2 shows human CD83 chimeric antigen receptor T cells reduce alloreactivity. Human T cells were cultured with allogeneic, cytokine matured, monocyte-derived dendritic cells (moDC) at a DC:T cell ratio of 1:30 (i.e., 100,000 T cells and 3333 moDCs). CD83 CART (autologous to the cultured T cells) were added at specific ratios to the moDCs (3:1 to 1:10, where the lowest amount of CART added was 333 cells). T cell proliferation was measured by Ki-67 expression at day +5. CAR T were gated out by their expression of GFP. Controls included T cells alone (i.e., no proliferation), mock transduced T cells, and CD19 CART cells. These mock transduced T cell did not express a chimeric antigen receptor but were treated in an identical fashion as the transduced CD83 cells. The CD19 CART cell used a 41BB co-stimulation domain, and targeted an irrelevant antigen in this system. 1 of 2 representative experiments is shown.

FIGS. 3A to 3D show CD83 is differentially expressed on human activated conventional CD4+ T cells (Tcon) compared to regulatory T cells (Tregs). Human T cells were stimulated by allogeneic moDCs (DC:T cell ratio 1:30) or CD3/CD28 beads (Bead:T cell ratio 1:30). CD83 expression on activated Tconv (CD4+, CD127+, CD25+) or Treg (CD4+, CD127−, CD25+, Foxp3+) was measured at baseline, 4 hours, 8 hours, 24 hours, and 48 hours post stimulation. Bar graphs show the amount of CD83+ Tconv or Treg (mean±SEM) after allogeneic DC (FIG. 3A) or CD3/CD28 bead (FIG. 3B) stimulation. n=5 independent experiments, Sidak's test. Human CD83 CAR or mock T cells were cultured with DC-allostimulated PBMCs at a ratio of 1:10 over 48 hours. Representative contour plots show the C) frequency of CD83+, CD3− and CD3+ target cells (FIG. 3C) and expression of CD83 among eGFP+ CART cells (FIG. 3D) over time. 1 representative experiment of 2 is shown. ****P<0.0001.

FIGS. 4A to 4J show human CD83 CART cells prevents xenogeneic GVHD. A) NSG mice received 25×106 human PBMCs and were inoculated with low (1×10⁶) or high dose (10×10⁶) CD83 CAR or (1-10×10⁶) mock transduced T cells. The CARs were autologous to the PBMC donor. An additional control group of mice received PBMCs alone. FIGS. 4A and 4B show survival (FIG. 4A) and GVHD (FIG. 4B) clinical scores. Clinical scores incorporate an aggregate assessment of activity, fur and skin condition, weight loss, and posture. Pooled data from 3 independent experiments, up to 9 mice per experimental arm. Log-rank test. In separate experiments, recipient mice were humanely euthanized at day +21 and tissue GVHD severity was evaluated by an expert, blinded pathologist. Xenogeneic GVHD path scores, representative H&E images, amount of Ki-67+, CD3+ T cells/HPF, and representative IHC images (CD3=red, Ki-67=brown) are shown for recipient lung (FIGS. 4C to 4F) and liver (FIGS. 4G to 4J). Pooled data from 2 independent experiments, up to 6 mice per experimental arm. Dunnett's test (group comparisons) or Mann-Whitney. **P=0.001-.01 and ***P=0.0001-0.001.

FIGS. 5A to 5C show human CD83-targeted CAR T cells significantly reduce CD83+ DCs. NSG mice received 25×10⁶ human PBMCs plus 1×10⁶ CD83 CAR or mock transduced T cells as described. Mice were humanely euthanized on day +21 and the spleens were harvested. FIG. 5A contains representative contour plots showing the frequency of human CD83+, CDIc+ DCs in the mouse spleens at day +21. FIG. 5B is a bar graph showing the absolute number (mean±SEM) of human CD83+, CDIc+ DCs in the mouse spleens at day +21, with bar graph depicting the absolute number (mean±SEM) of these cells (FIG. 5D), Dunn's test. FIG. 5C contains representative contour plots showing the percentage of MHC class II+, CDIc+ DCs in the recipient spleens at day +21. Pooled data from 2 independent experiments, up to 6 mice per experimental arm. **P=0.001-.01.

FIGS. 6A to 6J show human CD83-targeted CART cells significantly reduce CD4+, CD83+ T cells, while increasing the Treg:Activated Tconv ratio in vivo. NSG mice received 25×I06 human PBMCs plus 1×10⁶ CD83 CAR or mock transduced T cells as described. Mice were humanely euthanized on day +21 and the spleens were harvested. FIG. 6A contains representative contour plots showing the amount of eGFP+CD83 CAR T cells in the inoculated mice at day +21, compared to mice that received mock transduced T cells. FIG. 6B contains representative contour plots show the frequency of human CD4+ T cells in the recipient spleens. Bar graphs show the absolute numbers (mean±SEM) of CD4+(FIG. 6C) and CD4+, CD83+ T cells (FIG. 6D) in the mouse spleens at day +21, Dunn's test. FIG. 6E contains contour plots depicting the percentage of CD4+, CD12T, CD25+, Foxp3+ Tregs in the mouse spleens at day +21.

FIGS. 6F and 6G contain bar graphs showing the amount (mean±SEM) of Tregs (FIG. 6F) and the Treg:Activated Tconv (FIG. 6G) at day +21 in the recipient mice, Dunn's test. FIG. 6H contains contour plots depicting the frequency of CD4+, IFNy+ Thl cells and CD4+, IL-4+Th2 cells in the mouse spleens at day +21. FIGS. 6I and 6J bar graphs demonstrating the absolute numbers (mean±SEM) of Thl (FIG. 6I) and Th2 (FIG. 6J) cells in the recipient spleens, Dunn's test. Pooled data from 2 independent experiments, up to 6 mice per experimental arm. *P<0.05, **P=0.001-.01.

FIGS. 7A and 7B show human CD83 CART cells can still kill and proliferate in response to CD83+ target cells when exposed to tacrolimus. FIG. 7A show human CD83 CART cells or untransduced T cells from the same donor cultured with allogeneic, CD83+ cytokine-matured moDCs at various T cell to DC ratios for 24 hours. The cultures were exposed to a clinically relevant dose of tacrolimus (10 ng/ml) or DMSO control (<0.01%). Bar graph shows DC lysis at 24 hours per a colorimetric LDH assay. FIG. 7B shows human CD83 CAR T cells or untransduced T cells from the same donor cultured with allogeneic, CD83+ cytokinematured moDCs at a T:DC ratio of 1:30. Tacrolimus or DMSO control was added once on day 0, and proliferation was evaluated by a colorimetric assay after 3 days. 1 representative experiment of 2 is show for each, Sidak's test. ***P=0.0001-.001 and ****P<0.0001.

FIG. 8 shows human CD83 CART cells reduce the expansion of donor cells in vivo. NSG mice were transplanted with 25×10⁶ human PBMCs plus 1×10⁶ CD83 CAR or mock transduced T cells. Control groups consisted of mice that received no PBMCs (negative control) and mice that received PBMCs without modified T cells (secondary positive control). Recipient mice were humanely euthanized at day +21 and their spleens were removed for gross assessment. A representative image shows mice that received PBMCs and CD83 CAR T cells exhibit reduced spleen size, supporting suppression of donor T cell expansion in vivo. 1 representative experiment of 2.

FIGS. 9A to 9C show human CD83 CART cells eliminate CD83+ targets at day +21. NSG mice were transplanted with 25×10⁶ human PBMCs plus 1×10⁶ CD83 CAR or mock transduced T cells. Recipient mice were humanely euthanized at day +21 and the amount of eGFP+ CARs, CD83+, CDIc+DCs, and CD83+, CD4+ T cells were analyzed by flow cytometry. FIG. 9A is a bar graph showing the amount of eGFP+ CART cells in the recipient spleens at day +21, as well as the % reduction of CD83+ targets in the spleen normalized by mice injected with mock T cells. FIGS. 9B and 9C contain graphs showing the linear regression (dotted line) of CD83+ targets per the amount of eGFP+ CART cells recovered at day +21. Spearman rank-order correlation coefficient is shown. Pooled data from 2 independent experiments, up to 6 mice per experimental arm.

FIGS. 10A and 10B show DC-depletion does not prevent xenogeneic GVHD mediated by human T cells. NSG mice received 7.5×10⁶ purified human T cells alone or with 1.87×10⁵ autologous dendritic cells. The dendritic cells were isolated by magnetic bead purification (Miltenyi), and included plasmacytoid DCs, CD1c+ type-1 myeloid DCs, and CD1c−, CD141^{bright} type-2 myeloid DCs. FIGS. 10A and 10B show survival (FIG. 10A) and GVHD (FIG. 10B) clinical scores. A representative experiment is shown, 4 mice per experimental arm.

FIGS. 11A and 11B show human CD83 CAR T cells do not reduce the amount of donor Th17 cells. NSG mice received 25×10⁶ human PBMCs plus 1×10⁶ CD83 CAR or mock transduced T cells as described. Mice were humanely euthanized on day +21 and the spleens were harvested. FIG. 10A contains representative contour plots showing the frequency of human CD4+, IL-17+ Th17 cells in the mouse spleens at day +21. FIG. 11B contains a bar graph showing the absolute number (mean±SEM) of human Thl 7 cells in the mouse spleens at day +21. Pooled data from 2 independent experiments, up to 6 mice per experimental arm.

FIG. 12 shows human CD83 CAR T cells are present at day +100. NSG mice received 25×10⁶ human PBMCs plus 1-10×10⁶ CD83 CAR or 10×10⁶ mock transduced T cells. The contour plots show the amount of CD83+ target cells versus eGFP+CD83 CART cells from the spleens of representative mice that survived up to the day +100 endpoint. Data from 1 representative experiment of 3 is shown.

FIG. 13 shows human CD83 CAR T cells reduce the amount of donor CD8+ T cells in vivo. NSG mice received 25×10⁶ human PBMCs plus 1×10⁶ CD83 CAR or mock transduced T cells as described. On day +21, the amount of donor, human CD8+ T cells were enumerated, Dunn's test. Pooled data from 2 independent experiments, up to 6 mice per experimental arm.

FIG. 14 shows off-the-shelf CD83 CAR Tregs offer significantly enhanced suppression of alloreactive T cells. Human, regulatory T cells (Treg) expression a CD83 CAR or mock transduced Tregs were cultured with allogeneic mixed leukocyte reactions (T cell to DC ratio 30:1). The Tregs, T cells, and dendritic cells were entirely HLA-mismatched from each other. Graph shows alloreactive T cell proliferation as measured on day +5 of the culture. One representative experiment of 2 is shown, Sidak's test. *P<0.05, **P=0.001-0.1.

FIGS. 15A and 15B: Human CD83 CAR T cells treat and rescue mice from xenogeneic GVHD. NSG mice received 25×10⁶ human PBMCs on day 0, followed by 1×10⁶ autologous CD83 CAR T cells or mock transduced T cells on day +14. FIG. 15A shows GVHD clinical scores. FIG. 15B shows survival. A representative experiment is shown, 4 mice per experimental arm. ANOVA or Log-rank test. **P=0.001-0.01 and ****P<0.0001.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

Disclosed herein are chimeric antigen receptors (CAR) that target CD83 on antigen-presenting cells. Also disclosed are regulatory T cells that are engineered to express these CARs. CAR T cells expressing these CARs can suppress alloreactive donor cells, such as T cells. Therefore, also disclosed are methods for preventing GVHD in a subject that involves adoptive transfer of the disclosed regulatory T cells engineered to express the disclosed CD83-specific CARs.

### CD83-Specific Chimeric Antigen Receptors (CAR)

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). Disclosed herein is a CD83-specific chimeric antigen receptor (CAR) that can be that can be expressed in regulatory T cells to suppress alloreactive donor cells.

The disclosed CAR is generally made up of three domains: an ectodomain, a transmembrane domain, and an endodomain. The ectodomain comprises the CD83-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, a regulatory T cell containing the disclosed CAR is only activated if another CAR (or a T-cell receptor) containing the missing domain also binds its respective antigen.

In some embodiments, the disclosed CAR is defined by the formula:

SP-CD83-HG-TM-CSR-SD; or

SP-CD83-HG-TM-SD-CSR;
wherein “SP” represents an optional signal peptide,
wherein “CD83” represents a CD83-binding region,
wherein “HG” represents an optional hinge domain,
wherein “TM” represents a transmembrane domain,
wherein “CSR” represents one or more co-stimulatory signaling regions,
wherein “SD” represents a signaling domain, and
wherein “-” represents a peptide bond or linker.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

A tandem CAR (TanCAR) T cell expresses a single CAR consisting of two linked single-chain variable fragments (scFvs) that have different affinities fused to intracellular co-stimulatory domain(s) and a CD3ζdomain. TanCAR T cell activation is achieved only when target cells co-express both targets.

A dual CAR T cell expresses two separate CARs with different ligand binding targets; one CAR includes only the CD3ζdomain and the other CAR includes only the co-stimulatory domain(s). Dual CAR T cell activation requires co-expression of both targets.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIy, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζchain, which is the primary transmitter of signals from endogenous TCRs. Second-generation CARs add intracellular signaling domains from various costimulatory protein receptors (e.g., CD28, 41 BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζsignaling domain by itself or combined with any other desired cytoplasmic domain(s) useful in the context of the CAR of the invention. For example, the cytoplasmic domain of the CAR can comprise a CD3ζchain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD83 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

Tables 1, 2, and 3 below provide some example combinations of CD83-binding region, co-stimulatory signaling regions, and intracellular signaling domain that can occur in the disclosed CARs.

TABLE 1
First Generation CARs

| ScFv | Signal Domain |
|---|---|
| CD83 | CD8 |
| CD83 | CD3ζ |
| CD83 | CD3δ |
| CD83 | CD3γ |
| CD83 | CD3ε |
| CD83 | FcγRI-γ |
| CD83 | FcγRIII-γ |
| CD83 | FcεRIβ |
| CD83 | FcεRIγ |
| CD83 | DAP10 |
| CD83 | DAP12 |
| CD83 | CD32 |
| CD83 | CD79a |

TABLE 2
Second Generation CARs

| | Co-stimulatory | Signal | | Co-stimulatory | Signal |
|---|---|---|---|---|---|
| ScFv | Signal | Domain | ScFv | Signal | Domain |
| CD83 | CD28 | CD8 | CD83 | CD80 | FcεRIβ |
| CD83 | CD28 | CD3ζ | CD83 | CD80 | FcεRIγ |
| CD83 | CD28 | CD3δ | CD83 | CD80 | DAP10 |
| CD83 | CD28 | CD3γ | CD83 | CD80 | DAP12 |
| CD83 | CD28 | CD3ε | CD83 | CD80 | CD32 |
| CD83 | CD28 | FcγRI-γ | CD83 | CD80 | CD79a |
| CD83 | CD28 | FcγRIII-γ | CD83 | CD80 | CD79b |
| CD83 | CD28 | FcεRIβ | CD83 | CD86 | CD8 |
| CD83 | CD28 | FcεRIγ | CD83 | CD86 | CD3ζ |
| CD83 | CD28 | DAP10 | CD83 | CD86 | CD3δ |
| CD83 | CD28 | DAP12 | CD83 | CD86 | CD3γ |
| CD83 | CD28 | CD32 | CD83 | CD86 | CD3ε |
| CD83 | CD28 | CD79a | CD83 | CD86 | FcγRI-γ |
| CD83 | CD28 | CD79b | CD83 | CD86 | FcγRIII-γ |
| CD83 | CD8 | CD8 | CD83 | CD86 | FcεRIβ |
| CD83 | CD8 | CD3ζ | CD83 | CD86 | FcεRIγ |
| CD83 | CD8 | CD3δ | CD83 | CD86 | DAP10 |
| CD83 | CD8 | CD3γ | CD83 | CD86 | DAP12 |
| CD83 | CD8 | CD3ε | CD83 | CD86 | CD32 |
| CD83 | CD8 | FcγRI-γ | CD83 | CD86 | CD79a |
| CD83 | CD8 | FcγRIII-γ | CD83 | CD86 | CD79b |
| CD83 | CD8 | FcεRIβ | CD83 | OX40 | CD8 |
| CD83 | CD8 | FcεRIγ | CD83 | OX40 | CD3ζ |
| CD83 | CD8 | DAP10 | CD83 | OX40 | CD3δ |
| CD83 | CD8 | DAP12 | CD83 | OX40 | CD3γ |
| CD83 | CD8 | CD32 | CD83 | OX40 | CD3ε |
| CD83 | CD8 | CD79a | CD83 | OX40 | FcγRI-γ |
| CD83 | CD8 | CD79b | CD83 | OX40 | FcγRIII-γ |
| CD83 | CD4 | CD8 | CD83 | OX40 | FcεRIβ |
| CD83 | CD4 | CD3ζ | CD83 | OX40 | FcεRIγ |
| CD83 | CD4 | CD3δ | CD83 | OX40 | DAP10 |
| CD83 | CD4 | CD3γ | CD83 | OX40 | DAP12 |
| CD83 | CD4 | CD3ε | CD83 | OX40 | CD32 |
| CD83 | CD4 | FcγRI-γ | CD83 | OX40 | CD79a |
| CD83 | CD4 | FcγRIII-γ | CD83 | OX40 | CD79b |
| CD83 | CD4 | FcεRIβ | CD83 | DAP10 | CD8 |
| CD83 | CD4 | FcεRIγ | CD83 | DAP10 | CD3ζ |
| CD83 | CD4 | DAP10 | CD83 | DAP10 | CD3δ |
| CD83 | CD4 | DAP12 | CD83 | DAP10 | CD3γ |
| CD83 | CD4 | CD32 | CD83 | DAP10 | CD3ε |
| CD83 | CD4 | CD79a | CD83 | DAP10 | FcγRI-γ |
| CD83 | CD4 | CD79b | CD83 | DAP10 | FcγRIII-γ |
| CD83 | b2c | CD8 | CD83 | DAP10 | FcεRIβ |
| CD83 | b2c | CD3ζ | CD83 | DAP10 | FcεRIγ |
| CD83 | b2c | CD3δ | CD83 | DAP10 | DAP10 |
| CD83 | b2c | CD3γ | CD83 | DAP10 | DAP12 |
| CD83 | b2c | CD3ε | CD83 | DAP10 | CD32 |
| CD83 | b2c | FcγRI-γ | CD83 | DAP10 | CD79a |
| CD83 | b2c | FcγRIII-γ | CD83 | DAP10 | CD79b |
| CD83 | b2c | FcεRIβ | CD83 | DAP12 | CD8 |
| CD83 | b2c | FcεRIγ | CD83 | DAP12 | CD3ζ |
| CD83 | b2c | DAP10 | CD83 | DAP12 | CD3δ |
| CD83 | b2c | DAP12 | CD83 | DAP12 | CD3γ |
| CD83 | b2c | CD32 | CD83 | DAP12 | CD3ε |
| CD83 | b2c | CD79a | CD83 | DAP12 | FcγRI-γ |
| CD83 | b2c | CD79b | CD83 | DAP12 | FcγRIII-γ |
| CD83 | CD137/41BB | CD8 | CD83 | DAP12 | FcεRIβ |
| CD83 | CD137/41BB | CD3ζ | CD83 | DAP12 | FcεRIγ |
| CD83 | CD137/41BB | CD3δ | CD83 | DAP12 | DAP10 |
| CD83 | CD137/41BB | CD3γ | CD83 | DAP12 | DAP12 |
| CD83 | CD137/41BB | CD3ε | CD83 | DAP12 | CD32 |
| CD83 | CD137/41BB | FcγRI-γ | CD83 | DAP12 | CD79a |
| CD83 | CD137/41BB | FcγRIII-γ | CD83 | DAP12 | CD79b |
| CD83 | CD137/41BB | FcεRIβ | CD83 | MyD88 | CD8 |
| CD83 | CD137/41BB | FcεRIγ | CD83 | MyD88 | CD3ζ |
| CD83 | CD137/41BB | DAP10 | CD83 | MyD88 | CD3δ |
| CD83 | CD137/41BB | DAP12 | CD83 | MyD88 | CD3γ |
| CD83 | CD137/41BB | CD32 | CD83 | MyD88 | CD3ε |
| CD83 | CD137/41BB | CD79a | CD83 | MyD88 | FcγRI-γ |
| CD83 | CD137/41BB | CD79b | CD83 | MyD88 | FcγRIII-γ |
| CD83 | ICOS | CD8 | CD83 | MyD88 | FcεRIβ |
| CD83 | ICOS | CD3ζ | CD83 | MyD88 | FcεRIγ |
| CD83 | ICOS | CD3δ | CD83 | MyD88 | DAP10 |
| CD83 | ICOS | CD3γ | CD83 | MyD88 | DAP12 |
| CD83 | ICOS | CD3ε | CD83 | MyD88 | CD32 |
| CD83 | ICOS | FcγRI-γ | CD83 | MyD88 | CD79a |
| CD83 | ICOS | FcγRIII-γ | CD83 | MyD88 | CD79b |
| CD83 | ICOS | FcεRIβ | CD83 | CD7 | CD8 |
| CD83 | ICOS | FcεRIγ | CD83 | CD7 | CD3ζ |
| CD83 | ICOS | DAP10 | CD83 | CD7 | CD3δ |
| CD83 | ICOS | DAP12 | CD83 | CD7 | CD3γ |
| CD83 | ICOS | CD32 | CD83 | CD7 | CD3ε |
| CD83 | ICOS | CD79a | CD83 | CD7 | FcγRI-γ |
| CD83 | ICOS | CD79b | CD83 | CD7 | FcγRIII-γ |
| CD83 | CD27 | CD8 | CD83 | CD7 | FcεRIβ |
| CD83 | CD27 | CD3ζ | CD83 | CD7 | FcεRIγ |
| CD83 | CD27 | CD3δ | CD83 | CD7 | DAP10 |
| CD83 | CD27 | CD3γ | CD83 | CD7 | DAP12 |
| CD83 | CD27 | CD3ε | CD83 | CD7 | CD32 |
| CD83 | CD27 | FcγRI-γ | CD83 | CD7 | CD79a |
| CD83 | CD27 | FcγRIII-γ | CD83 | CD7 | CD79b |
| CD83 | CD27 | FcεRIβ | CD83 | BTNL3 | CD8 |
| CD83 | CD27 | FcεRIγ | CD83 | BTNL3 | CD3ζ |
| CD83 | CD27 | DAP10 | CD83 | BTNL3 | CD3δ |
| CD83 | CD27 | DAP12 | CD83 | BTNL3 | CD3γ |
| CD83 | CD27 | CD32 | CD83 | BTNL3 | CD3ε |
| CD83 | CD27 | CD79a | CD83 | BTNL3 | FcγRI-γ |
| CD83 | CD27 | CD79b | CD83 | BTNL3 | FcγRIII-γ |
| CD83 | CD28δ | CD8 | CD83 | BTNL3 | FcεRIβ |
| CD83 | CD28δ | CD3ζ | CD83 | BTNL3 | FcεRIγ |
| CD83 | CD28δ | CD3δ | CD83 | BTNL3 | DAP10 |
| CD83 | CD28δ | CD3γ | CD83 | BTNL3 | DAP12 |
| CD83 | CD28δ | CD3ε | CD83 | BTNL3 | CD32 |
| CD83 | CD28δ | FcγRI-γ | CD83 | BTNL3 | CD79a |
| CD83 | CD28δ | FcγRIII-γ | CD83 | BTNL3 | CD79b |
| CD83 | CD28δ | FcεRIβ | CD83 | NKG2D | CD8 |
| CD83 | CD28δ | FcεRIγ | CD83 | NKG2D | CD3ζ |
| CD83 | CD28δ | DAP10 | CD83 | NKG2D | CD3δ |
| CD83 | CD28δ | DAP12 | CD83 | NKG2D | CD3γ |
| CD83 | CD28δ | CD32 | CD83 | NKG2D | CD3ε |
| CD83 | CD28δ | CD79a | CD83 | NKG2D | FcγRI-γ |
| CD83 | CD28δ | CD79b | CD83 | NKG2D | FcγRIII-γ |
| CD83 | CD80 | CD8 | CD83 | NKG2D | FcεRIβ |
| CD83 | CD80 | CD3ζ | CD83 | NKG2D | FcεRIγ |
| CD83 | CD80 | CD3δ | CD83 | NKG2D | DAP10 |
| CD83 | CD80 | CD3γ | CD83 | NKG2D | DAP12 |
| CD83 | CD80 | CD3ε | CD83 | NKG2D | CD32 |
| CD83 | CD80 | FcγRI-γ | CD83 | NKG2D | CD79a |
| CD83 | CD80 | FcγRIII-γ | CD83 | NKG2D | CD79b |

TABLE 3
Third Generation CARs

| | Co-stimulatory | Co-stimulatory | Signal |
|---|---|---|---|
| ScFv | Signal | Signal | Domain |
| CD83 | CD28 | CD28 | CD8 |
| CD83 | CD28 | CD28 | CD3ζ |
| CD83 | CD28 | CD28 | CD3δ |
| CD83 | CD28 | CD28 | CD3γ |
| CD83 | CD28 | CD28 | CD3ε |
| CD83 | CD28 | CD28 | FcγRI-γ |
| CD83 | CD28 | CD28 | FcγRIII-γ |
| CD83 | CD28 | CD28 | FcεRIβ |
| CD83 | CD28 | CD28 | FcεRIγ |
| CD83 | CD28 | CD28 | DAP10 |
| CD83 | CD28 | CD28 | DAP12 |
| CD83 | CD28 | CD28 | CD32 |
| CD83 | CD28 | CD28 | CD79a |
| CD83 | CD28 | CD28 | CD79b |
| CD83 | CD28 | CD8 | CD8 |
| CD83 | CD28 | CD8 | CD3ζ |
| CD83 | CD28 | CD8 | CD3δ |
| CD83 | CD28 | CD8 | CD3γ |
| CD83 | CD28 | CD8 | CD3ε |
| CD83 | CD28 | CD8 | FcγRI-γ |
| CD83 | CD28 | CD8 | FcγRIII-γ |
| CD83 | CD28 | CD8 | FcεRIβ |
| CD83 | CD28 | CD8 | FcεRIγ |
| CD83 | CD28 | CD8 | DAP10 |
| CD83 | CD28 | CD8 | DAP12 |
| CD83 | CD28 | CD8 | CD32 |
| CD83 | CD28 | CD8 | CD79a |
| CD83 | CD28 | CD8 | CD79b |
| CD83 | CD28 | CD4 | CD8 |
| CD83 | CD28 | CD4 | CD3ζ |
| CD83 | CD28 | CD4 | CD3δ |
| CD83 | CD28 | CD4 | CD3γ |
| CD83 | CD28 | CD4 | CD3ε |
| CD83 | CD28 | CD4 | FcγRI-γ |
| CD83 | CD28 | CD4 | FcγRIII-γ |
| CD83 | CD28 | CD4 | FcεRIβ |
| CD83 | CD28 | CD4 | FcεRIγ |
| CD83 | CD28 | CD4 | DAP10 |
| CD83 | CD28 | CD4 | DAP12 |
| CD83 | CD28 | CD4 | CD32 |
| CD83 | CD28 | CD4 | CD79a |
| CD83 | CD28 | CD4 | CD79b |
| CD83 | CD28 | b2c | CD8 |
| CD83 | CD28 | b2c | CD3ζ |
| CD83 | CD28 | b2c | CD3δ |
| CD83 | CD28 | b2c | CD3γ |
| CD83 | CD28 | b2c | CD3ε |
| CD83 | CD28 | b2c | FcγRI-γ |
| CD83 | CD28 | b2c | FcγRIII-γ |
| CD83 | CD28 | b2c | FcεRIβ |
| CD83 | CD28 | b2c | FcεRIγ |
| CD83 | CD28 | b2c | DAP10 |
| CD83 | CD28 | b2c | DAP12 |
| CD83 | CD28 | b2c | CD32 |
| CD83 | CD28 | b2c | CD79a |
| CD83 | CD28 | b2c | CD79b |
| CD83 | CD28 | CD137/41BB | CD8 |
| CD83 | CD28 | CD137/41BB | CD3ζ |
| CD83 | CD28 | CD137/41BB | CD3δ |
| CD83 | CD28 | CD137/41BB | CD3γ |
| CD83 | CD28 | CD137/41BB | CD3ε |
| CD83 | CD28 | CD137/41BB | FcγRI-γ |
| CD83 | CD28 | CD137/41BB | FcγRIII-γ |
| CD83 | CD28 | CD137/41BB | FcεRIβ |
| CD83 | CD28 | CD137/41BB | FcεRIγ |
| CD83 | CD28 | CD137/41BB | DAP10 |
| CD83 | CD28 | CD137/41BB | DAP12 |
| CD83 | CD28 | CD137/41BB | CD32 |
| CD83 | CD28 | CD137/41BB | CD79a |
| CD83 | CD28 | CD137/41BB | CD79b |
| CD83 | CD28 | ICOS | CD8 |
| CD83 | CD28 | ICOS | CD3ζ |
| CD83 | CD28 | ICOS | CD3δ |
| CD83 | CD28 | ICOS | CD3γ |
| CD83 | CD28 | ICOS | CD3ε |
| CD83 | CD28 | ICOS | FcγRI-γ |
| CD83 | CD28 | ICOS | FcγRIII-γ |
| CD83 | CD28 | ICOS | FcεRIβ |
| CD83 | CD28 | ICOS | FcεRIγ |
| CD83 | CD28 | ICOS | DAP10 |
| CD83 | CD28 | ICOS | DAP12 |
| CD83 | CD28 | ICOS | CD32 |
| CD83 | CD28 | ICOS | CD79a |
| CD83 | CD28 | ICOS | CD79b |
| CD83 | CD28 | CD27 | CD8 |
| CD83 | CD28 | CD27 | CD3ζ |
| CD83 | CD28 | CD27 | CD3δ |
| CD83 | CD28 | CD27 | CD3γ |
| CD83 | CD28 | CD27 | CD3ε |
| CD83 | CD28 | CD27 | FcγRI-γ |
| CD83 | CD28 | CD27 | FcγRIII-γ |
| CD83 | CD28 | CD27 | FcεRIβ |
| CD83 | CD28 | CD27 | FcεRIγ |
| CD83 | CD28 | CD27 | DAP10 |
| CD83 | CD28 | CD27 | DAP12 |
| CD83 | CD28 | CD27 | CD32 |
| CD83 | CD28 | CD27 | CD79a |
| CD83 | CD28 | CD27 | CD79b |
| CD83 | CD28 | CD28δ | CD8 |
| CD83 | CD28 | CD28δ | CD3ζ |
| CD83 | CD28 | CD28δ | CD3δ |
| CD83 | CD28 | CD28δ | CD3γ |
| CD83 | CD28 | CD28δ | CD3ε |
| CD83 | CD28 | CD28δ | FcγRI-γ |
| CD83 | CD28 | CD28δ | FcγRIII-γ |
| CD83 | CD28 | CD28δ | FcεRIβ |
| CD83 | CD28 | CD28δ | FcεRIγ |
| CD83 | CD28 | CD28δ | DAP10 |
| CD83 | CD28 | CD28δ | DAP12 |
| CD83 | CD28 | CD28δ | CD32 |
| CD83 | CD28 | CD28δ | CD79a |
| CD83 | CD28 | CD28δ | CD79b |
| CD83 | CD28 | CD80 | CD8 |
| CD83 | CD28 | CD80 | CD3ζ |
| CD83 | CD28 | CD80 | CD3δ |
| CD83 | CD28 | CD80 | CD3γ |
| CD83 | CD28 | CD80 | CD3ε |
| CD83 | CD28 | CD80 | FcγRI-γ |
| CD83 | CD28 | CD80 | FcγRIII-γ |
| CD83 | CD28 | CD80 | FcεRIβ |
| CD83 | CD28 | CD80 | FcεRIγ |
| CD83 | CD28 | CD80 | DAP10 |
| CD83 | CD28 | CD80 | DAP12 |
| CD83 | CD28 | CD80 | CD32 |
| CD83 | CD28 | CD80 | CD79a |
| CD83 | CD28 | CD80 | CD79b |
| CD83 | CD28 | CD86 | CD8 |
| CD83 | CD28 | CD86 | CD3ζ |
| CD83 | CD28 | CD86 | CD3δ |
| CD83 | CD28 | CD86 | CD3γ |
| CD83 | CD28 | CD86 | CD3ε |
| CD83 | CD28 | CD86 | FcγRI-γ |
| CD83 | CD28 | CD86 | FcγRIII-γ |
| CD83 | CD28 | CD86 | FcεRIβ |
| CD83 | CD28 | CD86 | FcεRIγ |
| CD83 | CD28 | CD86 | DAP10 |
| CD83 | CD28 | CD86 | DAP12 |
| CD83 | CD28 | CD86 | CD32 |
| CD83 | CD28 | CD86 | CD79a |
| CD83 | CD28 | CD86 | CD79b |
| CD83 | CD28 | OX40 | CD8 |
| CD83 | CD28 | OX40 | CD3ζ |
| CD83 | CD28 | OX40 | CD3δ |
| CD83 | CD28 | OX40 | CD3γ |
| CD83 | CD28 | OX40 | CD3ε |
| CD83 | CD28 | OX40 | FcγRI-γ |
| CD83 | CD28 | OX40 | FcγRIII-γ |
| CD83 | CD28 | OX40 | FcεRIβ |
| CD83 | CD28 | OX40 | FcεRIγ |
| CD83 | CD28 | OX40 | DAP10 |
| CD83 | CD28 | OX40 | DAP12 |
| CD83 | CD28 | OX40 | CD32 |
| CD83 | CD28 | OX40 | CD79a |
| CD83 | CD28 | OX40 | CD79b |
| CD83 | CD28 | DAP10 | CD8 |
| CD83 | CD28 | DAP10 | CD3ζ |
| CD83 | CD28 | DAP10 | CD3δ |
| CD83 | CD28 | DAP10 | CD3γ |
| CD83 | CD28 | DAP10 | CD3ε |
| CD83 | CD28 | DAP10 | FcγRI-γ |
| CD83 | CD28 | DAP10 | FcγRIII-γ |
| CD83 | CD28 | DAP10 | FcεRIβ |
| CD83 | CD28 | DAP10 | FcεRIγ |
| CD83 | CD28 | DAP10 | DAP10 |
| CD83 | CD28 | DAP10 | DAP12 |
| CD83 | CD28 | DAP10 | CD32 |
| CD83 | CD28 | DAP10 | CD79a |
| CD83 | CD28 | DAP10 | CD79b |
| CD83 | CD28 | DAP12 | CD8 |
| CD83 | CD28 | DAP12 | CD3ζ |
| CD83 | CD28 | DAP12 | CD3δ |
| CD83 | CD28 | DAP12 | CD3γ |
| CD83 | CD28 | DAP12 | CD3ε |
| CD83 | CD28 | DAP12 | FcγRI-γ |
| CD83 | CD28 | DAP12 | FcγRIII-γ |
| CD83 | CD28 | DAP12 | FcεRIβ |
| CD83 | CD28 | DAP12 | FcεRIγ |
| CD83 | CD28 | DAP12 | DAP10 |
| CD83 | CD28 | DAP12 | DAP12 |
| CD83 | CD28 | DAP12 | CD32 |
| CD83 | CD28 | DAP12 | CD79a |
| CD83 | CD28 | DAP12 | CD79b |
| CD83 | CD28 | MyD88 | CD8 |
| CD83 | CD28 | MyD88 | CD3ζ |
| CD83 | CD28 | MyD88 | CD3δ |
| CD83 | CD28 | MyD88 | CD3γ |
| CD83 | CD28 | MyD88 | CD3ε |
| CD83 | CD28 | MyD88 | FcγRI-γ |
| CD83 | CD28 | MyD88 | FcγRIII-γ |
| CD83 | CD28 | MyD88 | FcεRIβ |
| CD83 | CD28 | MyD88 | FcεRIγ |
| CD83 | CD28 | MyD88 | DAP10 |
| CD83 | CD28 | MyD88 | DAP12 |
| CD83 | CD28 | MyD88 | CD32 |
| CD83 | CD28 | MyD88 | CD79a |
| CD83 | CD28 | MyD88 | CD79b |
| CD83 | CD28 | CD7 | CD8 |
| CD83 | CD28 | CD7 | CD3ζ |
| CD83 | CD28 | CD7 | CD3δ |
| CD83 | CD28 | CD7 | CD3γ |
| CD83 | CD28 | CD7 | CD3ε |
| CD83 | CD28 | CD7 | FcγRI-γ |
| CD83 | CD28 | CD7 | FcγRIII-γ |
| CD83 | CD28 | CD7 | FcεRIβ |
| CD83 | CD28 | CD7 | FcεRIγ |
| CD83 | CD28 | CD7 | DAP10 |
| CD83 | CD28 | CD7 | DAP12 |
| CD83 | CD28 | CD7 | CD32 |
| CD83 | CD28 | CD7 | CD79a |
| CD83 | CD28 | CD7 | CD79b |
| CD83 | CD28 | BTNL3 | CD8 |
| CD83 | CD28 | BTNL3 | CD3ζ |
| CD83 | CD28 | BTNL3 | CD3δ |
| CD83 | CD28 | BTNL3 | CD3γ |
| CD83 | CD28 | BTNL3 | CD3ε |
| CD83 | CD28 | BTNL3 | FcγRI-γ |
| CD83 | CD28 | BTNL3 | FcγRIII-γ |
| CD83 | CD28 | BTNL3 | FcεRIβ |
| CD83 | CD28 | BTNL3 | FcεRIγ |
| CD83 | CD28 | BTNL3 | DAP10 |
| CD83 | CD28 | BTNL3 | DAP12 |
| CD83 | CD28 | BTNL3 | CD32 |
| CD83 | CD28 | BTNL3 | CD79a |
| CD83 | CD28 | BTNL3 | CD79b |
| CD83 | CD28 | NKG2D | CD8 |
| CD83 | CD28 | NKG2D | CD3ζ |
| CD83 | CD28 | NKG2D | CD3δ |
| CD83 | CD28 | NKG2D | CD3γ |
| CD83 | CD28 | NKG2D | CD3ε |
| CD83 | CD28 | NKG2D | FcγRI-γ |
| CD83 | CD28 | NKG2D | FcγRIII-γ |
| CD83 | CD28 | NKG2D | FcεRIβ |
| CD83 | CD28 | NKG2D | FcεRIγ |
| CD83 | CD28 | NKG2D | DAP10 |
| CD83 | CD28 | NKG2D | DAP12 |
| CD83 | CD28 | NKG2D | CD32 |
| CD83 | CD28 | NKG2D | CD79a |
| CD83 | CD28 | NKG2D | CD79b |
| CD83 | CD8 | CD28 | CD8 |
| CD83 | CD8 | CD28 | CD3ζ |
| CD83 | CD8 | CD28 | CD3δ |
| CD83 | CD8 | CD28 | CD3γ |
| CD83 | CD8 | CD28 | CD3ε |
| CD83 | CD8 | CD28 | FcγRI-γ |
| CD83 | CD8 | CD28 | FcγRIII-γ |
| CD83 | CD8 | CD28 | FcεRIβ |
| CD83 | CD8 | CD28 | FcεRIγ |
| CD83 | CD8 | CD28 | DAP10 |
| CD83 | CD8 | CD28 | DAP12 |
| CD83 | CD8 | CD28 | CD32 |
| CD83 | CD8 | CD28 | CD79a |
| CD83 | CD8 | CD28 | CD79b |
| CD83 | CD8 | CD8 | CD8 |
| CD83 | CD8 | CD8 | CD3ζ |
| CD83 | CD8 | CD8 | CD3δ |
| CD83 | CD8 | CD8 | CD3γ |
| CD83 | CD8 | CD8 | CD3ε |
| CD83 | CD8 | CD8 | FcγRI-γ |
| CD83 | CD8 | CD8 | FcγRIII-γ |
| CD83 | CD8 | CD8 | FcεRIβ |
| CD83 | CD8 | CD8 | FcεRIγ |
| CD83 | CD8 | CD8 | DAP10 |
| CD83 | CD8 | CD8 | DAP12 |
| CD83 | CD8 | CD8 | CD32 |
| CD83 | CD8 | CD8 | CD79a |
| CD83 | CD8 | CD8 | CD79b |
| CD83 | CD8 | CD4 | CD8 |
| CD83 | CD8 | CD4 | CD3ζ |
| CD83 | CD8 | CD4 | CD3δ |
| CD83 | CD8 | CD4 | CD3γ |
| CD83 | CD8 | CD4 | CD3ε |
| CD83 | CD8 | CD4 | FcγRI-γ |
| CD83 | CD8 | CD4 | FcγRIII-γ |
| CD83 | CD8 | CD4 | FcεRIβ |
| CD83 | CD8 | CD4 | FcεRIγ |
| CD83 | CD8 | CD4 | DAP10 |
| CD83 | CD8 | CD4 | DAP12 |
| CD83 | CD8 | CD4 | CD32 |
| CD83 | CD8 | CD4 | CD79a |
| CD83 | CD8 | CD4 | CD79b |
| CD83 | CD8 | b2c | CD8 |
| CD83 | CD8 | b2c | CD3ζ |
| CD83 | CD8 | b2c | CD3δ |
| CD83 | CD8 | b2c | CD3γ |
| CD83 | CD8 | b2c | CD3ε |
| CD83 | CD8 | b2c | FcγRI-γ |
| CD83 | CD8 | b2c | FcγRIII-γ |
| CD83 | CD8 | b2c | FcεRIβ |
| CD83 | CD8 | b2c | FcεRIγ |
| CD83 | CD8 | b2c | DAP10 |
| CD83 | CD8 | b2c | DAP12 |
| CD83 | CD8 | b2c | CD32 |
| CD83 | CD8 | b2c | CD79a |
| CD83 | CD8 | b2c | CD79b |
| CD83 | CD8 | CD137/41BB | CD8 |
| CD83 | CD8 | CD137/41BB | CD3ζ |
| CD83 | CD8 | CD137/41BB | CD3δ |
| CD83 | CD8 | CD137/41BB | CD3γ |
| CD83 | CD8 | CD137/41BB | CD3ε |
| CD83 | CD8 | CD137/41BB | FcγRI-γ |
| CD83 | CD8 | CD137/41BB | FcγRIII-γ |
| CD83 | CD8 | CD137/41BB | FcεRIβ |
| CD83 | CD8 | CD137/41BB | FcεRIγ |
| CD83 | CD8 | CD137/41BB | DAP10 |
| CD83 | CD8 | CD137/41BB | DAP12 |
| CD83 | CD8 | CD137/41BB | CD32 |
| CD83 | CD8 | CD137/41BB | CD79a |
| CD83 | CD8 | CD137/41BB | CD79b |
| CD83 | CD8 | ICOS | CD8 |
| CD83 | CD8 | ICOS | CD3ζ |
| CD83 | CD8 | ICOS | CD3δ |
| CD83 | CD8 | ICOS | CD3γ |
| CD83 | CD8 | ICOS | CD3ε |
| CD83 | CD8 | ICOS | FcγRI-γ |
| CD83 | CD8 | ICOS | FcγRIII-γ |
| CD83 | CD8 | ICOS | FcεRIβ |
| CD83 | CD8 | ICOS | FcεRIγ |
| CD83 | CD8 | ICOS | DAP10 |
| CD83 | CD8 | ICOS | DAP12 |
| CD83 | CD8 | ICOS | CD32 |
| CD83 | CD8 | ICOS | CD79a |
| CD83 | CD8 | ICOS | CD79b |
| CD83 | CD8 | CD27 | CD8 |
| CD83 | CD8 | CD27 | CD3ζ |
| CD83 | CD8 | CD27 | CD3δ |
| CD83 | CD8 | CD27 | CD3γ |
| CD83 | CD8 | CD27 | CD3ε |
| CD83 | CD8 | CD27 | FcγRI-γ |
| CD83 | CD8 | CD27 | FcγRIII-γ |
| CD83 | CD8 | CD27 | FcεRIβ |
| CD83 | CD8 | CD27 | FcεRIγ |
| CD83 | CD8 | CD27 | DAP10 |
| CD83 | CD8 | CD27 | DAP12 |
| CD83 | CD8 | CD27 | CD32 |
| CD83 | CD8 | CD27 | CD79a |
| CD83 | CD8 | CD27 | CD79b |
| CD83 | CD8 | CD28δ | CD8 |
| CD83 | CD8 | CD28δ | CD3ζ |
| CD83 | CD8 | CD28δ | CD3δ |
| CD83 | CD8 | CD28δ | CD3γ |
| CD83 | CD8 | CD28δ | CD3ε |
| CD83 | CD8 | CD28δ | FcγRI-γ |
| CD83 | CD8 | CD28δ | FcγRIII-γ |
| CD83 | CD8 | CD28δ | FcεRIβ |
| CD83 | CD8 | CD28δ | FcεRIγ |
| CD83 | CD8 | CD28δ | DAP10 |
| CD83 | CD8 | CD28δ | DAP12 |
| CD83 | CD8 | CD28δ | CD32 |
| CD83 | CD8 | CD28δ | CD79a |
| CD83 | CD8 | CD28δ | CD79b |
| CD83 | CD8 | CD80 | CD8 |
| CD83 | CD8 | CD80 | CD3ζ |
| CD83 | CD8 | CD80 | CD3δ |
| CD83 | CD8 | CD80 | CD3γ |
| CD83 | CD8 | CD80 | CD3ε |
| CD83 | CD8 | CD80 | FcγRI-γ |
| CD83 | CD8 | CD80 | FcγRIII-γ |
| CD83 | CD8 | CD80 | FcεRIβ |
| CD83 | CD8 | CD80 | FcεRIγ |
| CD83 | CD8 | CD80 | DAP10 |
| CD83 | CD8 | CD80 | DAP12 |
| CD83 | CD8 | CD80 | CD32 |
| CD83 | CD8 | CD80 | CD79a |
| CD83 | CD8 | CD80 | CD79b |
| CD83 | CD8 | CD86 | CD8 |
| CD83 | CD8 | CD86 | CD3ζ |
| CD83 | CD8 | CD86 | CD3δ |
| CD83 | CD8 | CD86 | CD3γ |
| CD83 | CD8 | CD86 | CD3ε |
| CD83 | CD8 | CD86 | FcγRI-γ |
| CD83 | CD8 | CD86 | FcγRIII-γ |
| CD83 | CD8 | CD86 | FcεRIβ |
| CD83 | CD8 | CD86 | FcεRIγ |
| CD83 | CD8 | CD86 | DAP10 |
| CD83 | CD8 | CD86 | DAP12 |
| CD83 | CD8 | CD86 | CD32 |
| CD83 | CD8 | CD86 | CD79a |
| CD83 | CD8 | CD86 | CD79b |
| CD83 | CD8 | OX40 | CD8 |
| CD83 | CD8 | OX40 | CD3ζ |
| CD83 | CD8 | OX40 | CD3δ |
| CD83 | CD8 | OX40 | CD3γ |
| CD83 | CD8 | OX40 | CD3ε |
| CD83 | CD8 | OX40 | FcγRI-γ |
| CD83 | CD8 | OX40 | FcγRIII-γ |
| CD83 | CD8 | OX40 | FcεRIβ |
| CD83 | CD8 | OX40 | FcεRIγ |
| CD83 | CD8 | OX40 | DAP10 |
| CD83 | CD8 | OX40 | DAP12 |
| CD83 | CD8 | OX40 | CD32 |
| CD83 | CD8 | OX40 | CD79a |
| CD83 | CD8 | OX40 | CD79b |
| CD83 | CD8 | DAP10 | CD8 |
| CD83 | CD8 | DAP10 | CD3ζ |
| CD83 | CD8 | DAP10 | CD3δ |
| CD83 | CD8 | DAP10 | CD3γ |
| CD83 | CD8 | DAP10 | CD3ε |
| CD83 | CD8 | DAP10 | FcγRI-γ |
| CD83 | CD8 | DAP10 | FcγRIII-γ |
| CD83 | CD8 | DAP10 | FcεRIβ |
| CD83 | CD8 | DAP10 | FcεRIγ |
| CD83 | CD8 | DAP10 | DAP10 |
| CD83 | CD8 | DAP10 | DAP12 |
| CD83 | CD8 | DAP10 | CD32 |
| CD83 | CD8 | DAP10 | CD79a |
| CD83 | CD8 | DAP10 | CD79b |
| CD83 | CD8 | DAP12 | CD8 |
| CD83 | CD8 | DAP12 | CD3ζ |
| CD83 | CD8 | DAP12 | CD3δ |
| CD83 | CD8 | DAP12 | CD3γ |
| CD83 | CD8 | DAP12 | CD3ε |
| CD83 | CD8 | DAP12 | FcγRI-γ |
| CD83 | CD8 | DAP12 | FcγRIII-γ |
| CD83 | CD8 | DAP12 | FcεRIβ |
| CD83 | CD8 | DAP12 | FcεRIγ |
| CD83 | CD8 | DAP12 | DAP10 |
| CD83 | CD8 | DAP12 | DAP12 |
| CD83 | CD8 | DAP12 | CD32 |
| CD83 | CD8 | DAP12 | CD79a |
| CD83 | CD8 | DAP12 | CD79b |
| CD83 | CD8 | MyD88 | CD8 |
| CD83 | CD8 | MyD88 | CD3ζ |
| CD83 | CD8 | MyD88 | CD3δ |
| CD83 | CD8 | MyD88 | CD3γ |
| CD83 | CD8 | MyD88 | CD3ε |
| CD83 | CD8 | MyD88 | FcγRI-γ |
| CD83 | CD8 | MyD88 | FcγRIII-γ |
| CD83 | CD8 | MyD88 | FcεRIβ |
| CD83 | CD8 | MyD88 | FcεRIγ |
| CD83 | CD8 | MyD88 | DAP10 |
| CD83 | CD8 | MyD88 | DAP12 |
| CD83 | CD8 | MyD88 | CD32 |
| CD83 | CD8 | MyD88 | CD79a |
| CD83 | CD8 | MyD88 | CD79b |
| CD83 | CD8 | CD7 | CD8 |
| CD83 | CD8 | CD7 | CD3ζ |
| CD83 | CD8 | CD7 | CD3δ |
| CD83 | CD8 | CD7 | CD3γ |
| CD83 | CD8 | CD7 | CD3ε |
| CD83 | CD8 | CD7 | FcγRI-γ |
| CD83 | CD8 | CD7 | FcγRIII-γ |
| CD83 | CD8 | CD7 | FcεRIβ |
| CD83 | CD8 | CD7 | FcεRIγ |
| CD83 | CD8 | CD7 | DAP10 |
| CD83 | CD8 | CD7 | DAP12 |
| CD83 | CD8 | CD7 | CD32 |
| CD83 | CD8 | CD7 | CD79a |
| CD83 | CD8 | CD7 | CD79b |
| CD83 | CD8 | BTNL3 | CD8 |
| CD83 | CD8 | BTNL3 | CD3ζ |
| CD83 | CD8 | BTNL3 | CD3δ |
| CD83 | CD8 | BTNL3 | CD3γ |
| CD83 | CD8 | BTNL3 | CD3ε |
| CD83 | CD8 | BTNL3 | FcγRI-γ |
| CD83 | CD8 | BTNL3 | FcγRIII-γ |
| CD83 | CD8 | BTNL3 | FcεRIβ |
| CD83 | CD8 | BTNL3 | FcεRIγ |
| CD83 | CD8 | BTNL3 | DAP10 |
| CD83 | CD8 | BTNL3 | DAP12 |
| CD83 | CD8 | BTNL3 | CD32 |
| CD83 | CD8 | BTNL3 | CD79a |
| CD83 | CD8 | BTNL3 | CD79b |
| CD83 | CD8 | NKG2D | CD8 |
| CD83 | CD8 | NKG2D | CD3ζ |
| CD83 | CD8 | NKG2D | CD3δ |
| CD83 | CD8 | NKG2D | CD3γ |
| CD83 | CD8 | NKG2D | CD3ε |
| CD83 | CD8 | NKG2D | FcγRI-γ |
| CD83 | CD8 | NKG2D | FcγRIII-γ |
| CD83 | CD8 | NKG2D | FcεRIβ |
| CD83 | CD8 | NKG2D | FcεRIγ |
| CD83 | CD8 | NKG2D | DAP10 |
| CD83 | CD8 | NKG2D | DAP12 |
| CD83 | CD8 | NKG2D | CD32 |
| CD83 | CD8 | NKG2D | CD79a |
| CD83 | CD8 | NKG2D | CD79b |
| CD83 | CD4 | CD28 | CD8 |
| CD83 | CD4 | CD28 | CD3ζ |
| CD83 | CD4 | CD28 | CD3δ |
| CD83 | CD4 | CD28 | CD3γ |
| CD83 | CD4 | CD28 | CD3ε |
| CD83 | CD4 | CD28 | FcγRI-γ |
| CD83 | CD4 | CD28 | FcγRIII-γ |
| CD83 | CD4 | CD28 | FcεRIβ |
| CD83 | CD4 | CD28 | FcεRIγ |
| CD83 | CD4 | CD28 | DAP10 |
| CD83 | CD4 | CD28 | DAP12 |
| CD83 | CD4 | CD28 | CD32 |
| CD83 | CD4 | CD28 | CD79a |
| CD83 | CD4 | CD28 | CD79b |
| CD83 | CD4 | CD8 | CD8 |
| CD83 | CD4 | CD8 | CD3ζ |
| CD83 | CD4 | CD8 | CD3δ |
| CD83 | CD4 | CD8 | CD3γ |
| CD83 | CD4 | CD8 | CD3ε |
| CD83 | CD4 | CD8 | FcγRI-γ |
| CD83 | CD4 | CD8 | FcγRIII-γ |
| CD83 | CD4 | CD8 | FcεRIβ |
| CD83 | CD4 | CD8 | FcεRIγ |
| CD83 | CD4 | CD8 | DAP10 |
| CD83 | CD4 | CD8 | DAP12 |
| CD83 | CD4 | CD8 | CD32 |
| CD83 | CD4 | CD8 | CD79a |
| CD83 | CD4 | CD8 | CD79b |
| CD83 | CD4 | CD4 | CD8 |
| CD83 | CD4 | CD4 | CD3ζ |
| CD83 | CD4 | CD4 | CD3δ |
| CD83 | CD4 | CD4 | CD3γ |
| CD83 | CD4 | CD4 | CD3ε |
| CD83 | CD4 | CD4 | FcγRI-γ |
| CD83 | CD4 | CD4 | FcγRIII-γ |
| CD83 | CD4 | CD4 | FcεRIβ |
| CD83 | CD4 | CD4 | FcεRIγ |
| CD83 | CD4 | CD4 | DAP10 |
| CD83 | CD4 | CD4 | DAP12 |
| CD83 | CD4 | CD4 | CD32 |
| CD83 | CD4 | CD4 | CD79a |
| CD83 | CD4 | CD4 | CD79b |
| CD83 | CD4 | b2c | CD8 |
| CD83 | CD4 | b2c | CD3ζ |
| CD83 | CD4 | b2c | CD3δ |
| CD83 | CD4 | b2c | CD3γ |
| CD83 | CD4 | b2c | CD3ε |
| CD83 | CD4 | b2c | FcγRI-γ |
| CD83 | CD4 | b2c | FcγRIII-γ |
| CD83 | CD4 | b2c | FcεRIβ |
| CD83 | CD4 | b2c | FcεRIγ |
| CD83 | CD4 | b2c | DAP10 |
| CD83 | CD4 | b2c | DAP12 |
| CD83 | CD4 | b2c | CD32 |
| CD83 | CD4 | b2c | CD79a |
| CD83 | CD4 | b2c | CD79b |
| CD83 | CD4 | CD137/41BB | CD8 |
| CD83 | CD4 | CD137/41BB | CD3ζ |
| CD83 | CD4 | CD137/41BB | CD3δ |
| CD83 | CD4 | CD137/41BB | CD3γ |
| CD83 | CD4 | CD137/41BB | CD3ε |
| CD83 | CD4 | CD137/41BB | FcγRI-γ |
| CD83 | CD4 | CD137/41BB | FcγRIII-γ |
| CD83 | CD4 | CD137/41BB | FcεRIβ |
| CD83 | CD4 | CD137/41BB | FcεRIγ |
| CD83 | CD4 | CD137/41BB | DAP10 |
| CD83 | CD4 | CD137/41BB | DAP12 |
| CD83 | CD4 | CD137/41BB | CD32 |
| CD83 | CD4 | CD137/41BB | CD79a |
| CD83 | CD4 | CD137/41BB | CD79b |
| CD83 | CD4 | ICOS | CD8 |
| CD83 | CD4 | ICOS | CD3ζ |
| CD83 | CD4 | ICOS | CD3δ |
| CD83 | CD4 | ICOS | CD3γ |
| CD83 | CD4 | ICOS | CD3ε |
| CD83 | CD4 | ICOS | FcγRI-γ |
| CD83 | CD4 | ICOS | FcγRIII-γ |
| CD83 | CD4 | ICOS | FcεRIβ |
| CD83 | CD4 | ICOS | FcεRIγ |
| CD83 | CD4 | ICOS | DAP10 |
| CD83 | CD4 | ICOS | DAP12 |
| CD83 | CD4 | ICOS | CD32 |
| CD83 | CD4 | ICOS | CD79a |
| CD83 | CD4 | ICOS | CD79b |
| CD83 | CD4 | CD27 | CD8 |
| CD83 | CD4 | CD27 | CD3ζ |
| CD83 | CD4 | CD27 | CD3δ |
| CD83 | CD4 | CD27 | CD3γ |
| CD83 | CD4 | CD27 | CD3ε |
| CD83 | CD4 | CD27 | FcγRI-γ |
| CD83 | CD4 | CD27 | FcγRIII-γ |
| CD83 | CD4 | CD27 | FcεRIβ |
| CD83 | CD4 | CD27 | FcεRIγ |
| CD83 | CD4 | CD27 | DAP10 |
| CD83 | CD4 | CD27 | DAP12 |
| CD83 | CD4 | CD27 | CD32 |
| CD83 | CD4 | CD27 | CD79a |
| CD83 | CD4 | CD27 | CD79b |
| CD83 | CD4 | CD28δ | CD8 |
| CD83 | CD4 | CD28δ | CD3ζ |
| CD83 | CD4 | CD28δ | CD3δ |
| CD83 | CD4 | CD28δ | CD3γ |
| CD83 | CD4 | CD28δ | CD3ε |
| CD83 | CD4 | CD28δ | FcγRI-γ |
| CD83 | CD4 | CD28δ | FcγRIII-γ |
| CD83 | CD4 | CD28δ | FcεRIβ |
| CD83 | CD4 | CD28δ | FcεRIγ |
| CD83 | CD4 | CD28δ | DAP10 |
| CD83 | CD4 | CD28δ | DAP12 |
| CD83 | CD4 | CD28δ | CD32 |
| CD83 | CD4 | CD28δ | CD79a |
| CD83 | CD4 | CD28δ | CD79b |
| CD83 | CD4 | CD80 | CD8 |
| CD83 | CD4 | CD80 | CD3ζ |
| CD83 | CD4 | CD80 | CD3δ |
| CD83 | CD4 | CD80 | CD3γ |
| CD83 | CD4 | CD80 | CD3ε |
| CD83 | CD4 | CD80 | FcγRI-γ |
| CD83 | CD4 | CD80 | FcγRIII-γ |
| CD83 | CD4 | CD80 | FcεRIβ |
| CD83 | CD4 | CD80 | FcεRIγ |
| CD83 | CD4 | CD80 | DAP10 |
| CD83 | CD4 | CD80 | DAP12 |
| CD83 | CD4 | CD80 | CD32 |
| CD83 | CD4 | CD80 | CD79a |
| CD83 | CD4 | CD80 | CD79b |
| CD83 | CD4 | CD86 | CD8 |
| CD83 | CD4 | CD86 | CD3ζ |
| CD83 | CD4 | CD86 | CD3δ |
| CD83 | CD4 | CD86 | CD3γ |
| CD83 | CD4 | CD86 | CD3ε |
| CD83 | CD4 | CD86 | FcγRI-γ |
| CD83 | CD4 | CD86 | FcγRIII-γ |
| CD83 | CD4 | CD86 | FcεRIβ |
| CD83 | CD4 | CD86 | FcεRIγ |
| CD83 | CD4 | CD86 | DAP10 |
| CD83 | CD4 | CD86 | DAP12 |
| CD83 | CD4 | CD86 | CD32 |
| CD83 | CD4 | CD86 | CD79a |
| CD83 | CD4 | CD86 | CD79b |
| CD83 | CD4 | OX40 | CD8 |
| CD83 | CD4 | OX40 | CD3ζ |
| CD83 | CD4 | OX40 | CD3δ |
| CD83 | CD4 | OX40 | CD3γ |
| CD83 | CD4 | OX40 | CD3ε |
| CD83 | CD4 | OX40 | FcγRI-γ |
| CD83 | CD4 | OX40 | FcγRIII-γ |
| CD83 | CD4 | OX40 | FcεRIβ |
| CD83 | CD4 | OX40 | FcεRIγ |
| CD83 | CD4 | OX40 | DAP10 |
| CD83 | CD4 | OX40 | DAP12 |
| CD83 | CD4 | OX40 | CD32 |
| CD83 | CD4 | OX40 | CD79a |
| CD83 | CD4 | OX40 | CD79b |
| CD83 | CD4 | DAP10 | CD8 |
| CD83 | CD4 | DAP10 | CD3ζ |
| CD83 | CD4 | DAP10 | CD3δ |
| CD83 | CD4 | DAP10 | CD3γ |
| CD83 | CD4 | DAP10 | CD3ε |
| CD83 | CD4 | DAP10 | FcγRI-γ |
| CD83 | CD4 | DAP10 | FcγRIII-γ |
| CD83 | CD4 | DAP10 | FcεRIβ |
| CD83 | CD4 | DAP10 | FcεRIγ |
| CD83 | CD4 | DAP10 | DAP10 |
| CD83 | CD4 | DAP10 | DAP12 |
| CD83 | CD4 | DAP10 | CD32 |
| CD83 | CD4 | DAP10 | CD79a |
| CD83 | CD4 | DAP10 | CD79b |
| CD83 | CD4 | DAP12 | CD8 |
| CD83 | CD4 | DAP12 | CD3ζ |
| CD83 | CD4 | DAP12 | CD3δ |
| CD83 | CD4 | DAP12 | CD3γ |
| CD83 | CD4 | DAP12 | CD3ε |
| CD83 | CD4 | DAP12 | FcγRI-γ |
| CD83 | CD4 | DAP12 | FcγRIII-γ |
| CD83 | CD4 | DAP12 | FcεRIβ |
| CD83 | CD4 | DAP12 | FcεRIγ |
| CD83 | CD4 | DAP12 | DAP10 |
| CD83 | CD4 | DAP12 | DAP12 |
| CD83 | CD4 | DAP12 | CD32 |
| CD83 | CD4 | DAP12 | CD79a |
| CD83 | CD4 | DAP12 | CD79b |
| CD83 | CD4 | MyD88 | CD8 |
| CD83 | CD4 | MyD88 | CD3ζ |
| CD83 | CD4 | MyD88 | CD3δ |
| CD83 | CD4 | MyD88 | CD3γ |
| CD83 | CD4 | MyD88 | CD3ε |
| CD83 | CD4 | MyD88 | FcγRI-γ |
| CD83 | CD4 | MyD88 | FcγRIII-γ |
| CD83 | CD4 | MyD88 | FcεRIβ |
| CD83 | CD4 | MyD88 | FcεRIγ |
| CD83 | CD4 | MyD88 | DAP10 |
| CD83 | CD4 | MyD88 | DAP12 |
| CD83 | CD4 | MyD88 | CD32 |
| CD83 | CD4 | MyD88 | CD79a |
| CD83 | CD4 | MyD88 | CD79b |
| CD83 | CD4 | CD7 | CD8 |
| CD83 | CD4 | CD7 | CD3ζ |
| CD83 | CD4 | CD7 | CD3δ |
| CD83 | CD4 | CD7 | CD3γ |
| CD83 | CD4 | CD7 | CD3ε |
| CD83 | CD4 | CD7 | FcγRI-γ |
| CD83 | CD4 | CD7 | FcγRIII-γ |
| CD83 | CD4 | CD7 | FcεRIβ |
| CD83 | CD4 | CD7 | FcεRIγ |
| CD83 | CD4 | CD7 | DAP10 |
| CD83 | CD4 | CD7 | DAP12 |
| CD83 | CD4 | CD7 | CD32 |
| CD83 | CD4 | CD7 | CD79a |
| CD83 | CD4 | CD7 | CD79b |
| CD83 | CD4 | BTNL3 | CD8 |
| CD83 | CD4 | BTNL3 | CD3ζ |
| CD83 | CD4 | BTNL3 | CD3δ |
| CD83 | CD4 | BTNL3 | CD3γ |
| CD83 | CD4 | BTNL3 | CD3ε |
| CD83 | CD4 | BTNL3 | FcγRI-γ |
| CD83 | CD4 | BTNL3 | FcγRIII-γ |
| CD83 | CD4 | BTNL3 | FcεRIβ |
| CD83 | CD4 | BTNL3 | FcεRIγ |
| CD83 | CD4 | BTNL3 | DAP10 |
| CD83 | CD4 | BTNL3 | DAP12 |
| CD83 | CD4 | BTNL3 | CD32 |
| CD83 | CD4 | BTNL3 | CD79a |
| CD83 | CD4 | BTNL3 | CD79b |
| CD83 | CD4 | NKG2D | CD8 |
| CD83 | CD4 | NKG2D | CD3ζ |
| CD83 | CD4 | NKG2D | CD3δ |
| CD83 | CD4 | NKG2D | CD3γ |
| CD83 | CD4 | NKG2D | CD3ε |
| CD83 | CD4 | NKG2D | FcγRI-γ |
| CD83 | CD4 | NKG2D | FcγRIII-γ |
| CD83 | CD4 | NKG2D | FcεRIβ |
| CD83 | CD4 | NKG2D | FcεRIγ |
| CD83 | CD4 | NKG2D | DAP10 |
| CD83 | CD4 | NKG2D | DAP12 |
| CD83 | CD4 | NKG2D | CD32 |
| CD83 | CD4 | NKG2D | CD79a |
| CD83 | CD4 | NKG2D | CD79b |
| CD83 | b2c | CD28 | CD8 |
| CD83 | b2c | CD28 | CD3ζ |
| CD83 | b2c | CD28 | CD3δ |
| CD83 | b2c | CD28 | CD3γ |
| CD83 | b2c | CD28 | CD3ε |
| CD83 | b2c | CD28 | FcγRI-γ |
| CD83 | b2c | CD28 | FcγRIII-γ |
| CD83 | b2c | CD28 | FcεRIβ |
| CD83 | b2c | CD28 | FcεRIγ |
| CD83 | b2c | CD28 | DAP10 |
| CD83 | b2c | CD28 | DAP12 |
| CD83 | b2c | CD28 | CD32 |
| CD83 | b2c | CD28 | CD79a |
| CD83 | b2c | CD28 | CD79b |
| CD83 | b2c | CD8 | CD8 |
| CD83 | b2c | CD8 | CD3ζ |
| CD83 | b2c | CD8 | CD3δ |
| CD83 | b2c | CD8 | CD3γ |
| CD83 | b2c | CD8 | CD3ε |
| CD83 | b2c | CD8 | FcγRI-γ |
| CD83 | b2c | CD8 | FcγRIII-γ |
| CD83 | b2c | CD8 | FcεRIβ |
| CD83 | b2c | CD8 | FcεRIγ |
| CD83 | b2c | CD8 | DAP10 |
| CD83 | b2c | CD8 | DAP12 |
| CD83 | b2c | CD8 | CD32 |
| CD83 | b2c | CD8 | CD79a |
| CD83 | b2c | CD8 | CD79b |
| CD83 | b2c | CD4 | CD8 |
| CD83 | b2c | CD4 | CD3ζ |
| CD83 | b2c | CD4 | CD3δ |
| CD83 | b2c | CD4 | CD3γ |
| CD83 | b2c | CD4 | CD3ε |
| CD83 | b2c | CD4 | FcγRI-γ |
| CD83 | b2c | CD4 | FcγRIII-γ |
| CD83 | b2c | CD4 | FcεRIβ |
| CD83 | b2c | CD4 | FcεRIγ |
| CD83 | b2c | CD4 | DAP10 |
| CD83 | b2c | CD4 | DAP12 |
| CD83 | b2c | CD4 | CD32 |
| CD83 | b2c | CD4 | CD79a |
| CD83 | b2c | CD4 | CD79b |
| CD83 | b2c | b2c | CD8 |
| CD83 | b2c | b2c | CD3ζ |
| CD83 | b2c | b2c | CD3δ |
| CD83 | b2c | b2c | CD3γ |
| CD83 | b2c | b2c | CD3ε |
| CD83 | b2c | b2c | FcγRI-γ |
| CD83 | b2c | b2c | FcγRIII-γ |
| CD83 | b2c | b2c | FcεRIβ |
| CD83 | b2c | b2c | FcεRIγ |
| CD83 | b2c | b2c | DAP10 |
| CD83 | b2c | b2c | DAP12 |
| CD83 | b2c | b2c | CD32 |
| CD83 | b2c | b2c | CD79a |
| CD83 | b2c | b2c | CD79b |
| CD83 | b2c | CD137/41BB | CD8 |
| CD83 | b2c | CD137/41BB | CD3ζ |
| CD83 | b2c | CD137/41BB | CD3δ |
| CD83 | b2c | CD137/41BB | CD3γ |
| CD83 | b2c | CD137/41BB | CD3ε |
| CD83 | b2c | CD137/41BB | FcγRI-γ |
| CD83 | b2c | CD137/41BB | FcγRIII-γ |
| CD83 | b2c | CD137/41BB | FcεRIβ |
| CD83 | b2c | CD137/41BB | FcεRIγ |
| CD83 | b2c | CD137/41BB | DAP10 |
| CD83 | b2c | CD137/41BB | DAP12 |
| CD83 | b2c | CD137/41BB | CD32 |
| CD83 | b2c | CD137/41BB | CD79a |
| CD83 | b2c | CD137/41BB | CD79b |
| CD83 | b2c | ICOS | CD8 |
| CD83 | b2c | ICOS | CD3ζ |
| CD83 | b2c | ICOS | CD3δ |
| CD83 | b2c | ICOS | CD3γ |
| CD83 | b2c | ICOS | CD3ε |
| CD83 | b2c | ICOS | FcγRI-γ |
| CD83 | b2c | ICOS | FcγRIII-γ |
| CD83 | b2c | ICOS | FcεRIβ |
| CD83 | b2c | ICOS | FcεRIγ |
| CD83 | b2c | ICOS | DAP10 |
| CD83 | b2c | ICOS | DAP12 |
| CD83 | b2c | ICOS | CD32 |
| CD83 | b2c | ICOS | CD79a |
| CD83 | b2c | ICOS | CD79b |
| CD83 | b2c | CD27 | CD8 |
| CD83 | b2c | CD27 | CD3ζ |
| CD83 | b2c | CD27 | CD3δ |
| CD83 | b2c | CD27 | CD3γ |
| CD83 | b2c | CD27 | CD3ε |
| CD83 | b2c | CD27 | FcγRI-γ |
| CD83 | b2c | CD27 | FcγRIII-γ |
| CD83 | b2c | CD27 | FcεRIβ |
| CD83 | b2c | CD27 | FcεRIγ |
| CD83 | b2c | CD27 | DAP10 |
| CD83 | b2c | CD27 | DAP12 |
| CD83 | b2c | CD27 | CD32 |
| CD83 | b2c | CD27 | CD79a |
| CD83 | b2c | CD27 | CD79b |
| CD83 | b2c | CD28δ | CD8 |
| CD83 | b2c | CD28δ | CD3ζ |
| CD83 | b2c | CD28δ | CD3δ |
| CD83 | b2c | CD28δ | CD3γ |
| CD83 | b2c | CD28δ | CD3ε |
| CD83 | b2c | CD28δ | FcγRI-γ |
| CD83 | b2c | CD28δ | FcγRIII-γ |
| CD83 | b2c | CD28δ | FcεRIβ |
| CD83 | b2c | CD28δ | FcεRIγ |
| CD83 | b2c | CD28δ | DAP10 |
| CD83 | b2c | CD28δ | DAP12 |
| CD83 | b2c | CD28δ | CD32 |
| CD83 | b2c | CD28δ | CD79a |
| CD83 | b2c | CD28δ | CD79b |
| CD83 | b2c | CD80 | CD8 |
| CD83 | b2c | CD80 | CD3ζ |
| CD83 | b2c | CD80 | CD3δ |
| CD83 | b2c | CD80 | CD3γ |
| CD83 | b2c | CD80 | CD3ε |
| CD83 | b2c | CD80 | FcγRI-γ |
| CD83 | b2c | CD80 | FcγRIII-γ |
| CD83 | b2c | CD80 | FcεRIβ |
| CD83 | b2c | CD80 | FcεRIγ |
| CD83 | b2c | CD80 | DAP10 |
| CD83 | b2c | CD80 | DAP12 |
| CD83 | b2c | CD80 | CD32 |
| CD83 | b2c | CD80 | CD79a |
| CD83 | b2c | CD80 | CD79b |
| CD83 | b2c | CD86 | CD8 |
| CD83 | b2c | CD86 | CD3ζ |
| CD83 | b2c | CD86 | CD3δ |
| CD83 | b2c | CD86 | CD3γ |
| CD83 | b2c | CD86 | CD3ε |
| CD83 | b2c | CD86 | FcγRI-γ |
| CD83 | b2c | CD86 | FcγRIII-γ |
| CD83 | b2c | CD86 | FcεRIβ |
| CD83 | b2c | CD86 | FcεRIγ |
| CD83 | b2c | CD86 | DAP10 |
| CD83 | b2c | CD86 | DAP12 |
| CD83 | b2c | CD86 | CD32 |
| CD83 | b2c | CD86 | CD79a |
| CD83 | b2c | CD86 | CD79b |
| CD83 | b2c | OX40 | CD8 |
| CD83 | b2c | OX40 | CD3ζ |
| CD83 | b2c | OX40 | CD3δ |
| CD83 | b2c | OX40 | CD3γ |
| CD83 | b2c | OX40 | CD3ε |
| CD83 | b2c | OX40 | FcγRI-γ |
| CD83 | b2c | OX40 | FcγRIII-γ |
| CD83 | b2c | OX40 | FcεRIβ |
| CD83 | b2c | OX40 | FcεRIγ |
| CD83 | b2c | OX40 | DAP10 |
| CD83 | b2c | OX40 | DAP12 |
| CD83 | b2c | OX40 | CD32 |
| CD83 | b2c | OX40 | CD79a |
| CD83 | b2c | OX40 | CD79b |
| CD83 | b2c | DAP10 | CD8 |
| CD83 | b2c | DAP10 | CD3ζ |
| CD83 | b2c | DAP10 | CD3δ |
| CD83 | b2c | DAP10 | CD3γ |
| CD83 | b2c | DAP10 | CD3ε |
| CD83 | b2c | DAP10 | FcγRI-γ |
| CD83 | b2c | DAP10 | FcγRIII-γ |
| CD83 | b2c | DAP10 | FcεRIβ |
| CD83 | b2c | DAP10 | FcεRIγ |
| CD83 | b2c | DAP10 | DAP10 |
| CD83 | b2c | DAP10 | DAP12 |
| CD83 | b2c | DAP10 | CD32 |
| CD83 | b2c | DAP10 | CD79a |
| CD83 | b2c | DAP10 | CD79b |
| CD83 | b2c | DAP12 | CD8 |
| CD83 | b2c | DAP12 | CD3ζ |
| CD83 | b2c | DAP12 | CD3δ |
| CD83 | b2c | DAP12 | CD3γ |
| CD83 | b2c | DAP12 | CD3ε |
| CD83 | b2c | DAP12 | FcγRI-γ |
| CD83 | b2c | DAP12 | FcγRIII-γ |
| CD83 | b2c | DAP12 | FcεRIβ |
| CD83 | b2c | DAP12 | FcεRIγ |
| CD83 | b2c | DAP12 | DAP10 |
| CD83 | b2c | DAP12 | DAP12 |
| CD83 | b2c | DAP12 | CD32 |
| CD83 | b2c | DAP12 | CD79a |
| CD83 | b2c | DAP12 | CD79b |
| CD83 | b2c | MyD88 | CD8 |
| CD83 | b2c | MyD88 | CD3ζ |
| CD83 | b2c | MyD88 | CD3δ |
| CD83 | b2c | MyD88 | CD3γ |
| CD83 | b2c | MyD88 | CD3ε |
| CD83 | b2c | MyD88 | FcγRI-γ |
| CD83 | b2c | MyD88 | FcγRIII-γ |
| CD83 | b2c | MyD88 | FcεRIβ |
| CD83 | b2c | MyD88 | FcεRIγ |
| CD83 | b2c | MyD88 | DAP10 |
| CD83 | b2c | MyD88 | DAP12 |
| CD83 | b2c | MyD88 | CD32 |
| CD83 | b2c | MyD88 | CD79a |
| CD83 | b2c | MyD88 | CD79b |
| CD83 | b2c | CD7 | CD8 |
| CD83 | b2c | CD7 | CD3ζ |
| CD83 | b2c | CD7 | CD3δ |
| CD83 | b2c | CD7 | CD3γ |
| CD83 | b2c | CD7 | CD3ε |
| CD83 | b2c | CD7 | FcγRI-γ |
| CD83 | b2c | CD7 | FcγRIII-γ |
| CD83 | b2c | CD7 | FcεRIβ |
| CD83 | b2c | CD7 | FcεRIγ |
| CD83 | b2c | CD7 | DAP10 |
| CD83 | b2c | CD7 | DAP12 |
| CD83 | b2c | CD7 | CD32 |
| CD83 | b2c | CD7 | CD79a |
| CD83 | b2c | CD7 | CD79b |
| CD83 | b2c | BTNL3 | CD8 |
| CD83 | b2c | BTNL3 | CD3ζ |
| CD83 | b2c | BTNL3 | CD3δ |
| CD83 | b2c | BTNL3 | CD3γ |
| CD83 | b2c | BTNL3 | CD3ε |
| CD83 | b2c | BTNL3 | FcγRI-γ |
| CD83 | b2c | BTNL3 | FcγRIII-γ |
| CD83 | b2c | BTNL3 | FcεRIβ |
| CD83 | b2c | BTNL3 | FcεRIγ |
| CD83 | b2c | BTNL3 | DAP10 |
| CD83 | b2c | BTNL3 | DAP12 |
| CD83 | b2c | BTNL3 | CD32 |
| CD83 | b2c | BTNL3 | CD79a |
| CD83 | b2c | BTNL3 | CD79b |
| CD83 | b2c | NKG2D | CD8 |
| CD83 | b2c | NKG2D | CD3ζ |
| CD83 | b2c | NKG2D | CD3δ |
| CD83 | b2c | NKG2D | CD3γ |
| CD83 | b2c | NKG2D | CD3ε |
| CD83 | b2c | NKG2D | FcγRI-γ |
| CD83 | b2c | NKG2D | FcγRIII-γ |
| CD83 | b2c | NKG2D | FcεRIβ |
| CD83 | b2c | NKG2D | FcεRIγ |
| CD83 | b2c | NKG2D | DAP10 |
| CD83 | b2c | NKG2D | DAP12 |
| CD83 | b2c | NKG2D | CD32 |
| CD83 | b2c | NKG2D | CD79a |
| CD83 | b2c | NKG2D | CD79b |
| CD83 | CD137/41BB | CD28 | CD8 |
| CD83 | CD137/41BB | CD28 | CD3ζ |
| CD83 | CD137/41BB | CD28 | CD3δ |
| CD83 | CD137/41BB | CD28 | CD3γ |
| CD83 | CD137/41BB | CD28 | CD3ε |
| CD83 | CD137/41BB | CD28 | FcγRI-γ |
| CD83 | CD137/41BB | CD28 | FcγRIII-γ |
| CD83 | CD137/41BB | CD28 | FcεRIβ |
| CD83 | CD137/41BB | CD28 | FcεRIγ |
| CD83 | CD137/41BB | CD28 | DAP10 |
| CD83 | CD137/41BB | CD28 | DAP12 |
| CD83 | CD137/41BB | CD28 | CD32 |
| CD83 | CD137/41BB | CD28 | CD79a |
| CD83 | CD137/41BB | CD28 | CD79b |
| CD83 | CD137/41BB | CD8 | CD8 |
| CD83 | CD137/41BB | CD8 | CD3ζ |
| CD83 | CD137/41BB | CD8 | CD3δ |
| CD83 | CD137/41BB | CD8 | CD3γ |
| CD83 | CD137/41BB | CD8 | CD3ε |
| CD83 | CD137/41BB | CD8 | FcγRI-γ |
| CD83 | CD137/41BB | CD8 | FcγRIII-γ |
| CD83 | CD137/41BB | CD8 | FcεRIβ |
| CD83 | CD137/41BB | CD8 | FcεRIγ |
| CD83 | CD137/41BB | CD8 | DAP10 |
| CD83 | CD137/41BB | CD8 | DAP12 |
| CD83 | CD137/41BB | CD8 | CD32 |
| CD83 | CD137/41BB | CD8 | CD79a |
| CD83 | CD137/41BB | CD8 | CD79b |
| CD83 | CD137/41BB | CD4 | CD8 |
| CD83 | CD137/41BB | CD4 | CD3ζ |
| CD83 | CD137/41BB | CD4 | CD3δ |
| CD83 | CD137/41BB | CD4 | CD3γ |
| CD83 | CD137/41BB | CD4 | CD3ε |
| CD83 | CD137/41BB | CD4 | FcγRI-γ |
| CD83 | CD137/41BB | CD4 | FcγRIII-γ |
| CD83 | CD137/41BB | CD4 | FcεRIβ |
| CD83 | CD137/41BB | CD4 | FcεRIγ |
| CD83 | CD137/41BB | CD4 | DAP10 |
| CD83 | CD137/41BB | CD4 | DAP12 |
| CD83 | CD137/41BB | CD4 | CD32 |
| CD83 | CD137/41BB | CD4 | CD79a |
| CD83 | CD137/41BB | CD4 | CD79b |
| CD83 | CD137/41BB | b2c | CD8 |
| CD83 | CD137/41BB | b2c | CD3ζ |
| CD83 | CD137/41BB | b2c | CD3δ |
| CD83 | CD137/41BB | b2c | CD3γ |
| CD83 | CD137/41BB | b2c | CD3ε |
| CD83 | CD137/41BB | b2c | FcγRI-γ |
| CD83 | CD137/41BB | b2c | FcγRIII-γ |
| CD83 | CD137/41BB | b2c | FcεRIβ |
| CD83 | CD137/41BB | b2c | FcεRIγ |
| CD83 | CD137/41BB | b2c | DAP10 |
| CD83 | CD137/41BB | b2c | DAP12 |
| CD83 | CD137/41BB | b2c | CD32 |
| CD83 | CD137/41BB | b2c | CD79a |
| CD83 | CD137/41BB | b2c | CD79b |
| CD83 | CD137/41BB | CD137/41BB | CD8 |
| CD83 | CD137/41BB | CD137/41BB | CD3ζ |
| CD83 | CD137/41BB | CD137/41BB | CD3δ |
| CD83 | CD137/41BB | CD137/41BB | CD3γ |
| CD83 | CD137/41BB | CD137/41BB | CD3ε |
| CD83 | CD137/41BB | CD137/41BB | FcγRI-γ |
| CD83 | CD137/41BB | CD137/41BB | FcγRIII-γ |
| CD83 | CD137/41BB | CD137/41BB | FcεRIβ |
| CD83 | CD137/41BB | CD137/41BB | FcεRIγ |
| CD83 | CD137/41BB | CD137/41BB | DAP10 |
| CD83 | CD137/41BB | CD137/41BB | DAP12 |
| CD83 | CD137/41BB | CD137/41BB | CD32 |
| CD83 | CD137/41BB | CD137/41BB | CD79a |
| CD83 | CD137/41BB | CD137/41BB | CD79b |
| CD83 | CD137/41BB | ICOS | CD8 |
| CD83 | CD137/41BB | ICOS | CD3ζ |
| CD83 | CD137/41BB | ICOS | CD3δ |
| CD83 | CD137/41BB | ICOS | CD3γ |
| CD83 | CD137/41BB | ICOS | CD3ε |
| CD83 | CD137/41BB | ICOS | FcγRI-γ |
| CD83 | CD137/41BB | ICOS | FcγRIII-γ |
| CD83 | CD137/41BB | ICOS | FcεRIβ |
| CD83 | CD137/41BB | ICOS | FcεRIγ |
| CD83 | CD137/41BB | ICOS | DAP10 |
| CD83 | CD137/41BB | ICOS | DAP12 |
| CD83 | CD137/41BB | ICOS | CD32 |
| CD83 | CD137/41BB | ICOS | CD79a |
| CD83 | CD137/41BB | ICOS | CD79b |
| CD83 | CD137/41BB | CD27 | CD8 |
| CD83 | CD137/41BB | CD27 | CD3ζ |
| CD83 | CD137/41BB | CD27 | CD3δ |
| CD83 | CD137/41BB | CD27 | CD3γ |
| CD83 | CD137/41BB | CD27 | CD3ε |
| CD83 | CD137/41BB | CD27 | FcγRI-γ |
| CD83 | CD137/41BB | CD27 | FcγRIII-γ |
| CD83 | CD137/41BB | CD27 | FcεRIβ |
| CD83 | CD137/41BB | CD27 | FcεRIγ |
| CD83 | CD137/41BB | CD27 | DAP10 |
| CD83 | CD137/41BB | CD27 | DAP12 |
| CD83 | CD137/41BB | CD27 | CD32 |
| CD83 | CD137/41BB | CD27 | CD79a |
| CD83 | CD137/41BB | CD27 | CD79b |
| CD83 | CD137/41BB | CD28δ | CD8 |
| CD83 | CD137/41BB | CD28δ | CD3ζ |
| CD83 | CD137/41BB | CD28δ | CD3δ |
| CD83 | CD137/41BB | CD28δ | CD3γ |
| CD83 | CD137/41BB | CD28δ | CD3ε |
| CD83 | CD137/41BB | CD28δ | FcγRI-γ |
| CD83 | CD137/41BB | CD28δ | FcγRIII-γ |
| CD83 | CD137/41BB | CD28δ | FcεRIβ |
| CD83 | CD137/41BB | CD28δ | FcεRIγ |
| CD83 | CD137/41BB | CD28δ | DAP10 |
| CD83 | CD137/41BB | CD28δ | DAP12 |
| CD83 | CD137/41BB | CD28δ | CD32 |
| CD83 | CD137/41BB | CD28δ | CD79a |
| CD83 | CD137/41BB | CD28δ | CD79b |
| CD83 | CD137/41BB | CD80 | CD8 |
| CD83 | CD137/41BB | CD80 | CD3ζ |
| CD83 | CD137/41BB | CD80 | CD3δ |
| CD83 | CD137/41BB | CD80 | CD3γ |
| CD83 | CD137/41BB | CD80 | CD3ε |
| CD83 | CD137/41BB | CD80 | FcγRI-γ |
| CD83 | CD137/41BB | CD80 | FcγRIII-γ |
| CD83 | CD137/41BB | CD80 | FcεRIβ |
| CD83 | CD137/41BB | CD80 | FcεRIγ |
| CD83 | CD137/41BB | CD80 | DAP10 |
| CD83 | CD137/41BB | CD80 | DAP12 |
| CD83 | CD137/41BB | CD80 | CD32 |
| CD83 | CD137/41BB | CD80 | CD79a |
| CD83 | CD137/41BB | CD80 | CD79b |
| CD83 | CD137/41BB | CD86 | CD8 |
| CD83 | CD137/41BB | CD86 | CD3ζ |
| CD83 | CD137/41BB | CD86 | CD3δ |
| CD83 | CD137/41BB | CD86 | CD3γ |
| CD83 | CD137/41BB | CD86 | CD3ε |
| CD83 | CD137/41BB | CD86 | FcγRI-γ |
| CD83 | CD137/41BB | CD86 | FcγRIII-γ |
| CD83 | CD137/41BB | CD86 | FcεRIβ |
| CD83 | CD137/41BB | CD86 | FcεRIγ |
| CD83 | CD137/41BB | CD86 | DAP10 |
| CD83 | CD137/41BB | CD86 | DAP12 |
| CD83 | CD137/41BB | CD86 | CD32 |
| CD83 | CD137/41BB | CD86 | CD79a |
| CD83 | CD137/41BB | CD86 | CD79b |
| CD83 | CD137/41BB | OX40 | CD8 |
| CD83 | CD137/41BB | OX40 | CD3ζ |
| CD83 | CD137/41BB | OX40 | CD3δ |
| CD83 | CD137/41BB | OX40 | CD3γ |
| CD83 | CD137/41BB | OX40 | CD3ε |
| CD83 | CD137/41BB | OX40 | FcγRI-γ |
| CD83 | CD137/41BB | OX40 | FcγRIII-γ |
| CD83 | CD137/41BB | OX40 | FcεRIβ |
| CD83 | CD137/41BB | OX40 | FcεRIγ |
| CD83 | CD137/41BB | OX40 | DAP10 |
| CD83 | CD137/41BB | OX40 | DAP12 |
| CD83 | CD137/41BB | OX40 | CD32 |
| CD83 | CD137/41BB | OX40 | CD79a |
| CD83 | CD137/41BB | OX40 | CD79b |
| CD83 | CD137/41BB | DAP10 | CD8 |
| CD83 | CD137/41BB | DAP10 | CD3ζ |
| CD83 | CD137/41BB | DAP10 | CD3δ |
| CD83 | CD137/41BB | DAP10 | CD3γ |
| CD83 | CD137/41BB | DAP10 | CD3ε |
| CD83 | CD137/41BB | DAP10 | FcγRI-γ |
| CD83 | CD137/41BB | DAP10 | FcγRIII-γ |
| CD83 | CD137/41BB | DAP10 | FcεRIβ |
| CD83 | CD137/41BB | DAP10 | FcεRIγ |
| CD83 | CD137/41BB | DAP10 | DAP10 |
| CD83 | CD137/41BB | DAP10 | DAP12 |
| CD83 | CD137/41BB | DAP10 | CD32 |
| CD83 | CD137/41BB | DAP10 | CD79a |
| CD83 | CD137/41BB | DAP10 | CD79b |
| CD83 | CD137/41BB | DAP12 | CD8 |
| CD83 | CD137/41BB | DAP12 | CD3ζ |
| CD83 | CD137/41BB | DAP12 | CD3δ |
| CD83 | CD137/41BB | DAP12 | CD3γ |
| CD83 | CD137/41BB | DAP12 | CD3ε |
| CD83 | CD137/41BB | DAP12 | FcγRI-γ |
| CD83 | CD137/41BB | DAP12 | FcγRIII-γ |
| CD83 | CD137/41BB | DAP12 | FcεRIβ |
| CD83 | CD137/41BB | DAP12 | FcεRIγ |
| CD83 | CD137/41BB | DAP12 | DAP10 |
| CD83 | CD137/41BB | DAP12 | DAP12 |
| CD83 | CD137/41BB | DAP12 | CD32 |
| CD83 | CD137/41BB | DAP12 | CD79a |
| CD83 | CD137/41BB | DAP12 | CD79b |
| CD83 | CD137/41BB | MyD88 | CD8 |
| CD83 | CD137/41BB | MyD88 | CD3ζ |
| CD83 | CD137/41BB | MyD88 | CD3δ |
| CD83 | CD137/41BB | MyD88 | CD3γ |
| CD83 | CD137/41BB | MyD88 | CD3ε |
| CD83 | CD137/41BB | MyD88 | FcγRI-γ |
| CD83 | CD137/41BB | MyD88 | FcγRIII-γ |
| CD83 | CD137/41BB | MyD88 | FcεRIβ |
| CD83 | CD137/41BB | MyD88 | FcεRIγ |
| CD83 | CD137/41BB | MyD88 | DAP10 |
| CD83 | CD137/41BB | MyD88 | DAP12 |
| CD83 | CD137/41BB | MyD88 | CD32 |
| CD83 | CD137/41BB | MyD88 | CD79a |
| CD83 | CD137/41BB | MyD88 | CD79b |
| CD83 | CD137/41BB | CD7 | CD8 |
| CD83 | CD137/41BB | CD7 | CD3ζ |
| CD83 | CD137/41BB | CD7 | CD3δ |
| CD83 | CD137/41BB | CD7 | CD3γ |
| CD83 | CD137/41BB | CD7 | CD3ε |
| CD83 | CD137/41BB | CD7 | FcγRI-γ |
| CD83 | CD137/41BB | CD7 | FcγRIII-γ |
| CD83 | CD137/41BB | CD7 | FcεRIβ |
| CD83 | CD137/41BB | CD7 | FcεRIγ |
| CD83 | CD137/41BB | CD7 | DAP10 |
| CD83 | CD137/41BB | CD7 | DAP12 |
| CD83 | CD137/41BB | CD7 | CD32 |
| CD83 | CD137/41BB | CD7 | CD79a |
| CD83 | CD137/41BB | CD7 | CD79b |
| CD83 | CD137/41BB | BTNL3 | CD8 |
| CD83 | CD137/41BB | BTNL3 | CD3ζ |
| CD83 | CD137/41BB | BTNL3 | CD3δ |
| CD83 | CD137/41BB | BTNL3 | CD3γ |
| CD83 | CD137/41BB | BTNL3 | CD3ε |
| CD83 | CD137/41BB | BTNL3 | FcγRI-γ |
| CD83 | CD137/41BB | BTNL3 | FcγRIII-γ |
| CD83 | CD137/41BB | BTNL3 | FcεRIβ |
| CD83 | CD137/41BB | BTNL3 | FcεRIγ |
| CD83 | CD137/41BB | BTNL3 | DAP10 |
| CD83 | CD137/41BB | BTNL3 | DAP12 |
| CD83 | CD137/41BB | BTNL3 | CD32 |
| CD83 | CD137/41BB | BTNL3 | CD79a |
| CD83 | CD137/41BB | BTNL3 | CD79b |
| CD83 | CD137/41BB | NKG2D | CD8 |
| CD83 | CD137/41BB | NKG2D | CD3ζ |
| CD83 | CD137/41BB | NKG2D | CD3δ |
| CD83 | CD137/41BB | NKG2D | CD3γ |
| CD83 | CD137/41BB | NKG2D | CD3ε |
| CD83 | CD137/41BB | NKG2D | FcγRI-γ |
| CD83 | CD137/41BB | NKG2D | FcγRIII-γ |
| CD83 | CD137/41BB | NKG2D | FcεRIβ |
| CD83 | CD137/41BB | NKG2D | FcεRIγ |
| CD83 | CD137/41BB | NKG2D | DAP10 |
| CD83 | CD137/41BB | NKG2D | DAP12 |
| CD83 | CD137/41BB | NKG2D | CD32 |
| CD83 | CD137/41BB | NKG2D | CD79a |
| CD83 | CD137/41BB | NKG2D | CD79b |
| CD83 | ICOS | CD28 | CD8 |
| CD83 | ICOS | CD28 | CD3ζ |
| CD83 | ICOS | CD28 | CD3δ |
| CD83 | ICOS | CD28 | CD3γ |
| CD83 | ICOS | CD28 | CD3ε |
| CD83 | ICOS | CD28 | FcγRI-γ |
| CD83 | ICOS | CD28 | FcγRIII-γ |
| CD83 | ICOS | CD28 | FcεRIβ |
| CD83 | ICOS | CD28 | FcεRIγ |
| CD83 | ICOS | CD28 | DAP10 |
| CD83 | ICOS | CD28 | DAP12 |
| CD83 | ICOS | CD28 | CD32 |
| CD83 | ICOS | CD28 | CD79a |
| CD83 | ICOS | CD28 | CD79b |
| CD83 | ICOS | CD8 | CD8 |
| CD83 | ICOS | CD8 | CD3ζ |
| CD83 | ICOS | CD8 | CD3δ |
| CD83 | ICOS | CD8 | CD3γ |
| CD83 | ICOS | CD8 | CD3ε |
| CD83 | ICOS | CD8 | FcγRI-γ |
| CD83 | ICOS | CD8 | FcγRIII-γ |
| CD83 | ICOS | CD8 | FcεRIβ |
| CD83 | ICOS | CD8 | FcεRIγ |
| CD83 | ICOS | CD8 | DAP10 |
| CD83 | ICOS | CD8 | DAP12 |
| CD83 | ICOS | CD8 | CD32 |
| CD83 | ICOS | CD8 | CD79a |
| CD83 | ICOS | CD8 | CD79b |
| CD83 | ICOS | CD4 | CD8 |
| CD83 | ICOS | CD4 | CD3ζ |
| CD83 | ICOS | CD4 | CD3δ |
| CD83 | ICOS | CD4 | CD3γ |
| CD83 | ICOS | CD4 | CD3ε |
| CD83 | ICOS | CD4 | FcγRI-γ |
| CD83 | ICOS | CD4 | FcγRIII-γ |
| CD83 | ICOS | CD4 | FcεRIβ |
| CD83 | ICOS | CD4 | FcεRIγ |
| CD83 | ICOS | CD4 | DAP10 |
| CD83 | ICOS | CD4 | DAP12 |
| CD83 | ICOS | CD4 | CD32 |
| CD83 | ICOS | CD4 | CD79a |
| CD83 | ICOS | CD4 | CD79b |
| CD83 | ICOS | b2c | CD8 |
| CD83 | ICOS | b2c | CD3ζ |
| CD83 | ICOS | b2c | CD3δ |
| CD83 | ICOS | b2c | CD3γ |
| CD83 | ICOS | b2c | CD3ε |
| CD83 | ICOS | b2c | FcγRI-γ |
| CD83 | ICOS | b2c | FcγRIII-γ |
| CD83 | ICOS | b2c | FcεRIβ |
| CD83 | ICOS | b2c | FcεRIγ |
| CD83 | ICOS | b2c | DAP10 |
| CD83 | ICOS | b2c | DAP12 |
| CD83 | ICOS | b2c | CD32 |
| CD83 | ICOS | b2c | CD79a |
| CD83 | ICOS | b2c | CD79b |
| CD83 | ICOS | CD137/41BB | CD8 |
| CD83 | ICOS | CD137/41BB | CD3ζ |
| CD83 | ICOS | CD137/41BB | CD3δ |
| CD83 | ICOS | CD137/41BB | CD3γ |
| CD83 | ICOS | CD137/41BB | CD3ε |
| CD83 | ICOS | CD137/41BB | FcγRI-γ |
| CD83 | ICOS | CD137/41BB | FcγRIII-γ |
| CD83 | ICOS | CD137/41BB | FcεRIβ |
| CD83 | ICOS | CD137/41BB | FcεRIγ |
| CD83 | ICOS | CD137/41BB | DAP10 |
| CD83 | ICOS | CD137/41BB | DAP12 |
| CD83 | ICOS | CD137/41BB | CD32 |
| CD83 | ICOS | CD137/41BB | CD79a |
| CD83 | ICOS | CD137/41BB | CD79b |
| CD83 | ICOS | ICOS | CD8 |
| CD83 | ICOS | ICOS | CD3ζ |
| CD83 | ICOS | ICOS | CD3δ |
| CD83 | ICOS | ICOS | CD3γ |
| CD83 | ICOS | ICOS | CD3ε |
| CD83 | ICOS | ICOS | FcγRI-γ |
| CD83 | ICOS | ICOS | FcγRIII-γ |
| CD83 | ICOS | ICOS | FcεRIβ |
| CD83 | ICOS | ICOS | FcεRIγ |
| CD83 | ICOS | ICOS | DAP10 |
| CD83 | ICOS | ICOS | DAP12 |
| CD83 | ICOS | ICOS | CD32 |
| CD83 | ICOS | ICOS | CD79a |
| CD83 | ICOS | ICOS | CD79b |
| CD83 | ICOS | CD27 | CD8 |
| CD83 | ICOS | CD27 | CD3ζ |
| CD83 | ICOS | CD27 | CD3δ |
| CD83 | ICOS | CD27 | CD3γ |
| CD83 | ICOS | CD27 | CD3ε |
| CD83 | ICOS | CD27 | FcγRI-γ |
| CD83 | ICOS | CD27 | FcγRIII-γ |
| CD83 | ICOS | CD27 | FcεRIβ |
| CD83 | ICOS | CD27 | FcεRIγ |
| CD83 | ICOS | CD27 | DAP10 |
| CD83 | ICOS | CD27 | DAP12 |
| CD83 | ICOS | CD27 | CD32 |
| CD83 | ICOS | CD27 | CD79a |
| CD83 | ICOS | CD27 | CD79b |
| CD83 | ICOS | CD28δ | CD8 |
| CD83 | ICOS | CD28δ | CD3ζ |
| CD83 | ICOS | CD28δ | CD3δ |
| CD83 | ICOS | CD28δ | CD3γ |
| CD83 | ICOS | CD28δ | CD3ε |
| CD83 | ICOS | CD28δ | FcγRI-γ |
| CD83 | ICOS | CD28δ | FcγRIII-γ |
| CD83 | ICOS | CD28δ | FcεRIβ |
| CD83 | ICOS | CD28δ | FcεRIγ |
| CD83 | ICOS | CD28δ | DAP10 |
| CD83 | ICOS | CD28δ | DAP12 |
| CD83 | ICOS | CD28δ | CD32 |
| CD83 | ICOS | CD28δ | CD79a |
| CD83 | ICOS | CD28δ | CD79b |
| CD83 | ICOS | CD80 | CD8 |
| CD83 | ICOS | CD80 | CD3ζ |
| CD83 | ICOS | CD80 | CD3δ |
| CD83 | ICOS | CD80 | CD3γ |
| CD83 | ICOS | CD80 | CD3ε |
| CD83 | ICOS | CD80 | FcγRI-γ |
| CD83 | ICOS | CD80 | FcγRIII-γ |
| CD83 | ICOS | CD80 | FcεRIβ |
| CD83 | ICOS | CD80 | FcεRIγ |
| CD83 | ICOS | CD80 | DAP10 |
| CD83 | ICOS | CD80 | DAP12 |
| CD83 | ICOS | CD80 | CD32 |
| CD83 | ICOS | CD80 | CD79a |
| CD83 | ICOS | CD80 | CD79b |
| CD83 | ICOS | CD86 | CD8 |
| CD83 | ICOS | CD86 | CD3ζ |
| CD83 | ICOS | CD86 | CD3δ |
| CD83 | ICOS | CD86 | CD3γ |
| CD83 | ICOS | CD86 | CD3ε |
| CD83 | ICOS | CD86 | FcγRI-γ |
| CD83 | ICOS | CD86 | FcγRIII-γ |
| CD83 | ICOS | CD86 | FcεRIβ |
| CD83 | ICOS | CD86 | FcεRIγ |
| CD83 | ICOS | CD86 | DAP10 |
| CD83 | ICOS | CD86 | DAP12 |
| CD83 | ICOS | CD86 | CD32 |
| CD83 | ICOS | CD86 | CD79a |
| CD83 | ICOS | CD86 | CD79b |
| CD83 | ICOS | OX40 | CD8 |
| CD83 | ICOS | OX40 | CD3ζ |
| CD83 | ICOS | OX40 | CD3δ |
| CD83 | ICOS | OX40 | CD3γ |
| CD83 | ICOS | OX40 | CD3ε |
| CD83 | ICOS | OX40 | FcγRI-γ |
| CD83 | ICOS | OX40 | FcγRIII-γ |
| CD83 | ICOS | OX40 | FcεRIβ |
| CD83 | ICOS | OX40 | FcεRIγ |
| CD83 | ICOS | OX40 | DAP10 |
| CD83 | ICOS | OX40 | DAP12 |
| CD83 | ICOS | OX40 | CD32 |
| CD83 | ICOS | OX40 | CD79a |
| CD83 | ICOS | OX40 | CD79b |
| CD83 | ICOS | DAP10 | CD8 |
| CD83 | ICOS | DAP10 | CD3ζ |
| CD83 | ICOS | DAP10 | CD3δ |
| CD83 | ICOS | DAP10 | CD3γ |
| CD83 | ICOS | DAP10 | CD3ε |
| CD83 | ICOS | DAP10 | FcγRI-γ |
| CD83 | ICOS | DAP10 | FcγRIII-γ |
| CD83 | ICOS | DAP10 | FcεRIβ |
| CD83 | ICOS | DAP10 | FcεRIγ |
| CD83 | ICOS | DAP10 | DAP10 |
| CD83 | ICOS | DAP10 | DAP12 |
| CD83 | ICOS | DAP10 | CD32 |
| CD83 | ICOS | DAP10 | CD79a |
| CD83 | ICOS | DAP10 | CD79b |
| CD83 | ICOS | DAP12 | CD8 |
| CD83 | ICOS | DAP12 | CD3ζ |
| CD83 | ICOS | DAP12 | CD3δ |
| CD83 | ICOS | DAP12 | CD3γ |
| CD83 | ICOS | DAP12 | CD3ε |
| CD83 | ICOS | DAP12 | FcγRI-γ |
| CD83 | ICOS | DAP12 | FcγRIII-γ |
| CD83 | ICOS | DAP12 | FcεRIβ |
| CD83 | ICOS | DAP12 | FcεRIγ |
| CD83 | ICOS | DAP12 | DAP10 |
| CD83 | ICOS | DAP12 | DAP12 |
| CD83 | ICOS | DAP12 | CD32 |
| CD83 | ICOS | DAP12 | CD79a |
| CD83 | ICOS | DAP12 | CD79b |
| CD83 | ICOS | MyD88 | CD8 |
| CD83 | ICOS | MyD88 | CD3ζ |
| CD83 | ICOS | MyD88 | CD3δ |
| CD83 | ICOS | MyD88 | CD3γ |
| CD83 | ICOS | MyD88 | CD3ε |
| CD83 | ICOS | MyD88 | FcγRI-γ |
| CD83 | ICOS | MyD88 | FcγRIII-γ |
| CD83 | ICOS | MyD88 | FcεRIβ |
| CD83 | ICOS | MyD88 | FcεRIγ |
| CD83 | ICOS | MyD88 | DAP10 |
| CD83 | ICOS | MyD88 | DAP12 |
| CD83 | ICOS | MyD88 | CD32 |
| CD83 | ICOS | MyD88 | CD79a |
| CD83 | ICOS | MyD88 | CD79b |
| CD83 | ICOS | CD7 | CD8 |
| CD83 | ICOS | CD7 | CD3ζ |
| CD83 | ICOS | CD7 | CD3δ |
| CD83 | ICOS | CD7 | CD3γ |
| CD83 | ICOS | CD7 | CD3ε |
| CD83 | ICOS | CD7 | FcγRI-γ |
| CD83 | ICOS | CD7 | FcγRIII-γ |
| CD83 | ICOS | CD7 | FcεRIβ |
| CD83 | ICOS | CD7 | FcεRIγ |
| CD83 | ICOS | CD7 | DAP10 |
| CD83 | ICOS | CD7 | DAP12 |
| CD83 | ICOS | CD7 | CD32 |
| CD83 | ICOS | CD7 | CD79a |
| CD83 | ICOS | CD7 | CD79b |
| CD83 | ICOS | BTNL3 | CD8 |
| CD83 | ICOS | BTNL3 | CD3ζ |
| CD83 | ICOS | BTNL3 | CD3δ |
| CD83 | ICOS | BTNL3 | CD3γ |
| CD83 | ICOS | BTNL3 | CD3ε |
| CD83 | ICOS | BTNL3 | FcγRI-γ |
| CD83 | ICOS | BTNL3 | FcγRIII-γ |
| CD83 | ICOS | BTNL3 | FcεRIβ |
| CD83 | ICOS | BTNL3 | FcεRIγ |
| CD83 | ICOS | BTNL3 | DAP10 |
| CD83 | ICOS | BTNL3 | DAP12 |
| CD83 | ICOS | BTNL3 | CD32 |
| CD83 | ICOS | BTNL3 | CD79a |
| CD83 | ICOS | BTNL3 | CD79b |
| CD83 | ICOS | NKG2D | CD8 |
| CD83 | ICOS | NKG2D | CD3ζ |
| CD83 | ICOS | NKG2D | CD3δ |
| CD83 | ICOS | NKG2D | CD3γ |
| CD83 | ICOS | NKG2D | CD3ε |
| CD83 | ICOS | NKG2D | FcγRI-γ |
| CD83 | ICOS | NKG2D | FcγRIII-γ |
| CD83 | ICOS | NKG2D | FcεRIβ |
| CD83 | ICOS | NKG2D | FcεRIγ |
| CD83 | ICOS | NKG2D | DAP10 |
| CD83 | ICOS | NKG2D | DAP12 |
| CD83 | ICOS | NKG2D | CD32 |
| CD83 | ICOS | NKG2D | CD79a |
| CD83 | ICOS | NKG2D | CD79b |
| CD83 | CD27 | CD28 | CD8 |
| CD83 | CD27 | CD28 | CD3ζ |
| CD83 | CD27 | CD28 | CD3δ |
| CD83 | CD27 | CD28 | CD3γ |
| CD83 | CD27 | CD28 | CD3ε |
| CD83 | CD27 | CD28 | FcγRI-γ |
| CD83 | CD27 | CD28 | FcγRIII-γ |
| CD83 | CD27 | CD28 | FcεRIβ |
| CD83 | CD27 | CD28 | FcεRIγ |
| CD83 | CD27 | CD28 | DAP10 |
| CD83 | CD27 | CD28 | DAP12 |
| CD83 | CD27 | CD28 | CD32 |
| CD83 | CD27 | CD28 | CD79a |
| CD83 | CD27 | CD28 | CD79b |
| CD83 | CD27 | CD8 | CD8 |
| CD83 | CD27 | CD8 | CD3ζ |
| CD83 | CD27 | CD8 | CD3δ |
| CD83 | CD27 | CD8 | CD3γ |
| CD83 | CD27 | CD8 | CD3ε |
| CD83 | CD27 | CD8 | FcγRI-γ |
| CD83 | CD27 | CD8 | FcγRIII-γ |
| CD83 | CD27 | CD8 | FcεRIβ |
| CD83 | CD27 | CD8 | FcεRIγ |
| CD83 | CD27 | CD8 | DAP10 |
| CD83 | CD27 | CD8 | DAP12 |
| CD83 | CD27 | CD8 | CD32 |
| CD83 | CD27 | CD8 | CD79a |
| CD83 | CD27 | CD8 | CD79b |
| CD83 | CD27 | CD4 | CD8 |
| CD83 | CD27 | CD4 | CD3ζ |
| CD83 | CD27 | CD4 | CD3δ |
| CD83 | CD27 | CD4 | CD3γ |
| CD83 | CD27 | CD4 | CD3ε |
| CD83 | CD27 | CD4 | FcγRI-γ |
| CD83 | CD27 | CD4 | FcγRIII-γ |
| CD83 | CD27 | CD4 | FcεRIβ |
| CD83 | CD27 | CD4 | FcεRIγ |
| CD83 | CD27 | CD4 | DAP10 |
| CD83 | CD27 | CD4 | DAP12 |
| CD83 | CD27 | CD4 | CD32 |
| CD83 | CD27 | CD4 | CD79a |
| CD83 | CD27 | CD4 | CD79b |
| CD83 | CD27 | b2c | CD8 |
| CD83 | CD27 | b2c | CD3ζ |
| CD83 | CD27 | b2c | CD3δ |
| CD83 | CD27 | b2c | CD3γ |
| CD83 | CD27 | b2c | CD3ε |
| CD83 | CD27 | b2c | FcγRI-γ |
| CD83 | CD27 | b2c | FcγRIII-γ |
| CD83 | CD27 | b2c | FcεRIβ |
| CD83 | CD27 | b2c | FcεRIγ |
| CD83 | CD27 | b2c | DAP10 |
| CD83 | CD27 | b2c | DAP12 |
| CD83 | CD27 | b2c | CD32 |
| CD83 | CD27 | b2c | CD79a |
| CD83 | CD27 | b2c | CD79b |
| CD83 | CD27 | CD137/41BB | CD8 |
| CD83 | CD27 | CD137/41BB | CD3ζ |
| CD83 | CD27 | CD137/41BB | CD3δ |
| CD83 | CD27 | CD137/41BB | CD3γ |
| CD83 | CD27 | CD137/41BB | CD3ε |
| CD83 | CD27 | CD137/41BB | FcγRI-γ |
| CD83 | CD27 | CD137/41BB | FcγRIII-γ |
| CD83 | CD27 | CD137/41BB | FcεRIβ |
| CD83 | CD27 | CD137/41BB | FcεRIγ |
| CD83 | CD27 | CD137/41BB | DAP10 |
| CD83 | CD27 | CD137/41BB | DAP12 |
| CD83 | CD27 | CD137/41BB | CD32 |
| CD83 | CD27 | CD137/41BB | CD79a |
| CD83 | CD27 | CD137/41BB | CD79b |
| CD83 | CD27 | ICOS | CD8 |
| CD83 | CD27 | ICOS | CD3ζ |
| CD83 | CD27 | ICOS | CD3δ |
| CD83 | CD27 | ICOS | CD3γ |
| CD83 | CD27 | ICOS | CD3ε |
| CD83 | CD27 | ICOS | FcγRI-γ |
| CD83 | CD27 | ICOS | FcγRIII-γ |
| CD83 | CD27 | ICOS | FcεRIβ |
| CD83 | CD27 | ICOS | FcεRIγ |
| CD83 | CD27 | ICOS | DAP10 |
| CD83 | CD27 | ICOS | DAP12 |
| CD83 | CD27 | ICOS | CD32 |
| CD83 | CD27 | ICOS | CD79a |
| CD83 | CD27 | ICOS | CD79b |
| CD83 | CD27 | CD27 | CD8 |
| CD83 | CD27 | CD27 | CD3ζ |
| CD83 | CD27 | CD27 | CD3δ |
| CD83 | CD27 | CD27 | CD3γ |
| CD83 | CD27 | CD27 | CD3ε |
| CD83 | CD27 | CD27 | FcγRI-γ |
| CD83 | CD27 | CD27 | FcγRIII-γ |
| CD83 | CD27 | CD27 | FcεRIβ |
| CD83 | CD27 | CD27 | FcεRIγ |
| CD83 | CD27 | CD27 | DAP10 |
| CD83 | CD27 | CD27 | DAP12 |
| CD83 | CD27 | CD27 | CD32 |
| CD83 | CD27 | CD27 | CD79a |
| CD83 | CD27 | CD27 | CD79b |
| CD83 | CD27 | CD28δ | CD8 |
| CD83 | CD27 | CD28δ | CD3ζ |
| CD83 | CD27 | CD28δ | CD3δ |
| CD83 | CD27 | CD28δ | CD3γ |
| CD83 | CD27 | CD28δ | CD3ε |
| CD83 | CD27 | CD28δ | FcγRI-γ |
| CD83 | CD27 | CD28δ | FcγRIII-γ |
| CD83 | CD27 | CD28δ | FcεRIβ |
| CD83 | CD27 | CD28δ | FcεRIγ |
| CD83 | CD27 | CD28δ | DAP10 |
| CD83 | CD27 | CD28δ | DAP12 |
| CD83 | CD27 | CD28δ | CD32 |
| CD83 | CD27 | CD28δ | CD79a |
| CD83 | CD27 | CD28δ | CD79b |
| CD83 | CD27 | CD80 | CD8 |
| CD83 | CD27 | CD80 | CD3ζ |
| CD83 | CD27 | CD80 | CD3δ |
| CD83 | CD27 | CD80 | CD3γ |
| CD83 | CD27 | CD80 | CD3ε |
| CD83 | CD27 | CD80 | FcγRI-γ |
| CD83 | CD27 | CD80 | FcγRIII-γ |
| CD83 | CD27 | CD80 | FcεRIβ |
| CD83 | CD27 | CD80 | FcεRIγ |
| CD83 | CD27 | CD80 | DAP10 |
| CD83 | CD27 | CD80 | DAP12 |
| CD83 | CD27 | CD80 | CD32 |
| CD83 | CD27 | CD80 | CD79a |
| CD83 | CD27 | CD80 | CD79b |
| CD83 | CD27 | CD86 | CD8 |
| CD83 | CD27 | CD86 | CD3ζ |
| CD83 | CD27 | CD86 | CD3δ |
| CD83 | CD27 | CD86 | CD3γ |
| CD83 | CD27 | CD86 | CD3ε |
| CD83 | CD27 | CD86 | FcγRI-γ |
| CD83 | CD27 | CD86 | FcγRIII-γ |
| CD83 | CD27 | CD86 | FcεRIβ |
| CD83 | CD27 | CD86 | FcεRIγ |
| CD83 | CD27 | CD86 | DAP10 |
| CD83 | CD27 | CD86 | DAP12 |
| CD83 | CD27 | CD86 | CD32 |
| CD83 | CD27 | CD86 | CD79a |
| CD83 | CD27 | CD86 | CD79b |
| CD83 | CD27 | OX40 | CD8 |
| CD83 | CD27 | OX40 | CD3ζ |
| CD83 | CD27 | OX40 | CD3δ |
| CD83 | CD27 | OX40 | CD3γ |
| CD83 | CD27 | OX40 | CD3ε |
| CD83 | CD27 | OX40 | FcγRI-γ |
| CD83 | CD27 | OX40 | FcγRIII-γ |
| CD83 | CD27 | OX40 | FcεRIβ |
| CD83 | CD27 | OX40 | FcεRIγ |
| CD83 | CD27 | OX40 | DAP10 |
| CD83 | CD27 | OX40 | DAP12 |
| CD83 | CD27 | OX40 | CD32 |
| CD83 | CD27 | OX40 | CD79a |
| CD83 | CD27 | OX40 | CD79b |
| CD83 | CD27 | DAP10 | CD8 |
| CD83 | CD27 | DAP10 | CD3ζ |
| CD83 | CD27 | DAP10 | CD3δ |
| CD83 | CD27 | DAP10 | CD3γ |
| CD83 | CD27 | DAP10 | CD3ε |
| CD83 | CD27 | DAP10 | FcγRI-γ |
| CD83 | CD27 | DAP10 | FcγRIII-γ |
| CD83 | CD27 | DAP10 | FcεRIβ |
| CD83 | CD27 | DAP10 | FcεRIγ |
| CD83 | CD27 | DAP10 | DAP10 |
| CD83 | CD27 | DAP10 | DAP12 |
| CD83 | CD27 | DAP10 | CD32 |
| CD83 | CD27 | DAP10 | CD79a |
| CD83 | CD27 | DAP10 | CD79b |
| CD83 | CD27 | DAP12 | CD8 |
| CD83 | CD27 | DAP12 | CD3ζ |
| CD83 | CD27 | DAP12 | CD3δ |
| CD83 | CD27 | DAP12 | CD3γ |
| CD83 | CD27 | DAP12 | CD3ε |
| CD83 | CD27 | DAP12 | FcγRI-γ |
| CD83 | CD27 | DAP12 | FcγRIII-γ |
| CD83 | CD27 | DAP12 | FcεRIβ |
| CD83 | CD27 | DAP12 | FcεRIγ |
| CD83 | CD27 | DAP12 | DAP10 |
| CD83 | CD27 | DAP12 | DAP12 |
| CD83 | CD27 | DAP12 | CD32 |
| CD83 | CD27 | DAP12 | CD79a |
| CD83 | CD27 | DAP12 | CD79b |
| CD83 | CD27 | MyD88 | CD8 |
| CD83 | CD27 | MyD88 | CD3ζ |
| CD83 | CD27 | MyD88 | CD3δ |
| CD83 | CD27 | MyD88 | CD3γ |
| CD83 | CD27 | MyD88 | CD3ε |
| CD83 | CD27 | MyD88 | FcγRI-γ |
| CD83 | CD27 | MyD88 | FcγRIII-γ |
| CD83 | CD27 | MyD88 | FcεRIβ |
| CD83 | CD27 | MyD88 | FcεRIγ |
| CD83 | CD27 | MyD88 | DAP10 |
| CD83 | CD27 | MyD88 | DAP12 |
| CD83 | CD27 | MyD88 | CD32 |
| CD83 | CD27 | MyD88 | CD79a |
| CD83 | CD27 | MyD88 | CD79b |
| CD83 | CD27 | CD7 | CD8 |
| CD83 | CD27 | CD7 | CD3ζ |
| CD83 | CD27 | CD7 | CD3δ |
| CD83 | CD27 | CD7 | CD3γ |
| CD83 | CD27 | CD7 | CD3ε |
| CD83 | CD27 | CD7 | FcγRI-γ |
| CD83 | CD27 | CD7 | FcγRIII-γ |
| CD83 | CD27 | CD7 | FcεRIβ |
| CD83 | CD27 | CD7 | FcεRIγ |
| CD83 | CD27 | CD7 | DAP10 |
| CD83 | CD27 | CD7 | DAP12 |
| CD83 | CD27 | CD7 | CD32 |
| CD83 | CD27 | CD7 | CD79a |
| CD83 | CD27 | CD7 | CD79b |
| CD83 | CD27 | BTNL3 | CD8 |
| CD83 | CD27 | BTNL3 | CD3ζ |
| CD83 | CD27 | BTNL3 | CD3δ |
| CD83 | CD27 | BTNL3 | CD3γ |
| CD83 | CD27 | BTNL3 | CD3ε |
| CD83 | CD27 | BTNL3 | FcγRI-γ |
| CD83 | CD27 | BTNL3 | FcγRIII-γ |
| CD83 | CD27 | BTNL3 | FcεRIβ |
| CD83 | CD27 | BTNL3 | FcεRIγ |
| CD83 | CD27 | BTNL3 | DAP10 |
| CD83 | CD27 | BTNL3 | DAP12 |
| CD83 | CD27 | BTNL3 | CD32 |
| CD83 | CD27 | BTNL3 | CD79a |
| CD83 | CD27 | BTNL3 | CD79b |
| CD83 | CD27 | NKG2D | CD8 |
| CD83 | CD27 | NKG2D | CD3ζ |
| CD83 | CD27 | NKG2D | CD3δ |
| CD83 | CD27 | NKG2D | CD3γ |
| CD83 | CD27 | NKG2D | CD3ε |
| CD83 | CD27 | NKG2D | FcγRI-γ |
| CD83 | CD27 | NKG2D | FcγRIII-γ |
| CD83 | CD27 | NKG2D | FcεRIβ |
| CD83 | CD27 | NKG2D | FcεRIγ |
| CD83 | CD27 | NKG2D | DAP10 |
| CD83 | CD27 | NKG2D | DAP12 |
| CD83 | CD27 | NKG2D | CD32 |
| CD83 | CD27 | NKG2D | CD79a |
| CD83 | CD27 | NKG2D | CD79b |
| CD83 | CD28δ | CD28 | CD8 |
| CD83 | CD28δ | CD28 | CD3ζ |
| CD83 | CD28δ | CD28 | CD3δ |
| CD83 | CD28δ | CD28 | CD3γ |
| CD83 | CD28δ | CD28 | CD3ε |
| CD83 | CD28δ | CD28 | FcγRI-γ |
| CD83 | CD28δ | CD28 | FcγRIII-γ |
| CD83 | CD28δ | CD28 | FcεRIβ |
| CD83 | CD28δ | CD28 | FcεRIγ |
| CD83 | CD28δ | CD28 | DAP10 |
| CD83 | CD28δ | CD28 | DAP12 |
| CD83 | CD28δ | CD28 | CD32 |
| CD83 | CD28δ | CD28 | CD79a |
| CD83 | CD28δ | CD28 | CD79b |
| CD83 | CD28δ | CD8 | CD8 |
| CD83 | CD28δ | CD8 | CD3ζ |
| CD83 | CD28δ | CD8 | CD3δ |
| CD83 | CD28δ | CD8 | CD3γ |
| CD83 | CD28δ | CD8 | CD3ε |
| CD83 | CD28δ | CD8 | FcγRI-γ |
| CD83 | CD28δ | CD8 | FcγRIII-γ |
| CD83 | CD28δ | CD8 | FcεRIβ |
| CD83 | CD28δ | CD8 | FcεRIγ |
| CD83 | CD28δ | CD8 | DAP10 |
| CD83 | CD28δ | CD8 | DAP12 |
| CD83 | CD28δ | CD8 | CD32 |
| CD83 | CD28δ | CD8 | CD79a |
| CD83 | CD28δ | CD8 | CD79b |
| CD83 | CD28δ | CD4 | CD8 |
| CD83 | CD28δ | CD4 | CD3ζ |
| CD83 | CD28δ | CD4 | CD3δ |
| CD83 | CD28δ | CD4 | CD3γ |
| CD83 | CD28δ | CD4 | CD3ε |
| CD83 | CD28δ | CD4 | FcγRI-γ |
| CD83 | CD28δ | CD4 | FcγRIII-γ |
| CD83 | CD28δ | CD4 | FcεRIβ |
| CD83 | CD28δ | CD4 | FcεRIγ |
| CD83 | CD28δ | CD4 | DAP10 |
| CD83 | CD28δ | CD4 | DAP12 |
| CD83 | CD28δ | CD4 | CD32 |
| CD83 | CD28δ | CD4 | CD79a |
| CD83 | CD28δ | CD4 | CD79b |
| CD83 | CD28δ | b2c | CD8 |
| CD83 | CD28δ | b2c | CD3ζ |
| CD83 | CD28δ | b2c | CD3δ |
| CD83 | CD28δ | b2c | CD3γ |
| CD83 | CD28δ | b2c | CD3ε |
| CD83 | CD28δ | b2c | FcγRI-γ |
| CD83 | CD28δ | b2c | FcγRIII-γ |
| CD83 | CD28δ | b2c | FcεRIβ |
| CD83 | CD28δ | b2c | FcεRIγ |
| CD83 | CD28δ | b2c | DAP10 |
| CD83 | CD28δ | b2c | DAP12 |
| CD83 | CD28δ | b2c | CD32 |
| CD83 | CD28δ | b2c | CD79a |
| CD83 | CD28δ | b2c | CD79b |
| CD83 | CD28δ | CD137/41BB | CD8 |
| CD83 | CD28δ | CD137/41BB | CD3ζ |
| CD83 | CD28δ | CD137/41BB | CD3δ |
| CD83 | CD28δ | CD137/41BB | CD3γ |
| CD83 | CD28δ | CD137/41BB | CD3ε |
| CD83 | CD28δ | CD137/41BB | FcγRI-γ |
| CD83 | CD28δ | CD137/41BB | FcγRIII-γ |
| CD83 | CD28δ | CD137/41BB | FcεRIβ |
| CD83 | CD28δ | CD137/41BB | FcεRIγ |
| CD83 | CD28δ | CD137/41BB | DAP10 |
| CD83 | CD28δ | CD137/41BB | DAP12 |
| CD83 | CD28δ | CD137/41BB | CD32 |
| CD83 | CD28δ | CD137/41BB | CD79a |
| CD83 | CD28δ | CD137/41BB | CD79b |
| CD83 | CD28δ | ICOS | CD8 |
| CD83 | CD28δ | ICOS | CD3ζ |
| CD83 | CD28δ | ICOS | CD3δ |
| CD83 | CD28δ | ICOS | CD3γ |
| CD83 | CD28δ | ICOS | CD3ε |
| CD83 | CD28δ | ICOS | FcγRI-γ |
| CD83 | CD28δ | ICOS | FcγRIII-γ |
| CD83 | CD28δ | ICOS | FcεRIβ |
| CD83 | CD28δ | ICOS | FcεRIγ |
| CD83 | CD28δ | ICOS | DAP10 |
| CD83 | CD28δ | ICOS | DAP12 |
| CD83 | CD28δ | ICOS | CD32 |
| CD83 | CD28δ | ICOS | CD79a |
| CD83 | CD28δ | ICOS | CD79b |
| CD83 | CD28δ | CD27 | CD8 |
| CD83 | CD28δ | CD27 | CD3ζ |
| CD83 | CD28δ | CD27 | CD3δ |
| CD83 | CD28δ | CD27 | CD3γ |
| CD83 | CD28δ | CD27 | CD3ε |
| CD83 | CD28δ | CD27 | FcγRI-γ |
| CD83 | CD28δ | CD27 | FcγRIII-γ |
| CD83 | CD28δ | CD27 | FcεRIβ |
| CD83 | CD28δ | CD27 | FcεRIγ |
| CD83 | CD28δ | CD27 | DAP10 |
| CD83 | CD28δ | CD27 | DAP12 |
| CD83 | CD28δ | CD27 | CD32 |
| CD83 | CD28δ | CD27 | CD79a |
| CD83 | CD28δ | CD27 | CD79b |
| CD83 | CD28δ | CD28δ | CD8 |
| CD83 | CD28δ | CD28δ | CD3ζ |
| CD83 | CD28δ | CD28δ | CD3δ |
| CD83 | CD28δ | CD28δ | CD3γ |
| CD83 | CD28δ | CD28δ | CD3ε |
| CD83 | CD28δ | CD28δ | FcγRI-γ |
| CD83 | CD28δ | CD28δ | FcγRIII-γ |
| CD83 | CD28δ | CD28δ | FcεRIβ |
| CD83 | CD28δ | CD28δ | FcεRIγ |
| CD83 | CD28δ | CD28δ | DAP10 |
| CD83 | CD28δ | CD28δ | DAP12 |
| CD83 | CD28δ | CD28δ | CD32 |
| CD83 | CD28δ | CD28δ | CD79a |
| CD83 | CD28δ | CD28δ | CD79b |
| CD83 | CD28δ | CD80 | CD8 |
| CD83 | CD28δ | CD80 | CD3ζ |
| CD83 | CD28δ | CD80 | CD3δ |
| CD83 | CD28δ | CD80 | CD3γ |
| CD83 | CD28δ | CD80 | CD3ε |
| CD83 | CD28δ | CD80 | FcγRI-γ |
| CD83 | CD28δ | CD80 | FcγRIII-γ |
| CD83 | CD28δ | CD80 | FcεRIβ |
| CD83 | CD28δ | CD80 | FcεRIγ |
| CD83 | CD28δ | CD80 | DAP10 |
| CD83 | CD28δ | CD80 | DAP12 |
| CD83 | CD28δ | CD80 | CD32 |
| CD83 | CD28δ | CD80 | CD79a |
| CD83 | CD28δ | CD80 | CD79b |
| CD83 | CD28δ | CD86 | CD8 |
| CD83 | CD28δ | CD86 | CD3ζ |
| CD83 | CD28δ | CD86 | CD3δ |
| CD83 | CD28δ | CD86 | CD3γ |
| CD83 | CD28δ | CD86 | CD3ε |
| CD83 | CD28δ | CD86 | FcγRI-γ |
| CD83 | CD28δ | CD86 | FcγRIII-γ |
| CD83 | CD28δ | CD86 | FcεRIβ |
| CD83 | CD28δ | CD86 | FcεRIγ |
| CD83 | CD28δ | CD86 | DAP10 |
| CD83 | CD28δ | CD86 | DAP12 |
| CD83 | CD28δ | CD86 | CD32 |
| CD83 | CD28δ | CD86 | CD79a |
| CD83 | CD28δ | CD86 | CD79b |
| CD83 | CD28δ | OX40 | CD8 |
| CD83 | CD28δ | OX40 | CD3ζ |
| CD83 | CD28δ | OX40 | CD3δ |
| CD83 | CD28δ | OX40 | CD3γ |
| CD83 | CD28δ | OX40 | CD3ε |
| CD83 | CD28δ | OX40 | FcγRI-γ |
| CD83 | CD28δ | OX40 | FcγRIII-γ |
| CD83 | CD28δ | OX40 | FcεRIβ |
| CD83 | CD28δ | OX40 | FcεRIγ |
| CD83 | CD28δ | OX40 | DAP10 |
| CD83 | CD28δ | OX40 | DAP12 |
| CD83 | CD28δ | OX40 | CD32 |
| CD83 | CD28δ | OX40 | CD79a |
| CD83 | CD28δ | OX40 | CD79b |
| CD83 | CD28δ | DAP10 | CD8 |
| CD83 | CD28δ | DAP10 | CD3ζ |
| CD83 | CD28δ | DAP10 | CD3δ |
| CD83 | CD28δ | DAP10 | CD3γ |
| CD83 | CD28δ | DAP10 | CD3ε |
| CD83 | CD28δ | DAP10 | FcγRI-γ |
| CD83 | CD28δ | DAP10 | FcγRIII-γ |
| CD83 | CD28δ | DAP10 | FcεRIβ |
| CD83 | CD28δ | DAP10 | FcεRIγ |
| CD83 | CD28δ | DAP10 | DAP10 |
| CD83 | CD28δ | DAP10 | DAP12 |
| CD83 | CD28δ | DAP10 | CD32 |
| CD83 | CD28δ | DAP10 | CD79a |
| CD83 | CD28δ | DAP10 | CD79b |
| CD83 | CD28δ | DAP12 | CD8 |
| CD83 | CD28δ | DAP12 | CD3ζ |
| CD83 | CD28δ | DAP12 | CD3δ |
| CD83 | CD28δ | DAP12 | CD3γ |
| CD83 | CD28δ | DAP12 | CD3ε |
| CD83 | CD28δ | DAP12 | FcγRI-γ |
| CD83 | CD28δ | DAP12 | FcγRIII-γ |
| CD83 | CD28δ | DAP12 | FcεRIβ |
| CD83 | CD28δ | DAP12 | FcεRIγ |
| CD83 | CD28δ | DAP12 | DAP10 |
| CD83 | CD28δ | DAP12 | DAP12 |
| CD83 | CD28δ | DAP12 | CD32 |
| CD83 | CD28δ | DAP12 | CD79a |
| CD83 | CD28δ | DAP12 | CD79b |
| CD83 | CD28δ | MyD88 | CD8 |
| CD83 | CD28δ | MyD88 | CD3ζ |
| CD83 | CD28δ | MyD88 | CD3δ |
| CD83 | CD28δ | MyD88 | CD3γ |
| CD83 | CD28δ | MyD88 | CD3ε |
| CD83 | CD28δ | MyD88 | FcγRI-γ |
| CD83 | CD28δ | MyD88 | FcγRIII-γ |
| CD83 | CD28δ | MyD88 | FcεRIβ |
| CD83 | CD28δ | MyD88 | FcεRIγ |
| CD83 | CD28δ | MyD88 | DAP10 |
| CD83 | CD28δ | MyD88 | DAP12 |
| CD83 | CD28δ | MyD88 | CD32 |
| CD83 | CD28δ | MyD88 | CD79a |
| CD83 | CD28δ | MyD88 | CD79b |
| CD83 | CD28δ | CD7 | CD8 |
| CD83 | CD28δ | CD7 | CD3ζ |
| CD83 | CD28δ | CD7 | CD3δ |
| CD83 | CD28δ | CD7 | CD3γ |
| CD83 | CD28δ | CD7 | CD3ε |
| CD83 | CD28δ | CD7 | FcγRI-γ |
| CD83 | CD28δ | CD7 | FcγRIII-γ |
| CD83 | CD28δ | CD7 | FcεRIβ |
| CD83 | CD28δ | CD7 | FcεRIγ |
| CD83 | CD28δ | CD7 | DAP10 |
| CD83 | CD28δ | CD7 | DAP12 |
| CD83 | CD28δ | CD7 | CD32 |
| CD83 | CD28δ | CD7 | CD79a |
| CD83 | CD28δ | CD7 | CD79b |
| CD83 | CD28δ | BTNL3 | CD8 |
| CD83 | CD28δ | BTNL3 | CD3ζ |
| CD83 | CD28δ | BTNL3 | CD3δ |
| CD83 | CD28δ | BTNL3 | CD3γ |
| CD83 | CD28δ | BTNL3 | CD3ε |
| CD83 | CD28δ | BTNL3 | FcγRI-γ |
| CD83 | CD28δ | BTNL3 | FcγRIII-γ |
| CD83 | CD28δ | BTNL3 | FcεRIβ |
| CD83 | CD28δ | BTNL3 | FcεRIγ |
| CD83 | CD28δ | BTNL3 | DAP10 |
| CD83 | CD28δ | BTNL3 | DAP12 |
| CD83 | CD28δ | BTNL3 | CD32 |
| CD83 | CD28δ | BTNL3 | CD79a |
| CD83 | CD28δ | BTNL3 | CD79b |
| CD83 | CD28δ | NKG2D | CD8 |
| CD83 | CD28δ | NKG2D | CD3ζ |
| CD83 | CD28δ | NKG2D | CD3δ |
| CD83 | CD28δ | NKG2D | CD3γ |
| CD83 | CD28δ | NKG2D | CD3ε |
| CD83 | CD28δ | NKG2D | FcγRI-γ |
| CD83 | CD28δ | NKG2D | FcγRIII-γ |
| CD83 | CD28δ | NKG2D | FcεRIβ |
| CD83 | CD28δ | NKG2D | FcεRIγ |
| CD83 | CD28δ | NKG2D | DAP10 |
| CD83 | CD28δ | NKG2D | DAP12 |
| CD83 | CD28δ | NKG2D | CD32 |
| CD83 | CD28δ | NKG2D | CD79a |
| CD83 | CD28δ | NKG2D | CD79b |
| CD83 | CD80 | CD28 | CD8 |
| CD83 | CD80 | CD28 | CD3ζ |
| CD83 | CD80 | CD28 | CD3δ |
| CD83 | CD80 | CD28 | CD3γ |
| CD83 | CD80 | CD28 | CD3ε |
| CD83 | CD80 | CD28 | FcγRI-γ |
| CD83 | CD80 | CD28 | FcγRIII-γ |
| CD83 | CD80 | CD28 | FcεRIβ |
| CD83 | CD80 | CD28 | FcεRIγ |
| CD83 | CD80 | CD28 | DAP10 |
| CD83 | CD80 | CD28 | DAP12 |
| CD83 | CD80 | CD28 | CD32 |
| CD83 | CD80 | CD28 | CD79a |
| CD83 | CD80 | CD28 | CD79b |
| CD83 | CD80 | CD8 | CD8 |
| CD83 | CD80 | CD8 | CD3ζ |
| CD83 | CD80 | CD8 | CD3δ |
| CD83 | CD80 | CD8 | CD3γ |
| CD83 | CD80 | CD8 | CD3ε |
| CD83 | CD80 | CD8 | FcγRI-γ |
| CD83 | CD80 | CD8 | FcγRIII-γ |
| CD83 | CD80 | CD8 | FcεRIβ |
| CD83 | CD80 | CD8 | FcεRIγ |
| CD83 | CD80 | CD8 | DAP10 |
| CD83 | CD80 | CD8 | DAP12 |
| CD83 | CD80 | CD8 | CD32 |
| CD83 | CD80 | CD8 | CD79a |
| CD83 | CD80 | CD8 | CD79b |
| CD83 | CD80 | CD4 | CD8 |
| CD83 | CD80 | CD4 | CD3ζ |
| CD83 | CD80 | CD4 | CD3δ |
| CD83 | CD80 | CD4 | CD3γ |
| CD83 | CD80 | CD4 | CD3ε |
| CD83 | CD80 | CD4 | FcγRI-γ |
| CD83 | CD80 | CD4 | FcγRIII-γ |
| CD83 | CD80 | CD4 | FcεRIβ |
| CD83 | CD80 | CD4 | FcεRIγ |
| CD83 | CD80 | CD4 | DAP10 |
| CD83 | CD80 | CD4 | DAP12 |
| CD83 | CD80 | CD4 | CD32 |
| CD83 | CD80 | CD4 | CD79a |
| CD83 | CD80 | CD4 | CD79b |
| CD83 | CD80 | b2c | CD8 |
| CD83 | CD80 | b2c | CD3ζ |
| CD83 | CD80 | b2c | CD3δ |
| CD83 | CD80 | b2c | CD3γ |
| CD83 | CD80 | b2c | CD3ε |
| CD83 | CD80 | b2c | FcγRI-γ |
| CD83 | CD80 | b2c | FcγRIII-γ |
| CD83 | CD80 | b2c | FcεRIβ |
| CD83 | CD80 | b2c | FcεRIγ |
| CD83 | CD80 | b2c | DAP10 |
| CD83 | CD80 | b2c | DAP12 |
| CD83 | CD80 | b2c | CD32 |
| CD83 | CD80 | b2c | CD79a |
| CD83 | CD80 | b2c | CD79b |
| CD83 | CD80 | CD137/41BB | CD8 |
| CD83 | CD80 | CD137/41BB | CD3ζ |
| CD83 | CD80 | CD137/41BB | CD3δ |
| CD83 | CD80 | CD137/41BB | CD3γ |
| CD83 | CD80 | CD137/41BB | CD3ε |
| CD83 | CD80 | CD137/41BB | FcγRI-γ |
| CD83 | CD80 | CD137/41BB | FcγRIII-γ |
| CD83 | CD80 | CD137/41BB | FcεRIβ |
| CD83 | CD80 | CD137/41BB | FcεRIγ |
| CD83 | CD80 | CD137/41BB | DAP10 |
| CD83 | CD80 | CD137/41BB | DAP12 |
| CD83 | CD80 | CD137/41BB | CD32 |
| CD83 | CD80 | CD137/41BB | CD79a |
| CD83 | CD80 | CD137/41BB | CD79b |
| CD83 | CD80 | ICOS | CD8 |
| CD83 | CD80 | ICOS | CD3ζ |
| CD83 | CD80 | ICOS | CD3δ |
| CD83 | CD80 | ICOS | CD3γ |
| CD83 | CD80 | ICOS | CD3ε |
| CD83 | CD80 | ICOS | FcγRI-γ |
| CD83 | CD80 | ICOS | FcγRIII-γ |
| CD83 | CD80 | ICOS | FcεRIβ |
| CD83 | CD80 | ICOS | FcεRIγ |
| CD83 | CD80 | ICOS | DAP10 |
| CD83 | CD80 | ICOS | DAP12 |
| CD83 | CD80 | ICOS | CD32 |
| CD83 | CD80 | ICOS | CD79a |
| CD83 | CD80 | ICOS | CD79b |
| CD83 | CD80 | CD27 | CD8 |
| CD83 | CD80 | CD27 | CD3ζ |
| CD83 | CD80 | CD27 | CD3δ |
| CD83 | CD80 | CD27 | CD3γ |
| CD83 | CD80 | CD27 | CD3ε |
| CD83 | CD80 | CD27 | FcγRI-γ |
| CD83 | CD80 | CD27 | FcγRIII-γ |
| CD83 | CD80 | CD27 | FcεRIβ |
| CD83 | CD80 | CD27 | FcεRIγ |
| CD83 | CD80 | CD27 | DAP10 |
| CD83 | CD80 | CD27 | DAP12 |
| CD83 | CD80 | CD27 | CD32 |
| CD83 | CD80 | CD27 | CD79a |
| CD83 | CD80 | CD27 | CD79b |
| CD83 | CD80 | CD28δ | CD8 |
| CD83 | CD80 | CD28δ | CD3ζ |
| CD83 | CD80 | CD28δ | CD3δ |
| CD83 | CD80 | CD28δ | CD3γ |
| CD83 | CD80 | CD28δ | CD3ε |
| CD83 | CD80 | CD28δ | FcγRI-γ |
| CD83 | CD80 | CD28δ | FcγRIII-γ |
| CD83 | CD80 | CD28δ | FcεRIβ |
| CD83 | CD80 | CD28δ | FcεRIγ |
| CD83 | CD80 | CD28δ | DAP10 |
| CD83 | CD80 | CD28δ | DAP12 |
| CD83 | CD80 | CD28δ | CD32 |
| CD83 | CD80 | CD28δ | CD79a |
| CD83 | CD80 | CD28δ | CD79b |
| CD83 | CD80 | CD80 | CD8 |
| CD83 | CD80 | CD80 | CD3ζ |
| CD83 | CD80 | CD80 | CD3δ |
| CD83 | CD80 | CD80 | CD3γ |
| CD83 | CD80 | CD80 | CD3ε |
| CD83 | CD80 | CD80 | FcγRI-γ |
| CD83 | CD80 | CD80 | FcγRIII-γ |
| CD83 | CD80 | CD80 | FcεRIβ |
| CD83 | CD80 | CD80 | FcεRIγ |
| CD83 | CD80 | CD80 | DAP10 |
| CD83 | CD80 | CD80 | DAP12 |
| CD83 | CD80 | CD80 | CD32 |
| CD83 | CD80 | CD80 | CD79a |
| CD83 | CD80 | CD80 | CD79b |
| CD83 | CD80 | CD86 | CD8 |
| CD83 | CD80 | CD86 | CD3ζ |
| CD83 | CD80 | CD86 | CD3δ |
| CD83 | CD80 | CD86 | CD3γ |
| CD83 | CD80 | CD86 | CD3ε |
| CD83 | CD80 | CD86 | FcγRI-γ |
| CD83 | CD80 | CD86 | FcγRIII-γ |
| CD83 | CD80 | CD86 | FcεRIβ |
| CD83 | CD80 | CD86 | FcεRIγ |
| CD83 | CD80 | CD86 | DAP10 |
| CD83 | CD80 | CD86 | DAP12 |
| CD83 | CD80 | CD86 | CD32 |
| CD83 | CD80 | CD86 | CD79a |
| CD83 | CD80 | CD86 | CD79b |
| CD83 | CD80 | OX40 | CD8 |
| CD83 | CD80 | OX40 | CD3ζ |
| CD83 | CD80 | OX40 | CD3δ |
| CD83 | CD80 | OX40 | CD3γ |
| CD83 | CD80 | OX40 | CD3ε |
| CD83 | CD80 | OX40 | FcγRI-γ |
| CD83 | CD80 | OX40 | FcγRIII-γ |
| CD83 | CD80 | OX40 | FcεRIβ |
| CD83 | CD80 | OX40 | FcεRIγ |
| CD83 | CD80 | OX40 | DAP10 |
| CD83 | CD80 | OX40 | DAP12 |
| CD83 | CD80 | OX40 | CD32 |
| CD83 | CD80 | OX40 | CD79a |
| CD83 | CD80 | OX40 | CD79b |
| CD83 | CD80 | DAP10 | CD8 |
| CD83 | CD80 | DAP10 | CD3ζ |
| CD83 | CD80 | DAP10 | CD3δ |
| CD83 | CD80 | DAP10 | CD3γ |
| CD83 | CD80 | DAP10 | CD3ε |
| CD83 | CD80 | DAP10 | FcγRI-γ |
| CD83 | CD80 | DAP10 | FcγRIII-γ |
| CD83 | CD80 | DAP10 | FcεRIβ |
| CD83 | CD80 | DAP10 | FcεRIγ |
| CD83 | CD80 | DAP10 | DAP10 |
| CD83 | CD80 | DAP10 | DAP12 |
| CD83 | CD80 | DAP10 | CD32 |
| CD83 | CD80 | DAP10 | CD79a |
| CD83 | CD80 | DAP10 | CD79b |
| CD83 | CD80 | DAP12 | CD8 |
| CD83 | CD80 | DAP12 | CD3ζ |
| CD83 | CD80 | DAP12 | CD3δ |
| CD83 | CD80 | DAP12 | CD3γ |
| CD83 | CD80 | DAP12 | CD3ε |
| CD83 | CD80 | DAP12 | FcγRI-γ |
| CD83 | CD80 | DAP12 | FcγRIII-γ |
| CD83 | CD80 | DAP12 | FcεRIβ |
| CD83 | CD80 | DAP12 | FcεRIγ |
| CD83 | CD80 | DAP12 | DAP10 |
| CD83 | CD80 | DAP12 | DAP12 |
| CD83 | CD80 | DAP12 | CD32 |
| CD83 | CD80 | DAP12 | CD79a |
| CD83 | CD80 | DAP12 | CD79b |
| CD83 | CD80 | MyD88 | CD8 |
| CD83 | CD80 | MyD88 | CD3ζ |
| CD83 | CD80 | MyD88 | CD3δ |
| CD83 | CD80 | MyD88 | CD3γ |
| CD83 | CD80 | MyD88 | CD3ε |
| CD83 | CD80 | MyD88 | FcγRI-γ |
| CD83 | CD80 | MyD88 | FcγRIII-γ |
| CD83 | CD80 | MyD88 | FcεRIβ |
| CD83 | CD80 | MyD88 | FcεRIγ |
| CD83 | CD80 | MyD88 | DAP10 |
| CD83 | CD80 | MyD88 | DAP12 |
| CD83 | CD80 | MyD88 | CD32 |
| CD83 | CD80 | MyD88 | CD79a |
| CD83 | CD80 | MyD88 | CD79b |
| CD83 | CD80 | CD7 | CD8 |
| CD83 | CD80 | CD7 | CD3ζ |
| CD83 | CD80 | CD7 | CD3δ |
| CD83 | CD80 | CD7 | CD3γ |
| CD83 | CD80 | CD7 | CD3ε |
| CD83 | CD80 | CD7 | FcγRI-γ |
| CD83 | CD80 | CD7 | FcγRIII-γ |
| CD83 | CD80 | CD7 | FcεRIβ |
| CD83 | CD80 | CD7 | FcεRIγ |
| CD83 | CD80 | CD7 | DAP10 |
| CD83 | CD80 | CD7 | DAP12 |
| CD83 | CD80 | CD7 | CD32 |
| CD83 | CD80 | CD7 | CD79a |
| CD83 | CD80 | CD7 | CD79b |
| CD83 | CD80 | BTNL3 | CD8 |
| CD83 | CD80 | BTNL3 | CD3ζ |
| CD83 | CD80 | BTNL3 | CD3δ |
| CD83 | CD80 | BTNL3 | CD3γ |
| CD83 | CD80 | BTNL3 | CD3ε |
| CD83 | CD80 | BTNL3 | FcγRI-γ |
| CD83 | CD80 | BTNL3 | FcγRIII-γ |
| CD83 | CD80 | BTNL3 | FcεRIβ |
| CD83 | CD80 | BTNL3 | FcεRIγ |
| CD83 | CD80 | BTNL3 | DAP10 |
| CD83 | CD80 | BTNL3 | DAP12 |
| CD83 | CD80 | BTNL3 | CD32 |
| CD83 | CD80 | BTNL3 | CD79a |
| CD83 | CD80 | BTNL3 | CD79b |
| CD83 | CD80 | NKG2D | CD8 |
| CD83 | CD80 | NKG2D | CD3ζ |
| CD83 | CD80 | NKG2D | CD3δ |
| CD83 | CD80 | NKG2D | CD3γ |
| CD83 | CD80 | NKG2D | CD3ε |
| CD83 | CD80 | NKG2D | FcγRI-γ |
| CD83 | CD80 | NKG2D | FcγRIII-γ |
| CD83 | CD80 | NKG2D | FcεRIβ |
| CD83 | CD80 | NKG2D | FcεRIγ |
| CD83 | CD80 | NKG2D | DAP10 |
| CD83 | CD80 | NKG2D | DAP12 |
| CD83 | CD80 | NKG2D | CD32 |
| CD83 | CD80 | NKG2D | CD79a |
| CD83 | CD80 | NKG2D | CD79b |
| CD83 | CD86 | CD28 | CD8 |
| CD83 | CD86 | CD28 | CD3ζ |
| CD83 | CD86 | CD28 | CD3δ |
| CD83 | CD86 | CD28 | CD3γ |
| CD83 | CD86 | CD28 | CD3ε |
| CD83 | CD86 | CD28 | FcγRI-γ |
| CD83 | CD86 | CD28 | FcγRIII-γ |
| CD83 | CD86 | CD28 | FcεRIβ |
| CD83 | CD86 | CD28 | FcεRIγ |
| CD83 | CD86 | CD28 | DAP10 |
| CD83 | CD86 | CD28 | DAP12 |
| CD83 | CD86 | CD28 | CD32 |
| CD83 | CD86 | CD28 | CD79a |
| CD83 | CD86 | CD28 | CD79b |
| CD83 | CD86 | CD8 | CD8 |
| CD83 | CD86 | CD8 | CD3ζ |
| CD83 | CD86 | CD8 | CD3δ |
| CD83 | CD86 | CD8 | CD3γ |
| CD83 | CD86 | CD8 | CD3ε |
| CD83 | CD86 | CD8 | FcγRI-γ |
| CD83 | CD86 | CD8 | FcγRIII-γ |
| CD83 | CD86 | CD8 | FcεRIβ |
| CD83 | CD86 | CD8 | FcεRIγ |
| CD83 | CD86 | CD8 | DAP10 |
| CD83 | CD86 | CD8 | DAP12 |
| CD83 | CD86 | CD8 | CD32 |
| CD83 | CD86 | CD8 | CD79a |
| CD83 | CD86 | CD8 | CD79b |
| CD83 | CD86 | CD4 | CD8 |
| CD83 | CD86 | CD4 | CD3ζ |
| CD83 | CD86 | CD4 | CD3δ |
| CD83 | CD86 | CD4 | CD3γ |
| CD83 | CD86 | CD4 | CD3ε |
| CD83 | CD86 | CD4 | FcγRI-γ |
| CD83 | CD86 | CD4 | FcγRIII-γ |
| CD83 | CD86 | CD4 | FcεRIβ |
| CD83 | CD86 | CD4 | FcεRIγ |
| CD83 | CD86 | CD4 | DAP10 |
| CD83 | CD86 | CD4 | DAP12 |
| CD83 | CD86 | CD4 | CD32 |
| CD83 | CD86 | CD4 | CD79a |
| CD83 | CD86 | CD4 | CD79b |
| CD83 | CD86 | b2c | CD8 |
| CD83 | CD86 | b2c | CD3ζ |
| CD83 | CD86 | b2c | CD3δ |
| CD83 | CD86 | b2c | CD3γ |
| CD83 | CD86 | b2c | CD3ε |
| CD83 | CD86 | b2c | FcγRI-γ |
| CD83 | CD86 | b2c | FcγRIII-γ |
| CD83 | CD86 | b2c | FcεRIβ |
| CD83 | CD86 | b2c | FcεRIγ |
| CD83 | CD86 | b2c | DAP10 |
| CD83 | CD86 | b2c | DAP12 |
| CD83 | CD86 | b2c | CD32 |
| CD83 | CD86 | b2c | CD79a |
| CD83 | CD86 | b2c | CD79b |
| CD83 | CD86 | CD137/41BB | CD8 |
| CD83 | CD86 | CD137/41BB | CD3ζ |
| CD83 | CD86 | CD137/41BB | CD3δ |
| CD83 | CD86 | CD137/41BB | CD3γ |
| CD83 | CD86 | CD137/41BB | CD3ε |
| CD83 | CD86 | CD137/41BB | FcγRI-γ |
| CD83 | CD86 | CD137/41BB | FcγRIII-γ |
| CD83 | CD86 | CD137/41BB | FcεRIβ |
| CD83 | CD86 | CD137/41BB | FcεRIγ |
| CD83 | CD86 | CD137/41BB | DAP10 |
| CD83 | CD86 | CD137/41BB | DAP12 |
| CD83 | CD86 | CD137/41BB | CD32 |
| CD83 | CD86 | CD137/41BB | CD79a |
| CD83 | CD86 | CD137/41BB | CD79b |
| CD83 | CD86 | ICOS | CD8 |
| CD83 | CD86 | ICOS | CD3ζ |
| CD83 | CD86 | ICOS | CD3δ |
| CD83 | CD86 | ICOS | CD3γ |
| CD83 | CD86 | ICOS | CD3ε |
| CD83 | CD86 | ICOS | FcγRI-γ |
| CD83 | CD86 | ICOS | FcγRIII-γ |
| CD83 | CD86 | ICOS | FcεRIβ |
| CD83 | CD86 | ICOS | FcεRIγ |
| CD83 | CD86 | ICOS | DAP10 |
| CD83 | CD86 | ICOS | DAP12 |
| CD83 | CD86 | ICOS | CD32 |
| CD83 | CD86 | ICOS | CD79a |
| CD83 | CD86 | ICOS | CD79b |
| CD83 | CD86 | CD27 | CD8 |
| CD83 | CD86 | CD27 | CD3ζ |
| CD83 | CD86 | CD27 | CD3δ |
| CD83 | CD86 | CD27 | CD3γ |
| CD83 | CD86 | CD27 | CD3ε |
| CD83 | CD86 | CD27 | FcγRI-γ |
| CD83 | CD86 | CD27 | FcγRIII-γ |
| CD83 | CD86 | CD27 | FcεRIβ |
| CD83 | CD86 | CD27 | FcεRIγ |
| CD83 | CD86 | CD27 | DAP10 |
| CD83 | CD86 | CD27 | DAP12 |
| CD83 | CD86 | CD27 | CD32 |
| CD83 | CD86 | CD27 | CD79a |
| CD83 | CD86 | CD27 | CD79b |
| CD83 | CD86 | CD28δ | CD8 |
| CD83 | CD86 | CD28δ | CD3ζ |
| CD83 | CD86 | CD28δ | CD3δ |
| CD83 | CD86 | CD28δ | CD3γ |
| CD83 | CD86 | CD28δ | CD3ε |
| CD83 | CD86 | CD28δ | FcγRI-γ |
| CD83 | CD86 | CD28δ | FcγRIII-γ |
| CD83 | CD86 | CD28δ | FcεRIβ |
| CD83 | CD86 | CD28δ | FcεRIγ |
| CD83 | CD86 | CD28δ | DAP10 |
| CD83 | CD86 | CD28δ | DAP12 |
| CD83 | CD86 | CD28δ | CD32 |
| CD83 | CD86 | CD28δ | CD79a |
| CD83 | CD86 | CD28δ | CD79b |
| CD83 | CD86 | CD80 | CD8 |
| CD83 | CD86 | CD80 | CD3ζ |
| CD83 | CD86 | CD80 | CD3δ |
| CD83 | CD86 | CD80 | CD3γ |
| CD83 | CD86 | CD80 | CD3ε |
| CD83 | CD86 | CD80 | FcγRI-γ |
| CD83 | CD86 | CD80 | FcγRIII-γ |
| CD83 | CD86 | CD80 | FcεRIβ |
| CD83 | CD86 | CD80 | FcεRIγ |
| CD83 | CD86 | CD80 | DAP10 |
| CD83 | CD86 | CD80 | DAP12 |
| CD83 | CD86 | CD80 | CD32 |
| CD83 | CD86 | CD80 | CD79a |
| CD83 | CD86 | CD80 | CD79b |
| CD83 | CD86 | CD86 | CD8 |
| CD83 | CD86 | CD86 | CD3ζ |
| CD83 | CD86 | CD86 | CD3δ |
| CD83 | CD86 | CD86 | CD3γ |
| CD83 | CD86 | CD86 | CD3ε |
| CD83 | CD86 | CD86 | FcγRI-γ |
| CD83 | CD86 | CD86 | FcγRIII-γ |
| CD83 | CD86 | CD86 | FcεRIβ |
| CD83 | CD86 | CD86 | FcεRIγ |
| CD83 | CD86 | CD86 | DAP10 |
| CD83 | CD86 | CD86 | DAP12 |
| CD83 | CD86 | CD86 | CD32 |
| CD83 | CD86 | CD86 | CD79a |
| CD83 | CD86 | CD86 | CD79b |
| CD83 | CD86 | OX40 | CD8 |
| CD83 | CD86 | OX40 | CD3ζ |
| CD83 | CD86 | OX40 | CD3δ |
| CD83 | CD86 | OX40 | CD3γ |
| CD83 | CD86 | OX40 | CD3ε |
| CD83 | CD86 | OX40 | FcγRI-γ |
| CD83 | CD86 | OX40 | FcγRIII-γ |
| CD83 | CD86 | OX40 | FcεRIβ |
| CD83 | CD86 | OX40 | FcεRIγ |
| CD83 | CD86 | OX40 | DAP10 |
| CD83 | CD86 | OX40 | DAP12 |
| CD83 | CD86 | OX40 | CD32 |
| CD83 | CD86 | OX40 | CD79a |
| CD83 | CD86 | OX40 | CD79b |
| CD83 | CD86 | DAP10 | CD8 |
| CD83 | CD86 | DAP10 | CD3ζ |
| CD83 | CD86 | DAP10 | CD3δ |
| CD83 | CD86 | DAP10 | CD3γ |
| CD83 | CD86 | DAP10 | CD3ε |
| CD83 | CD86 | DAP10 | FcγRI-γ |
| CD83 | CD86 | DAP10 | FcγRIII-γ |
| CD83 | CD86 | DAP10 | FcεRIβ |
| CD83 | CD86 | DAP10 | FcεRIγ |
| CD83 | CD86 | DAP10 | DAP10 |
| CD83 | CD86 | DAP10 | DAP12 |
| CD83 | CD86 | DAP10 | CD32 |
| CD83 | CD86 | DAP10 | CD79a |
| CD83 | CD86 | DAP10 | CD79b |
| CD83 | CD86 | DAP12 | CD8 |
| CD83 | CD86 | DAP12 | CD3ζ |
| CD83 | CD86 | DAP12 | CD3δ |
| CD83 | CD86 | DAP12 | CD3γ |
| CD83 | CD86 | DAP12 | CD3ε |
| CD83 | CD86 | DAP12 | FcγRI-γ |
| CD83 | CD86 | DAP12 | FcγRIII-γ |
| CD83 | CD86 | DAP12 | FcεRIβ |
| CD83 | CD86 | DAP12 | FcεRIγ |
| CD83 | CD86 | DAP12 | DAP10 |
| CD83 | CD86 | DAP12 | DAP12 |
| CD83 | CD86 | DAP12 | CD32 |
| CD83 | CD86 | DAP12 | CD79a |
| CD83 | CD86 | DAP12 | CD79b |
| CD83 | CD86 | MyD88 | CD8 |
| CD83 | CD86 | MyD88 | CD3ζ |
| CD83 | CD86 | MyD88 | CD3δ |
| CD83 | CD86 | MyD88 | CD3γ |
| CD83 | CD86 | MyD88 | CD3ε |
| CD83 | CD86 | MyD88 | FcγRI-γ |
| CD83 | CD86 | MyD88 | FcγRIII-γ |
| CD83 | CD86 | MyD88 | FcεRIβ |
| CD83 | CD86 | MyD88 | FcεRIγ |
| CD83 | CD86 | MyD88 | DAP10 |
| CD83 | CD86 | MyD88 | DAP12 |
| CD83 | CD86 | MyD88 | CD32 |
| CD83 | CD86 | MyD88 | CD79a |
| CD83 | CD86 | MyD88 | CD79b |
| CD83 | CD86 | CD7 | CD8 |
| CD83 | CD86 | CD7 | CD3ζ |
| CD83 | CD86 | CD7 | CD3δ |
| CD83 | CD86 | CD7 | CD3γ |
| CD83 | CD86 | CD7 | CD3ε |
| CD83 | CD86 | CD7 | FcγRI-γ |
| CD83 | CD86 | CD7 | FcγRIII-γ |
| CD83 | CD86 | CD7 | FcεRIβ |
| CD83 | CD86 | CD7 | FcεRIγ |
| CD83 | CD86 | CD7 | DAP10 |
| CD83 | CD86 | CD7 | DAP12 |
| CD83 | CD86 | CD7 | CD32 |
| CD83 | CD86 | CD7 | CD79a |
| CD83 | CD86 | CD7 | CD79b |
| CD83 | CD86 | BTNL3 | CD8 |
| CD83 | CD86 | BTNL3 | CD3ζ |
| CD83 | CD86 | BTNL3 | CD3δ |
| CD83 | CD86 | BTNL3 | CD3γ |
| CD83 | CD86 | BTNL3 | CD3ε |
| CD83 | CD86 | BTNL3 | FcγRI-γ |
| CD83 | CD86 | BTNL3 | FcγRIII-γ |
| CD83 | CD86 | BTNL3 | FcεRIβ |
| CD83 | CD86 | BTNL3 | FcεRIγ |
| CD83 | CD86 | BTNL3 | DAP10 |
| CD83 | CD86 | BTNL3 | DAP12 |
| CD83 | CD86 | BTNL3 | CD32 |
| CD83 | CD86 | BTNL3 | CD79a |
| CD83 | CD86 | BTNL3 | CD79b |
| CD83 | CD86 | NKG2D | CD8 |
| CD83 | CD86 | NKG2D | CD3ζ |
| CD83 | CD86 | NKG2D | CD3δ |
| CD83 | CD86 | NKG2D | CD3γ |
| CD83 | CD86 | NKG2D | CD3ε |
| CD83 | CD86 | NKG2D | FcγRI-γ |
| CD83 | CD86 | NKG2D | FcγRIII-γ |
| CD83 | CD86 | NKG2D | FcεRIβ |
| CD83 | CD86 | NKG2D | FcεRIγ |
| CD83 | CD86 | NKG2D | DAP10 |
| CD83 | CD86 | NKG2D | DAP12 |
| CD83 | CD86 | NKG2D | CD32 |
| CD83 | CD86 | NKG2D | CD79a |
| CD83 | CD86 | NKG2D | CD79b |
| CD83 | OX40 | CD28 | CD8 |
| CD83 | OX40 | CD28 | CD3ζ |
| CD83 | OX40 | CD28 | CD3δ |
| CD83 | OX40 | CD28 | CD3γ |
| CD83 | OX40 | CD28 | CD3ε |
| CD83 | OX40 | CD28 | FcγRI-γ |
| CD83 | OX40 | CD28 | FcγRIII-γ |
| CD83 | OX40 | CD28 | FcεRIβ |
| CD83 | OX40 | CD28 | FcεRIγ |
| CD83 | OX40 | CD28 | DAP10 |
| CD83 | OX40 | CD28 | DAP12 |
| CD83 | OX40 | CD28 | CD32 |
| CD83 | OX40 | CD28 | CD79a |
| CD83 | OX40 | CD28 | CD79b |
| CD83 | OX40 | CD8 | CD8 |
| CD83 | OX40 | CD8 | CD3ζ |
| CD83 | OX40 | CD8 | CD3δ |
| CD83 | OX40 | CD8 | CD3γ |
| CD83 | OX40 | CD8 | CD3ε |
| CD83 | OX40 | CD8 | FcγRI-γ |
| CD83 | OX40 | CD8 | FcγRIII-γ |
| CD83 | OX40 | CD8 | FcεRIβ |
| CD83 | OX40 | CD8 | FcεRIγ |
| CD83 | OX40 | CD8 | DAP10 |
| CD83 | OX40 | CD8 | DAP12 |
| CD83 | OX40 | CD8 | CD32 |
| CD83 | OX40 | CD8 | CD79a |
| CD83 | OX40 | CD8 | CD79b |
| CD83 | OX40 | CD4 | CD8 |
| CD83 | OX40 | CD4 | CD3ζ |
| CD83 | OX40 | CD4 | CD3δ |
| CD83 | OX40 | CD4 | CD3γ |
| CD83 | OX40 | CD4 | CD3ε |
| CD83 | OX40 | CD4 | FcγRI-γ |
| CD83 | OX40 | CD4 | FcγRIII-γ |
| CD83 | OX40 | CD4 | FcεRIβ |
| CD83 | OX40 | CD4 | FcεRIγ |
| CD83 | OX40 | CD4 | DAP10 |
| CD83 | OX40 | CD4 | DAP12 |
| CD83 | OX40 | CD4 | CD32 |
| CD83 | OX40 | CD4 | CD79a |
| CD83 | OX40 | CD4 | CD79b |
| CD83 | OX40 | b2c | CD8 |
| CD83 | OX40 | b2c | CD3ζ |
| CD83 | OX40 | b2c | CD3δ |
| CD83 | OX40 | b2c | CD3γ |
| CD83 | OX40 | b2c | CD3ε |
| CD83 | OX40 | b2c | FcγRI-γ |
| CD83 | OX40 | b2c | FcγRIII-γ |
| CD83 | OX40 | b2c | FcεRIβ |
| CD83 | OX40 | b2c | FcεRIγ |
| CD83 | OX40 | b2c | DAP10 |
| CD83 | OX40 | b2c | DAP12 |
| CD83 | OX40 | b2c | CD32 |
| CD83 | OX40 | b2c | CD79a |
| CD83 | OX40 | b2c | CD79b |
| CD83 | OX40 | CD137/41BB | CD8 |
| CD83 | OX40 | CD137/41BB | CD3ζ |
| CD83 | OX40 | CD137/41BB | CD3δ |
| CD83 | OX40 | CD137/41BB | CD3γ |
| CD83 | OX40 | CD137/41BB | CD3ε |
| CD83 | OX40 | CD137/41BB | FcγRI-γ |
| CD83 | OX40 | CD137/41BB | FcγRIII-γ |
| CD83 | OX40 | CD137/41BB | FcεRIβ |
| CD83 | OX40 | CD137/41BB | FcεRIγ |
| CD83 | OX40 | CD137/41BB | DAP10 |
| CD83 | OX40 | CD137/41BB | DAP12 |
| CD83 | OX40 | CD137/41BB | CD32 |
| CD83 | OX40 | CD137/41BB | CD79a |
| CD83 | OX40 | CD137/41BB | CD79b |
| CD83 | OX40 | ICOS | CD8 |
| CD83 | OX40 | ICOS | CD3ζ |
| CD83 | OX40 | ICOS | CD3δ |
| CD83 | OX40 | ICOS | CD3γ |
| CD83 | OX40 | ICOS | CD3ε |
| CD83 | OX40 | ICOS | FcγRI-γ |
| CD83 | OX40 | ICOS | FcγRIII-γ |
| CD83 | OX40 | ICOS | FcεRIβ |
| CD83 | OX40 | ICOS | FcεRIγ |
| CD83 | OX40 | ICOS | DAP10 |
| CD83 | OX40 | ICOS | DAP12 |
| CD83 | OX40 | ICOS | CD32 |
| CD83 | OX40 | ICOS | CD79a |
| CD83 | OX40 | ICOS | CD79b |
| CD83 | OX40 | CD27 | CD8 |
| CD83 | OX40 | CD27 | CD3ζ |
| CD83 | OX40 | CD27 | CD3δ |
| CD83 | OX40 | CD27 | CD3γ |
| CD83 | OX40 | CD27 | CD3ε |
| CD83 | OX40 | CD27 | FcγRI-γ |
| CD83 | OX40 | CD27 | FcγRIII-γ |
| CD83 | OX40 | CD27 | FcεRIβ |
| CD83 | OX40 | CD27 | FcεRIγ |
| CD83 | OX40 | CD27 | DAP10 |
| CD83 | OX40 | CD27 | DAP12 |
| CD83 | OX40 | CD27 | CD32 |
| CD83 | OX40 | CD27 | CD79a |
| CD83 | OX40 | CD27 | CD79b |
| CD83 | OX40 | CD28δ | CD8 |
| CD83 | OX40 | CD28δ | CD3ζ |
| CD83 | OX40 | CD28δ | CD3δ |
| CD83 | OX40 | CD28δ | CD3γ |
| CD83 | OX40 | CD28δ | CD3ε |
| CD83 | OX40 | CD28δ | FcγRI-γ |
| CD83 | OX40 | CD28δ | FcγRIII-γ |
| CD83 | OX40 | CD28δ | FcεRIβ |
| CD83 | OX40 | CD28δ | FcεRIγ |
| CD83 | OX40 | CD28δ | DAP10 |
| CD83 | OX40 | CD28δ | DAP12 |
| CD83 | OX40 | CD28δ | CD32 |
| CD83 | OX40 | CD28δ | CD79a |
| CD83 | OX40 | CD28δ | CD79b |
| CD83 | OX40 | CD80 | CD8 |
| CD83 | OX40 | CD80 | CD3ζ |
| CD83 | OX40 | CD80 | CD3δ |
| CD83 | OX40 | CD80 | CD3γ |
| CD83 | OX40 | CD80 | CD3ε |
| CD83 | OX40 | CD80 | FcγRI-γ |
| CD83 | OX40 | CD80 | FcγRIII-γ |
| CD83 | OX40 | CD80 | FcεRIβ |
| CD83 | OX40 | CD80 | FcεRIγ |
| CD83 | OX40 | CD80 | DAP10 |
| CD83 | OX40 | CD80 | DAP12 |
| CD83 | OX40 | CD80 | CD32 |
| CD83 | OX40 | CD80 | CD79a |
| CD83 | OX40 | CD80 | CD79b |
| CD83 | OX40 | CD86 | CD8 |
| CD83 | OX40 | CD86 | CD3ζ |
| CD83 | OX40 | CD86 | CD3δ |
| CD83 | OX40 | CD86 | CD3γ |
| CD83 | OX40 | CD86 | CD3ε |
| CD83 | OX40 | CD86 | FcγRI-γ |
| CD83 | OX40 | CD86 | FcγRIII-γ |
| CD83 | OX40 | CD86 | FcεRIβ |
| CD83 | OX40 | CD86 | FcεRIγ |
| CD83 | OX40 | CD86 | DAP10 |
| CD83 | OX40 | CD86 | DAP12 |
| CD83 | OX40 | CD86 | CD32 |
| CD83 | OX40 | CD86 | CD79a |
| CD83 | OX40 | CD86 | CD79b |
| CD83 | OX40 | OX40 | CD8 |
| CD83 | OX40 | OX40 | CD3ζ |
| CD83 | OX40 | OX40 | CD3δ |
| CD83 | OX40 | OX40 | CD3γ |
| CD83 | OX40 | OX40 | CD3ε |
| CD83 | OX40 | OX40 | FcγRI-γ |
| CD83 | OX40 | OX40 | FcγRIII-γ |
| CD83 | OX40 | OX40 | FcεRIβ |
| CD83 | OX40 | OX40 | FcεRIγ |
| CD83 | OX40 | OX40 | DAP10 |
| CD83 | OX40 | OX40 | DAP12 |
| CD83 | OX40 | OX40 | CD32 |
| CD83 | OX40 | OX40 | CD79a |
| CD83 | OX40 | OX40 | CD79b |
| CD83 | OX40 | DAP10 | CD8 |
| CD83 | OX40 | DAP10 | CD3ζ |
| CD83 | OX40 | DAP10 | CD3δ |
| CD83 | OX40 | DAP10 | CD3γ |
| CD83 | OX40 | DAP10 | CD3ε |
| CD83 | OX40 | DAP10 | FcγRI-γ |
| CD83 | OX40 | DAP10 | FcγRIII-γ |
| CD83 | OX40 | DAP10 | FcεRIβ |
| CD83 | OX40 | DAP10 | FcεRIγ |
| CD83 | OX40 | DAP10 | DAP10 |
| CD83 | OX40 | DAP10 | DAP12 |
| CD83 | OX40 | DAP10 | CD32 |
| CD83 | OX40 | DAP10 | CD79a |
| CD83 | OX40 | DAP10 | CD79b |
| CD83 | OX40 | DAP12 | CD8 |
| CD83 | OX40 | DAP12 | CD3ζ |
| CD83 | OX40 | DAP12 | CD3δ |
| CD83 | OX40 | DAP12 | CD3γ |
| CD83 | OX40 | DAP12 | CD3ε |
| CD83 | OX40 | DAP12 | FcγRI-γ |
| CD83 | OX40 | DAP12 | FcγRIII-γ |
| CD83 | OX40 | DAP12 | FcεRIβ |
| CD83 | OX40 | DAP12 | FcεRIγ |
| CD83 | OX40 | DAP12 | DAP10 |
| CD83 | OX40 | DAP12 | DAP12 |
| CD83 | OX40 | DAP12 | CD32 |
| CD83 | OX40 | DAP12 | CD79a |
| CD83 | OX40 | DAP12 | CD79b |
| CD83 | OX40 | MyD88 | CD8 |
| CD83 | OX40 | MyD88 | CD3ζ |
| CD83 | OX40 | MyD88 | CD3δ |
| CD83 | OX40 | MyD88 | CD3γ |
| CD83 | OX40 | MyD88 | CD3ε |
| CD83 | OX40 | MyD88 | FcγRI-γ |
| CD83 | OX40 | MyD88 | FcγRIII-γ |
| CD83 | OX40 | MyD88 | FcεRIβ |
| CD83 | OX40 | MyD88 | FcεRIγ |
| CD83 | OX40 | MyD88 | DAP10 |
| CD83 | OX40 | MyD88 | DAP12 |
| CD83 | OX40 | MyD88 | CD32 |
| CD83 | OX40 | MyD88 | CD79a |
| CD83 | OX40 | MyD88 | CD79b |
| CD83 | OX40 | CD7 | CD8 |
| CD83 | OX40 | CD7 | CD3ζ |
| CD83 | OX40 | CD7 | CD3δ |
| CD83 | OX40 | CD7 | CD3γ |
| CD83 | OX40 | CD7 | CD3ε |
| CD83 | OX40 | CD7 | FcγRI-γ |
| CD83 | OX40 | CD7 | FcγRIII-γ |
| CD83 | OX40 | CD7 | FcεRIβ |
| CD83 | OX40 | CD7 | FcεRIγ |
| CD83 | OX40 | CD7 | DAP10 |
| CD83 | OX40 | CD7 | DAP12 |
| CD83 | OX40 | CD7 | CD32 |
| CD83 | OX40 | CD7 | CD79a |
| CD83 | OX40 | CD7 | CD79b |
| CD83 | OX40 | BTNL3 | CD8 |
| CD83 | OX40 | BTNL3 | CD3ζ |
| CD83 | OX40 | BTNL3 | CD3δ |
| CD83 | OX40 | BTNL3 | CD3γ |
| CD83 | OX40 | BTNL3 | CD3ε |
| CD83 | OX40 | BTNL3 | FcγRI-γ |
| CD83 | OX40 | BTNL3 | FcγRIII-γ |
| CD83 | OX40 | BTNL3 | FcεRIβ |
| CD83 | OX40 | BTNL3 | FcεRIγ |
| CD83 | OX40 | BTNL3 | DAP10 |
| CD83 | OX40 | BTNL3 | DAP12 |
| CD83 | OX40 | BTNL3 | CD32 |
| CD83 | OX40 | BTNL3 | CD79a |
| CD83 | OX40 | BTNL3 | CD79b |
| CD83 | OX40 | NKG2D | CD8 |
| CD83 | OX40 | NKG2D | CD3ζ |
| CD83 | OX40 | NKG2D | CD3δ |
| CD83 | OX40 | NKG2D | CD3γ |
| CD83 | OX40 | NKG2D | CD3ε |
| CD83 | OX40 | NKG2D | FcγRI-γ |
| CD83 | OX40 | NKG2D | FcγRIII-γ |
| CD83 | OX40 | NKG2D | FcεRIβ |
| CD83 | OX40 | NKG2D | FcεRIγ |
| CD83 | OX40 | NKG2D | DAP10 |
| CD83 | OX40 | NKG2D | DAP12 |
| CD83 | OX40 | NKG2D | CD32 |
| CD83 | OX40 | NKG2D | CD79a |
| CD83 | OX40 | NKG2D | CD79b |
| CD83 | DAP10 | CD28 | CD8 |
| CD83 | DAP10 | CD28 | CD3ζ |
| CD83 | DAP10 | CD28 | CD3δ |
| CD83 | DAP10 | CD28 | CD3γ |
| CD83 | DAP10 | CD28 | CD3ε |
| CD83 | DAP10 | CD28 | FcγRI-γ |
| CD83 | DAP10 | CD28 | FcγRIII-γ |
| CD83 | DAP10 | CD28 | FcεRIβ |
| CD83 | DAP10 | CD28 | FcεRIγ |
| CD83 | DAP10 | CD28 | DAP10 |
| CD83 | DAP10 | CD28 | DAP12 |
| CD83 | DAP10 | CD28 | CD32 |
| CD83 | DAP10 | CD28 | CD79a |
| CD83 | DAP10 | CD28 | CD79b |
| CD83 | DAP10 | CD8 | CD8 |
| CD83 | DAP10 | CD8 | CD3ζ |
| CD83 | DAP10 | CD8 | CD3δ |
| CD83 | DAP10 | CD8 | CD3γ |
| CD83 | DAP10 | CD8 | CD3ε |
| CD83 | DAP10 | CD8 | FcγRI-γ |
| CD83 | DAP10 | CD8 | FcγRIII-γ |
| CD83 | DAP10 | CD8 | FcεRIβ |
| CD83 | DAP10 | CD8 | FcεRIγ |
| CD83 | DAP10 | CD8 | DAP10 |
| CD83 | DAP10 | CD8 | DAP12 |
| CD83 | DAP10 | CD8 | CD32 |
| CD83 | DAP10 | CD8 | CD79a |
| CD83 | DAP10 | CD8 | CD79b |
| CD83 | DAP10 | CD4 | CD8 |
| CD83 | DAP10 | CD4 | CD3ζ |
| CD83 | DAP10 | CD4 | CD3δ |
| CD83 | DAP10 | CD4 | CD3γ |
| CD83 | DAP10 | CD4 | CD3ε |
| CD83 | DAP10 | CD4 | FcγRI-γ |
| CD83 | DAP10 | CD4 | FcγRIII-γ |
| CD83 | DAP10 | CD4 | FcεRIβ |
| CD83 | DAP10 | CD4 | FcεRIγ |
| CD83 | DAP10 | CD4 | DAP10 |
| CD83 | DAP10 | CD4 | DAP12 |
| CD83 | DAP10 | CD4 | CD32 |
| CD83 | DAP10 | CD4 | CD79a |
| CD83 | DAP10 | CD4 | CD79b |
| CD83 | DAP10 | b2c | CD8 |
| CD83 | DAP10 | b2c | CD3ζ |
| CD83 | DAP10 | b2c | CD3δ |
| CD83 | DAP10 | b2c | CD3γ |
| CD83 | DAP10 | b2c | CD3ε |
| CD83 | DAP10 | b2c | FcγRI-γ |
| CD83 | DAP10 | b2c | FcγRIII-γ |
| CD83 | DAP10 | b2c | FcεRIβ |
| CD83 | DAP10 | b2c | FcεRIγ |
| CD83 | DAP10 | b2c | DAP10 |
| CD83 | DAP10 | b2c | DAP12 |
| CD83 | DAP10 | b2c | CD32 |
| CD83 | DAP10 | b2c | CD79a |
| CD83 | DAP10 | b2c | CD79b |
| CD83 | DAP10 | CD137/41BB | CD8 |
| CD83 | DAP10 | CD137/41BB | CD3ζ |
| CD83 | DAP10 | CD137/41BB | CD3δ |
| CD83 | DAP10 | CD137/41BB | CD3γ |
| CD83 | DAP10 | CD137/41BB | CD3ε |
| CD83 | DAP10 | CD137/41BB | FcγRI-γ |
| CD83 | DAP10 | CD137/41BB | FcγRIII-γ |
| CD83 | DAP10 | CD137/41BB | FcεRIβ |
| CD83 | DAP10 | CD137/41BB | FcεRIγ |
| CD83 | DAP10 | CD137/41BB | DAP10 |
| CD83 | DAP10 | CD137/41BB | DAP12 |
| CD83 | DAP10 | CD137/41BB | CD32 |
| CD83 | DAP10 | CD137/41BB | CD79a |
| CD83 | DAP10 | CD137/41BB | CD79b |
| CD83 | DAP10 | ICOS | CD8 |
| CD83 | DAP10 | ICOS | CD3ζ |
| CD83 | DAP10 | ICOS | CD3δ |
| CD83 | DAP10 | ICOS | CD3γ |
| CD83 | DAP10 | ICOS | CD3ε |
| CD83 | DAP10 | ICOS | FcγRI-γ |
| CD83 | DAP10 | ICOS | FcγRIII-γ |
| CD83 | DAP10 | ICOS | FcεRIβ |
| CD83 | DAP10 | ICOS | FcεRIγ |
| CD83 | DAP10 | ICOS | DAP10 |
| CD83 | DAP10 | ICOS | DAP12 |
| CD83 | DAP10 | ICOS | CD32 |
| CD83 | DAP10 | ICOS | CD79a |
| CD83 | DAP10 | ICOS | CD79b |
| CD83 | DAP10 | CD27 | CD8 |
| CD83 | DAP10 | CD27 | CD3ζ |
| CD83 | DAP10 | CD27 | CD3δ |
| CD83 | DAP10 | CD27 | CD3γ |
| CD83 | DAP10 | CD27 | CD3ε |
| CD83 | DAP10 | CD27 | FcγRI-γ |
| CD83 | DAP10 | CD27 | FcγRIII-γ |
| CD83 | DAP10 | CD27 | FcεRIβ |
| CD83 | DAP10 | CD27 | FcεRIγ |
| CD83 | DAP10 | CD27 | DAP10 |
| CD83 | DAP10 | CD27 | DAP12 |
| CD83 | DAP10 | CD27 | CD32 |
| CD83 | DAP10 | CD27 | CD79a |
| CD83 | DAP10 | CD27 | CD79b |
| CD83 | DAP10 | CD28δ | CD8 |
| CD83 | DAP10 | CD28δ | CD3ζ |
| CD83 | DAP10 | CD28δ | CD3δ |
| CD83 | DAP10 | CD28δ | CD3γ |
| CD83 | DAP10 | CD28δ | CD3ε |
| CD83 | DAP10 | CD28δ | FcγRI-γ |
| CD83 | DAP10 | CD28δ | FcγRIII-γ |
| CD83 | DAP10 | CD28δ | FcεRIβ |
| CD83 | DAP10 | CD28δ | FcεRIγ |
| CD83 | DAP10 | CD28δ | DAP10 |
| CD83 | DAP10 | CD28δ | DAP12 |
| CD83 | DAP10 | CD28δ | CD32 |
| CD83 | DAP10 | CD28δ | CD79a |
| CD83 | DAP10 | CD28δ | CD79b |
| CD83 | DAP10 | CD80 | CD8 |
| CD83 | DAP10 | CD80 | CD3ζ |
| CD83 | DAP10 | CD80 | CD3δ |
| CD83 | DAP10 | CD80 | CD3γ |
| CD83 | DAP10 | CD80 | CD3ε |
| CD83 | DAP10 | CD80 | FcγRI-γ |
| CD83 | DAP10 | CD80 | FcγRIII-γ |
| CD83 | DAP10 | CD80 | FcεRIβ |
| CD83 | DAP10 | CD80 | FcεRIγ |
| CD83 | DAP10 | CD80 | DAP10 |
| CD83 | DAP10 | CD80 | DAP12 |
| CD83 | DAP10 | CD80 | CD32 |
| CD83 | DAP10 | CD80 | CD79a |
| CD83 | DAP10 | CD80 | CD79b |
| CD83 | DAP10 | CD86 | CD8 |
| CD83 | DAP10 | CD86 | CD3ζ |
| CD83 | DAP10 | CD86 | CD3δ |
| CD83 | DAP10 | CD86 | CD3γ |
| CD83 | DAP10 | CD86 | CD3ε |
| CD83 | DAP10 | CD86 | FcγRI-γ |
| CD83 | DAP10 | CD86 | FcγRIII-γ |
| CD83 | DAP10 | CD86 | FcεRIβ |
| CD83 | DAP10 | CD86 | FcεRIγ |
| CD83 | DAP10 | CD86 | DAP10 |
| CD83 | DAP10 | CD86 | DAP12 |
| CD83 | DAP10 | CD86 | CD32 |
| CD83 | DAP10 | CD86 | CD79a |
| CD83 | DAP10 | CD86 | CD79b |
| CD83 | DAP10 | OX40 | CD8 |
| CD83 | DAP10 | OX40 | CD3ζ |
| CD83 | DAP10 | OX40 | CD3δ |
| CD83 | DAP10 | OX40 | CD3γ |
| CD83 | DAP10 | OX40 | CD3ε |
| CD83 | DAP10 | OX40 | FcγRI-γ |
| CD83 | DAP10 | OX40 | FcγRIII-γ |
| CD83 | DAP10 | OX40 | FcεRIβ |
| CD83 | DAP10 | OX40 | FcεRIγ |
| CD83 | DAP10 | OX40 | DAP10 |
| CD83 | DAP10 | OX40 | DAP12 |
| CD83 | DAP10 | OX40 | CD32 |
| CD83 | DAP10 | OX40 | CD79a |
| CD83 | DAP10 | OX40 | CD79b |
| CD83 | DAP10 | DAP10 | CD8 |
| CD83 | DAP10 | DAP10 | CD3ζ |
| CD83 | DAP10 | DAP10 | CD3δ |
| CD83 | DAP10 | DAP10 | CD3γ |
| CD83 | DAP10 | DAP10 | CD3ε |
| CD83 | DAP10 | DAP10 | FcγRI-γ |
| CD83 | DAP10 | DAP10 | FcγRIII-γ |
| CD83 | DAP10 | DAP10 | FcεRIβ |
| CD83 | DAP10 | DAP10 | FcεRIγ |
| CD83 | DAP10 | DAP10 | DAP10 |
| CD83 | DAP10 | DAP10 | DAP12 |
| CD83 | DAP10 | DAP10 | CD32 |
| CD83 | DAP10 | DAP10 | CD79a |
| CD83 | DAP10 | DAP10 | CD79b |
| CD83 | DAP10 | DAP12 | CD8 |
| CD83 | DAP10 | DAP12 | CD3ζ |
| CD83 | DAP10 | DAP12 | CD3δ |
| CD83 | DAP10 | DAP12 | CD3γ |
| CD83 | DAP10 | DAP12 | CD3ε |
| CD83 | DAP10 | DAP12 | FcγRI-γ |
| CD83 | DAP10 | DAP12 | FcγRIII-γ |
| CD83 | DAP10 | DAP12 | FcεRIβ |
| CD83 | DAP10 | DAP12 | FcεRIγ |
| CD83 | DAP10 | DAP12 | DAP10 |
| CD83 | DAP10 | DAP12 | DAP12 |
| CD83 | DAP10 | DAP12 | CD32 |
| CD83 | DAP10 | DAP12 | CD79a |
| CD83 | DAP10 | DAP12 | CD79b |
| CD83 | DAP10 | MyD88 | CD8 |
| CD83 | DAP10 | MyD88 | CD3ζ |
| CD83 | DAP10 | MyD88 | CD3δ |
| CD83 | DAP10 | MyD88 | CD3γ |
| CD83 | DAP10 | MyD88 | CD3ε |
| CD83 | DAP10 | MyD88 | FcγRI-γ |
| CD83 | DAP10 | MyD88 | FcγRIII-γ |
| CD83 | DAP10 | MyD88 | FcεRIβ |
| CD83 | DAP10 | MyD88 | FcεRIγ |
| CD83 | DAP10 | MyD88 | DAP10 |
| CD83 | DAP10 | MyD88 | DAP12 |
| CD83 | DAP10 | MyD88 | CD32 |
| CD83 | DAP10 | MyD88 | CD79a |
| CD83 | DAP10 | MyD88 | CD79b |
| CD83 | DAP10 | CD7 | CD8 |
| CD83 | DAP10 | CD7 | CD3ζ |
| CD83 | DAP10 | CD7 | CD3δ |
| CD83 | DAP10 | CD7 | CD3γ |
| CD83 | DAP10 | CD7 | CD3ε |
| CD83 | DAP10 | CD7 | FcγRI-γ |
| CD83 | DAP10 | CD7 | FcγRIII-γ |
| CD83 | DAP10 | CD7 | FcεRIβ |
| CD83 | DAP10 | CD7 | FcεRIγ |
| CD83 | DAP10 | CD7 | DAP10 |
| CD83 | DAP10 | CD7 | DAP12 |
| CD83 | DAP10 | CD7 | CD32 |
| CD83 | DAP10 | CD7 | CD79a |
| CD83 | DAP10 | CD7 | CD79b |
| CD83 | DAP10 | BTNL3 | CD8 |
| CD83 | DAP10 | BTNL3 | CD3ζ |
| CD83 | DAP10 | BTNL3 | CD3δ |
| CD83 | DAP10 | BTNL3 | CD3γ |
| CD83 | DAP10 | BTNL3 | CD3ε |
| CD83 | DAP10 | BTNL3 | FcγRI-γ |
| CD83 | DAP10 | BTNL3 | FcγRIII-γ |
| CD83 | DAP10 | BTNL3 | FcεRIβ |
| CD83 | DAP10 | BTNL3 | FcεRIγ |
| CD83 | DAP10 | BTNL3 | DAP10 |
| CD83 | DAP10 | BTNL3 | DAP12 |
| CD83 | DAP10 | BTNL3 | CD32 |
| CD83 | DAP10 | BTNL3 | CD79a |
| CD83 | DAP10 | BTNL3 | CD79b |
| CD83 | DAP10 | NKG2D | CD8 |
| CD83 | DAP10 | NKG2D | CD3ζ |
| CD83 | DAP10 | NKG2D | CD3δ |
| CD83 | DAP10 | NKG2D | CD3γ |
| CD83 | DAP10 | NKG2D | CD3ε |
| CD83 | DAP10 | NKG2D | FcγRI-γ |
| CD83 | DAP10 | NKG2D | FcγRIII-γ |
| CD83 | DAP10 | NKG2D | FcεRIβ |
| CD83 | DAP10 | NKG2D | FcεRIγ |
| CD83 | DAP10 | NKG2D | DAP10 |
| CD83 | DAP10 | NKG2D | DAP12 |
| CD83 | DAP10 | NKG2D | CD32 |
| CD83 | DAP10 | NKG2D | CD79a |
| CD83 | DAP10 | NKG2D | CD79b |
| CD83 | DAP12 | CD28 | CD8 |
| CD83 | DAP12 | CD28 | CD3ζ |
| CD83 | DAP12 | CD28 | CD3δ |
| CD83 | DAP12 | CD28 | CD3γ |
| CD83 | DAP12 | CD28 | CD3ε |
| CD83 | DAP12 | CD28 | FcγRI-γ |
| CD83 | DAP12 | CD28 | FcγRIII-γ |
| CD83 | DAP12 | CD28 | FcεRIβ |
| CD83 | DAP12 | CD28 | FcεRIγ |
| CD83 | DAP12 | CD28 | DAP10 |
| CD83 | DAP12 | CD28 | DAP12 |
| CD83 | DAP12 | CD28 | CD32 |
| CD83 | DAP12 | CD28 | CD79a |
| CD83 | DAP12 | CD28 | CD79b |
| CD83 | DAP12 | CD8 | CD8 |
| CD83 | DAP12 | CD8 | CD3ζ |
| CD83 | DAP12 | CD8 | CD3δ |
| CD83 | DAP12 | CD8 | CD3γ |
| CD83 | DAP12 | CD8 | CD3ε |
| CD83 | DAP12 | CD8 | FcγRI-γ |
| CD83 | DAP12 | CD8 | FcγRIII-γ |
| CD83 | DAP12 | CD8 | FcεRIβ |
| CD83 | DAP12 | CD8 | FcεRIγ |
| CD83 | DAP12 | CD8 | DAP10 |
| CD83 | DAP12 | CD8 | DAP12 |
| CD83 | DAP12 | CD8 | CD32 |
| CD83 | DAP12 | CD8 | CD79a |
| CD83 | DAP12 | CD8 | CD79b |
| CD83 | DAP12 | CD4 | CD8 |
| CD83 | DAP12 | CD4 | CD3ζ |
| CD83 | DAP12 | CD4 | CD3δ |
| CD83 | DAP12 | CD4 | CD3γ |
| CD83 | DAP12 | CD4 | CD3ε |
| CD83 | DAP12 | CD4 | FcγRI-γ |
| CD83 | DAP12 | CD4 | FcγRIII-γ |
| CD83 | DAP12 | CD4 | FcεRIβ |
| CD83 | DAP12 | CD4 | FcεRIγ |
| CD83 | DAP12 | CD4 | DAP10 |
| CD83 | DAP12 | CD4 | DAP12 |
| CD83 | DAP12 | CD4 | CD32 |
| CD83 | DAP12 | CD4 | CD79a |
| CD83 | DAP12 | CD4 | CD79b |
| CD83 | DAP12 | b2c | CD8 |
| CD83 | DAP12 | b2c | CD3ζ |
| CD83 | DAP12 | b2c | CD3δ |
| CD83 | DAP12 | b2c | CD3γ |
| CD83 | DAP12 | b2c | CD3ε |
| CD83 | DAP12 | b2c | FcγRI-γ |
| CD83 | DAP12 | b2c | FcγRIII-γ |
| CD83 | DAP12 | b2c | FcεRIβ |
| CD83 | DAP12 | b2c | FcεRIγ |
| CD83 | DAP12 | b2c | DAP10 |
| CD83 | DAP12 | b2c | DAP12 |
| CD83 | DAP12 | b2c | CD32 |
| CD83 | DAP12 | b2c | CD79a |
| CD83 | DAP12 | b2c | CD79b |
| CD83 | DAP12 | CD137/41BB | CD8 |
| CD83 | DAP12 | CD137/41BB | CD3ζ |
| CD83 | DAP12 | CD137/41BB | CD3δ |
| CD83 | DAP12 | CD137/41BB | CD3γ |
| CD83 | DAP12 | CD137/41BB | CD3ε |
| CD83 | DAP12 | CD137/41BB | FcγRI-γ |
| CD83 | DAP12 | CD137/41BB | FcγRIII-γ |
| CD83 | DAP12 | CD137/41BB | FcεRIβ |
| CD83 | DAP12 | CD137/41BB | FcεRIγ |
| CD83 | DAP12 | CD137/41BB | DAP10 |
| CD83 | DAP12 | CD137/41BB | DAP12 |
| CD83 | DAP12 | CD137/41BB | CD32 |
| CD83 | DAP12 | CD137/41BB | CD79a |
| CD83 | DAP12 | CD137/41BB | CD79b |
| CD83 | DAP12 | ICOS | CD8 |
| CD83 | DAP12 | ICOS | CD3ζ |
| CD83 | DAP12 | ICOS | CD3δ |
| CD83 | DAP12 | ICOS | CD3γ |
| CD83 | DAP12 | ICOS | CD3ε |
| CD83 | DAP12 | ICOS | FcγRI-γ |
| CD83 | DAP12 | ICOS | FcγRIII-γ |
| CD83 | DAP12 | ICOS | FcεRIβ |
| CD83 | DAP12 | ICOS | FcεRIγ |
| CD83 | DAP12 | ICOS | DAP10 |
| CD83 | DAP12 | ICOS | DAP12 |
| CD83 | DAP12 | ICOS | CD32 |
| CD83 | DAP12 | ICOS | CD79a |
| CD83 | DAP12 | ICOS | CD79b |
| CD83 | DAP12 | CD27 | CD8 |
| CD83 | DAP12 | CD27 | CD3ζ |
| CD83 | DAP12 | CD27 | CD3δ |
| CD83 | DAP12 | CD27 | CD3γ |
| CD83 | DAP12 | CD27 | CD3ε |
| CD83 | DAP12 | CD27 | FcγRI-γ |
| CD83 | DAP12 | CD27 | FcγRIII-γ |
| CD83 | DAP12 | CD27 | FcεRIβ |
| CD83 | DAP12 | CD27 | FcεRIγ |
| CD83 | DAP12 | CD27 | DAP10 |
| CD83 | DAP12 | CD27 | DAP12 |
| CD83 | DAP12 | CD27 | CD32 |
| CD83 | DAP12 | CD27 | CD79a |
| CD83 | DAP12 | CD27 | CD79b |
| CD83 | DAP12 | CD28δ | CD8 |
| CD83 | DAP12 | CD28δ | CD3ζ |
| CD83 | DAP12 | CD28δ | CD3δ |
| CD83 | DAP12 | CD28δ | CD3γ |
| CD83 | DAP12 | CD28δ | CD3ε |
| CD83 | DAP12 | CD28δ | FcγRI-γ |
| CD83 | DAP12 | CD28δ | FcγRIII-γ |
| CD83 | DAP12 | CD28δ | FcεRIβ |
| CD83 | DAP12 | CD28δ | FcεRIγ |
| CD83 | DAP12 | CD28δ | DAP10 |
| CD83 | DAP12 | CD28δ | DAP12 |
| CD83 | DAP12 | CD28δ | CD32 |
| CD83 | DAP12 | CD28δ | CD79a |
| CD83 | DAP12 | CD28δ | CD79b |
| CD83 | DAP12 | CD80 | CD8 |
| CD83 | DAP12 | CD80 | CD3ζ |
| CD83 | DAP12 | CD80 | CD3δ |
| CD83 | DAP12 | CD80 | CD3γ |
| CD83 | DAP12 | CD80 | CD3ε |
| CD83 | DAP12 | CD80 | FcγRI-γ |
| CD83 | DAP12 | CD80 | FcγRIII-γ |
| CD83 | DAP12 | CD80 | FcεRIβ |
| CD83 | DAP12 | CD80 | FcεRIγ |
| CD83 | DAP12 | CD80 | DAP10 |
| CD83 | DAP12 | CD80 | DAP12 |
| CD83 | DAP12 | CD80 | CD32 |
| CD83 | DAP12 | CD80 | CD79a |
| CD83 | DAP12 | CD80 | CD79b |
| CD83 | DAP12 | CD86 | CD8 |
| CD83 | DAP12 | CD86 | CD3ζ |
| CD83 | DAP12 | CD86 | CD3δ |
| CD83 | DAP12 | CD86 | CD3γ |
| CD83 | DAP12 | CD86 | CD3ε |
| CD83 | DAP12 | CD86 | FcγRI-γ |
| CD83 | DAP12 | CD86 | FcγRIII-γ |
| CD83 | DAP12 | CD86 | FcεRIβ |
| CD83 | DAP12 | CD86 | FcεRIγ |
| CD83 | DAP12 | CD86 | DAP10 |
| CD83 | DAP12 | CD86 | DAP12 |
| CD83 | DAP12 | CD86 | CD32 |
| CD83 | DAP12 | CD86 | CD79a |
| CD83 | DAP12 | CD86 | CD79b |
| CD83 | DAP12 | OX40 | CD8 |
| CD83 | DAP12 | OX40 | CD3ζ |
| CD83 | DAP12 | OX40 | CD3δ |
| CD83 | DAP12 | OX40 | CD3γ |
| CD83 | DAP12 | OX40 | CD3ε |
| CD83 | DAP12 | OX40 | FcγRI-γ |
| CD83 | DAP12 | OX40 | FcγRIII-γ |
| CD83 | DAP12 | OX40 | FcεRIβ |
| CD83 | DAP12 | OX40 | FcεRIγ |
| CD83 | DAP12 | OX40 | DAP10 |
| CD83 | DAP12 | OX40 | DAP12 |
| CD83 | DAP12 | OX40 | CD32 |
| CD83 | DAP12 | OX40 | CD79a |
| CD83 | DAP12 | OX40 | CD79b |
| CD83 | DAP12 | DAP10 | CD8 |
| CD83 | DAP12 | DAP10 | CD3ζ |
| CD83 | DAP12 | DAP10 | CD3δ |
| CD83 | DAP12 | DAP10 | CD3γ |
| CD83 | DAP12 | DAP10 | CD3ε |
| CD83 | DAP12 | DAP10 | FcγRI-γ |
| CD83 | DAP12 | DAP10 | FcγRIII-γ |
| CD83 | DAP12 | DAP10 | FcεRIβ |
| CD83 | DAP12 | DAP10 | FcεRIγ |
| CD83 | DAP12 | DAP10 | DAP10 |
| CD83 | DAP12 | DAP10 | DAP12 |
| CD83 | DAP12 | DAP10 | CD32 |
| CD83 | DAP12 | DAP10 | CD79a |
| CD83 | DAP12 | DAP10 | CD79b |
| CD83 | DAP12 | DAP12 | CD8 |
| CD83 | DAP12 | DAP12 | CD3ζ |
| CD83 | DAP12 | DAP12 | CD3δ |
| CD83 | DAP12 | DAP12 | CD3γ |
| CD83 | DAP12 | DAP12 | CD3ε |
| CD83 | DAP12 | DAP12 | FcγRI-γ |
| CD83 | DAP12 | DAP12 | FcγRIII-γ |
| CD83 | DAP12 | DAP12 | FcεRIβ |
| CD83 | DAP12 | DAP12 | FcεRIγ |
| CD83 | DAP12 | DAP12 | DAP10 |
| CD83 | DAP12 | DAP12 | DAP12 |
| CD83 | DAP12 | DAP12 | CD32 |
| CD83 | DAP12 | DAP12 | CD79a |
| CD83 | DAP12 | DAP12 | CD79b |
| CD83 | DAP12 | MyD88 | CD8 |
| CD83 | DAP12 | MyD88 | CD3ζ |
| CD83 | DAP12 | MyD88 | CD3δ |
| CD83 | DAP12 | MyD88 | CD3γ |
| CD83 | DAP12 | MyD88 | CD3ε |
| CD83 | DAP12 | MyD88 | FcγRI-γ |
| CD83 | DAP12 | MyD88 | FcγRIII-γ |
| CD83 | DAP12 | MyD88 | FcεRIβ |
| CD83 | DAP12 | MyD88 | FcεRIγ |
| CD83 | DAP12 | MyD88 | DAP10 |
| CD83 | DAP12 | MyD88 | DAP12 |
| CD83 | DAP12 | MyD88 | CD32 |
| CD83 | DAP12 | MyD88 | CD79a |
| CD83 | DAP12 | MyD88 | CD79b |
| CD83 | DAP12 | CD7 | CD8 |
| CD83 | DAP12 | CD7 | CD3ζ |
| CD83 | DAP12 | CD7 | CD3δ |
| CD83 | DAP12 | CD7 | CD3γ |
| CD83 | DAP12 | CD7 | CD3ε |
| CD83 | DAP12 | CD7 | FcγRI-γ |
| CD83 | DAP12 | CD7 | FcγRIII-γ |
| CD83 | DAP12 | CD7 | FcεRIβ |
| CD83 | DAP12 | CD7 | FcεRIγ |
| CD83 | DAP12 | CD7 | DAP10 |
| CD83 | DAP12 | CD7 | DAP12 |
| CD83 | DAP12 | CD7 | CD32 |
| CD83 | DAP12 | CD7 | CD79a |
| CD83 | DAP12 | CD7 | CD79b |
| CD83 | DAP12 | BTNL3 | CD8 |
| CD83 | DAP12 | BTNL3 | CD3ζ |
| CD83 | DAP12 | BTNL3 | CD3δ |
| CD83 | DAP12 | BTNL3 | CD3γ |
| CD83 | DAP12 | BTNL3 | CD3ε |
| CD83 | DAP12 | BTNL3 | FcγRI-γ |
| CD83 | DAP12 | BTNL3 | FcγRIII-γ |
| CD83 | DAP12 | BTNL3 | FcεRIβ |
| CD83 | DAP12 | BTNL3 | FcεRIγ |
| CD83 | DAP12 | BTNL3 | DAP10 |
| CD83 | DAP12 | BTNL3 | DAP12 |
| CD83 | DAP12 | BTNL3 | CD32 |
| CD83 | DAP12 | BTNL3 | CD79a |
| CD83 | DAP12 | BTNL3 | CD79b |
| CD83 | DAP12 | NKG2D | CD8 |
| CD83 | DAP12 | NKG2D | CD3ζ |
| CD83 | DAP12 | NKG2D | CD3δ |
| CD83 | DAP12 | NKG2D | CD3γ |
| CD83 | DAP12 | NKG2D | CD3ε |
| CD83 | DAP12 | NKG2D | FcγRI-γ |
| CD83 | DAP12 | NKG2D | FcγRIII-γ |
| CD83 | DAP12 | NKG2D | FcεRIβ |
| CD83 | DAP12 | NKG2D | FcεRIγ |
| CD83 | DAP12 | NKG2D | DAP10 |
| CD83 | DAP12 | NKG2D | DAP12 |
| CD83 | DAP12 | NKG2D | CD32 |
| CD83 | DAP12 | NKG2D | CD79a |
| CD83 | DAP12 | NKG2D | CD79b |
| CD83 | MyD88 | CD28 | CD8 |
| CD83 | MyD88 | CD28 | CD3ζ |
| CD83 | MyD88 | CD28 | CD3δ |
| CD83 | MyD88 | CD28 | CD3γ |
| CD83 | MyD88 | CD28 | CD3ε |
| CD83 | MyD88 | CD28 | FcγRI-γ |
| CD83 | MyD88 | CD28 | FcγRIII-γ |
| CD83 | MyD88 | CD28 | FcεRIβ |
| CD83 | MyD88 | CD28 | FcεRIγ |
| CD83 | MyD88 | CD28 | DAP10 |
| CD83 | MyD88 | CD28 | DAP12 |
| CD83 | MyD88 | CD28 | CD32 |
| CD83 | MyD88 | CD28 | CD79a |
| CD83 | MyD88 | CD28 | CD79b |
| CD83 | MyD88 | CD8 | CD8 |
| CD83 | MyD88 | CD8 | CD3ζ |
| CD83 | MyD88 | CD8 | CD3δ |
| CD83 | MyD88 | CD8 | CD3γ |
| CD83 | MyD88 | CD8 | CD3ε |
| CD83 | MyD88 | CD8 | FcγRI-γ |
| CD83 | MyD88 | CD8 | FcγRIII-γ |
| CD83 | MyD88 | CD8 | FcεRIβ |
| CD83 | MyD88 | CD8 | FcεRIγ |
| CD83 | MyD88 | CD8 | DAP10 |
| CD83 | MyD88 | CD8 | DAP12 |
| CD83 | MyD88 | CD8 | CD32 |
| CD83 | MyD88 | CD8 | CD79a |
| CD83 | MyD88 | CD8 | CD79b |
| CD83 | MyD88 | CD4 | CD8 |
| CD83 | MyD88 | CD4 | CD3ζ |
| CD83 | MyD88 | CD4 | CD3δ |
| CD83 | MyD88 | CD4 | CD3γ |
| CD83 | MyD88 | CD4 | CD3ε |
| CD83 | MyD88 | CD4 | FcγRI-γ |
| CD83 | MyD88 | CD4 | FcγRIII-γ |
| CD83 | MyD88 | CD4 | FcεRIβ |
| CD83 | MyD88 | CD4 | FcεRIγ |
| CD83 | MyD88 | CD4 | DAP10 |
| CD83 | MyD88 | CD4 | DAP12 |
| CD83 | MyD88 | CD4 | CD32 |
| CD83 | MyD88 | CD4 | CD79a |
| CD83 | MyD88 | CD4 | CD79b |
| CD83 | MyD88 | b2c | CD8 |
| CD83 | MyD88 | b2c | CD3ζ |
| CD83 | MyD88 | b2c | CD3δ |
| CD83 | MyD88 | b2c | CD3γ |
| CD83 | MyD88 | b2c | CD3ε |
| CD83 | MyD88 | b2c | FcγRI-γ |
| CD83 | MyD88 | b2c | FcγRIII-γ |
| CD83 | MyD88 | b2c | FcεRIβ |
| CD83 | MyD88 | b2c | FcεRIγ |
| CD83 | MyD88 | b2c | DAP10 |
| CD83 | MyD88 | b2c | DAP12 |
| CD83 | MyD88 | b2c | CD32 |
| CD83 | MyD88 | b2c | CD79a |
| CD83 | MyD88 | b2c | CD79b |
| CD83 | MyD88 | CD137/41BB | CD8 |
| CD83 | MyD88 | CD137/41BB | CD3ζ |
| CD83 | MyD88 | CD137/41BB | CD3δ |
| CD83 | MyD88 | CD137/41BB | CD3γ |
| CD83 | MyD88 | CD137/41BB | CD3ε |
| CD83 | MyD88 | CD137/41BB | FcγRI-γ |
| CD83 | MyD88 | CD137/41BB | FcγRIII-γ |
| CD83 | MyD88 | CD137/41BB | FcεRIβ |
| CD83 | MyD88 | CD137/41BB | FcεRIγ |
| CD83 | MyD88 | CD137/41BB | DAP10 |
| CD83 | MyD88 | CD137/41BB | DAP12 |
| CD83 | MyD88 | CD137/41BB | CD32 |
| CD83 | MyD88 | CD137/41BB | CD79a |
| CD83 | MyD88 | CD137/41BB | CD79b |
| CD83 | MyD88 | ICOS | CD8 |
| CD83 | MyD88 | ICOS | CD3ζ |
| CD83 | MyD88 | ICOS | CD3δ |
| CD83 | MyD88 | ICOS | CD3γ |
| CD83 | MyD88 | ICOS | CD3ε |
| CD83 | MyD88 | ICOS | FcγRI-γ |
| CD83 | MyD88 | ICOS | FcγRIII-γ |
| CD83 | MyD88 | ICOS | FcεRIβ |
| CD83 | MyD88 | ICOS | FcεRIγ |
| CD83 | MyD88 | ICOS | DAP10 |
| CD83 | MyD88 | ICOS | DAP12 |
| CD83 | MyD88 | ICOS | CD32 |
| CD83 | MyD88 | ICOS | CD79a |
| CD83 | MyD88 | ICOS | CD79b |
| CD83 | MyD88 | CD27 | CD8 |
| CD83 | MyD88 | CD27 | CD3ζ |
| CD83 | MyD88 | CD27 | CD3δ |
| CD83 | MyD88 | CD27 | CD3γ |
| CD83 | MyD88 | CD27 | CD3ε |
| CD83 | MyD88 | CD27 | FcγRI-γ |
| CD83 | MyD88 | CD27 | FcγRIII-γ |
| CD83 | MyD88 | CD27 | FcεRIβ |
| CD83 | MyD88 | CD27 | FcεRIγ |
| CD83 | MyD88 | CD27 | DAP10 |
| CD83 | MyD88 | CD27 | DAP12 |
| CD83 | MyD88 | CD27 | CD32 |
| CD83 | MyD88 | CD27 | CD79a |
| CD83 | MyD88 | CD27 | CD79b |
| CD83 | MyD88 | CD28δ | CD8 |
| CD83 | MyD88 | CD28δ | CD3ζ |
| CD83 | MyD88 | CD28δ | CD3δ |
| CD83 | MyD88 | CD28δ | CD3γ |
| CD83 | MyD88 | CD28δ | CD3ε |
| CD83 | MyD88 | CD28δ | FcγRI-γ |
| CD83 | MyD88 | CD28δ | FcγRIII-γ |
| CD83 | MyD88 | CD28δ | FcεRIβ |
| CD83 | MyD88 | CD28δ | FcεRIγ |
| CD83 | MyD88 | CD28δ | DAP10 |
| CD83 | MyD88 | CD28δ | DAP12 |
| CD83 | MyD88 | CD28δ | CD32 |
| CD83 | MyD88 | CD28δ | CD79a |
| CD83 | MyD88 | CD28δ | CD79b |
| CD83 | MyD88 | CD80 | CD8 |
| CD83 | MyD88 | CD80 | CD3ζ |
| CD83 | MyD88 | CD80 | CD3δ |
| CD83 | MyD88 | CD80 | CD3γ |
| CD83 | MyD88 | CD80 | CD3ε |
| CD83 | MyD88 | CD80 | FcγRI-γ |
| CD83 | MyD88 | CD80 | FcγRIII-γ |
| CD83 | MyD88 | CD80 | FcεRIβ |
| CD83 | MyD88 | CD80 | FcεRIγ |
| CD83 | MyD88 | CD80 | DAP10 |
| CD83 | MyD88 | CD80 | DAP12 |
| CD83 | MyD88 | CD80 | CD32 |
| CD83 | MyD88 | CD80 | CD79a |
| CD83 | MyD88 | CD80 | CD79b |
| CD83 | MyD88 | CD86 | CD8 |
| CD83 | MyD88 | CD86 | CD3ζ |
| CD83 | MyD88 | CD86 | CD3δ |
| CD83 | MyD88 | CD86 | CD3γ |
| CD83 | MyD88 | CD86 | CD3ε |
| CD83 | MyD88 | CD86 | FcγRI-γ |
| CD83 | MyD88 | CD86 | FcγRIII-γ |
| CD83 | MyD88 | CD86 | FcεRIβ |
| CD83 | MyD88 | CD86 | FcεRIγ |
| CD83 | MyD88 | CD86 | DAP10 |
| CD83 | MyD88 | CD86 | DAP12 |
| CD83 | MyD88 | CD86 | CD32 |
| CD83 | MyD88 | CD86 | CD79a |
| CD83 | MyD88 | CD86 | CD79b |
| CD83 | MyD88 | OX40 | CD8 |
| CD83 | MyD88 | OX40 | CD3ζ |
| CD83 | MyD88 | OX40 | CD3δ |
| CD83 | MyD88 | OX40 | CD3γ |
| CD83 | MyD88 | OX40 | CD3ε |
| CD83 | MyD88 | OX40 | FcγRI-γ |
| CD83 | MyD88 | OX40 | FcγRIII-γ |
| CD83 | MyD88 | OX40 | FcεRIβ |
| CD83 | MyD88 | OX40 | FcεRIγ |
| CD83 | MyD88 | OX40 | DAP10 |
| CD83 | MyD88 | OX40 | DAP12 |
| CD83 | MyD88 | OX40 | CD32 |
| CD83 | MyD88 | OX40 | CD79a |
| CD83 | MyD88 | OX40 | CD79b |
| CD83 | MyD88 | DAP10 | CD8 |
| CD83 | MyD88 | DAP10 | CD3ζ |
| CD83 | MyD88 | DAP10 | CD3δ |
| CD83 | MyD88 | DAP10 | CD3γ |
| CD83 | MyD88 | DAP10 | CD3ε |
| CD83 | MyD88 | DAP10 | FcγRI-γ |
| CD83 | MyD88 | DAP10 | FcγRIII-γ |
| CD83 | MyD88 | DAP10 | FcεRIβ |
| CD83 | MyD88 | DAP10 | FcεRIγ |
| CD83 | MyD88 | DAP10 | DAP10 |
| CD83 | MyD88 | DAP10 | DAP12 |
| CD83 | MyD88 | DAP10 | CD32 |
| CD83 | MyD88 | DAP10 | CD79a |
| CD83 | MyD88 | DAP10 | CD79b |
| CD83 | MyD88 | DAP12 | CD8 |
| CD83 | MyD88 | DAP12 | CD3ζ |
| CD83 | MyD88 | DAP12 | CD3δ |
| CD83 | MyD88 | DAP12 | CD3γ |
| CD83 | MyD88 | DAP12 | CD3ε |
| CD83 | MyD88 | DAP12 | FcγRI-γ |
| CD83 | MyD88 | DAP12 | FcγRIII-γ |
| CD83 | MyD88 | DAP12 | FcεRIβ |
| CD83 | MyD88 | DAP12 | FcεRIγ |
| CD83 | MyD88 | DAP12 | DAP10 |
| CD83 | MyD88 | DAP12 | DAP12 |
| CD83 | MyD88 | DAP12 | CD32 |
| CD83 | MyD88 | DAP12 | CD79a |
| CD83 | MyD88 | DAP12 | CD79b |
| CD83 | MyD88 | MyD88 | CD8 |
| CD83 | MyD88 | MyD88 | CD3ζ |
| CD83 | MyD88 | MyD88 | CD3δ |
| CD83 | MyD88 | MyD88 | CD3γ |
| CD83 | MyD88 | MyD88 | CD3ε |
| CD83 | MyD88 | MyD88 | FcγRI-γ |
| CD83 | MyD88 | MyD88 | FcγRIII-γ |
| CD83 | MyD88 | MyD88 | FcεRIβ |
| CD83 | MyD88 | MyD88 | FcεRIγ |
| CD83 | MyD88 | MyD88 | DAP10 |
| CD83 | MyD88 | MyD88 | DAP12 |
| CD83 | MyD88 | MyD88 | CD32 |
| CD83 | MyD88 | MyD88 | CD79a |
| CD83 | MyD88 | MyD88 | CD79b |
| CD83 | MyD88 | CD7 | CD8 |
| CD83 | MyD88 | CD7 | CD3ζ |
| CD83 | MyD88 | CD7 | CD3δ |
| CD83 | MyD88 | CD7 | CD3γ |
| CD83 | MyD88 | CD7 | CD3ε |
| CD83 | MyD88 | CD7 | FcγRI-γ |
| CD83 | MyD88 | CD7 | FcγRIII-γ |
| CD83 | MyD88 | CD7 | FcεRIβ |
| CD83 | MyD88 | CD7 | FcεRIγ |
| CD83 | MyD88 | CD7 | DAP10 |
| CD83 | MyD88 | CD7 | DAP12 |
| CD83 | MyD88 | CD7 | CD32 |
| CD83 | MyD88 | CD7 | CD79a |
| CD83 | MyD88 | CD7 | CD79b |
| CD83 | MyD88 | BTNL3 | CD8 |
| CD83 | MyD88 | BTNL3 | CD3ζ |
| CD83 | MyD88 | BTNL3 | CD3δ |
| CD83 | MyD88 | BTNL3 | CD3γ |
| CD83 | MyD88 | BTNL3 | CD3ε |
| CD83 | MyD88 | BTNL3 | FcγRI-γ |
| CD83 | MyD88 | BTNL3 | FcγRIII-γ |
| CD83 | MyD88 | BTNL3 | FcεRIβ |
| CD83 | MyD88 | BTNL3 | FcεRIγ |
| CD83 | MyD88 | BTNL3 | DAP10 |
| CD83 | MyD88 | BTNL3 | DAP12 |
| CD83 | MyD88 | BTNL3 | CD32 |
| CD83 | MyD88 | BTNL3 | CD79a |
| CD83 | MyD88 | BTNL3 | CD79b |
| CD83 | MyD88 | NKG2D | CD8 |
| CD83 | MyD88 | NKG2D | CD3ζ |
| CD83 | MyD88 | NKG2D | CD3δ |
| CD83 | MyD88 | NKG2D | CD3γ |
| CD83 | MyD88 | NKG2D | CD3ε |
| CD83 | MyD88 | NKG2D | FcγRI-γ |
| CD83 | MyD88 | NKG2D | FcγRIII-γ |
| CD83 | MyD88 | NKG2D | FcεRIβ |
| CD83 | MyD88 | NKG2D | FcεRIγ |
| CD83 | MyD88 | NKG2D | DAP10 |
| CD83 | MyD88 | NKG2D | DAP12 |
| CD83 | MyD88 | NKG2D | CD32 |
| CD83 | MyD88 | NKG2D | CD79a |
| CD83 | MyD88 | NKG2D | CD79b |
| CD83 | CD7 | CD28 | CD8 |
| CD83 | CD7 | CD28 | CD3ζ |
| CD83 | CD7 | CD28 | CD3δ |
| CD83 | CD7 | CD28 | CD3γ |
| CD83 | CD7 | CD28 | CD3ε |
| CD83 | CD7 | CD28 | FcγRI-γ |
| CD83 | CD7 | CD28 | FcγRIII-γ |
| CD83 | CD7 | CD28 | FcεRIβ |
| CD83 | CD7 | CD28 | FcεRIγ |
| CD83 | CD7 | CD28 | DAP10 |
| CD83 | CD7 | CD28 | DAP12 |
| CD83 | CD7 | CD28 | CD32 |
| CD83 | CD7 | CD28 | CD79a |
| CD83 | CD7 | CD28 | CD79b |
| CD83 | CD7 | CD8 | CD8 |
| CD83 | CD7 | CD8 | CD3ζ |
| CD83 | CD7 | CD8 | CD3δ |
| CD83 | CD7 | CD8 | CD3γ |
| CD83 | CD7 | CD8 | CD3ε |
| CD83 | CD7 | CD8 | FcγRI-γ |
| CD83 | CD7 | CD8 | FcγRIII-γ |
| CD83 | CD7 | CD8 | FcεRIβ |
| CD83 | CD7 | CD8 | FcεRIγ |
| CD83 | CD7 | CD8 | DAP10 |
| CD83 | CD7 | CD8 | DAP12 |
| CD83 | CD7 | CD8 | CD32 |
| CD83 | CD7 | CD8 | CD79a |
| CD83 | CD7 | CD8 | CD79b |
| CD83 | CD7 | CD4 | CD8 |
| CD83 | CD7 | CD4 | CD3ζ |
| CD83 | CD7 | CD4 | CD3δ |
| CD83 | CD7 | CD4 | CD3γ |
| CD83 | CD7 | CD4 | CD3ε |
| CD83 | CD7 | CD4 | FcγRI-γ |
| CD83 | CD7 | CD4 | FcγRIII-γ |
| CD83 | CD7 | CD4 | FcεRIβ |
| CD83 | CD7 | CD4 | FcεRIγ |
| CD83 | CD7 | CD4 | DAP10 |
| CD83 | CD7 | CD4 | DAP12 |
| CD83 | CD7 | CD4 | CD32 |
| CD83 | CD7 | CD4 | CD79a |
| CD83 | CD7 | CD4 | CD79b |
| CD83 | CD7 | b2c | CD8 |
| CD83 | CD7 | b2c | CD3ζ |
| CD83 | CD7 | b2c | CD3δ |
| CD83 | CD7 | b2c | CD3γ |
| CD83 | CD7 | b2c | CD3ε |
| CD83 | CD7 | b2c | FcγRI-γ |
| CD83 | CD7 | b2c | FcγRIII-γ |
| CD83 | CD7 | b2c | FcεRIβ |
| CD83 | CD7 | b2c | FcεRIγ |
| CD83 | CD7 | b2c | DAP10 |
| CD83 | CD7 | b2c | DAP12 |
| CD83 | CD7 | b2c | CD32 |
| CD83 | CD7 | b2c | CD79a |
| CD83 | CD7 | b2c | CD79b |
| CD83 | CD7 | CD137/41BB | CD8 |
| CD83 | CD7 | CD137/41BB | CD3ζ |
| CD83 | CD7 | CD137/41BB | CD3δ |
| CD83 | CD7 | CD137/41BB | CD3γ |
| CD83 | CD7 | CD137/41BB | CD3ε |
| CD83 | CD7 | CD137/41BB | FcγRI-γ |
| CD83 | CD7 | CD137/41BB | FcγRIII-γ |
| CD83 | CD7 | CD137/41BB | FcεRIβ |
| CD83 | CD7 | CD137/41BB | FcεRIγ |
| CD83 | CD7 | CD137/41BB | DAP10 |
| CD83 | CD7 | CD137/41BB | DAP12 |
| CD83 | CD7 | CD137/41BB | CD32 |
| CD83 | CD7 | CD137/41BB | CD79a |
| CD83 | CD7 | CD137/41BB | CD79b |
| CD83 | CD7 | ICOS | CD8 |
| CD83 | CD7 | ICOS | CD3ζ |
| CD83 | CD7 | ICOS | CD3δ |
| CD83 | CD7 | ICOS | CD3γ |
| CD83 | CD7 | ICOS | CD3ε |
| CD83 | CD7 | ICOS | FcγRI-γ |
| CD83 | CD7 | ICOS | FcγRIII-γ |
| CD83 | CD7 | ICOS | FcεRIβ |
| CD83 | CD7 | ICOS | FcεRIγ |
| CD83 | CD7 | ICOS | DAP10 |
| CD83 | CD7 | ICOS | DAP12 |
| CD83 | CD7 | ICOS | CD32 |
| CD83 | CD7 | ICOS | CD79a |
| CD83 | CD7 | ICOS | CD79b |
| CD83 | CD7 | CD27 | CD8 |
| CD83 | CD7 | CD27 | CD3ζ |
| CD83 | CD7 | CD27 | CD3δ |
| CD83 | CD7 | CD27 | CD3γ |
| CD83 | CD7 | CD27 | CD3ε |
| CD83 | CD7 | CD27 | FcγRI-γ |
| CD83 | CD7 | CD27 | FcγRIII-γ |
| CD83 | CD7 | CD27 | FcεRIβ |
| CD83 | CD7 | CD27 | FcεRIγ |
| CD83 | CD7 | CD27 | DAP10 |
| CD83 | CD7 | CD27 | DAP12 |
| CD83 | CD7 | CD27 | CD32 |
| CD83 | CD7 | CD27 | CD79a |
| CD83 | CD7 | CD27 | CD79b |
| CD83 | CD7 | CD28δ | CD8 |
| CD83 | CD7 | CD28δ | CD3ζ |
| CD83 | CD7 | CD28δ | CD3δ |
| CD83 | CD7 | CD28δ | CD3γ |
| CD83 | CD7 | CD28δ | CD3ε |
| CD83 | CD7 | CD28δ | FcγRI-γ |
| CD83 | CD7 | CD28δ | FcγRIII-γ |
| CD83 | CD7 | CD28δ | FcεRIβ |
| CD83 | CD7 | CD28δ | FcεRIγ |
| CD83 | CD7 | CD28δ | DAP10 |
| CD83 | CD7 | CD28δ | DAP12 |
| CD83 | CD7 | CD28δ | CD32 |
| CD83 | CD7 | CD28δ | CD79a |
| CD83 | CD7 | CD28δ | CD79b |
| CD83 | CD7 | CD80 | CD8 |
| CD83 | CD7 | CD80 | CD3ζ |
| CD83 | CD7 | CD80 | CD3δ |
| CD83 | CD7 | CD80 | CD3γ |
| CD83 | CD7 | CD80 | CD3ε |
| CD83 | CD7 | CD80 | FcγRI-γ |
| CD83 | CD7 | CD80 | FcγRIII-γ |
| CD83 | CD7 | CD80 | FcεRIβ |
| CD83 | CD7 | CD80 | FcεRIγ |
| CD83 | CD7 | CD80 | DAP10 |
| CD83 | CD7 | CD80 | DAP12 |
| CD83 | CD7 | CD80 | CD32 |
| CD83 | CD7 | CD80 | CD79a |
| CD83 | CD7 | CD80 | CD79b |
| CD83 | CD7 | CD86 | CD8 |
| CD83 | CD7 | CD86 | CD3ζ |
| CD83 | CD7 | CD86 | CD3δ |
| CD83 | CD7 | CD86 | CD3γ |
| CD83 | CD7 | CD86 | CD3ε |
| CD83 | CD7 | CD86 | FcγRI-γ |
| CD83 | CD7 | CD86 | FcγRIII-γ |
| CD83 | CD7 | CD86 | FcεRIβ |
| CD83 | CD7 | CD86 | FcεRIγ |
| CD83 | CD7 | CD86 | DAP10 |
| CD83 | CD7 | CD86 | DAP12 |
| CD83 | CD7 | CD86 | CD32 |
| CD83 | CD7 | CD86 | CD79a |
| CD83 | CD7 | CD86 | CD79b |
| CD83 | CD7 | OX40 | CD8 |
| CD83 | CD7 | OX40 | CD3ζ |
| CD83 | CD7 | OX40 | CD3δ |
| CD83 | CD7 | OX40 | CD3γ |
| CD83 | CD7 | OX40 | CD3ε |
| CD83 | CD7 | OX40 | FcγRI-γ |
| CD83 | CD7 | OX40 | FcγRIII-γ |
| CD83 | CD7 | OX40 | FcεRIβ |
| CD83 | CD7 | OX40 | FcεRIγ |
| CD83 | CD7 | OX40 | DAP10 |
| CD83 | CD7 | OX40 | DAP12 |
| CD83 | CD7 | OX40 | CD32 |
| CD83 | CD7 | OX40 | CD79a |
| CD83 | CD7 | OX40 | CD79b |
| CD83 | CD7 | DAP10 | CD8 |
| CD83 | CD7 | DAP10 | CD3ζ |
| CD83 | CD7 | DAP10 | CD3δ |
| CD83 | CD7 | DAP10 | CD3γ |
| CD83 | CD7 | DAP10 | CD3ε |
| CD83 | CD7 | DAP10 | FcγRI-γ |
| CD83 | CD7 | DAP10 | FcγRIII-γ |
| CD83 | CD7 | DAP10 | FcεRIβ |
| CD83 | CD7 | DAP10 | FcεRIγ |
| CD83 | CD7 | DAP10 | DAP10 |
| CD83 | CD7 | DAP10 | DAP12 |
| CD83 | CD7 | DAP10 | CD32 |
| CD83 | CD7 | DAP10 | CD79a |
| CD83 | CD7 | DAP10 | CD79b |
| CD83 | CD7 | DAP12 | CD8 |
| CD83 | CD7 | DAP12 | CD3ζ |
| CD83 | CD7 | DAP12 | CD3δ |
| CD83 | CD7 | DAP12 | CD3γ |
| CD83 | CD7 | DAP12 | CD3ε |
| CD83 | CD7 | DAP12 | FcγRI-γ |
| CD83 | CD7 | DAP12 | FcγRIII-γ |
| CD83 | CD7 | DAP12 | FcεRIβ |
| CD83 | CD7 | DAP12 | FcεRIγ |
| CD83 | CD7 | DAP12 | DAP10 |
| CD83 | CD7 | DAP12 | DAP12 |
| CD83 | CD7 | DAP12 | CD32 |
| CD83 | CD7 | DAP12 | CD79a |
| CD83 | CD7 | DAP12 | CD79b |
| CD83 | CD7 | MyD88 | CD8 |
| CD83 | CD7 | MyD88 | CD3ζ |
| CD83 | CD7 | MyD88 | CD3δ |
| CD83 | CD7 | MyD88 | CD3γ |
| CD83 | CD7 | MyD88 | CD3ε |
| CD83 | CD7 | MyD88 | FcγRI-γ |
| CD83 | CD7 | MyD88 | FcγRIII-γ |
| CD83 | CD7 | MyD88 | FcεRIβ |
| CD83 | CD7 | MyD88 | FcεRIγ |
| CD83 | CD7 | MyD88 | DAP10 |
| CD83 | CD7 | MyD88 | DAP12 |
| CD83 | CD7 | MyD88 | CD32 |
| CD83 | CD7 | MyD88 | CD79a |
| CD83 | CD7 | MyD88 | CD79b |
| CD83 | CD7 | CD7 | CD8 |
| CD83 | CD7 | CD7 | CD3ζ |
| CD83 | CD7 | CD7 | CD3δ |
| CD83 | CD7 | CD7 | CD3γ |
| CD83 | CD7 | CD7 | CD3ε |
| CD83 | CD7 | CD7 | FcγRI-γ |
| CD83 | CD7 | CD7 | FcγRIII-γ |
| CD83 | CD7 | CD7 | FcεRIβ |
| CD83 | CD7 | CD7 | FcεRIγ |
| CD83 | CD7 | CD7 | DAP10 |
| CD83 | CD7 | CD7 | DAP12 |
| CD83 | CD7 | CD7 | CD32 |
| CD83 | CD7 | CD7 | CD79a |
| CD83 | CD7 | CD7 | CD79b |
| CD83 | CD7 | BTNL3 | CD8 |
| CD83 | CD7 | BTNL3 | CD3ζ |
| CD83 | CD7 | BTNL3 | CD3δ |
| CD83 | CD7 | BTNL3 | CD3γ |
| CD83 | CD7 | BTNL3 | CD3ε |
| CD83 | CD7 | BTNL3 | FcγRI-γ |
| CD83 | CD7 | BTNL3 | FcγRIII-γ |
| CD83 | CD7 | BTNL3 | FcεRIβ |
| CD83 | CD7 | BTNL3 | FcεRIγ |
| CD83 | CD7 | BTNL3 | DAP10 |
| CD83 | CD7 | BTNL3 | DAP12 |
| CD83 | CD7 | BTNL3 | CD32 |
| CD83 | CD7 | BTNL3 | CD79a |
| CD83 | CD7 | BTNL3 | CD79b |
| CD83 | CD7 | NKG2D | CD8 |
| CD83 | CD7 | NKG2D | CD3ζ |
| CD83 | CD7 | NKG2D | CD3δ |
| CD83 | CD7 | NKG2D | CD3γ |
| CD83 | CD7 | NKG2D | CD3ε |
| CD83 | CD7 | NKG2D | FcγRI-γ |
| CD83 | CD7 | NKG2D | FcγRIII-γ |
| CD83 | CD7 | NKG2D | FcεRIβ |
| CD83 | CD7 | NKG2D | FcεRIγ |
| CD83 | CD7 | NKG2D | DAP10 |
| CD83 | CD7 | NKG2D | DAP12 |
| CD83 | CD7 | NKG2D | CD32 |
| CD83 | CD7 | NKG2D | CD79a |
| CD83 | CD7 | NKG2D | CD79b |
| CD83 | BTNL3 | CD28 | CD8 |
| CD83 | BTNL3 | CD28 | CD3ζ |
| CD83 | BTNL3 | CD28 | CD3δ |
| CD83 | BTNL3 | CD28 | CD3γ |
| CD83 | BTNL3 | CD28 | CD3ε |
| CD83 | BTNL3 | CD28 | FcγRI-γ |
| CD83 | BTNL3 | CD28 | FcγRIII-γ |
| CD83 | BTNL3 | CD28 | FcεRIβ |
| CD83 | BTNL3 | CD28 | FcεRIγ |
| CD83 | BTNL3 | CD28 | DAP10 |
| CD83 | BTNL3 | CD28 | DAP12 |
| CD83 | BTNL3 | CD28 | CD32 |
| CD83 | BTNL3 | CD28 | CD79a |
| CD83 | BTNL3 | CD28 | CD79b |
| CD83 | BTNL3 | CD8 | CD8 |
| CD83 | BTNL3 | CD8 | CD3ζ |
| CD83 | BTNL3 | CD8 | CD3δ |
| CD83 | BTNL3 | CD8 | CD3γ |
| CD83 | BTNL3 | CD8 | CD3ε |
| CD83 | BTNL3 | CD8 | FcγRI-γ |
| CD83 | BTNL3 | CD8 | FcγRIII-γ |
| CD83 | BTNL3 | CD8 | FcεRIβ |
| CD83 | BTNL3 | CD8 | FcεRIγ |
| CD83 | BTNL3 | CD8 | DAP10 |
| CD83 | BTNL3 | CD8 | DAP12 |
| CD83 | BTNL3 | CD8 | CD32 |
| CD83 | BTNL3 | CD8 | CD79a |
| CD83 | BTNL3 | CD8 | CD79b |
| CD83 | BTNL3 | CD4 | CD8 |
| CD83 | BTNL3 | CD4 | CD3ζ |
| CD83 | BTNL3 | CD4 | CD3δ |
| CD83 | BTNL3 | CD4 | CD3γ |
| CD83 | BTNL3 | CD4 | CD3ε |
| CD83 | BTNL3 | CD4 | FcγRI-γ |
| CD83 | BTNL3 | CD4 | FcγRIII-γ |
| CD83 | BTNL3 | CD4 | FcεRIβ |
| CD83 | BTNL3 | CD4 | FcεRIγ |
| CD83 | BTNL3 | CD4 | DAP10 |
| CD83 | BTNL3 | CD4 | DAP12 |
| CD83 | BTNL3 | CD4 | CD32 |
| CD83 | BTNL3 | CD4 | CD79a |
| CD83 | BTNL3 | CD4 | CD79b |
| CD83 | BTNL3 | b2c | CD8 |
| CD83 | BTNL3 | b2c | CD3ζ |
| CD83 | BTNL3 | b2c | CD3δ |
| CD83 | BTNL3 | b2c | CD3γ |
| CD83 | BTNL3 | b2c | CD3ε |
| CD83 | BTNL3 | b2c | FcγRI-γ |
| CD83 | BTNL3 | b2c | FcγRIII-γ |
| CD83 | BTNL3 | b2c | FcεRIβ |
| CD83 | BTNL3 | b2c | FcεRIγ |
| CD83 | BTNL3 | b2c | DAP10 |
| CD83 | BTNL3 | b2c | DAP12 |
| CD83 | BTNL3 | b2c | CD32 |
| CD83 | BTNL3 | b2c | CD79a |
| CD83 | BTNL3 | b2c | CD79b |
| CD83 | BTNL3 | CD137/41BB | CD8 |
| CD83 | BTNL3 | CD137/41BB | CD3ζ |
| CD83 | BTNL3 | CD137/41BB | CD3δ |
| CD83 | BTNL3 | CD137/41BB | CD3γ |
| CD83 | BTNL3 | CD137/41BB | CD3ε |
| CD83 | BTNL3 | CD137/41BB | FcγRI-γ |
| CD83 | BTNL3 | CD137/41BB | FcγRIII-γ |
| CD83 | BTNL3 | CD137/41BB | FcεRIβ |
| CD83 | BTNL3 | CD137/41BB | FcεRIγ |
| CD83 | BTNL3 | CD137/41BB | DAP10 |
| CD83 | BTNL3 | CD137/41BB | DAP12 |
| CD83 | BTNL3 | CD137/41BB | CD32 |
| CD83 | BTNL3 | CD137/41BB | CD79a |
| CD83 | BTNL3 | CD137/41BB | CD79b |
| CD83 | BTNL3 | ICOS | CD8 |
| CD83 | BTNL3 | ICOS | CD3ζ |
| CD83 | BTNL3 | ICOS | CD3δ |
| CD83 | BTNL3 | ICOS | CD3γ |
| CD83 | BTNL3 | ICOS | CD3ε |
| CD83 | BTNL3 | ICOS | FcγRI-γ |
| CD83 | BTNL3 | ICOS | FcγRIII-γ |
| CD83 | BTNL3 | ICOS | FcεRIβ |
| CD83 | BTNL3 | ICOS | FcεRIγ |
| CD83 | BTNL3 | ICOS | DAP10 |
| CD83 | BTNL3 | ICOS | DAP12 |
| CD83 | BTNL3 | ICOS | CD32 |
| CD83 | BTNL3 | ICOS | CD79a |
| CD83 | BTNL3 | ICOS | CD79b |
| CD83 | BTNL3 | CD27 | CD8 |
| CD83 | BTNL3 | CD27 | CD3ζ |
| CD83 | BTNL3 | CD27 | CD3δ |
| CD83 | BTNL3 | CD27 | CD3γ |
| CD83 | BTNL3 | CD27 | CD3ε |
| CD83 | BTNL3 | CD27 | FcγRI-γ |
| CD83 | BTNL3 | CD27 | FcγRIII-γ |
| CD83 | BTNL3 | CD27 | FcεRIβ |
| CD83 | BTNL3 | CD27 | FcεRIγ |
| CD83 | BTNL3 | CD27 | DAP10 |
| CD83 | BTNL3 | CD27 | DAP12 |
| CD83 | BTNL3 | CD27 | CD32 |
| CD83 | BTNL3 | CD27 | CD79a |
| CD83 | BTNL3 | CD27 | CD79b |
| CD83 | BTNL3 | CD28δ | CD8 |
| CD83 | BTNL3 | CD28δ | CD3ζ |
| CD83 | BTNL3 | CD28δ | CD3δ |
| CD83 | BTNL3 | CD28δ | CD3γ |
| CD83 | BTNL3 | CD28δ | CD3ε |
| CD83 | BTNL3 | CD28δ | FcγRI-γ |
| CD83 | BTNL3 | CD28δ | FcγRIII-γ |
| CD83 | BTNL3 | CD28δ | FcεRIβ |
| CD83 | BTNL3 | CD28δ | FcεRIγ |
| CD83 | BTNL3 | CD28δ | DAP10 |
| CD83 | BTNL3 | CD28δ | DAP12 |
| CD83 | BTNL3 | CD28δ | CD32 |
| CD83 | BTNL3 | CD28δ | CD79a |
| CD83 | BTNL3 | CD28δ | CD79b |
| CD83 | BTNL3 | CD80 | CD8 |
| CD83 | BTNL3 | CD80 | CD3ζ |
| CD83 | BTNL3 | CD80 | CD3δ |
| CD83 | BTNL3 | CD80 | CD3γ |
| CD83 | BTNL3 | CD80 | CD3ε |
| CD83 | BTNL3 | CD80 | FcγRI-γ |
| CD83 | BTNL3 | CD80 | FcγRIII-γ |
| CD83 | BTNL3 | CD80 | FcεRIβ |
| CD83 | BTNL3 | CD80 | FcεRIγ |
| CD83 | BTNL3 | CD80 | DAP10 |
| CD83 | BTNL3 | CD80 | DAP12 |
| CD83 | BTNL3 | CD80 | CD32 |
| CD83 | BTNL3 | CD80 | CD79a |
| CD83 | BTNL3 | CD80 | CD79b |
| CD83 | BTNL3 | CD86 | CD8 |
| CD83 | BTNL3 | CD86 | CD3ζ |
| CD83 | BTNL3 | CD86 | CD3δ |
| CD83 | BTNL3 | CD86 | CD3γ |
| CD83 | BTNL3 | CD86 | CD3ε |
| CD83 | BTNL3 | CD86 | FcγRI-γ |
| CD83 | BTNL3 | CD86 | FcγRIII-γ |
| CD83 | BTNL3 | CD86 | FcεRIβ |
| CD83 | BTNL3 | CD86 | FcεRIγ |
| CD83 | BTNL3 | CD86 | DAP10 |
| CD83 | BTNL3 | CD86 | DAP12 |
| CD83 | BTNL3 | CD86 | CD32 |
| CD83 | BTNL3 | CD86 | CD79a |
| CD83 | BTNL3 | CD86 | CD79b |
| CD83 | BTNL3 | OX40 | CD8 |
| CD83 | BTNL3 | OX40 | CD3ζ |
| CD83 | BTNL3 | OX40 | CD3δ |
| CD83 | BTNL3 | OX40 | CD3γ |
| CD83 | BTNL3 | OX40 | CD3ε |
| CD83 | BTNL3 | OX40 | FcγRI-γ |
| CD83 | BTNL3 | OX40 | FcγRIII-γ |
| CD83 | BTNL3 | OX40 | FcεRIβ |
| CD83 | BTNL3 | OX40 | FcεRIγ |
| CD83 | BTNL3 | OX40 | DAP10 |
| CD83 | BTNL3 | OX40 | DAP12 |
| CD83 | BTNL3 | OX40 | CD32 |
| CD83 | BTNL3 | OX40 | CD79a |
| CD83 | BTNL3 | OX40 | CD79b |
| CD83 | BTNL3 | DAP10 | CD8 |
| CD83 | BTNL3 | DAP10 | CD3ζ |
| CD83 | BTNL3 | DAP10 | CD3δ |
| CD83 | BTNL3 | DAP10 | CD3γ |
| CD83 | BTNL3 | DAP10 | CD3ε |
| CD83 | BTNL3 | DAP10 | FcγRI-γ |
| CD83 | BTNL3 | DAP10 | FcγRIII-γ |
| CD83 | BTNL3 | DAP10 | FcεRIβ |
| CD83 | BTNL3 | DAP10 | FcεRIγ |
| CD83 | BTNL3 | DAP10 | DAP10 |
| CD83 | BTNL3 | DAP10 | DAP12 |
| CD83 | BTNL3 | DAP10 | CD32 |
| CD83 | BTNL3 | DAP10 | CD79a |
| CD83 | BTNL3 | DAP10 | CD79b |
| CD83 | BTNL3 | DAP12 | CD8 |
| CD83 | BTNL3 | DAP12 | CD3ζ |
| CD83 | BTNL3 | DAP12 | CD3δ |
| CD83 | BTNL3 | DAP12 | CD3γ |
| CD83 | BTNL3 | DAP12 | CD3ε |
| CD83 | BTNL3 | DAP12 | FcγRI-γ |
| CD83 | BTNL3 | DAP12 | FcγRIII-γ |
| CD83 | BTNL3 | DAP12 | FcεRIβ |
| CD83 | BTNL3 | DAP12 | FcεRIγ |
| CD83 | BTNL3 | DAP12 | DAP10 |
| CD83 | BTNL3 | DAP12 | DAP12 |
| CD83 | BTNL3 | DAP12 | CD32 |
| CD83 | BTNL3 | DAP12 | CD79a |
| CD83 | BTNL3 | DAP12 | CD79b |
| CD83 | BTNL3 | MyD88 | CD8 |
| CD83 | BTNL3 | MyD88 | CD3ζ |
| CD83 | BTNL3 | MyD88 | CD3δ |
| CD83 | BTNL3 | MyD88 | CD3γ |
| CD83 | BTNL3 | MyD88 | CD3ε |
| CD83 | BTNL3 | MyD88 | FcγRI-γ |
| CD83 | BTNL3 | MyD88 | FcγRIII-γ |
| CD83 | BTNL3 | MyD88 | FcεRIβ |
| CD83 | BTNL3 | MyD88 | FcεRIγ |
| CD83 | BTNL3 | MyD88 | DAP10 |
| CD83 | BTNL3 | MyD88 | DAP12 |
| CD83 | BTNL3 | MyD88 | CD32 |
| CD83 | BTNL3 | MyD88 | CD79a |
| CD83 | BTNL3 | MyD88 | CD79b |
| CD83 | BTNL3 | CD7 | CD8 |
| CD83 | BTNL3 | CD7 | CD3ζ |
| CD83 | BTNL3 | CD7 | CD3δ |
| CD83 | BTNL3 | CD7 | CD3γ |
| CD83 | BTNL3 | CD7 | CD3ε |
| CD83 | BTNL3 | CD7 | FcγRI-γ |
| CD83 | BTNL3 | CD7 | FcγRIII-γ |
| CD83 | BTNL3 | CD7 | FcεRIβ |
| CD83 | BTNL3 | CD7 | FcεRIγ |
| CD83 | BTNL3 | CD7 | DAP10 |
| CD83 | BTNL3 | CD7 | DAP12 |
| CD83 | BTNL3 | CD7 | CD32 |
| CD83 | BTNL3 | CD7 | CD79a |
| CD83 | BTNL3 | CD7 | CD79b |
| CD83 | BTNL3 | BTNL3 | CD8 |
| CD83 | BTNL3 | BTNL3 | CD3ζ |
| CD83 | BTNL3 | BTNL3 | CD3δ |
| CD83 | BTNL3 | BTNL3 | CD3γ |
| CD83 | BTNL3 | BTNL3 | CD3ε |
| CD83 | BTNL3 | BTNL3 | FcγRI-γ |
| CD83 | BTNL3 | BTNL3 | FcγRIII-γ |
| CD83 | BTNL3 | BTNL3 | FcεRIβ |
| CD83 | BTNL3 | BTNL3 | FcεRIγ |
| CD83 | BTNL3 | BTNL3 | DAP10 |
| CD83 | BTNL3 | BTNL3 | DAP12 |
| CD83 | BTNL3 | BTNL3 | CD32 |
| CD83 | BTNL3 | BTNL3 | CD79a |
| CD83 | BTNL3 | BTNL3 | CD79b |
| CD83 | BTNL3 | NKG2D | CD8 |
| CD83 | BTNL3 | NKG2D | CD3ζ |
| CD83 | BTNL3 | NKG2D | CD3δ |
| CD83 | BTNL3 | NKG2D | CD3γ |
| CD83 | BTNL3 | NKG2D | CD3ε |
| CD83 | BTNL3 | NKG2D | FcγRI-γ |
| CD83 | BTNL3 | NKG2D | FcγRIII-γ |
| CD83 | BTNL3 | NKG2D | FcεRIβ |
| CD83 | BTNL3 | NKG2D | FcεRIγ |
| CD83 | BTNL3 | NKG2D | DAP10 |
| CD83 | BTNL3 | NKG2D | DAP12 |
| CD83 | BTNL3 | NKG2D | CD32 |
| CD83 | BTNL3 | NKG2D | CD79a |
| CD83 | BTNL3 | NKG2D | CD79b |
| CD83 | NKG2D | CD28 | CD8 |
| CD83 | NKG2D | CD28 | CD3ζ |
| CD83 | NKG2D | CD28 | CD3δ |
| CD83 | NKG2D | CD28 | CD3γ |
| CD83 | NKG2D | CD28 | CD3ε |
| CD83 | NKG2D | CD28 | FcγRI-γ |
| CD83 | NKG2D | CD28 | FcγRIII-γ |
| CD83 | NKG2D | CD28 | FcεRIβ |
| CD83 | NKG2D | CD28 | FcεRIγ |
| CD83 | NKG2D | CD28 | DAP10 |
| CD83 | NKG2D | CD28 | DAP12 |
| CD83 | NKG2D | CD28 | CD32 |
| CD83 | NKG2D | CD28 | CD79a |
| CD83 | NKG2D | CD28 | CD79b |
| CD83 | NKG2D | CD8 | CD8 |
| CD83 | NKG2D | CD8 | CD3ζ |
| CD83 | NKG2D | CD8 | CD3δ |
| CD83 | NKG2D | CD8 | CD3γ |
| CD83 | NKG2D | CD8 | CD3ε |
| CD83 | NKG2D | CD8 | FcγRI-γ |
| CD83 | NKG2D | CD8 | FcγRIII-γ |
| CD83 | NKG2D | CD8 | FcεRIβ |
| CD83 | NKG2D | CD8 | FcεRIγ |
| CD83 | NKG2D | CD8 | DAP10 |
| CD83 | NKG2D | CD8 | DAP12 |
| CD83 | NKG2D | CD8 | CD32 |
| CD83 | NKG2D | CD8 | CD79a |
| CD83 | NKG2D | CD8 | CD79b |
| CD83 | NKG2D | CD4 | CD8 |
| CD83 | NKG2D | CD4 | CD3ζ |
| CD83 | NKG2D | CD4 | CD3δ |
| CD83 | NKG2D | CD4 | CD3γ |
| CD83 | NKG2D | CD4 | CD3ε |
| CD83 | NKG2D | CD4 | FcγRI-γ |
| CD83 | NKG2D | CD4 | FcγRIII-γ |
| CD83 | NKG2D | CD4 | FcεRIβ |
| CD83 | NKG2D | CD4 | FcεRIγ |
| CD83 | NKG2D | CD4 | DAP10 |
| CD83 | NKG2D | CD4 | DAP12 |
| CD83 | NKG2D | CD4 | CD32 |
| CD83 | NKG2D | CD4 | CD79a |
| CD83 | NKG2D | CD4 | CD79b |
| CD83 | NKG2D | b2c | CD8 |
| CD83 | NKG2D | b2c | CD3ζ |
| CD83 | NKG2D | b2c | CD3δ |
| CD83 | NKG2D | b2c | CD3γ |
| CD83 | NKG2D | b2c | CD3ε |
| CD83 | NKG2D | b2c | FcγRI-γ |
| CD83 | NKG2D | b2c | FcγRIII-γ |
| CD83 | NKG2D | b2c | FcεRIβ |
| CD83 | NKG2D | b2c | FcεRIγ |
| CD83 | NKG2D | b2c | DAP10 |
| CD83 | NKG2D | b2c | DAP12 |
| CD83 | NKG2D | b2c | CD32 |
| CD83 | NKG2D | b2c | CD79a |
| CD83 | NKG2D | b2c | CD79b |
| CD83 | NKG2D | CD137/41BB | CD8 |
| CD83 | NKG2D | CD137/41BB | CD3ζ |
| CD83 | NKG2D | CD137/41BB | CD3δ |
| CD83 | NKG2D | CD137/41BB | CD3γ |
| CD83 | NKG2D | CD137/41BB | CD3ε |
| CD83 | NKG2D | CD137/41BB | FcγRI-γ |
| CD83 | NKG2D | CD137/41BB | FcγRIII-γ |
| CD83 | NKG2D | CD137/41BB | FcεRIβ |
| CD83 | NKG2D | CD137/41BB | FcεRIγ |
| CD83 | NKG2D | CD137/41BB | DAP10 |
| CD83 | NKG2D | CD137/41BB | DAP12 |
| CD83 | NKG2D | CD137/41BB | CD32 |
| CD83 | NKG2D | CD137/41BB | CD79a |
| CD83 | NKG2D | CD137/41BB | CD79b |
| CD83 | NKG2D | ICOS | CD8 |
| CD83 | NKG2D | ICOS | CD3ζ |
| CD83 | NKG2D | ICOS | CD3δ |
| CD83 | NKG2D | ICOS | CD3γ |
| CD83 | NKG2D | ICOS | CD3ε |
| CD83 | NKG2D | ICOS | FcγRI-γ |
| CD83 | NKG2D | ICOS | FcγRIII-γ |
| CD83 | NKG2D | ICOS | FcεRIβ |
| CD83 | NKG2D | ICOS | FcεRIγ |
| CD83 | NKG2D | ICOS | DAP10 |
| CD83 | NKG2D | ICOS | DAP12 |
| CD83 | NKG2D | ICOS | CD32 |
| CD83 | NKG2D | ICOS | CD79a |
| CD83 | NKG2D | ICOS | CD79b |
| CD83 | NKG2D | CD27 | CD8 |
| CD83 | NKG2D | CD27 | CD3ζ |
| CD83 | NKG2D | CD27 | CD3δ |
| CD83 | NKG2D | CD27 | CD3γ |
| CD83 | NKG2D | CD27 | CD3ε |
| CD83 | NKG2D | CD27 | FcγRI-γ |
| CD83 | NKG2D | CD27 | FcγRIII-γ |
| CD83 | NKG2D | CD27 | FcεRIβ |
| CD83 | NKG2D | CD27 | FcεRIγ |
| CD83 | NKG2D | CD27 | DAP10 |
| CD83 | NKG2D | CD27 | DAP12 |
| CD83 | NKG2D | CD27 | CD32 |
| CD83 | NKG2D | CD27 | CD79a |
| CD83 | NKG2D | CD27 | CD79b |
| CD83 | NKG2D | CD28δ | CD8 |
| CD83 | NKG2D | CD28δ | CD3ζ |
| CD83 | NKG2D | CD28δ | CD3δ |
| CD83 | NKG2D | CD28δ | CD3γ |
| CD83 | NKG2D | CD28δ | CD3ε |
| CD83 | NKG2D | CD28δ | FcγRI-γ |
| CD83 | NKG2D | CD28δ | FcγRIII-γ |
| CD83 | NKG2D | CD28δ | FcεRIβ |
| CD83 | NKG2D | CD28δ | FcεRIγ |
| CD83 | NKG2D | CD28δ | DAP10 |
| CD83 | NKG2D | CD28δ | DAP12 |
| CD83 | NKG2D | CD28δ | CD32 |
| CD83 | NKG2D | CD28δ | CD79a |
| CD83 | NKG2D | CD28δ | CD79b |
| CD83 | NKG2D | CD80 | CD8 |
| CD83 | NKG2D | CD80 | CD3ζ |
| CD83 | NKG2D | CD80 | CD3δ |
| CD83 | NKG2D | CD80 | CD3γ |
| CD83 | NKG2D | CD80 | CD3ε |
| CD83 | NKG2D | CD80 | FcγRI-γ |
| CD83 | NKG2D | CD80 | FcγRIII-γ |
| CD83 | NKG2D | CD80 | FcεRIβ |
| CD83 | NKG2D | CD80 | FcεRIγ |
| CD83 | NKG2D | CD80 | DAP10 |
| CD83 | NKG2D | CD80 | DAP12 |
| CD83 | NKG2D | CD80 | CD32 |
| CD83 | NKG2D | CD80 | CD79a |
| CD83 | NKG2D | CD80 | CD79b |
| CD83 | NKG2D | CD86 | CD8 |
| CD83 | NKG2D | CD86 | CD3ζ |
| CD83 | NKG2D | CD86 | CD3δ |
| CD83 | NKG2D | CD86 | CD3γ |
| CD83 | NKG2D | CD86 | CD3ε |
| CD83 | NKG2D | CD86 | FcγRI-γ |
| CD83 | NKG2D | CD86 | FcγRIII-γ |
| CD83 | NKG2D | CD86 | FcεRIβ |
| CD83 | NKG2D | CD86 | FcεRIγ |
| CD83 | NKG2D | CD86 | DAP10 |
| CD83 | NKG2D | CD86 | DAP12 |
| CD83 | NKG2D | CD86 | CD32 |
| CD83 | NKG2D | CD86 | CD79a |
| CD83 | NKG2D | CD86 | CD79b |
| CD83 | NKG2D | OX40 | CD8 |
| CD83 | NKG2D | OX40 | CD3ζ |
| CD83 | NKG2D | OX40 | CD3δ |
| CD83 | NKG2D | OX40 | CD3γ |
| CD83 | NKG2D | OX40 | CD3ε |
| CD83 | NKG2D | OX40 | FcγRI-γ |
| CD83 | NKG2D | OX40 | FcγRIII-γ |
| CD83 | NKG2D | OX40 | FcεRIβ |
| CD83 | NKG2D | OX40 | FcεRIγ |
| CD83 | NKG2D | OX40 | DAP10 |
| CD83 | NKG2D | OX40 | DAP12 |
| CD83 | NKG2D | OX40 | CD32 |
| CD83 | NKG2D | OX40 | CD79a |
| CD83 | NKG2D | OX40 | CD79b |
| CD83 | NKG2D | DAP10 | CD8 |
| CD83 | NKG2D | DAP10 | CD3ζ |
| CD83 | NKG2D | DAP10 | CD3δ |
| CD83 | NKG2D | DAP10 | CD3γ |
| CD83 | NKG2D | DAP10 | CD3ε |
| CD83 | NKG2D | DAP10 | FcγRI-γ |
| CD83 | NKG2D | DAP10 | FcγRIII-γ |
| CD83 | NKG2D | DAP10 | FcεRIβ |
| CD83 | NKG2D | DAP10 | FcεRIγ |
| CD83 | NKG2D | DAP10 | DAP10 |
| CD83 | NKG2D | DAP10 | DAP12 |
| CD83 | NKG2D | DAP10 | CD32 |
| CD83 | NKG2D | DAP10 | CD79a |
| CD83 | NKG2D | DAP10 | CD79b |
| CD83 | NKG2D | DAP12 | CD8 |
| CD83 | NKG2D | DAP12 | CD3ζ |
| CD83 | NKG2D | DAP12 | CD3δ |
| CD83 | NKG2D | DAP12 | CD3γ |
| CD83 | NKG2D | DAP12 | CD3ε |
| CD83 | NKG2D | DAP12 | FcγRI-γ |
| CD83 | NKG2D | DAP12 | FcγRIII-γ |
| CD83 | NKG2D | DAP12 | FcεRIβ |
| CD83 | NKG2D | DAP12 | FcεRIγ |
| CD83 | NKG2D | DAP12 | DAP10 |
| CD83 | NKG2D | DAP12 | DAP12 |
| CD83 | NKG2D | DAP12 | CD32 |
| CD83 | NKG2D | DAP12 | CD79a |
| CD83 | NKG2D | DAP12 | CD79b |
| CD83 | NKG2D | MyD88 | CD8 |
| CD83 | NKG2D | MyD88 | CD3ζ |
| CD83 | NKG2D | MyD88 | CD3δ |
| CD83 | NKG2D | MyD88 | CD3γ |
| CD83 | NKG2D | MyD88 | CD3ε |
| CD83 | NKG2D | MyD88 | FcγRI-γ |
| CD83 | NKG2D | MyD88 | FcγRIII-γ |
| CD83 | NKG2D | MyD88 | FcεRIβ |
| CD83 | NKG2D | MyD88 | FcεRIγ |
| CD83 | NKG2D | MyD88 | DAP10 |
| CD83 | NKG2D | MyD88 | DAP12 |
| CD83 | NKG2D | MyD88 | CD32 |
| CD83 | NKG2D | MyD88 | CD79a |
| CD83 | NKG2D | MyD88 | CD79b |
| CD83 | NKG2D | CD7 | CD8 |
| CD83 | NKG2D | CD7 | CD3ζ |
| CD83 | NKG2D | CD7 | CD3δ |
| CD83 | NKG2D | CD7 | CD3γ |
| CD83 | NKG2D | CD7 | CD3ε |
| CD83 | NKG2D | CD7 | FcγRI-γ |
| CD83 | NKG2D | CD7 | FcγRIII-γ |
| CD83 | NKG2D | CD7 | FcεRIβ |
| CD83 | NKG2D | CD7 | FcεRIγ |
| CD83 | NKG2D | CD7 | DAP10 |
| CD83 | NKG2D | CD7 | DAP12 |
| CD83 | NKG2D | CD7 | CD32 |
| CD83 | NKG2D | CD7 | CD79a |
| CD83 | NKG2D | CD7 | CD79b |
| CD83 | NKG2D | BTNL3 | CD8 |
| CD83 | NKG2D | BTNL3 | CD3ζ |
| CD83 | NKG2D | BTNL3 | CD3δ |
| CD83 | NKG2D | BTNL3 | CD3γ |
| CD83 | NKG2D | BTNL3 | CD3ε |
| CD83 | NKG2D | BTNL3 | FcγRI-γ |
| CD83 | NKG2D | BTNL3 | FcγRIII-γ |
| CD83 | NKG2D | BTNL3 | FcεRIβ |
| CD83 | NKG2D | BTNL3 | FcεRIγ |
| CD83 | NKG2D | BTNL3 | DAP10 |
| CD83 | NKG2D | BTNL3 | DAP12 |
| CD83 | NKG2D | BTNL3 | CD32 |
| CD83 | NKG2D | BTNL3 | CD79a |
| CD83 | NKG2D | BTNL3 | CD79b |
| CD83 | NKG2D | NKG2D | CD8 |
| CD83 | NKG2D | NKG2D | CD3ζ |
| CD83 | NKG2D | NKG2D | CD3δ |
| CD83 | NKG2D | NKG2D | CD3γ |
| CD83 | NKG2D | NKG2D | CD3ε |
| CD83 | NKG2D | NKG2D | FcγRI-γ |
| CD83 | NKG2D | NKG2D | FcγRIII-γ |
| CD83 | NKG2D | NKG2D | FcεRIβ |
| CD83 | NKG2D | NKG2D | FcεRIγ |
| CD83 | NKG2D | NKG2D | DAP10 |
| CD83 | NKG2D | NKG2D | DAP12 |
| CD83 | NKG2D | NKG2D | CD32 |
| CD83 | NKG2D | NKG2D | CD79a |
| CD83 | NKG2D | NKG2D | CD79b |

TABLE 4
CARs lacking Co-Simulatory Signal (for dual CAR approach)

| ScFv | Co-stimulatory Signal | Signal Domain |
|---|---|---|
| CD83 | none | CD8 |
| CD83 | none | CD3ζ |
| CD83 | none | CD3δ |
| CD83 | none | CD3γ |
| CD83 | none | CD3ε |
| CD83 | none | FcγRI-γ |
| CD83 | none | FcγRIII-γ |
| CD83 | none | FcεRIβ |
| CD83 | none | FcεRIγ |
| CD83 | none | DAP10 |
| CD83 | none | DAP12 |
| CD83 | none | CD32 |
| CD83 | none | CD79a |
| CD83 | none | CD8 |
| CD83 | none | CD3ζ |
| CD83 | none | CD3δ |
| CD83 | none | CD3γ |
| CD83 | none | CD3ε |
| CD83 | none | FcγRI-γ |

TABLE 5
CARs lacking Signal Domain (for dual CAR approach)

| ScFv | Co-stimulatory Signal | Signal Domain |
|---|---|---|
| CD83 | CD28 | none |
| CD83 | CD8 | none |
| CD83 | CD4 | none |
| CD83 | b2c | none |
| CD83 | CD137/41BB | none |
| CD83 | ICOS | none |
| CD83 | CD27 | none |
| CD83 | CD28δ | none |
| CD83 | CD80 | none |
| CD83 | CD86 | none |
| CD83 | OX40 | none |
| CD83 | DAP10 | none |
| CD83 | MyD88 | none |
| CD83 | CD7 | none |
| CD83 | DAP12 | none |
| CD83 | MyD88 | none |
| CD83 | CD7 | none |
| CD83 | BTNL3 | none |
| CD83 | NKG2D | none |

TABLE 6
Third Generation CARs lacking Signal Domain (for dual CAR approach)

| | Co-stimulatory | Co-stimulatory | Signal |
|---|---|---|---|
| ScFv | Signal | Signal | Domain |
| CD83 | CD28 | CD28 | none |
| CD83 | CD28 | CD8 | none |
| CD83 | CD28 | CD4 | none |
| CD83 | CD28 | b2c | none |
| CD83 | CD28 | CD137/41BB | none |
| CD83 | CD28 | ICOS | none |
| CD83 | CD28 | CD27 | none |
| CD83 | CD28 | CD28δ | none |
| CD83 | CD28 | CD80 | none |
| CD83 | CD28 | CD86 | none |
| CD83 | CD28 | OX40 | none |
| CD83 | CD28 | DAP10 | none |
| CD83 | CD28 | MyD88 | none |
| CD83 | CD28 | CD7 | none |
| CD83 | CD28 | DAP12 | none |
| CD83 | CD28 | MyD88 | none |
| CD83 | CD28 | CD7 | none |
| CD83 | CD8 | CD28 | none |
| CD83 | CD8 | CD8 | none |
| CD83 | CD8 | CD4 | none |
| CD83 | CD8 | b2c | none |
| CD83 | CD8 | CD137/41BB | none |
| CD83 | CD8 | ICOS | none |
| CD83 | CD8 | CD27 | none |
| CD83 | CD8 | CD28δ | none |
| CD83 | CD8 | CD80 | none |
| CD83 | CD8 | CD86 | none |
| CD83 | CD8 | OX40 | none |
| CD83 | CD8 | DAP10 | none |
| CD83 | CD8 | MyD88 | none |
| CD83 | CD8 | CD7 | none |
| CD83 | CD8 | DAP12 | none |
| CD83 | CD8 | MyD88 | none |
| CD83 | CD8 | CD7 | none |
| CD83 | CD4 | CD28 | none |
| CD83 | CD4 | CD8 | none |
| CD83 | CD4 | CD4 | none |
| CD83 | CD4 | b2c | none |
| CD83 | CD4 | CD137/41BB | none |
| CD83 | CD4 | ICOS | none |
| CD83 | CD4 | CD27 | none |
| CD83 | CD4 | CD28δ | none |
| CD83 | CD4 | CD80 | none |
| CD83 | CD4 | CD86 | none |
| CD83 | CD4 | OX40 | none |
| CD83 | CD4 | DAP10 | none |
| CD83 | CD4 | MyD88 | none |
| CD83 | CD4 | CD7 | none |
| CD83 | CD4 | DAP12 | none |
| CD83 | CD4 | MyD88 | none |
| CD83 | CD4 | CD7 | none |
| CD83 | b2c | CD28 | none |
| CD83 | b2c | CD8 | none |
| CD83 | b2c | CD4 | none |
| CD83 | b2c | b2c | none |
| CD83 | b2c | CD137/41BB | none |
| CD83 | b2c | ICOS | none |
| CD83 | b2c | CD27 | none |
| CD83 | b2c | CD28δ | none |
| CD83 | b2c | CD80 | none |
| CD83 | b2c | CD86 | none |
| CD83 | b2c | OX40 | none |
| CD83 | b2c | DAP10 | none |
| CD83 | b2c | MyD88 | none |
| CD83 | b2c | CD7 | none |
| CD83 | b2c | DAP12 | none |
| CD83 | b2c | MyD88 | none |
| CD83 | b2c | CD7 | none |
| CD83 | CD137/41BB | CD28 | none |
| CD83 | CD137/41BB | CD8 | none |
| CD83 | CD137/41BB | CD4 | none |
| CD83 | CD137/41BB | b2c | none |
| CD83 | CD137/41BB | CD137/41BB | none |
| CD83 | CD137/41BB | ICOS | none |
| CD83 | CD137/41BB | CD27 | none |
| CD83 | CD137/41BB | CD28δ | none |
| CD83 | CD137/41BB | CD80 | none |
| CD83 | CD137/41BB | CD86 | none |
| CD83 | CD137/41BB | OX40 | none |
| CD83 | CD137/41BB | DAP10 | none |
| CD83 | CD137/41BB | MyD88 | none |
| CD83 | CD137/41BB | CD7 | none |
| CD83 | CD137/41BB | DAP12 | none |
| CD83 | CD137/41BB | MyD88 | none |
| CD83 | CD137/41BB | CD7 | none |
| CD83 | ICOS | CD28 | none |
| CD83 | ICOS | CD8 | none |
| CD83 | ICOS | CD4 | none |
| CD83 | ICOS | b2c | none |
| CD83 | ICOS | CD137/41BB | none |
| CD83 | ICOS | ICOS | none |
| CD83 | ICOS | CD27 | none |
| CD83 | ICOS | CD28δ | none |
| CD83 | ICOS | CD80 | none |
| CD83 | ICOS | CD86 | none |
| CD83 | ICOS | OX40 | none |
| CD83 | ICOS | DAP10 | none |
| CD83 | ICOS | MyD88 | none |
| CD83 | ICOS | CD7 | none |
| CD83 | ICOS | DAP12 | none |
| CD83 | ICOS | MyD88 | none |
| CD83 | ICOS | CD7 | none |
| CD83 | ICOS | CD28 | none |
| CD83 | ICOS | CD8 | none |
| CD83 | ICOS | CD4 | none |
| CD83 | ICOS | b2c | none |
| CD83 | ICOS | CD137/41BB | none |
| CD83 | ICOS | ICOS | none |
| CD83 | ICOS | CD27 | none |
| CD83 | ICOS | CD28δ | none |
| CD83 | ICOS | CD80 | none |
| CD83 | ICOS | CD86 | none |
| CD83 | ICOS | OX40 | none |
| CD83 | ICOS | DAP10 | none |
| CD83 | ICOS | MyD88 | none |
| CD83 | ICOS | CD7 | none |
| CD83 | ICOS | DAP12 | none |
| CD83 | ICOS | MyD88 | none |
| CD83 | ICOS | CD7 | none |
| CD83 | CD27 | CD28 | none |
| CD83 | CD27 | CD8 | none |
| CD83 | CD27 | CD4 | none |
| CD83 | CD27 | b2c | none |
| CD83 | CD27 | CD137/41BB | none |
| CD83 | CD27 | ICOS | none |
| CD83 | CD27 | CD27 | none |
| CD83 | CD27 | CD28δ | none |
| CD83 | CD27 | CD80 | none |
| CD83 | CD27 | CD86 | none |
| CD83 | CD27 | OX40 | none |
| CD83 | CD27 | DAP10 | none |
| CD83 | CD27 | MyD88 | none |
| CD83 | CD27 | CD7 | none |
| CD83 | CD27 | DAP12 | none |
| CD83 | CD27 | MyD88 | none |
| CD83 | CD27 | CD7 | none |
| CD83 | CD28δ | CD28 | none |
| CD83 | CD28δ | CD8 | none |
| CD83 | CD28δ | CD4 | none |
| CD83 | CD28δ | b2c | none |
| CD83 | CD28δ | CD137/41BB | none |
| CD83 | CD28δ | ICOS | none |
| CD83 | CD28δ | CD27 | none |
| CD83 | CD28δ | CD28δ | none |
| CD83 | CD28δ | CD80 | none |
| CD83 | CD28δ | CD86 | none |
| CD83 | CD28δ | OX40 | none |
| CD83 | CD28δ | DAP10 | none |
| CD83 | CD28δ | MyD88 | none |
| CD83 | CD28δ | CD7 | none |
| CD83 | CD28δ | DAP12 | none |
| CD83 | CD28δ | MyD88 | none |
| CD83 | CD28δ | CD7 | none |
| CD83 | CD80 | CD28 | none |
| CD83 | CD80 | CD8 | none |
| CD83 | CD80 | CD4 | none |
| CD83 | CD80 | b2c | none |
| CD83 | CD80 | CD137/41BB | none |
| CD83 | CD80 | ICOS | none |
| CD83 | CD80 | CD27 | none |
| CD83 | CD80 | CD28δ | none |
| CD83 | CD80 | CD80 | none |
| CD83 | CD80 | CD86 | none |
| CD83 | CD80 | OX40 | none |
| CD83 | CD80 | DAP10 | none |
| CD83 | CD80 | MyD88 | none |
| CD83 | CD80 | CD7 | none |
| CD83 | CD80 | DAP12 | none |
| CD83 | CD80 | MyD88 | none |
| CD83 | CD80 | CD7 | none |
| CD83 | CD86 | CD28 | none |
| CD83 | CD86 | CD8 | none |
| CD83 | CD86 | CD4 | none |
| CD83 | CD86 | b2c | none |
| CD83 | CD86 | CD137/41BB | none |
| CD83 | CD86 | ICOS | none |
| CD83 | CD86 | CD27 | none |
| CD83 | CD86 | CD28δ | none |
| CD83 | CD86 | CD80 | none |
| CD83 | CD86 | CD86 | none |
| CD83 | CD86 | OX40 | none |
| CD83 | CD86 | DAP10 | none |
| CD83 | CD86 | MyD88 | none |
| CD83 | CD86 | CD7 | none |
| CD83 | CD86 | DAP12 | none |
| CD83 | CD86 | MyD88 | none |
| CD83 | CD86 | CD7 | none |
| CD83 | OX40 | CD28 | none |
| CD83 | OX40 | CD8 | none |
| CD83 | OX40 | CD4 | none |
| CD83 | OX40 | b2c | none |
| CD83 | OX40 | CD137/41BB | none |
| CD83 | OX40 | ICOS | none |
| CD83 | OX40 | CD27 | none |
| CD83 | OX40 | CD28δ | none |
| CD83 | OX40 | CD80 | none |
| CD83 | OX40 | CD86 | none |
| CD83 | OX40 | OX40 | none |
| CD83 | OX40 | DAP10 | none |
| CD83 | OX40 | MyD88 | none |
| CD83 | OX40 | CD7 | none |
| CD83 | OX40 | DAP12 | none |
| CD83 | OX40 | MyD88 | none |
| CD83 | OX40 | CD7 | none |
| CD83 | DAP10 | CD28 | none |
| CD83 | DAP10 | CD8 | none |
| CD83 | DAP10 | CD4 | none |
| CD83 | DAP10 | b2c | none |
| CD83 | DAP10 | CD137/41BB | none |
| CD83 | DAP10 | ICOS | none |
| CD83 | DAP10 | CD27 | none |
| CD83 | DAP10 | CD28δ | none |
| CD83 | DAP10 | CD80 | none |
| CD83 | DAP10 | CD86 | none |
| CD83 | DAP10 | OX40 | none |
| CD83 | DAP10 | DAP10 | none |
| CD83 | DAP10 | MyD88 | none |
| CD83 | DAP10 | CD7 | none |
| CD83 | DAP10 | DAP12 | none |
| CD83 | DAP10 | MyD88 | none |
| CD83 | DAP10 | CD7 | none |
| CD83 | DAP12 | CD28 | none |
| CD83 | DAP12 | CD8 | none |
| CD83 | DAP12 | CD4 | none |
| CD83 | DAP12 | b2c | none |
| CD83 | DAP12 | CD137/41BB | none |
| CD83 | DAP12 | ICOS | none |
| CD83 | DAP12 | CD27 | none |
| CD83 | DAP12 | CD28δ | none |
| CD83 | DAP12 | CD80 | none |
| CD83 | DAP12 | CD86 | none |
| CD83 | DAP12 | OX40 | none |
| CD83 | DAP12 | DAP10 | none |
| CD83 | DAP12 | MyD88 | none |
| CD83 | DAP12 | CD7 | none |
| CD83 | DAP12 | DAP12 | none |
| CD83 | DAP12 | MyD88 | none |
| CD83 | DAP12 | CD7 | none |
| CD83 | MyD88 | CD28 | none |
| CD83 | MyD88 | CD8 | none |
| CD83 | MyD88 | CD4 | none |
| CD83 | MyD88 | b2c | none |
| CD83 | MyD88 | CD137/41BB | none |
| CD83 | MyD88 | ICOS | none |
| CD83 | MyD88 | CD27 | none |
| CD83 | MyD88 | CD28δ | none |
| CD83 | MyD88 | CD80 | none |
| CD83 | MyD88 | CD86 | none |
| CD83 | MyD88 | OX40 | none |
| CD83 | MyD88 | DAP10 | none |
| CD83 | MyD88 | MyD88 | none |
| CD83 | MyD88 | CD7 | none |
| CD83 | MyD88 | DAP12 | none |
| CD83 | MyD88 | MyD88 | none |
| CD83 | MyD88 | CD7 | none |
| CD83 | CD7 | CD28 | none |
| CD83 | CD7 | CD8 | none |
| CD83 | CD7 | CD4 | none |
| CD83 | CD7 | b2c | none |
| CD83 | CD7 | CD137/41BB | none |
| CD83 | CD7 | ICOS | none |
| CD83 | CD7 | CD27 | none |
| CD83 | CD7 | CD28δ | none |
| CD83 | CD7 | CD80 | none |
| CD83 | CD7 | CD86 | none |
| CD83 | CD7 | OX40 | none |
| CD83 | CD7 | DAP10 | none |
| CD83 | CD7 | MyD88 | none |
| CD83 | CD7 | CD7 | none |
| CD83 | CD7 | DAP12 | none |
| CD83 | CD7 | MyD88 | none |
| CD83 | CD7 | CD7 | none |
| CD83 | BTNL3 | CD28 | none |
| CD83 | BTNL3 | CD8 | none |
| CD83 | BTNL3 | CD4 | none |
| CD83 | BTNL3 | b2c | none |
| CD83 | BTNL3 | CD137/41BB | none |
| CD83 | BTNL3 | ICOS | none |
| CD83 | BTNL3 | CD27 | none |
| CD83 | BTNL3 | CD28δ | none |
| CD83 | BTNL3 | CD80 | none |
| CD83 | BTNL3 | CD86 | none |
| CD83 | BTNL3 | OX40 | none |
| CD83 | BTNL3 | DAP10 | none |
| CD83 | BTNL3 | MyD88 | none |
| CD83 | BTNL3 | CD7 | none |
| CD83 | BTNL3 | DAP12 | none |
| CD83 | BTNL3 | MyD88 | none |
| CD83 | BTNL3 | CD7 | none |
| CD83 | NKG2D | CD28 | none |
| CD83 | NKG2D | CD8 | none |
| CD83 | NKG2D | CD4 | none |
| CD83 | NKG2D | b2c | none |
| CD83 | NKG2D | CD137/41BB | none |
| CD83 | NKG2D | ICOS | none |
| CD83 | NKG2D | CD27 | none |
| CD83 | NKG2D | CD28δ | none |
| CD83 | NKG2D | CD80 | none |
| CD83 | NKG2D | CD86 | none |
| CD83 | NKG2D | OX40 | none |
| CD83 | NKG2D | DAP10 | none |
| CD83 | NKG2D | MyD88 | none |
| CD83 | NKG2D | CD7 | none |
| CD83 | NKG2D | DAP12 | none |
| CD83 | NKG2D | MyD88 | none |
| CD83 | NKG2D | CD7 | none |

In some embodiments, the anti-CD83 binding agent is single chain variable fragment (scFv) antibody. The affinity/specificity of an anti-CD83 scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the anti-CD83 binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target CD83 and at least one additional antigen. Also disclosed are CARs designed to work only in conjunction with another CAR that binds a different antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

### Nucleic Acids and Vectors

Also disclosed are polynucleotides and polynucleotide vectors encoding the disclosed CD83-specific CARs that allow expression of the CD83-specific CARs in the disclosed regulatory T cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Regulatory T Cells

Also disclosed are regulatory T cells that are engineered to express the disclosed CARs (also referred to herein as “CAR-T cells.” These cells are preferably obtained from the subject to be treated (i.e. are autologous). However, in some embodiments, regulatory T cell lines or allogeneic cells are used. Regulatory T cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, regulatory T cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, regulatory T cells can be isolated using a combination of antibodies directed to surface markers unique to the positively selected cells, e.g., by incubation with antibody-conjugated beads for a time period sufficient for positive selection of the desired regulatory T cells. Alternatively, enrichment of regulatory T cell population can be accomplished by negative selection using a combination of antibodies directed to surface markers unique to the negatively selected cells.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ T_{reg} cells have been described—naturally occurring T_{reg} cells and adaptive T_{reg} cells.

### Therapeutic Methods

Regulatory T cells expressing the disclosed CARs suppress alloreactive donor cells, such as T-cells, and prevent GVHD. Therefore, the disclosed CARs can be administered to any subject at risk for GVHD. In some embodiments, the subject receives a bone marrow transplant and the disclosed CAR-modified regulatory T cells suppress alloreactivity of donor T-cells or dendritic cells.

The disclosed CAR-modified regulatory T cells may be administered either alone, or as a pharmaceutical composition in combination with diluents and/or with other components such as IL-2, IL-15, or other cytokines or cell populations.

In some embodiments, the disclosed CAR-modified regulatory T cells are administered in combination with ER stress blockade (compounds to target the IRE-1/XBP-1 pathway (e.g., B-I09). In some embodiments, the disclosed CAR-modified regulatory T cells are administered in combination with a JAK2 inhibitor, a STAT3 inhibitor, an Aurora kinase inhibitor, an mTOR inhibitor, or any combination thereof.

Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When a “therapeutic amount” is indicated, the precise amount of the compositions of the present invention to be administered can be determined by a physician with consideration of individual differences in age, weight, extent of transplantation, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a site of transplantation.

In certain embodiments, the disclosed CAR-modified regulatory T cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified regulatory T cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified regulatory T cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a first antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tissue, and is advantageous in situations where certain normal tissues may express very low levels of a antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

### EXAMPLES

### Example 1: CD83-Targeted Chimeric Antigen Receptor T Cell Prevents GVHD

### Materials and Methods

### Study Design

This is a preclinical study of the design, production, and efficacy of a human CD83 CAR T cell for GVHD prophylaxis. The first part of the study describes the CAR construct as well as the in vitro activity of the CD83 CAR T cell with regard to phenotype, cytokine production, on-target killing, and proliferation in response to CD83+ targets. The immune suppressive effect of the CD83 CAR T cell is then demonstrated in vitro using standard alloMLRs. Additionally, CD83 expression was measured among human T cells showing differential expression of CD83 on Tconv versus Treg cells. In a human T cell mediated xenogeneic GVHD model (Betts B. C. et al., Science translational medicine 9:eaai8269 (2017)), the preclinical efficacy of the CD83 CAR in GVHD prophylaxis was demonstrated. This includes a thorough evaluation of in vivo target killing of CD83+ dendritic cells and Tconv. Also shown are the effects of the CD83 CAR T cell on various T cell subsets in vivo. It is demonstrated that CD83 is expressed on human malignant myeloid cell lines, and they are effectively killed by the CD83 CAR T cells using the xCELLigence RTCA (real-time cell analysis) system (Li G. et al., JCI Insight 3 (2018)). For GVHD experiments, a humane pre-moribund endpoint was used. Mice were monitored frequently for GVHD clinical scores. GVHD histopathology was evaluated and scored by a blinded expert pathologist (Betts B. C. et al., Science translational medicine 9:eaai8269 (2017); Betts B. C. et al., Proc Natl Acad Sci USA., 201712452 (2018); Betts B. C. et al., Front Immunol 9:2887 (2018)). Murine in vivo data were pooled from at least two independent experiments with 6-9 mice per experimental group.

CD83 CAR T cell Construct and Production

CD83 CAR was synthesized and cloned into SFG retroviral construct by GENEWIZ (Li, G. et al., Methods Mol Biol 1514:111-118 (2017); Li G. et al., JCI Insight 3 (2018)). The CD83 SFG cloned construct was then transfected into H29 cells using calcium phosphate, and retroviral supernatants from transfected H29 cells was used to transduce RD114. Retroviral supernatant of RD114 cells was filtered through 0.45 μm strainer (MilliporeSigma) to purify gamma retrovirus. Specifically CD83 CAR T cells were generated by transduction of human T cells as described (Li G. et al., JCI Insight 3 (2018)). Briefly, Leukocytes obtained from apheresis from a healthy human donor (All Cells) were isolated by density gradient centrifugation. T cells were isolated using magnetic beads (Stem Cells Inc.) and stimulated with human Dynabeads CD3 and CD28 (Thermo fisher) in RPMI with recombinant human IL-2. Activated T cells were transduced with CD83 gamma retrovirus on RetroNectin (TaKaRa Bio Inc.) coated plates. CD83 CAR T cells were debeaded after 7-8 days of activation. Gene transfer or transduction efficiency was estimated by GFP+ cells as detected by flow cytometry.

### Monoclonal Antibodies and Flow Cytometry

Fluorochrome-conjugated mouse anti-human monoclonal antibodies included anti-CD3, CD4, CD8, CD25, CD83, CD1c, CD127, MHCII, Foxp3, Ki-67, IFN-γ, IL-17A, and IL-4 (BD Biosciences, San Jose, CA. USA; eBioscience San Jose, CA. USA; Cell Signaling Technology, Boston, MA. USA). LIVE/DEAD Fixable Yellow or Aqua Dead Cell Stain (Life Technologies, Grand Island, NY) was used to determine viability. Live events were acquired on a BD FACSCanto II or LSRII flow cytometer (FlowJo software, ver. 7.6.4; TreeStar, Ashland, OR, USA).

### Cytokine Immunoassays

CD83 CAR and mock transduced T cells (1×10⁵) were co-cultured with CD83+ moDCs (1×10⁴) for 24 hours. Supernatants were harvested and analyzed using a human luminex assay kit (R&D Systems) on a Luminex 100 system (Luminex) and Simple Plex Assay Kit (Biotechne) on an Ella instrument (Biotechne). Manufacturers' instructions were followed (Li G. et al., JCI Insight 3 (2018)).

Human CD83 CAR T cell Cytotoxicity and In Vitro Proliferation.

Normalized CD83 CART cells (1×10⁵ cells) were cultured with CD83+ moDCs, K562, or Thp-1 cells at an ET ratio of 10:1 in duplicates in E-Plate 96. Cytotoxicity assay was run on an xCELLigence RTCA (real-time cell analysis) instrument (ACEA Biosciences) according to manufacture's instruction. Similarly, human CD83 CAR T cells were co-cultured with moDCs at and ET ratio of 1:1 in non-tissue-culture-treated 6-well plates in triplicate. Cells were grown in human T cell complete medium supplemented with 60 IU/ml IL-2. Cell viability and total cell numbers in each well were measured on day +1, +7 and +14 on a cell counter (Bio-Rad) with trypan blue staining.

In Vitro alloMLRs

Human monocyte-derived dendritic cells (moDC) were cytokine-generated, differentiated, and matured as described (Betts B. C. et al., Science translational medicine 9:eaai8269 (2017)). T cells purified (10⁵) purified from leukocyte concentrates (OneBlood or Memorial Blood Center) were cultured with allogeneic moDCs (T cell:DC ratio 30:1) in 100 μl complete RPMI supplemented with 10% heat-inactivated, pooled human serum (Betts B. C. et al., Science translational medicine 9:eaai8269 (2017); Betts B. C. et al., Proc Natl Acad Sci USA., 201712452 (2018); Betts B. C. et al., Front Immunol 9:2887 (2018)). CD83 CAR, CD19 CAR, or mock transduced T cells (autologous to the T cell donor) were added to the alloMLR at a range of CAR to DC ratios. T cell proliferation was measured after 5 days by Ki-67 expression.

CD83 Expression Time Course

Purified human T cells were stimulated with either allogeneic moDCs (T cell:DC ratio 30:1) or CD3/CD28 beads (T cell:bead ratio 30:1). T cells were harvested from triplicate wells in a 96-well plate at 4, 8, 24, and 48 hours of culture. The T cells were stained for CD3, CD4, CD127, CD25, and CD83, then fixed. CD83 expression was evaluated in activated Tconv (CD3+, CD4+, CD127+, CD25+) (Betts B. C. et al., Science translational medicine 9:eaai8269 (2017)), Tregs (CD3+, CD4+, CD127−, CD25+) (Betts B. C. et al., Science translational medicine 9:eaai8269 (2017)), and CD8 T cells (CD3+, CD4−). Where indicated, CD83 CAR or mock T cells were cultured with DC-allostimulated PBMCs, and CD83 expression was evaluated among the CD3− and CD3+ target cells over 48 hours.

### Colony Forming Units

CD34+ cells isolated from normal human bone marrow were purchased from AllCells. 10³ cells were co-cultured with either CAR T cells transduced with CD83 viruses, mock T cells, or media alone. Cells were incubated for 4 hours at an E:T ratio of 10:1. Following incubation, cells were plated in MethoCult medium (StemCell) in 6-well SmartDish plates (StemCell) according to manufacture instructions and cultured for 14 days. At the end of the culture period, colonies were imaged, analyzed, and counted using the STEMvision software.

### Xenogeneic GVHD Model

NOD scid gamma (NSG) mice (male or female, 6-24 weeks old) were raised within an IACU-Capproved colony maintained at the Moffitt/USF vivarium. Recipient mice received 25×10⁶ fresh, human PBMCs (OneBlood) once on day 0 of the transplant. As indicated, mice either received PBMCs alone, PBMCs plus CD83 CAR T cells (low dose: 1×10⁶ or high dose: 10×10⁶), or PBMCs plus mock transduced T cells (10×10⁶). Each independent experiment was performed with a different human PBMC donor, where the CAR T cells and mock transduced T cells were derived from the PBMC donor. Mice were monitored for GVHD clinical scores and pre-moribund status. Where indicated, short term experiments were completed on day +21 via humane euthanasia to evaluate blinded GVHD target organ pathology, tissue-resident lymphocytes, and the content of human DCs and T cell subsets within the murine spleens (Betts B. C. et al., Science translational medicine 9:eaai8269 (2017); Betts B. C. et al., Proc Natl Acad Sci USA., 201712452 (2018); Betts B. C. et al., Front Immunol 9:2887 (2018)). Tissue samples were prepared, stained (Ventana Medical Systems), and imaged (Vista) to identify human Ki67+ T cells as previously described (Betts B. C. et al., Science translational medicine 9:eaai8269 (2017)). These mice were transplanted with PBMCs (25×10⁶) with or without CD83 CAR (1×10⁶) or mock transduced T cells (1×10⁶). All vertebrate animal work was performed under an AICUC-approved protocol.

### Statistical Analysis

Data are reported as mean values ±SEM. ANOVA was used for group comparisons, including a Dunnett's or Sidak's post-test with correction for multiple-comparisons. Mann-Whitney was used for all others. For comparison of survival curves, a Log-rank test was used. The statistical analysis was conducted using Prism software version 5.04 (GraphPad). Statistical significance was defined by a two-tailed P<0.05 (two-tailed).

### Results

### Schema of the Human CD83 CAR Construct.

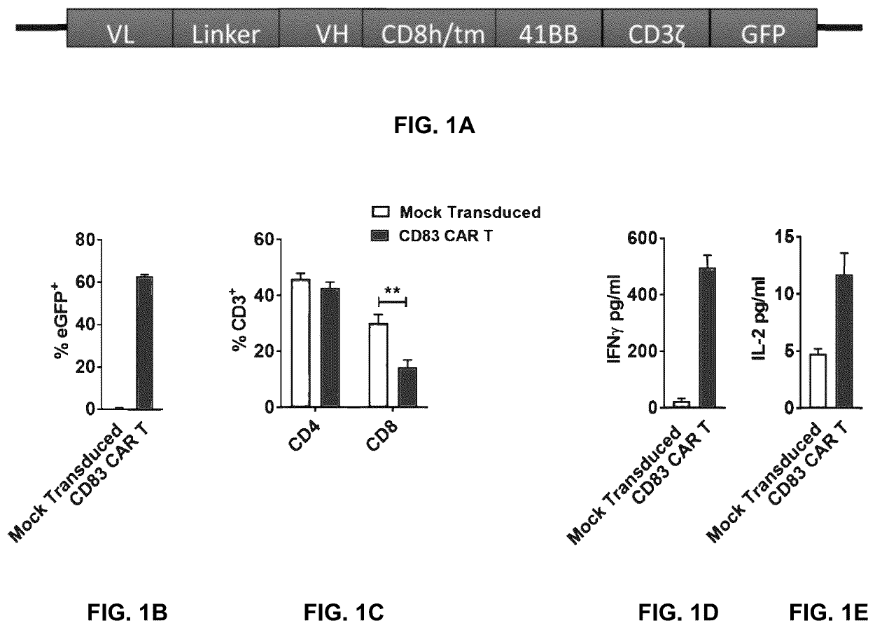

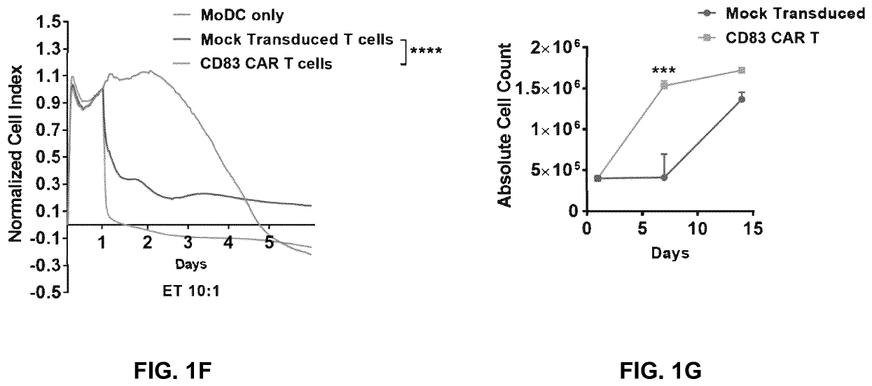

The anti-CD83 single chain variable fragment (scFv) was paired to the CD8 hinge and transmembrane domain, followed by the intracellular 41BB co-stimulatory domain and CD3ζactivation domain (FIG. 1A). To facilitate tracking of CAR T cells, the construct contains an eGFP tag, which can be used to identify the CAR T cell among normal non-CAR T cells (FIG. 1A). CD83-targeted CAR T cells were retrovirally transduced and generated as we have published (FIG. 1A) (Li, G. et al., Methods Mol Biol 1514:111-118 (2017); Li G. et al., JCI Insight 3 (2018)).

### Characterization of the Human CD83 CAR T Cell.

The CD83 CAR construct exhibited a high degree of transduction efficiency, with over 60% of T cells expressing eGFP (FIG. 1B). While CD4 expression was similar among both groups, a significant reduction in CD8 expression was observed among CD83 CAR T cells compared to mock transduced T cells (FIG. 1C). However, the CD83 CAR T cells demonstrated robust IFNγ and IL-2 production when cultured with CD83+ target cells; such as cytokine-matured human, monocyte-derived DCs (moDC) (FIGS. 1D,E). Additionally, CD83 CAR T cells demonstrated potent killing of and proliferation against CD83+ moDCs, compared to mock transduced T cells (FIGS. 1F,1G). The target moDCs in these experiments were allogeneic to the T cells, therefore the lysis and proliferation by mock transduced T cells represent baseline alloreactivity (FIGS. 1F,1G).

### Human CD83 CAR T Cells Reduce Alloreactivity.

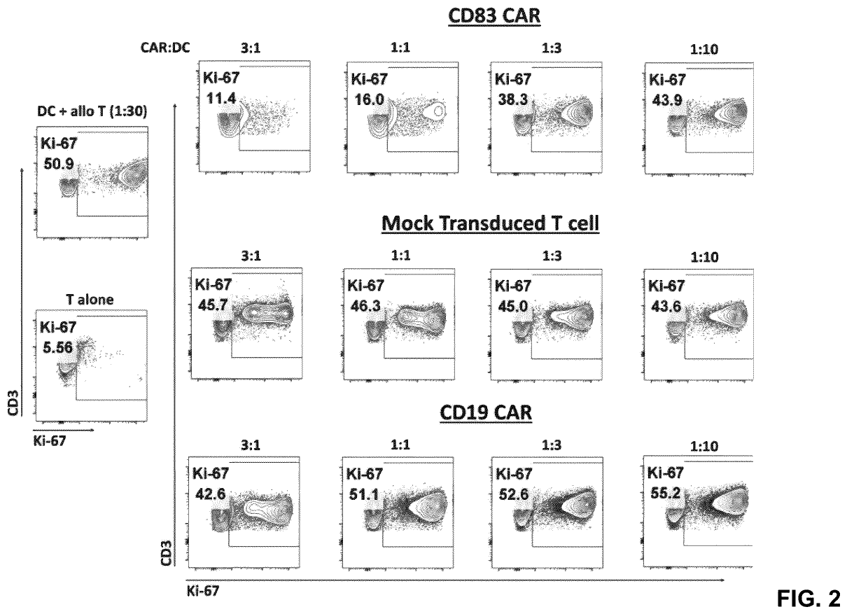

To test whether human CD83 CAR T cells reduce alloreactivity in vitro, their suppressive function in allogeneic mixed leukocyte reactions (alloMLR) was investigated. CD83 and mock transduced CAR T cells were generated from healthy donor, human T cells. CD19 CAR T cells target B cells, an irrelevant cell type in the alloMLR, and were used as an additional control. Furthermore, CD19 and CD83 CAR T cells were similar in that they both receive co-stimulation via 41 BB. CAR T cells were added to 5-day alloMLRs consisting of autologous T cells (1×10⁵) and allogeneic, cytokine-matured, CD83+ moDCs (3.33×10³). The CAR T cell: moDC ratio ranged from 3:1 to 1:10. The CD83 CAR T cells potently reduced alloreactive T cell proliferation (FIG. 2, upper panel). Conversely, mock transduced and CD19-targeted CAR T cells had no suppressive effect against alloreactive T cells (FIG. 2, middle and lower panels).

### CD83 is Differentially Expressed on Activated Human Tconv Compared to Treg.

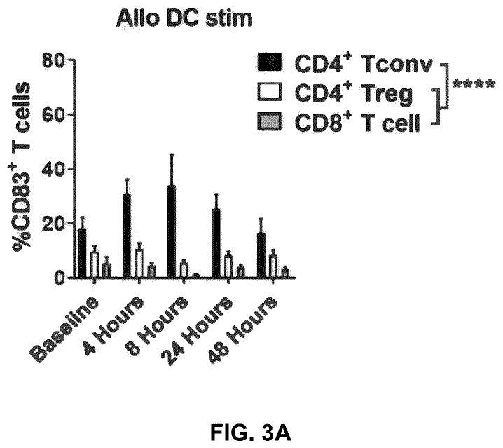

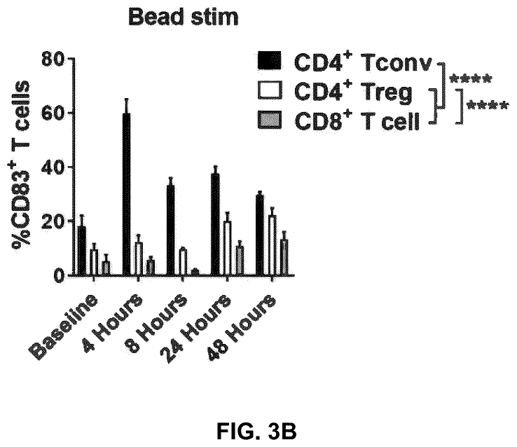

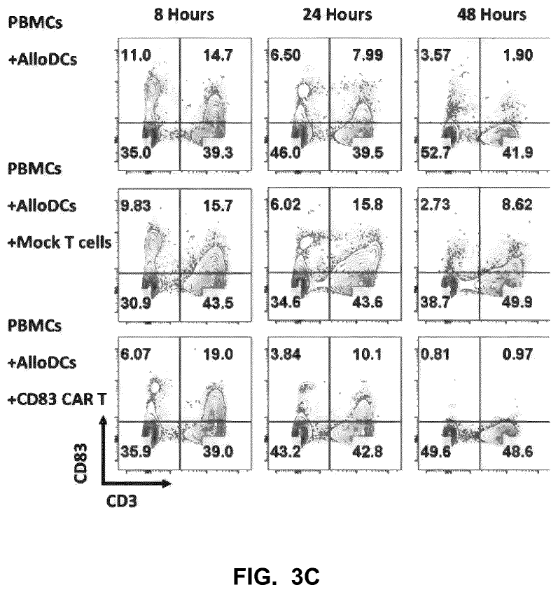

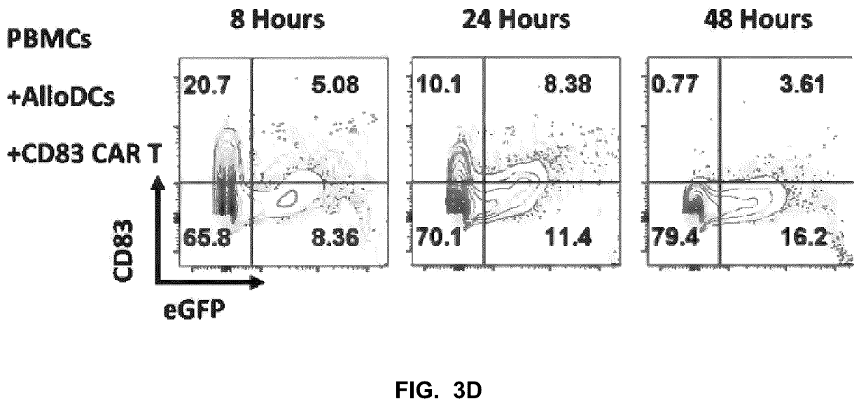

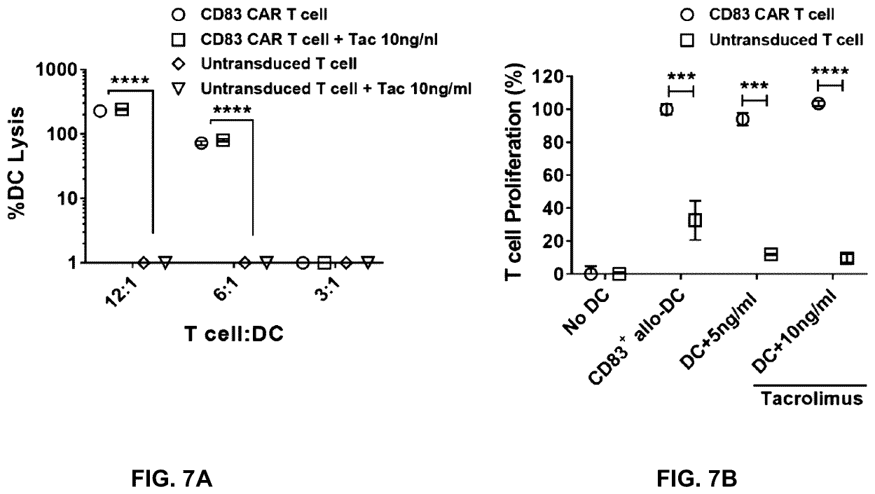

CD83 is an established marker of human dendritic cell maturation and is also expressed on activated human B cells (Szabolcs P. et al., Blood 87:4520-4530 (1996); Krzyzak L. et al., J Immunol 196:3581-3594 (2016)). Using a CD83 reporter mouse system, it was previously shown that activated murine T cells also express CD83 (Lechmann, M. et al., Proc Natl Acad Sci USA 105:11887-11892 (2008)). It is known that CD83 is expressed on human T cells after stimulation, and is detectable on circulating T cells from patients with acute GVHD (Ju X. et al., J Immunol 197:4613-4625 (2016)). However, the precise expression of CD83 on CD4+ Tregs versus CD4+ Tconv or CD8+ T cells is unclear. Experiments confirmed that human T cell expression of CD83 occurs with stimulation, including allogeneic dendritic cells or CD3/CD28 beads (FIGS. 3A,3B). Importantly, it was demonstrated that CD83 is differentially expressed on human CD4+ Tconv (CD127+, CD25+) compared to immune suppressive CD4+ Tregs (CD127−, CD25+) or cytolytic CD8+ T cells in response to DC-alloactivation (FIG. 3A). CD4+ Tconv expression of CD83 peaks at 4-8 hours of DC-allostimulation and declines to baseline levels by 48 hours, with minimal amounts observed on Tregs or CD8+ T cells (FIG. 3A). The expression of CD83 is more abundant with supraphysiologic CD3/CD28 bead stimulation, which also causes a late increase in CD83 expression on Tregs and CD8+ T cells by 48 hours of activation (FIG. 3B). Given that CD83 expression is shared among proinflammatory, mature DCs as well as alloreactive Tconv, whether the CD83 CAR T cell could deplete either target cells in culture was investigated. Human CD83 CAR or mock T cells were cultured with autologous peripheral blood mononuclear cells (PBMC) stimulated by allogeneic moDCs, and the amount of CD83+ target cells were evaluated at 4, 8, 24, and 48 hours of culture. We observed a similar spike in CD83 expression by CD3− and CD3+ target cells at 8 hours (FIG. 3C). However, CD83+ target cells were essentially eliminated at 48 hours of culture by the CD83 CAR T cells, and well below their baseline amounts from 8 hours post culture (FIG. 3C). Moreover, CD83− T cells were still present in all experimental groups (FIG. 3C), supporting that the T cells were not indiscriminately destroyed. Next, the expression of CD83 on the eGFP+ CAR T cells over 48 hours was evaluated. CD83 expression on the CAR T cells was modest, and an increase in the proportion of eGFP+ CAR T cells was still observed by 48 hours of culture (FIG. 3D), providing evidence that the CD83 CAR T cells do not overtly succumb to CD83-mediated fratricide. To parallel clinical practice, the functional capacity of the CD83 CAR T cells in the presence of clinically relevant doses of tacrolimus (5-10 ng/ml) was tested. Interestingly, the CD83 CAR T cells could still kill and proliferate in response to CD83+ target cells, despite exposure to tacrolimus (FIGS. 7A,7B).

### Human CD83-Targeted CAR T Cells Prevent Xenogeneic GVHD.

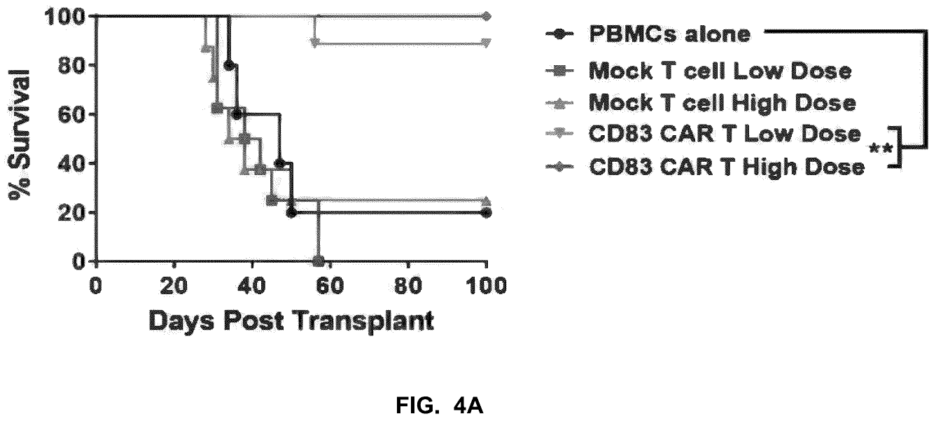

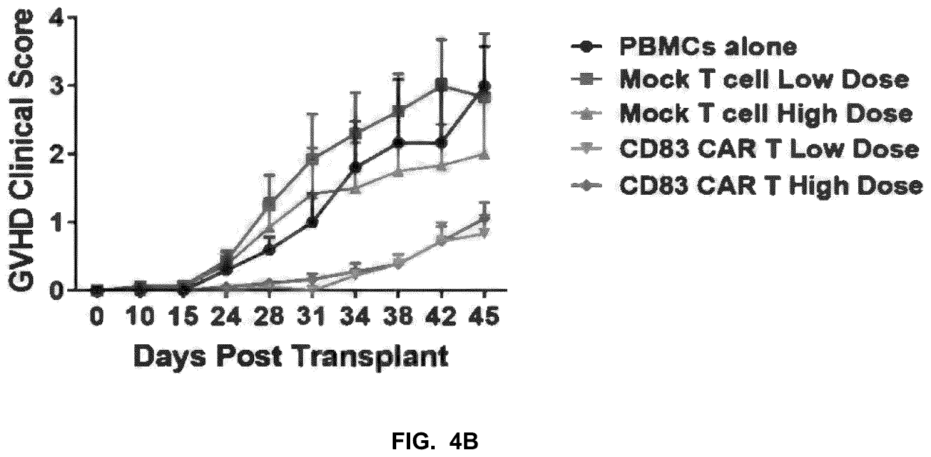

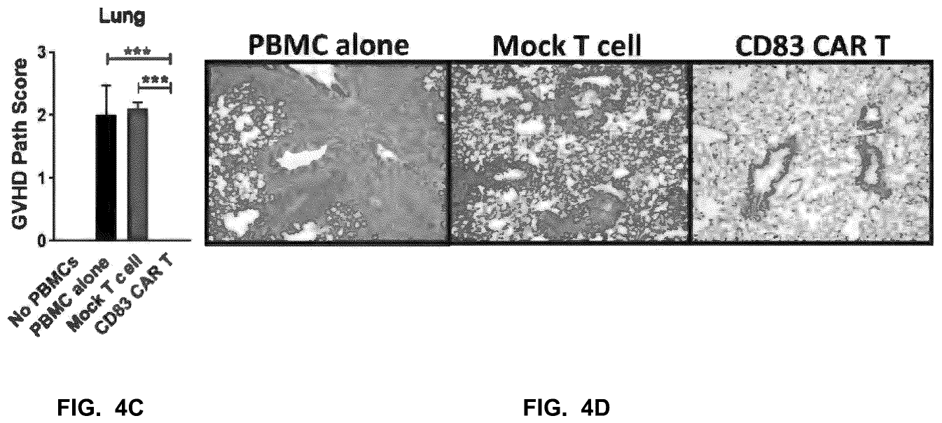

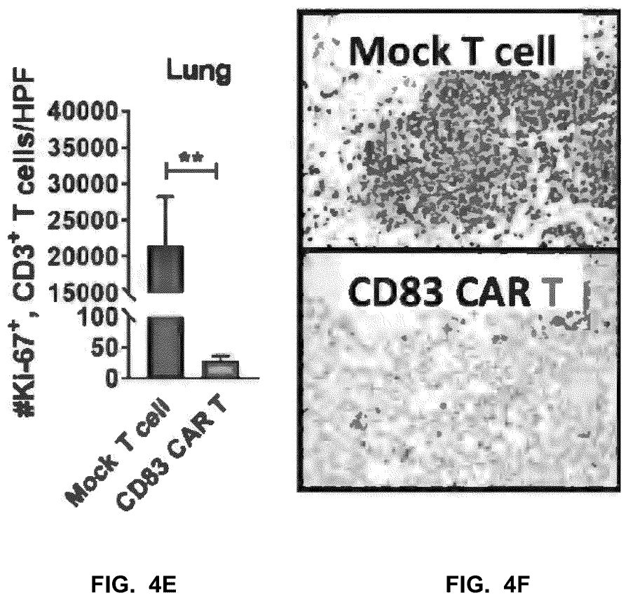

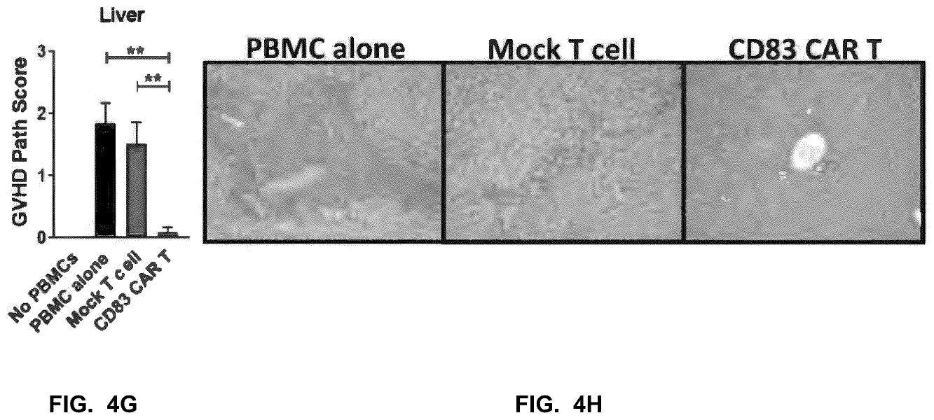

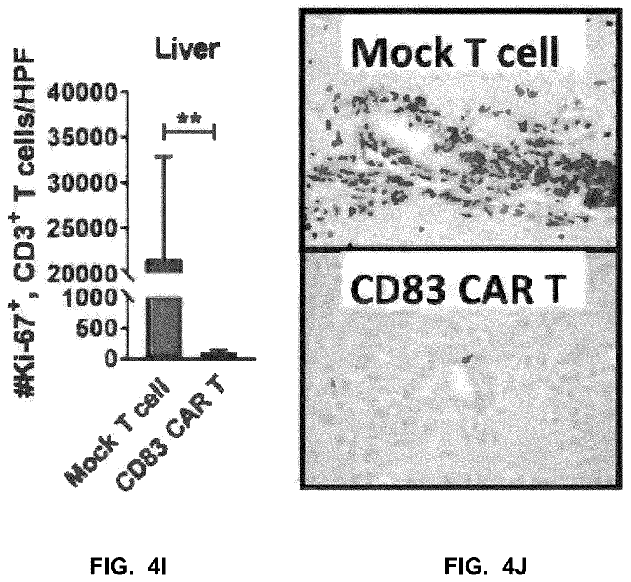

A xenogeneic GVHD model was used to evaluate the efficacy of human CD83 CAR T cells in vivo. An established NSG mouse model was used (Betts B. C. et al., Science translational medicine 9:eaai8269 (2017)), where recipients were inoculated with 25×10⁶ human PBMCs plus either 1-10×10⁶ autologous CD83 or mock transduced CAR T cells all on day 0. Transplanted mice were monitored daily for clinical signs of xenogeneic GVHD up to day +100. NSG mice infused with CD83 or mock transduced CAR T had no evidence of early GVHD or toxicity compared to PBMCs alone (FIGS. 4A,4B). However, CD83 CAR T cells significantly improved xenogeneic GVHD survival after transplant, compared to PBMCs alone or mock transduced CAR T cells (FIG. 4A). Additionally, xenogeneic GVHD clinical severity was reduced by CD83-targeted CAR T cells (FIG. 4B). Remarkably, mice in both dose cohorts of CD83-targeted CAR T cells demonstrated 3-month survival of 90% or better (FIG. 4A). In separate experiments, transplanted NSG mice received PBMCs alone or with mock transduced T cells (1×10⁶) or CD83-targeted CAR T cells (1×10⁶) and were humanely euthanized at day +21 to evaluate target organ GVHD severity. GVHD path scores were determined by a blinded expert pathologist (Betts B. C. et al., Science translational medicine 9:eaai8269 (2017); Betts B. C. et al., Proc Natl Acad Sci USA., 201712452 (2018); Betts B. C. et al., Front Immunol 9:2887 (2018)). CD83 CAR T cells eliminated xenogeneic GVHD target organ tissue damage by human T cells in the recipient lung (FIGS. 4C-4E) and liver (FIGS. 4G-J), compared to PBMCs alone or mock transduced T cells. Moreover, few human T cells directly infiltrated the murine target organs, and they were not proliferative based on Ki-67 staining (FIGS. 4E,4F,4I,4J).

### Human CD83-Targeted CAR T Cells Significantly Reduce CD83+ DCs In Vivo.

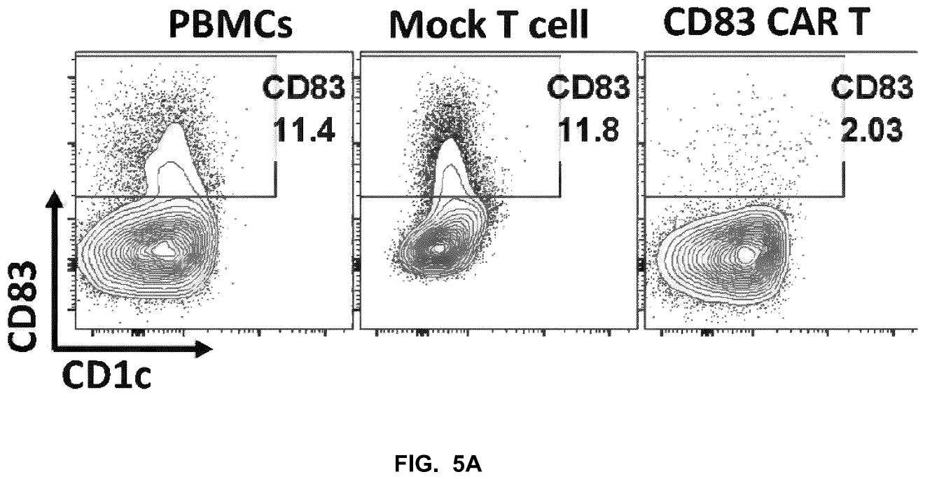

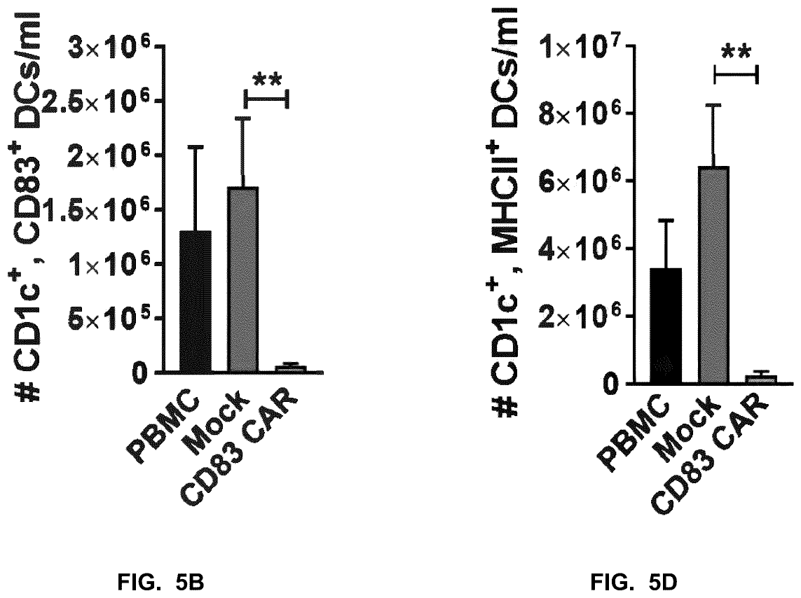

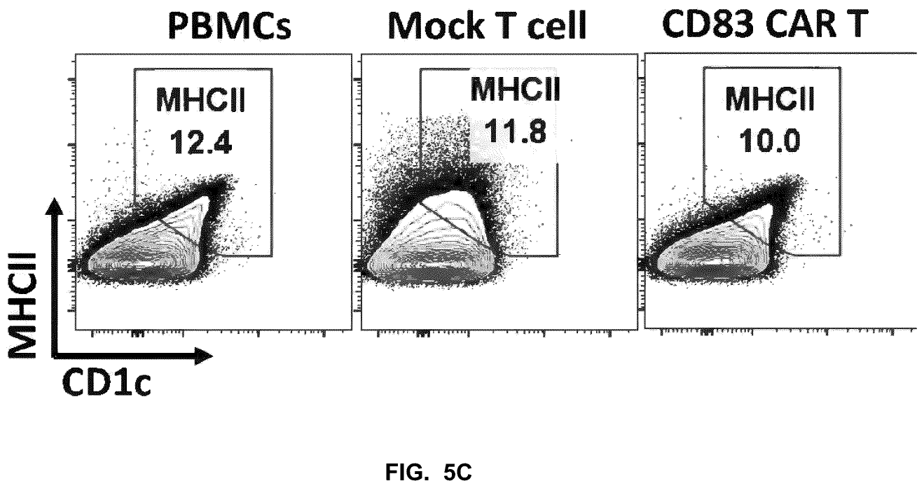

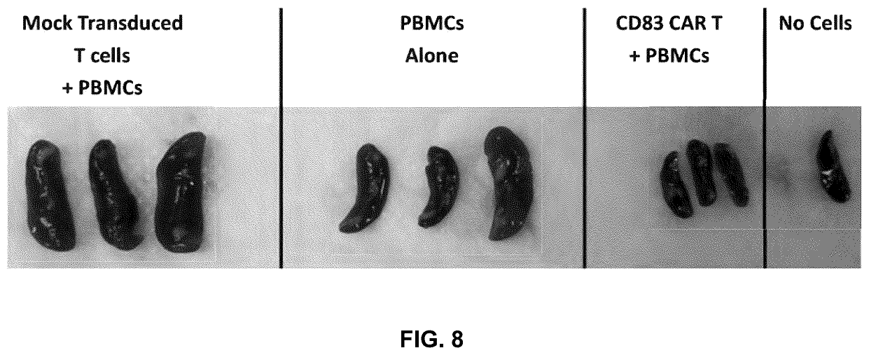

Mature, CD83+ dendritic cells are implicated in the sensitization of alloreactive donor T cells. As such, the effect of CD83 CAR T cells on the immune recovery of human CD1c+ DCs in transplanted mice was determined. NSG mice transplanted with human PBMCs plus CD83 CAR or mock transduced T cells were euthanized on day +21. Upon harvesting recipient spleens, it was determined that CD83-targeted CAR T cells reduced the expansion of donor cells in vivo as indicted by much smaller spleens in this treatment group (FIG. 8). CD83− targeted CAR T cells significantly reduced the amount of human CD1c+, CD83+ DCs in recipient mice (FIGS. 5A,5B). While the proportion of CD1c+ DCs expressing MHC class II was similar among experimental groups, mice transplanted with CD83 CAR T cells exhibited significantly fewer DCs altogether (FIGS. 5C,5D).

Human CD83-Targeted CAR T Cells Significantly Reduce CD4+, CD83+ T Cells, while Increasing the Treg:Activated Tconv Ratio In Vivo.

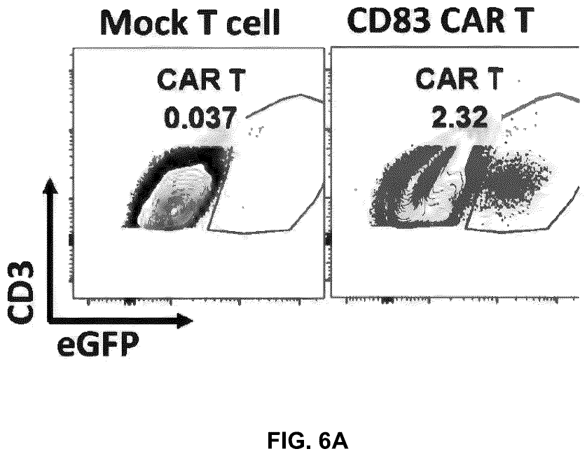

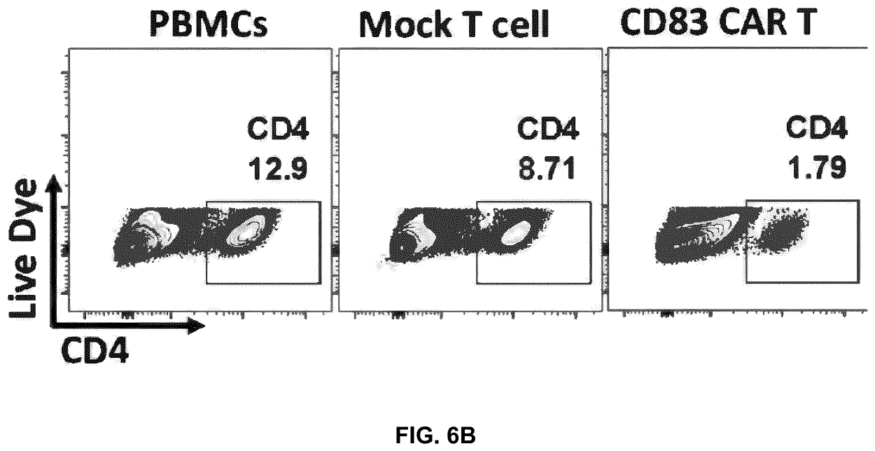

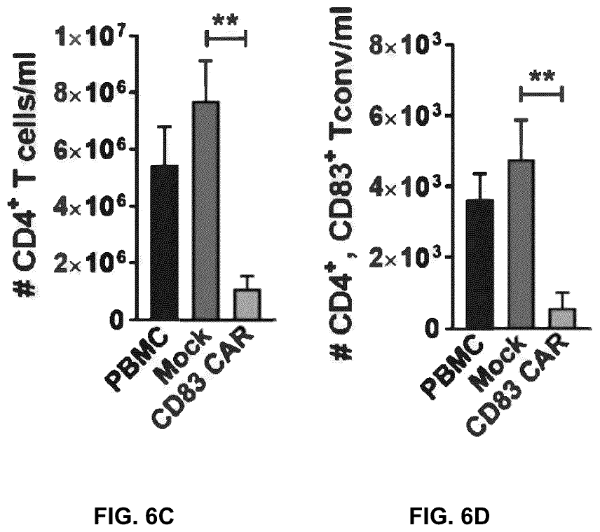

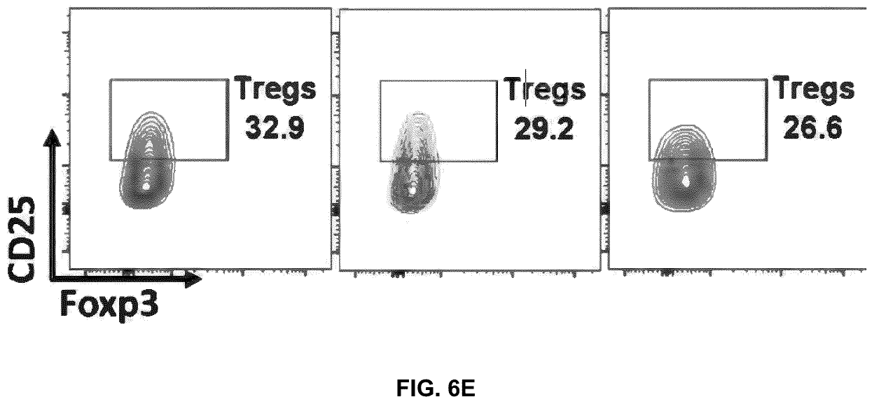

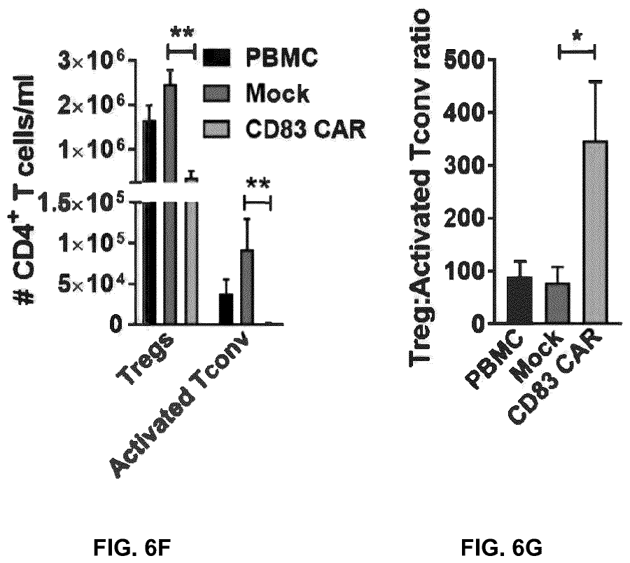

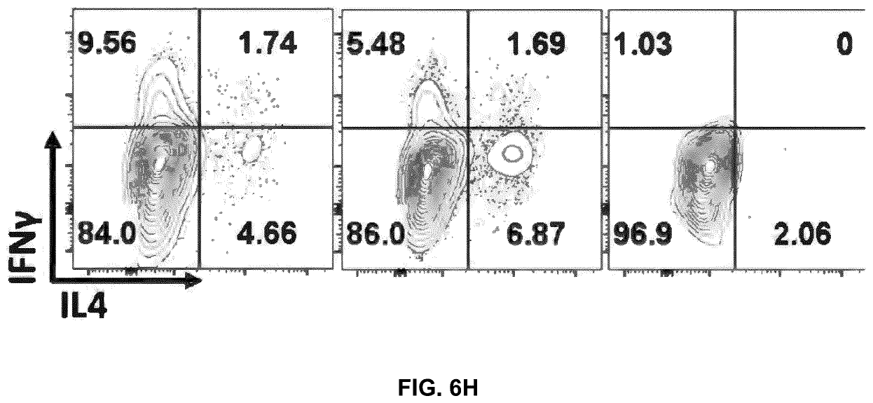

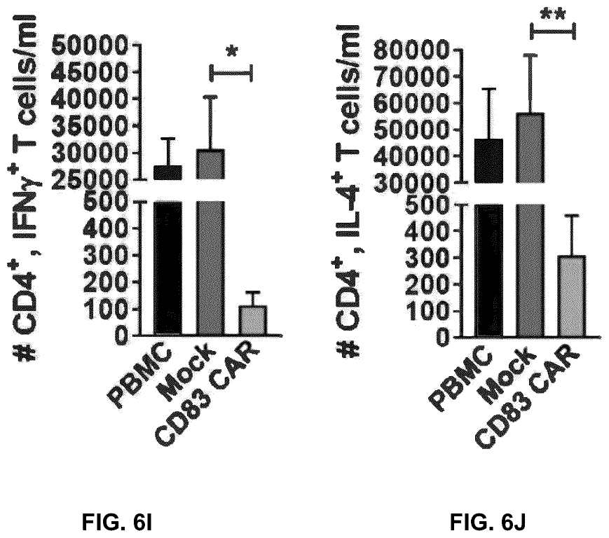

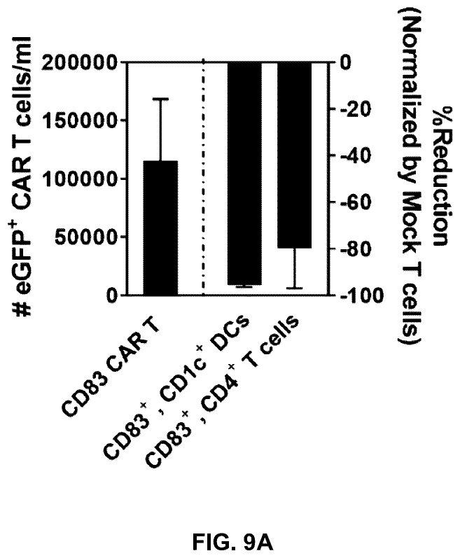

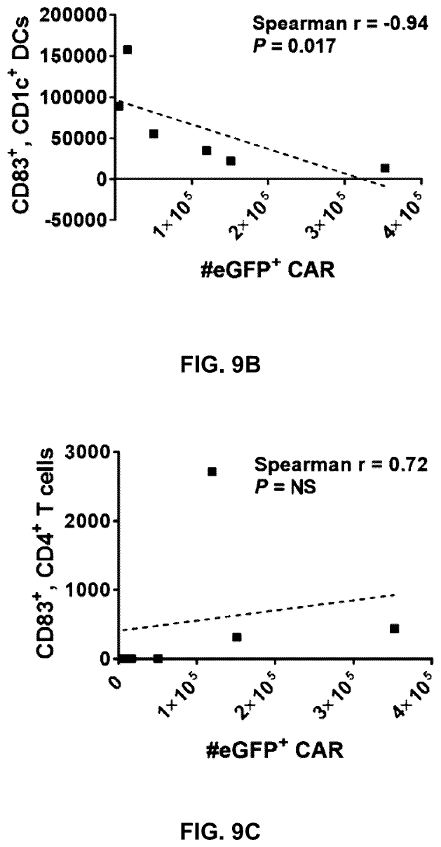

The eGFP tag was used to confirm that infused human CD83 CAR T cells were detectable in murine spleens at day +21 (FIG. 6A). At day +21, the total amount of human CD4+ T cells in the spleens of mice treated with CD83-targeted CAR T cells were significantly reduced (FIGS. 6B,6C). As significant amounts of CD83+CD4+ Tconv after DC-allostimulation were observed in vitro, experiments were conducted to confirm that CD83+ Tconv were increased at day +21 among mice treated with PBMCs alone or with mock transduced T cells (FIG. 6D). Moreover, the amount of CD83+ Tconv was significantly decreased in recipients of CD83 CAR T cells in vivo (FIG. 6D). Overall, the CD83 CAR T cells provided robust elimination of CD83+ target cells by day +21, compared to mock T cells (FIG. 9A). While higher numbers of circulating eGFP+ CAR T cells was linked to fewer CD83+ DCs at day +21, the reduction in CD83+ T cells was uniform across CAR T cell numbers in vivo (FIGS. 9B,9C).

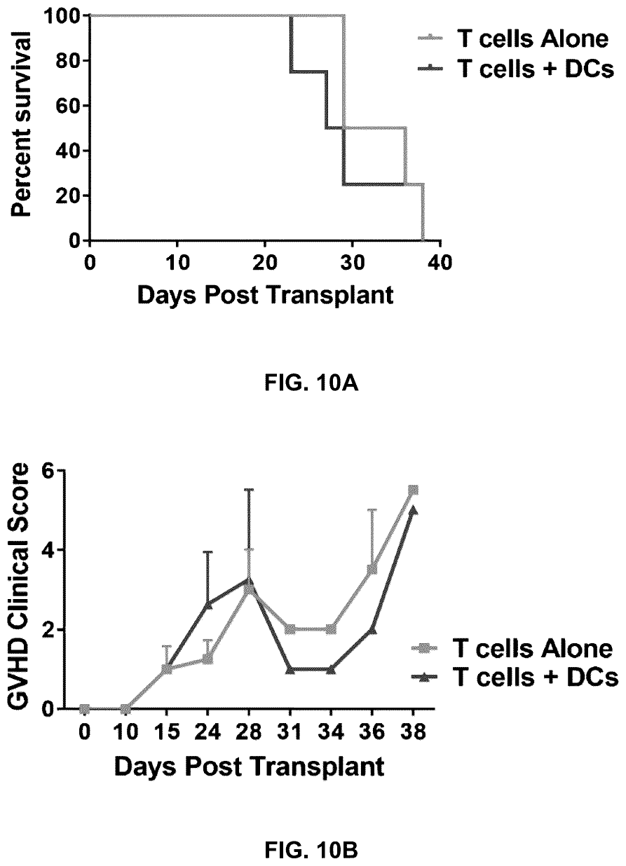

In separate experiments, NSG mice were transplanted with human T cells alone or T cells plus dendritic cells. While the lack of dendritic cells slightly delayed GVHD onset, the median GVHD survival was similar among both groups (FIGS. 10A,10B). This is consistent with work from others, showing purified human T cells are sufficient to induce xenogeneic GVHD (Li W. et al., JCI Insight 1 (2016)).

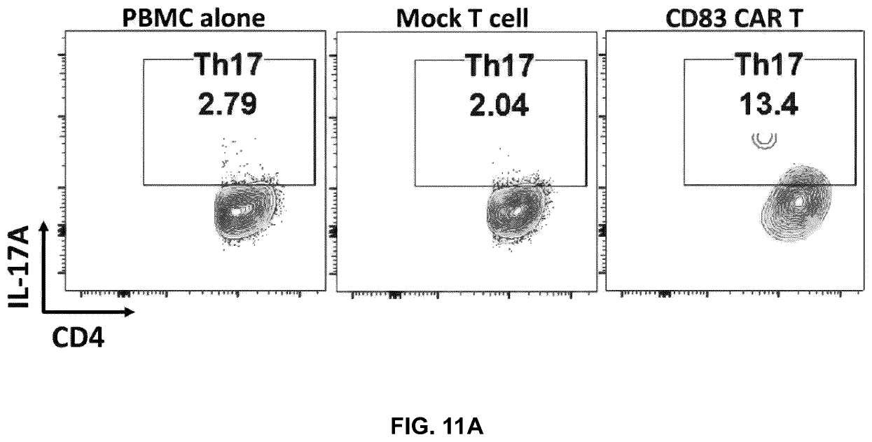

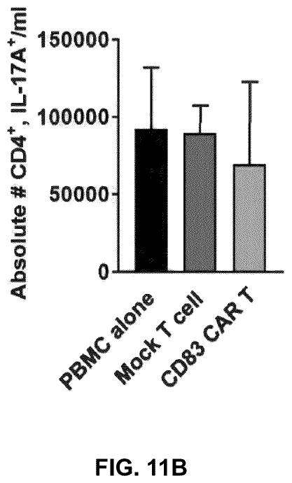

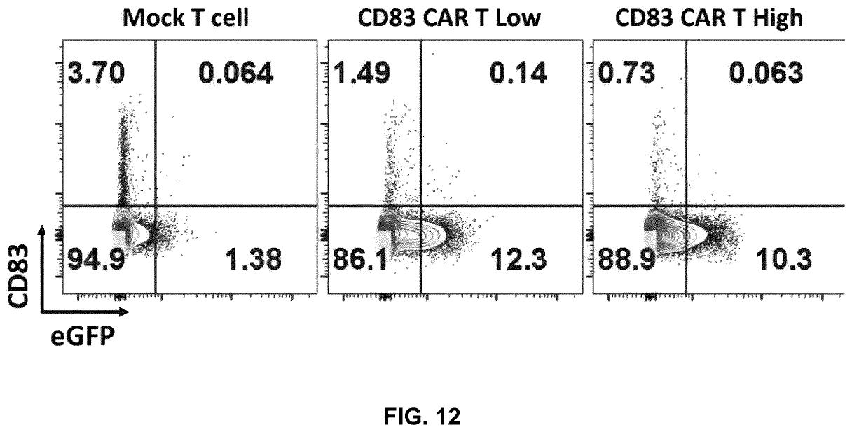

It was surmised that CD83-targeted CAR T cells protect recipients from GVHD primarily by eliminating alloreactive Tconv implicated in GVHD, while enhancing the ratio of Treg to alloreactive Tconv (FIGS. 6E-6G). The frequency of human Tregs in murine spleens was similar among all experimental groups at day +21 (FIG. 6E). Similar to the reduction in total CD4+ T cells, the absolute number of Tregs was significantly decreased in mice treated with CD83-targeted CAR T cells (FIG. 6F). However, the ratio of Treg (CD4+, CD127−, CD25+, Foxp3+) to activated Tconv (CD4+, CD127+, CD25+) (Betts B. C. et al., Science translational medicine 9:eaai8269 (2017)) was significantly increased in mice that receive CD83-targeted CAR T cells (FIG. 6G). Th1 cells contribute toward GVHD pathogenesis. Importantly, mice treated with CD83 CAR T cells exhibited a profound reduction in human CD4+, IFNγ+ Th1 cells (FIGS. 6H,6I). Additionally, the amount of spleen-resident, human Th2 cells (CD4+, IL-4+) were also significantly decreased in the mice injected with CD83 CAR T cells (FIGS. 6H,6J). Conversely, CD83-targeted CAR T cells did not suppress the amount of human Th17 cells (FIGS. 11A,11B) in recipient spleens, compared to PBMCs alone or mock transduced CAR T cells. Interestingly, eGFP+CD83 CAR T cells were also detected in the spleens of mice surviving to the day +100 endpoint in long-term experiments (FIG. 12). Over 3 months post-transplant, a dose-dependent reduction in circulating CD83+ target cells was observed among mice treated with a low (1×10⁶) or high (10×10⁶) dose of CD83 CAR T cells (FIG. 12).

### DISCUSSION

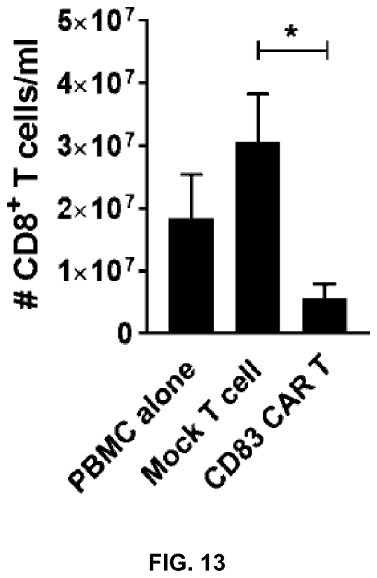

The use of CAR T cells as cellular immunotherapy to prevent GVHD is an innovative strategy, distinct from pharmacologic immune suppression or adoptive transfer of donor Tregs. Targeting cells that express CD83 efficiently depletes transplant recipients of inflammatory, mature DCs as well as alloreactive CD4+ Tcovnv. Donor CD8+ T cells can also mediate GVHD (Okiyama N. et al., J Invest Dermatol 134: 992-1000 (2014); Shindo T. et al., Blood 121:4617-4626 (2013)). Though few human CD8+ T cells express CD83, the CD83 CAR T cells significantly reduced the amount of donor CD8+ T cells as well (FIG. 13). Mechanistically, it was surmised the in vivo elimination of alloreactive T cells drives the efficacy of these CAR T cells, as dendritic cell-depletion did not reduce xenogeneic GVHD. The in vivo depletion of alloreactive T effectors by the CD83 CAR T cells also mediates a significant rise in the Treg:activated Tconv ratio, which is clinically relevant index in controlling GVHD (Koreth J. et al., N Engl J Med 365:2055-2066 (2011)).

The CD83 CAR T cells significantly reduce pathogenic, human Th1 and Th2 cells in vivo. Experiments using STAT4 and STAT6 knock out donor T cells have shown that Th1 and Th2 cells independently mediate lethal GVHD in mice (Nikolic, B. et al., J Clin Invest 105:1289-1298 (2000)). Additionally, the combination of Th1 and Th2 cells in vivo cooperatively worsen murine GVHD (Nikolic, B. et al., J Clin Invest 105:1289-1298 (2000)). In part, Th1 and Th2 cells cause tissue-specific damage to the intestine and lungs respectively (Yi T. et al., Blood 114:3101-3112 (2009)). Strategies to target donor Th1 responses currently exist, and are largely driven by p40 cytokine neutralization or inhibition of relevant downstream receptor signal transduction (Betts B. C. et al., Science translational medicine 9:eaai8269 (2017); Betts B. C. et al., Proc Natl Acad Sci USA., 201712452 (2018); Betts B. C. et al., Front Immunol 9:2887 (2018); Pidala J. et al., Haematologica 2017.171199 (2017); Yu Y. et al., Blood 118:5011-5020 (2011)). However, few approaches concurrently target pathogenic Th1 and Th2 cells. Thus, human CD83 CAR T cells represent a cell product to simultaneously suppress donor Th1/Th2 responses after allo-HCT. Human Th17 cells were largely unaffected by the CD83 CAR T cells, though the treated mice were clearly protected from GVHD. While donor Th17 cells have the potential to contribute toward GVHD (Iclozan C. et al., Biol Blood Marrow Transplant 16:170-178 (2010)), the lack of available Th1 cells likely mitigated the pathogenicity of the surviving Th17 cells (Yu Y. et al., Blood 118:5011-5020 (2011)).

The disclosed data support that human CD83 CAR T cells provide durable protection from activated Tconv and GVHD mortality. Though CD83 is not significantly expressed on human Tregs, mice treated with the human CD83 CAR T cells exhibited reduced amounts of Tregs. This may be due to limited availability of CD4+ T cell precursors for Treg differentiation or diminished IL-2 concentrations by the overall reduction in circulating donor T cells. In rodents, CD83 participates in Treg stability in vivo and mice bearing CD83-deficient Tregs are susceptible to autoimmune syndromes (Doebbeler M. et al., JCI Insight 3 (2018)). However, in the xenotransplantation experiments the ratio of human Treg to activated Tconv was significantly increased in mice treated with CD83 CAR T cells compared to controls. The increased ratio of Treg to Tconv is a clinically relevant immune indicator, and even correlates with response to Treg-directed GVHD therapy such as low-dose IL-2 (Koreth J. et al., N Engl J Med 365:2055-2066 (2011); Koreth J. et al., Blood 128:130-137 (2016)). Moreover, the human CD83 CAR T cells were well tolerated and eliminated immune-mediated organ damage in vivo. Thus, the role of CD83 may differ among murine and human Tregs.

CD83 is a unique immune regulatory molecule. In mice, soluble CD83 mediates immune suppressive effects by enhancing Treg responses through indoleamine 2,3-dioxygenase- and TGFβ-mechanisms (Bock F. et al., J Immunol 191:1965-1975 (2013)). The extracellular domain of human CD83 was also shown to impair alloreactive T cell proliferation in vitro (Lechmann M. et al., J Exp Med 194:1813-1821 (2001)). Conversely, direct neutralization of CD83 with monoclonal antibody, 3C12C, significantly reduces xenogeneic GVHD mediated by human T cells in vivo (Wilson J. et al., J Exp Med 206:387-398 (2009)). The CD83 antibody also preserved Treg and antiviral responses by donor, human CD8+ T cells (Seldon T. A. et al., Leukemia 30:692-700 (2016)). This suggests that while soluble CD83 may have immune suppressive properties, targeting the cell surface expression of CD83 can prevent GVHD while retaining key effector and Treg function. Distinct from monoclonal antibody, the CD83 CAR T cell elicits robust target cell killing alone; without the need for NK-cell mediated antibody-dependent cellular cytotoxicity (Seldon T. A. et al., Leukemia 30:692-700 (2016)). This is an advantage when rapid, efficient elimination of alloreactive T cells is needed to prevent GVHD. Indeed, the human CD83-targeted CAR T cells provided lasting GVHD prophylaxis and were detectable in mice up to day +100 even after a single infusion.

In conclusion, the CD83 CAR T cell represents the first human, programmed cytolytic effector cell designed to prevent GVHD. The translational potential of the CD83 CAR T cell was demonstrate tin GVHD prophylaxis, though it is expected it to have merit in preventing rejection after solid organ or vascularized composite allograft transplantation too. Furthermore, the CD83 CAR T cells retain their killing activity even when expose to calcineurin-inhibitors. The CD83 CAR T cell may overcome the barriers of HLA disparity in hematopoietic cell and solid organ donor selection, and greatly extend the application of curative transplantation procedures to patients in need. Importantly, the CD83 CAR T cell provides a platform to eliminate alloreactive T cells without the need for broadly suppressive, nonselective calcineurin-inhibitors or glucocorticoids.

### Example 2: Off-the-Shelf CD83 CAR Tregs Offer Significantly Enhanced Suppression of Alloreactive T Cells

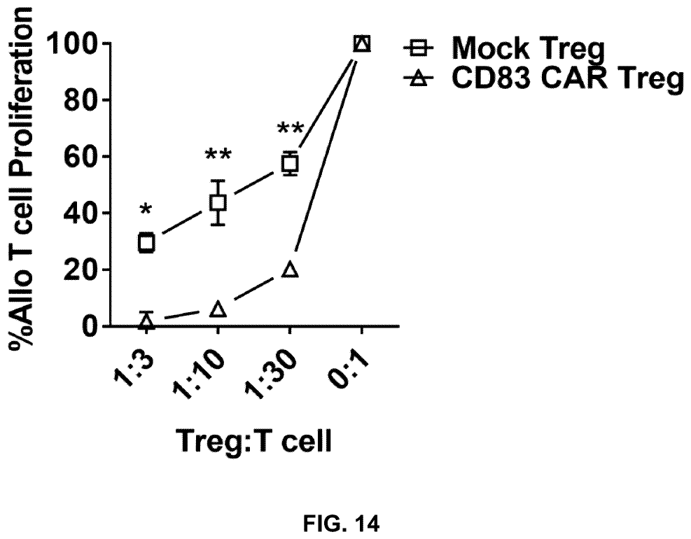

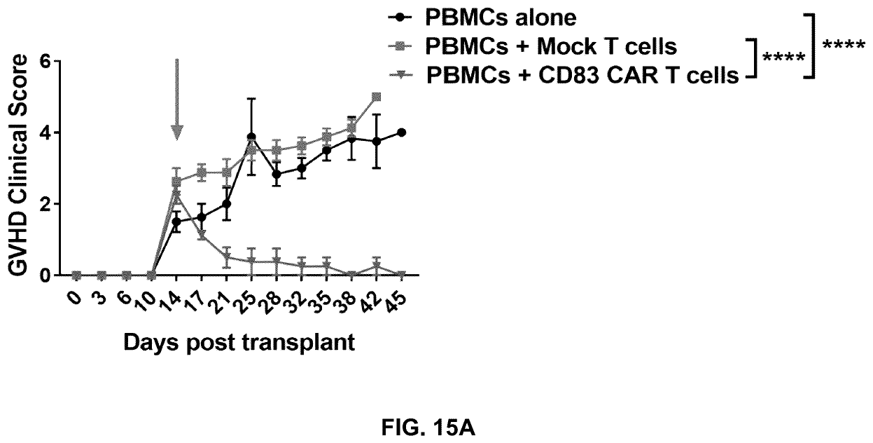

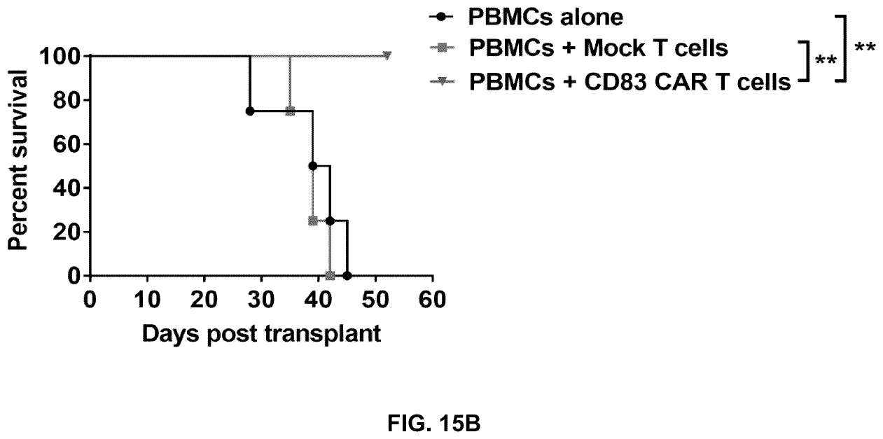

Human, regulatory T cells (Treg) expression a CD83 CAR or mock transduced Tregs were cultured with allogeneic mixed leukocyte reactions (T cell to DC ratio 30:1). The Tregs, T cells, and dendritic cells were entirely HLA-mismatched from each other. Graph in FIG. 14 shows alloreactive T cell proliferation as measured on day +5 of the culture. One representative experiment of 2 is shown, Sidak's test. *P<0.05, **P=0.001-0.1.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of suppressing alloreactive donor cells in a subject receiving transplant donor cells, the method comprising administering to the subject an effective amount of a first immune effector cell genetically modified to express a first chimeric antigen receptor (CAR) polypeptide, comprising a CD83 antigen binding domain, a transmembrane domain, an intracellular signaling domain, and a co-stimulatory signaling region, thereby suppressing alloreactive donor cells in the subject.

2. The method of claim 1, wherein the first immune effector cell is selected from the group consisting of a T cell, a Natural Killer (NK) cell, a B cell, an innate lymphoid cell (ILC), a cytokine induced killer (CIK) cell, a cytotoxic T lymphocyte (CTL), a lymphokine activated killer (LAK) cell, and a regulatory T (Treg) cell.

3. The method of claim 1, wherein the donor cells are bone marrow cells comprising alloreactive T-cells, dendritic cells, or a combination thereof.

4. The method of claim 1, wherein the transplant donor cells comprise a second immune effector cell genetically modified to express a second CAR polypeptide.

5. The method of claim 4, wherein the second immune effector cell is selected from the group consisting of a T cell, a Natural Killer (NK) cell, a B cell, an innate lymphoid cell (ILC), a cytokine induced killer (CIK) cell, a cytotoxic T lymphocyte (CTL), a lymphokine activated killer (LAK) cell, and a regulatory T (Treg) cell.

6. The method of claim 4, wherein the first CAR polypeptide comprises in an ectodomain a targeting agent that binds an antigen selected from the group consisting of CD19, CD22, CD123, CD38, CLL-1, TEM8/ANTXR1, CD56, NKG2D, B7H6, CD4, Gp120, Erb-B, SLAMF7, CD7, EGFR, TAG-72, MMG49, Integrin β7, FLT3, MESO, CD70, FOLR1, CD33, CD171, CD20, GD2, HER2, EGFRvIII, CSPG4, DNAX, BCMA, LeY, CD30, GPC3, CS1, CD138, Nectin4/FAP, CEA, EpCAM, PSCA, MUC1, CD80, CD86, CTLA-4, PD-1, CD10, EGFR806, IL 12, TLR-9, CD83, CLEC12A, CD99, IL13Rα2, SSTR2, and GPC3.

7. The method of claim 1, wherein the transplant donor cells are not HLA matched to the subject.

8. The method of claim 7, wherein the transplant donor cells have less than 3, 4, 5, or 6 HLA-matched markers as the subject.

9. The method of claim 1, wherein the subject has not received an immunosuppressant.

10. The method of claim 1, wherein the CD83 antigen binding domain is a single-chain variable fragment (scFv) of an antibody that specifically binds CD83.

11. The method of claim 9, wherein the anti-CD83 scFv comprises a variable heavy (V_{H}) domain having CDR1, CDR2 and CDR3 sequences and a variable light (V_{L}) domain having CDR1, CDR2 and CDR3 sequences, wherein the CDR1 sequence of the V_{H} domain comprises the amino acid sequence SEQ ID NO:1, SEQ ID NO:7, or SEQ ID NO:13; the CDR2 sequence of the V_{H} domain comprises the amino acid sequence SEQ ID NO:2, SEQ ID NO:8, or SEQ ID NO:14; the CDR3 sequence of the V_{H} domain comprises the amino acid sequence SEQ ID NO:3, SEQ ID NO:9, or SEQ ID NO:15; the CDR1 sequence of the V_{L} comprises the amino acid sequence SEQ ID NO:4, SEQ ID NO:10, or SEQ ID NO:16; the CDR2 sequence of the V_{L} domain comprises the amino acid sequence SEQ ID NO:5, SEQ ID NO:11, or SEQ ID NO:17; and the CDR3 sequence of the V_{L} domain comprises the amino acid sequence SEQ ID NO:6, SEQ ID NO:12, or SEQ ID NO:18.

12. The method of claim 10, wherein the anti-CD83 scFv V_{H} domain comprises the amino acid sequence SEQ ID NO:19, SEQ ID NO:48, SEQ ID NO:49, SEQ ID NO:50, SEQ ID NO:51, SEQ ID NO:52, or SEQ ID NO:53, and wherein the anti-CD83 scFv V_{L} domain comprises the amino acid sequence SEQ ID NO:20, SEQ ID NO:54, or SEQ ID NO:55.

13. The method of claim 11, wherein the anti-CD83 scFv comprises the amino acid sequence SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, or SEQ ID NO:71.

14. The method of claim 1, wherein the costimulatory signaling region comprises the cytoplasmic domain of a costimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.

15. The method of claim 1, further comprising administering to the subject a checkpoint inhibitor.

16. The method of claim 14, wherein the checkpoint inhibitor comprises an anti-PD-1 antibody, anti-PD-L1 antibody, anti-CTLA-4 antibody, or a combination thereof.

17. A method for treating a subject, comprising co-administering to the subject a therapeutic amount of a first non-autologous, non-HLA-matched immune effector cell genetically modified to express a first chimeric antigen receptor (CAR) polypeptide and a second non-autologous, non-HLA-matched immune effector cell genetically modified to express a second CAR polypeptide comprising a CD83 antigen binding domain.

18. The method of claim 17, wherein the second immune effector cell is selected from the group consisting of a T cell, a Natural Killer (NK) cell, a B cell, an innate lymphoid cell (ILC), a cytokine induced killer (CIK) cell, a cytotoxic T lymphocyte (CTL), a lymphokine activated killer (LAK) cell, and a regulatory T (Treg) cell.

19. The method of claim 17, wherein the first CAR polypeptide comprises in an ectodomain a targeting agent that binds an antigen selected from the group consisting of CD19, CD22, CD123, CD38, CLL-1, TEM8/ANTXR1, CD56, NKG2D, B7H6, CD4, Gp120, Erb-B, SLAMF7, CD7, EGFR, TAG-72, MMG49, Integrin β7, FLT3, MESO, CD70, FOLR1, CD33, CD171, CD20, GD2, HER2, EGFRvIII, CSPG4, DNAX, BCMA, LeY, CD30, GPC3, CS1, CD138, Nectin4/FAP, CEA, EpCAM, PSCA, MUC1, CD80, CD86, CTLA-4, PD-1, CD10, EGFR806, IL 12, TLR-9, CD83, CLEC12A, CD99, IL13Rα2, SSTR2, and GPC3.

20. The method of claim 17, wherein the subject has not received an immunosuppressant.
